SEQUENCE LISTING (49 sequences)

SEQ ID NO: 1 - protein, 4 aa, Artificial sequence
  Synthetic peptide
APAR

SEQ ID NO: 2 - protein, 4 aa, Artificial Sequence
  Synthetic peptide
RAPA

SEQ ID NO: 3 - protein, 6 aa, Artificial sequence
  Synthetic peptide
HWRGWA

SEQ ID NO: 4 - protein, 4 aa, Artificial sequence
  Synthetic peptide
WGRR

SEQ ID NO: 5 - protein, 4 aa, Artificial sequence
  Synthetic peptide
RRGW

SEQ ID NO: 6 - protein, 8 aa, Artificial sequence
  Synthetic peptide
NKFRGKYK

SEQ ID NO: 7 - protein, 8 aa, Artificial sequence
  Synthetic peptide
NRFRGKYK

SEQ ID NO: 8 - protein, 7 aa, Artificial sequence
  Synthetic peptide
NARKFYK

SEQ ID NO: 9 - protein, 8 aa, Artificial sequence
  Synthetic peptide
NARKFYKG

SEQ ID NO: 10 - protein, 6 aa, Artificial sequence
  Synthetic peptide
HWRGWV

SEQ ID NO: 11 - protein, 7 aa, Artificial sequence
  Synthetic peptide
KHFRNKD

SEQ ID NO: 12 - protein, 7 aa, Artificial sequence
  Synthetic peptide
RHRFNKD

SEQ ID NO: 13 - protein, 4 aa, Artificial sequence
  Synthetic peptide
RTYK

SEQ ID NO: 14 - protein, 4 aa, Artificial sequence
  Synthetic peptide
ELVW

SEQ ID NO: 15 - protein, 9 aa, Artificial sequence
  Synthetic peptide
AWHLGELVW

SEQ ID NO: 16 - protein, 13 aa, Artificial sequence
  Synthetic peptide
  DISULFID (2)..(12): May or may not include a disulfide bond
DCAWHLGELVWCT

SEQ ID NO: 17 - protein, 15 aa, Artificial sequence
  Synthetic peptide
  DISULFID (1)..(15): May or may not form a disulfide bond.
  DISULFID (3)..(13): May or may not form a disulfide bond.
CDCAWHLGELVWCTC

SEQ ID NO: 18 - protein, 4 aa, Artificial sequence
  Synthetic peptide
TFLL

SEQ ID NO: 19 - protein, 6 aa, Artificial sequence
  Synthetic peptide
TFLLKY

SEQ ID NO: 20 - protein, 4 aa, Artificial sequence
  Synthetic peptide
  VARIANT (4)..(4): X can be any amino acid
LKYX

SEQ ID NO: 21 - protein, 4 aa, Artificial sequence
  Synthetic peptide
LKYN

SEQ ID NO: 22 - protein, 4 aa, Artificial sequence
  Synthetic peptide
  VARIANT (4)..(4): X can be any alpha amino acid
GTIX

SEQ ID NO: 23 - protein, 6 aa, Artificial sequence
  Synthetic peptide
  VARIANT (4)..(4): X can be any alpha amino acid
GTIXDA

SEQ ID NO: 24 - protein, 5 aa, Artificial sequence
  Synthetic peptide
  VARIANT (2)..(2): X can be any amino acid
  VARIANT (5)..(5): X can be any alpha amino acid
GXITX

SEQ ID NO: 25 - protein, 6 aa, Artificial sequence
  Synthetic peptide
GTITDA

SEQ ID NO: 26 - protein, 4 aa, Artificial sequence
  Synthetic peptide
AVAD

SEQ ID NO: 27 - protein, 23 aa, Artificial sequence
  Synthetic peptide
IEEQAKTFLDKFNHEAEDLFYQS

SEQ ID NO: 28 - protein, 36 aa, Artificial sequence
  Synthetic peptide
DISGINASVVNIQKEIDRLNEVAKNLNESLIDLQEL

SEQ ID NO: 29 - protein, 7 aa, Artificial sequence
  Synthetic peptide
TFLLCYN

SEQ ID NO: 30 - protein, 8 aa, Artificial sequence
  Synthetic peptide
  MOD_RES (8)..(8): Aib
GTITCAVX

SEQ ID NO: 31 - protein, 5 aa, Artificial sequence
  Synthetic peptide
GTITC

SEQ ID NO: 32 - protein, 7 aa, Artificial sequence
  Synthetic peptide
  DISULFID (1)..(7)
  SITE (2)..(2): D-serine
CSEGTIC

SEQ ID NO: 33 - protein, 17 aa, Artificial sequence
  Synthetic peptides
  DISULFID (7)..(13)
  SITE (8)..(8): D-serine
  MOD_RES (17)..(17): Aib
TFLLKYCSEGTICDAVX

SEQ ID NO: 34 - protein, 14 aa, Artificial sequence
  Synthetic peptide
  DISULFID (1)..(14)
  SITE (6)..(6): D-serine
CLKYNSEGTITDAC

SEQ ID NO: 35 - protein, 17 aa, Artificial sequence
  Synthetic peptide
  DISULFID (3)..(16)
  SITE (8)..(8): D-serine
  MOD_RES (17)..(17): Aib
TFCLKYNSEGTITDACX

SEQ ID NO: 36 - protein, 20 aa, Artificial sequence
  Synthetic peptide
  VARIANT (8)..(8): N can be replaced by K
  VARIANT (20)..(20): S can be replaced by L
FKLPLGINITNFRAILTAFS

SEQ ID NO: 37 - protein, 20 aa, Artificial sequence
  Synthetic peptide
  VARIANT (3)..(3): T can be replaced by K
PTTFMLKYDENGTITDAVDC

SEQ ID NO: 38 - protein, 20 aa, Artificial sequence
  Synthetic peptide
  VARIANT (7)..(7): F can be replaced by S
VLYNSTFFSTFKCYGVSATK

SEQ ID NO: 39 - protein, 20 aa, Artificial sequence
  Synthetic peptide
  VARIANT (10)..(10): N can be replaced by K
PALNCYWPLNDYGFYTTSGI

SEQ ID NO: 40 - protein, 20 aa, Artificial sequence
  Synthetic peptide
  VARIANT (6)..(6): F can be replaced by I
RDVSDFTDSVRDPKTSEILD

SEQ ID NO: 41 - protein, 20 aa, Artificial sequence
  Synthetic peptide
  VARIANT (10)..(10): S can be replaced by P
YQDVNCTDVSTAIHADQLTP

SEQ ID NO: 42 - protein, 19 aa, Artificial sequence
  Synthetic peptide
  VARIANT (12)..(12): S can be replaced by L
SNNTIAIPTNFSISTTEVM

SEQ ID NO: 43 - protein, 20 aa, Artificial sequence
  Synthetic peptide
  VARIANT (7)..(7): T can be replaced by A
  VARIANT (18)..(18): A can be replaced by V
QYGSFCTQLNRALSGIAAEQ

SEQ ID NO: 44 - protein, 20 aa, Artificial sequence
  Synthetic peptide
  VARIANT (5)..(5): T can be replaced by A
GIGVTQNVLYENQKQIANQF

SEQ ID NO: 45 - protein, 20 aa, Artificial sequence
  Synthetic peptide
  VARIANT (3)..(3): K can be replaced by E
IQKEIDRLNEVAKNLNESLI

SEQ ID NO: 46 - protein, 11 aa, Artificial sequence
  Synthetic peptide
  SITE (1)..(1): D-proline
  SITE (2)..(2): L-proline
PPAWHLGELVW

SEQ ID NO: 47 - protein, 15 aa, Artificial sequence
  Synthetic peptide
  SITE (1)..(1): D-proline
  SITE (2)..(2): L-proline
PPDCAWHLGELVWCT

SEQ ID NO: 48 - protein, 18 aa, Artificial sequence
  Synthetic peptide
  DISULFID (7)..(13)
  SITE (8)..(8): D-serine
  MOD_RES (18)..(18): Aib
TFLLKYCSEGTICDAVAX

SEQ ID NO: 49 - protein, 9 aa, Artificial sequence
  Synthetic peptide
  MOD_RES (9)..(9): Aib
GTITCAVAX

(19) United States
(12) Patent Application Publication
(10) Pub. No.: US 2023/0330240 A1
(43) Pub. Date: Oct. 19, 2023

(54) TECHNOLOGIES FOR PREVENTING OR TREATING INFECTIONS

(71) Applicant: Biohaven Therapeutics Ltd., New Haven, CT (US)

(72) Inventors: Alexander Sergei BAYDEN, Hamden, CT (US); Tetyana BERBASOVA, Hamden, CT (US); Lawrence Emerson FISHER, Danbury, CT (US); Wieslaw KAZMIERSKI, Raleigh, NC (US); Luca RASTELLI, Madison, CT (US); Tomi K. SAWYER, New Haven, CT (US); David Adam SPIEGEL, Hamden, CT (US); Wayne Charles WIDDISON, New Haven, CT (US)

(21) Appl. No.: 17/912,563

(22) PCT Filed: Mar. 25, 2021
(86) PCT No.: PCT/US2021/024186

§ 371 Date: Sep. 19, 2022

Related U.S. Application Data
(60) Provisional application No. 62/994,779, filed on Mar. 25, 2020.
(60) Provisional application No. 63/001,455, filed on Mar. 29, 2020.
(60) Provisional application No. 63/055,860, filed on Jul. 23, 2020.

(51) Int. Cl. A61K 47/68; A61P 31/14; A61K 47/18; A61K 47/08

(52) U.S. Cl. CPC A61K 47/6801; A61K 47/08; A61K 47/18; A61P 31/14

(57) ABSTRACT

Among other things, the present disclosure provides agents that can bind to viruses such as SARS-CoV-2 and/or cells infected thereby. In some embodiments, the present disclosure provides methods for preventing and/or treating conditions, disorders or diseases associated with SARS-CoV-2 infection. In some embodiments, the present disclosure provides methods for preventing and/ or treating COVID-19.

### CROSS REFERENCE TO RELATED APPLICATIONS

This application is a national stage application of PCT/US2021/024186, filed Mar. 25, 2021, which claims priority to U.S. Provisional Application No. 62/994,779, filed Mar. 25, 2020 and to U.S. Provisional Application No. 63/001,455, filed Mar. 29, 2020 and to U.S. Provisional Application No. 63/055,860, filed Jul. 23, 2020, each of which is hereby incorporated by reference in its entirety.

### SUMMARY

Among other things, the present disclosure provides technologies (e.g., agents, compositions, methods, etc.) for preventing and/or treating conditions, disorders or diseases associated with SARS-CoV-2. In some embodiments, a condition, disorder or disease is Coronavirus disease 2019, COVID-19. In some embodiments, provided technologies disrupts or reduces interaction between a cell and a SARS-CoV-2 virus. In some embodiments, provided technologies disrupts or reduces interactions between a spike protein (S protein) of SARS-CoV-2 and a receptor, e.g., ACE2, or a cell. In some embodiments, provided technologies disrupting or reducing an infection of a SARS-CoV-2 virus of a cell. In some embodiments, provided technologies inhibit, kill or remove SARS-CoV-2 viruses. In some embodiments, provided technologies inhibit, kill or remove cells infected by SARS-CoV-2 viruses. In some embodiments, provided technologies inhibit, kill or remove a cell expressing a spike protein of SARS-CoV-2 or a fragment thereof. In some embodiments, a cell is a mammalian cell that can be infected by SARS-CoV-2. In some embodiments, a cell is a human cell.

In some embodiments, the present disclosure provides agents that comprise a moiety, e.g., a target binding moiety described herein, that targets SARS-CoV-2. In some embodiments, a moiety binds to a spike protein of a SARS-CoV-2 virus. In some embodiments, provided moieties are or comprise -(Xaa)y- as described herein. In some embodiments, a provided agent has the structure of formula T-I,

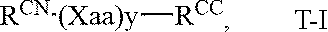

or a salt form thereof, wherein:
R^{CN} and R^{CC} is independently R^{C};
each Xaa is independently a residue of an amino acid or an amino acid analog;
y is 5-50;
each R^{C} is independently —L^{a}—R′;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂— —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a provided moiety, e.g., a target binding moiety, is a moiety of an agent having the structure of T-I or a salt thereof (e.g., as appreciated by those skilled in the art, by removing one or more —H to form a monovalent, bivalent or polyvalent moiety). In some embodiments, a moiety has the structure of —(R^{CN}—(Xaa)y—R^{CC}).

In some embodiments, the present disclosure provides an agent comprising:
an antibody binding moiety,
a target binding moiety, and
optionally a linker moiety linking an antibody binding moiety and a target binding moiety.

In some embodiments, the present disclosure provides an agent has the structure of formula I:

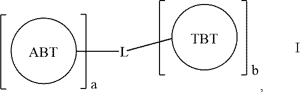

or a pharmaceutically acceptable salt thereof, wherein:
each of a and b is independently 1-200;
each ABT is independently an antibody binding moiety;
L is a linker moiety that connects ABT with TBT; and
each TBT is independently a target binding moiety.

In some embodiments, the present disclosure provides an agent has the structure of:

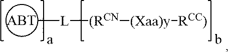

or a pharmaceutically acceptable salt thereof, wherein:
each of a and b is independently 1-200;
each ABT is independently an antibody binding moiety;
L is a linker moiety;
each Xaa is independently a residue of an amino acid or an amino acid analog;
y is 5-50;
each R^{c} is independently —L^{a}—R′;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides an agent comprising:
an antibody moiety,
a target binding moiety, and
optionally a linker moiety linking an antibody moiety and a target binding moiety.

In some embodiments, the present disclosure provides an agent has the structure of formula I′ :

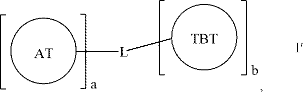

or a pharmaceutically acceptable salt thereof, wherein:
each of a and b is independently 1-200;
each AT is independently an antibody moiety;
L is a linker moiety that connects ABT with TBT; and
each TBT is independently a target binding moiety.

In some embodiments, the present disclosure provides an agent has the structure of:

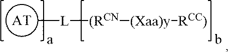

or a pharmaceutically acceptable salt thereof, wherein:
each of a and b is independently 1-200;
each AT is independently an antibody moiety;
L is a linker moiety;
each Xaa is independently a residue of an amino acid or an amino acid analog;
y is 5-50;
each R^{c} is independently —L^{a}—R′;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a target binding moiety has a structure that comprises -(Xaa)y as described herein. In some embodiments, -(Xaa)y- is or comprises:
-(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12-,
wherein:
y0 is 0-20;
each Xaa^{T0} is independently a residue of an amino acid or an amino acid analog;
y1 is 0-2;
each Xaa^{T1} is independently a residue of an amino acid or an amino acid analog;
Xaa^{T2} is a residue of an amino acid or an amino acid analog whose side chain comprises 3 or more non-hydrogen atoms;
y3 is 0-10;
each of Xaa^{T3} is independently a residue of an amino acid or an amino acid analog;
each of Xaa^{T4} and Xaa^{T9} is independently a residue of an amino acid or an amino acid analog, wherein Xaa^{T4} is optionally connected to Xaa^{T9} through a linker;
y5 is 0-10;
each Xaa^{T5} is independently a residue of an amino acid or an amino acid analog;
y6 is 0-2;
each Xaa^{T6} is independently a residue of an amino acid or an amino acid analog;
y7 is 0-1;
Xaa^{T7} is a negatively-charged residue of an amino acid or an amino acid analog;
y8 is 0-10;
each Xaa^{T8} is independently a residue of an amino acid or an amino acid analog;
y10 is 0-10;
each of Xaa^{T10} is independently a residue of an amino acid or an amino acid analog;
y11 is 1-5;
each Xaa^{T11} is independently a residue of an amino acid or an amino acid analog;
y12 is 0-20; and
each Xaa^{T12} is independently a residue of an amino acid or an amino acid analog.

Useful residues for each of Xaa^{T0}, Xaa^{T1}, Xaa^{T2}, Xaa^{T3}, Xaa^{T4}, Xaa^{T5}, Xaa^{T6}, Xaa^{T7}, Xaa^{T8}, Xaa^{T9}, Xaa^{T10}, Xaa^{T11}, and Xaa^{T12} are described as described herein, both individually and in combination.

Various antibody binding moieties can be utilized in accordance with the present disclosure. Certain antibody binding moieties are described herein as examples. Those skilled in the art also appreciate that many antibodies, e.g., IVIG, can be utilized for antibody moieties in provided technologies in accordance with the present disclosure. Among other things, IVIG is readily available and is approved for treating several diseases. In some embodiments, antibody moieties are a subject’s own IgG or fragments thereof. In some embodiments, antibody moieties are a pooled IgG preparation, e.g., certain IVIG preparations, or fragments thereof.

Without the intention to be bound by any theory, in some embodiments, provided technologies can recruit antibodies to an entity expressing a SARS-CoV-2 spike protein (unless otherwise indicated, including mutants thereof (e.g., those in viruses and/or infected cells)) or a fragment thereof (e.g., a SARS-CoV-2 virus, a cell infected by a SARS-CoV-2 virus, etc.). In some embodiments, recruited antibodies reduces, inhibits or prevents interaction of SARS-CoV-2 viruses with other cells (e.g., mammalian cells that can be infected), in some embodiments, through disrupting, inhibiting or preventing interactions between SARS-CoV-2 spike proteins and cell proteins, e.g., receptors such as ACE2. In some embodiments, recruited antibodies can induce, recruit, promote, encourage, or enhance one or more immune activities to inhibit, suppress, kill, or remove SARS-CoV-2 viruses and/or celled infected thereby. In some embodiments, as appreciated by those skilled in the art, recruited antibodies recruit various types of immune cells.

In some embodiments, provided agents recruit antibodies or comprise antibody moieties. In some embodiments, provided agents bind spike proteins (e.g., at S½ domain) on virus surfaces, preventing viruses from binding to cells (e.g., preventing viruses from binding to ACE2 receptors on human cells). In some embodiments, provided technologies inhibit viruses from infecting cells. In some embodiments, provided technologies neutralize SARS-CoV-2 viruses. In some embodiments, provided technologies provide direct virus neutralization and/or killing. In some embodiments, provided technologies block virus entry into cells (e.g., human cells).

In some embodiments, provided technologies recruit antibodies, or comprise antibody moieties, that can interact with various Fc receptors, recruit various effector cells and provide various immune activities. In some embodiments, antibodies or antibody moieties effectively interact with FcyRII and/or FcyRIII, e.g., those expressed by macrophages, NK cells, etc. In some embodiments, recruited antibodies or agents comprising antibody moieties recruit macrophages. In some embodiments, recruited antibodies or agents comprising antibody moieties recruit NK cells. In some embodiments, recruited antibodies or agents comprising antibody moieties recruit macrophages and NK cells. In some embodiments, agents of the present disclosure provides inhibition, killing, and removal of SARS-CoV-2 viruses and/or cells infected thereby. Recruited immune cells can provide various immune activities. In some embodiments, macrophages can remove viral particles, e.g., through phagocytosis. In some embodiments, NK cells can kill infected cells. In some embodiments, provided technology provide immune-mediated virus killing (of viruses and/or cells infected thereby).

In some embodiments, provided technologies (e.g., through antibody moieties of provided agents or recruited antibodies by provided agents) can recruit antigen presenting cells, e.g., dendritic cells. In some embodiments, recruited dendritic cells express FcyRII. In some embodiments, provided technologies can deliver viral proteins (e.g., expressed by viruses and/or infected cells) to antigen presenting cells. In some embodiments, provided technologies can provide antigen presentation to various immune cells, e.g., B cell, T cells, etc. In some embodiments, provided technologies can induce, recruit, promote, facilitate, encourage, or enhance priming and activation of immune memory cells (e.g., B-cells and T-cells). In some embodiments, provided technologies can instill long-term immunity (e.g., in some embodiments, like one or more aspects of a vaccine). In some embodiments, provided technologies provide long-term vaccination effect.

In some embodiments, provided agents comprising antibody moieties bind to FcRn. In some embodiments, provided agents comprising antibody moieties bind to FcRn for antibody recycle and/or prolonged half life.

In some embodiments, an immune activity is associated with immune cells. In some embodiments, an immune activity is associated with macrophages. In some embodiments, immune cells are or comprise macrophages. In some embodiments, an immune activity is associated with NK cells. In some embodiments, immune cells are or comprise NK cells. In some embodiments, immune cells are engineered cells. In some embodiments, immune cells are prepared in vitro. For example, in some embodiments, NK cells are or comprise engineered cells. In some embodiments, NK cells are or comprise autologous NK cells. In some embodiments, NK cells are collected, expanded and/or stored autologous NK cells. In some embodiments, NK cells are or comprise allogeneic NK cells. In some embodiments, NK cells are or comprises peripheral blood-derived NK cells. In some embodiments, NK cells are or comprises cord blood-derived NK cells. In some embodiments, provided technologies comprise immune cells in addition to provided agents. In some embodiments, immune cells are administered concurrently with provided agents; in certain embodiments, in the same composition. In some embodiments, immune cells are administered prior to or subsequently to provided agents.

In some embodiments, the present disclosure provides a method for treating a condition, disorder or disease associated with SARS-CoV-2 infection, comprising administering to a subject suffering therefrom a provided agent or composition. In some embodiments, the present disclosure provides a method for treating COVID-19, comprising administering to a subject suffering therefrom a provided agent or composition. In some embodiments, the present disclosure provides a method for inhibiting, killing or removing a SARS-CoV-2, comprising contacting a SARS-CoV-2 with a provided agent or composition. In some embodiments, the present disclosure provides a method for disrupting or reducing an interaction between a cell and a SARS-CoV-2, comprising contacting a SARS-CoV-2 with a provided agent or composition. In some embodiments, the present disclosure provides a method for disrupting or reducing an infection of a SARS-CoV-2 of a cell, comprising contacting a SARS-CoV-2 with a provided agent or composition. In some embodiments, the present disclosure provides a method for inhibiting, killing or removing a cell infected by a SARS-CoV-2, comprising contacting the cell with a provided agent or composition. In some embodiments, provided agents or compositions are utilized in amounts effective to provide desired effects. As described herein, in some embodiments, immune cells, such as various NK cells, may be utilized together with provided agents and/or compositions, and may be administered prior to, concurrently with, or subsequently to provided agents and/or compositions.

In some embodiments, the present disclosure provides pharmaceutical compositions comprising or delivering a provided agent or a pharmaceutically acceptable salt thereof and a pharmaceutically acceptable carrier. In some embodiments, provided technologies are administered to subjects in pharmaceutical compositions.

Provided technologies can provide various benefits and advantages. In some embodiments, provided agents (e.g., certain ARM agents) can be produced through chemical synthesis with both speed and quantity. In some embodiments, provided agents are more stable that therapeutic agents such as antibodies and/or serums, and can be readily stored and distributed in complex global logistical networks. In some embodiments, provided agents are sufficiently stable and do not require cold-chain distribution. In some embodiments, provided agents can be stockpiled (which can be particularly useful for fighting pandemics). In some embodiments, provided agents (e.g., certain ARM agents) are smaller in size than many therapeutic antibodies, and can penetrate and be delivered to locations that cannot be readily reached by therapeutic antibodies. In some embodiments, provided agents can penetrate tissues more quickly and/or at higher levels than other agents (e.g., therapeutic antibodies). In some embodiments, provided agents provide suitable safety profile, and in some embodiments, have been demonstrated to be safer in animal models (e.g., monkeys) than certain therapeutic monoclonal antibodies. In some embodiments, provided agents (e.g., ARM agents) can be safely administered at concentrations up to 60-100 times greater than certain monoclonal antibodies. In some embodiments, agents of the present disclosure provide high efficacy.

### BRIEF DESCRIPTION OF THE DRAWINGS

These and/or other aspects will become apparent and more readily appreciated from the following description of the embodiments, taken in conjunction with the accompanying drawings in which:

FIG. 1. Synthetic scheme for preparation of Agent 1-23.

FIG. 2. Synthetic scheme for preparation of Agent 1-25. (A) Solid phase peptide synthesis of spike protein coupling domain and linker for 1-25 (B) Solid phase peptide synthesis of antibody binding moiety for 1-25.

FIG. 3. Synthetic scheme for preparation of Agent 1-27. (A) Solid phase peptide synthesis of antibody binding moiety and reactive group for 1-27 (B) Linker Synthesis (C) Solid phase peptide synthesis of spike protein coupling domain for 1-27 (D) Assembly of 1-27.

### DETAILED DESCRIPTION OF CERTAIN EMBODIMENTS

### 1. General Description of Certain Embodiments

In some embodiments, the present disclosure provides agents, e.g., antibody-recruiting molecules (ARMs) and antibody conjugates (e.g., agents comprising antibody moieties), that comprise target binding moieties that can bind to entities expressing SARS-CoV-2 spike protein or a fragment thereof (e.g., SARS-CoV-2 viruses and cells infected thereby). In some embodiments, provided agents, e.g., ARMs, comprise universal antibody binding moieties that can bind to antibodies with different Fab structures. In some embodiments, the present disclosure provides agents, e.g., ARMs, that comprises antibody binding moieties that bind to antibodies, e.g., Fc regions of antibodies, and such binding of antibodies do not interfere one or more immune activities of the antibodies, e.g., interaction with Fc receptors (e.g., CD 16a), recruitment of effector cells like NK cells for ADCC, macrophage for ADCP, etc. As those skilled in the art will appreciate, provided technologies (agents, compounds, compositions, methods, etc.) of the present disclosure can provide various advantages, for example, provided technologies can utilize antibodies having various Fab regions in the immune system to avoid or minimize undesired effects of antibody variations among a patient population, can trigger, and/or enhance, immune activities toward targets, e.g., killing target entities such as SARS-CoV-2 viruses and cells infected thereby. In some embodiments, provided technologies can target one or more or all variants of SARS-CoV-2.

In some embodiments, provided technologies are useful for reducing, suppressing, inhibiting, blocking or preventing interactions of SARS-CoV-2 viruses with cells, e.g., those may be infected. In some embodiments, provided technologies are useful for reducing, suppressing, inhibiting, blocking or preventing infection of cells, tissues, organs, or subjects by SARS-CoV-2 viruses. In some embodiments, provided technologies are useful for modulating immune activities against targets (e.g., viruses, infected cells, etc.) expressing a SARS-CoV-2 spike protein or a fragment thereof. In some embodiments, technologies of the present disclosure are useful for recruiting antibodies to targets, particularly those expressing a SARS-CoV-2 spike protein or a fragment thereof. In some embodiments, provided agents can inhibit protein activities and/or interactions, e.g., those of a spike protein (e.g., expressed by a SARS-CoV-2 or a cell infected thereby). In some embodiments, a target binding moiety is an inhibitor moiety.

In some embodiments, the present disclosure provide an agent comprising: an antibody binding moiety, a target binding moiety which can bind a SARS-CoV-2 spike protein or a fragment thereof, and optionally a linker moiety, wherein the antibody binding moiety can bind to two or more antibodies which have different Fab regions.

In some embodiments, the present disclosure provide an agent comprising: an antibody binding moiety, a target binding moiety which can bind a SARS-CoV-2 spike protein or a fragment thereof, and optionally a linker moiety, wherein the antibody binding moiety can bind to two or more antibodies toward different antigens.

In some embodiments, the present disclosure provide an agent comprising: an antibody moiety, a target binding moiety which can bind a SARS-CoV-2 spike protein or a fragment thereof, and optionally a linker moiety,

In some embodiments, provided agents comprise one and only one antibody binding moiety. In some embodiments, provided agents comprise two or more antibody binding moieties. In some embodiments, provided agents comprise one and only one target binding moiety. In some embodiments, provided agents comprise two or more target binding moieties.

An antibody binding moiety may interact with any portion of an antibody. In some embodiments, an antibody binding moiety binds to an Fc region of an antibody. In some embodiments, an antibody binding moiety binds to a conserved Fc region of an antibody. In some embodiments, an antibody binding moiety binds to an Fc region of an IgG antibody. As appreciated by those skilled in the art, various antibody binding moieties, linkers, and target binding moieties can be utilized in accordance with the present disclosure.

In some embodiments, the present disclosure provides antibody binding moieties and/or agents (e.g., compounds of various formulae described in the present disclosure, ARM molecules of the present disclosure, etc.) comprising antibody binding moieties that can bind to a Fc region that is bound to Fc receptors, e.g., FcyRIIIa, CD16a, etc. In some embodiments, provided moieties and/or agents comprising antibody binding moieties that bind to a complex comprising an Fc region and an Fc receptor. In some embodiments, the present disclosure provides a complex comprising:
an agent comprising:
   an antibody binding moiety,
   a target binding moiety, and
   optionally a linker moiety,
an Fc region, and
an Fc receptor.

In some embodiments, an Fc region is an Fc region of an endogenous antibody of a subject. In some embodiments, an Fc region is an Fc region of an exogenous antibody. In some embodiments, an Fc region is an Fc region of an administered agent. In some embodiments, an Fc receptor is of a diseased cell in a subject.

In some embodiments, the present disclosure provides agents having a structure of:

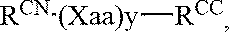

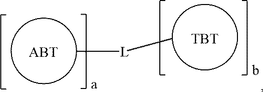

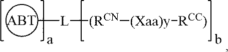

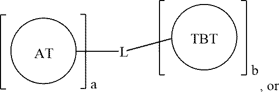

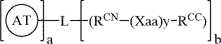

or a salt thereof.

In some embodiments, an antibody binding moiety is a universal antibody binding moiety.

In some embodiments, an antibody binding moiety comprises one or more amino acid residues. In some embodiments, an antibody binding moiety is or comprises a peptide moiety. In some embodiments, an antibody binding moiety is or comprises a cyclic peptide moiety. In some embodiments, such antibody binding moiety comprises one or more natural amino acid residues. In some embodiments, such antibody binding moiety comprises one or more unnatural natural amino acid residues.

In some embodiments, an amino acid has the structure of formula A-I:

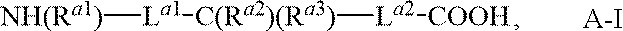

or a salt thereof, wherein:
each of R^{a1}, R^{a2}, R^{a3} is independently —L^{a}—R′;
each of L^{a1} and L^{a2} is independently L^{a};
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a residue has the structure of —N(R^{a1})—L^{a1}—C(R^{a2})(R^{a3})—L^{a2}—COO— or a salt form thereof.

In some embodiments, an amino acid analog is a compound in which the amino group and/or carboxylic acid group are independently replaced with an optionally substituted aliphatic or heteroaliphatic moiety. As those skilled in the art will appreciate, many amino acid analogs, which mimics structures, properties and/or functions of amino acids, are described in the art and can be utilized in accordance with the present disclosure. In some embodiments, one or more peptide groups are optionally and independently replaced with non-peptide groups.

In some embodiments, an antibody-binding moiety is a cyclic peptide moiety. In some embodiments, an antibody binding moiety is or comprises

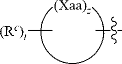

or a salt form thereof.

In some embodiments, the present disclosure provides a compound of formula I-a:

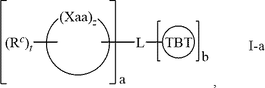

or a salt thereof, wherein:
each Xaa is independently a residue of an amino acid or an amino acid analog;
t is 0-50;
z is 1-50;
L is a linker moiety;
TBT is a target binding moiety;
each R^{c} is independently —L^{a}—R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently -R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a is 1. In some embodiments, b is 1. In some embodiments, a is 1 and b is 1, and a compound of formula I-a has the structure of

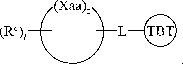

In some embodiments, each residue, e.g., Xaa, is independently a residue of an amino acid or an amino acid analog, wherein the amino acid or the amino acid analog has the structure of H—L^{a1}—L^{a1}—C(R^{a2})(R^{a3})—L^{a2}—L^{a2}—H or a salt thereof. In some embodiments, an amino acid has the structure of NH(R^{a1})—L^{a1}—C(R^{a2})(R^{a3})—L^{a2}—COOH or a salt thereof. In some embodiments, an amino acid analog has the structure of H—L^{a1}—L^{a1}—C(R^{a2})(R^{a3})—L^{a2}—L^{a2}—H or a salt thereof. In some embodiments, in such an amino acid analog, the first —L^{a1}— (bonded to —H in the formula) is not — N(R^{a1})— (e.g., is optionally substituted bivalent C₁₋₆ aliphatic). In some embodiments, in H—L^{a1}—L^{a1}—, —L^{a1}—L^{a1}— bonds to the —H through an atom that is not nitrogen. In some embodiments, in —L^{a2}—L^{a2}—H, —L^{a2}—L^{a2}— is not bonded to the —H through —C(O)O—. In some embodiments, each residue, e.g., each Xaa in formula I-a, is independently a residue of an amino acid having the structure of formula A-I.

In some embodiments, each Xaa independently has the structure of —L^{a1}—L^{a1}—C(R^{a2})(R^{a3})—L^{a2}—L^{a2}—. In some embodiments, each Xaa independently has the structure of —L^{aX1}—L^{a1}—C(R^{a2})(R^{a3})—L^{a2}—L^{aX2}—, wherein L^{aX1} is optionally substituted —NH—, optionally substituted —CH₂—, — N(R^{a1})—, or —S—, L^{aX2} is optionally substituted —NH—, optionally substituted —CH₂—, — N(R^{a1})—, or —S—, and each other variable is independently as described herein. In some embodiments, L^{aX1} is optionally substituted —NH—, or — N(R^{a1})—. In some embodiments, L^{aX1} is optionally substituted —CH₂—, or —S—. In some embodiments, L^{aX2} is optionally substituted —NH—, optionally substituted —CH₂—, —N(R^{a1})—, or —S—. In some embodiments, optionally substituted —CH₂— is —C(O)—. In some embodiments, optionally substituted —CH₂— is not —C(O)—. In some embodiments, L^{aX2} is —C(O)—. In some embodiments, each Xaa independently has the structure of —N(R^{a1})—L^{a1}—C(R^{a2})(R^{a3})—L^{a2}—CO—.

In many embodiments, two or more residues, e.g., two or more Xaa residues, are linked together such that one or more cyclic structures are formed. For example, various compounds in Table 1 comprises linked residues. Residues can be linked, optionally through a linker (e.g., L^{T}) at any suitable positions. For example, a linkage between two residues can connect each residue independently at its N-terminus, C-terminus, a point on the backbone, or a point on a side chain, etc. In some embodiments, two or more side chains of residues, e.g., in compounds of formula I-a, (e.g., R^{a2} or R^{a3} of one amino acid residue with R^{a2} or R^{a3} of another amino acid residue) are optionally take together to form a bridge (e.g., in various compounds in Table 1, etc.), e.g., in some embodiments, two cysteine residues form a —S—S—bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-a through a side chain of an amino acid residue (e.g., Xaa in formula I-a). In some embodiments, such a side chain comprises an amino group or a carboxylic acid group. In some embodiments, L^{T} is L^{b} as described herein. In some embodiments, a linkage, e.g., L^{b} or L^{T}, connects a side chain with a N-terminus or a C-terminus of a residue. In some embodiments, a linkage connects a side chain with an amino group of a residue. In some embodiments, a linkage connects a side chain with an alpha-amino group of a residue. In some embodiments, as illustrated herein, a linkage, e.g., L^{b} or L^{T}, is —CH₂—C(O)—. In some embodiments, the —CH₂— is bonded to a side chain, e.g., boned to —S— of a cysteine residue, and the —C(O)— is bonded to an amino group, e.g., an alpha-amino group of a residue. In some embodiments, a linkage, e.g., L^{b} or L^{T}, is optionally substituted —CH₂—S—CH₂—C(O)—NH—, wherein each end is bonded to the alpha-carbon of a residue. In some embodiments, the —NH— is of an alpha-amino group of a residue, e.g., of a N-terminal residue.

In some embodiments,

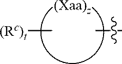

is an antibody binding moiety (

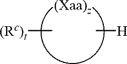

binds to an antibody). In some embodiments,

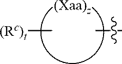

is a universal antibody binding moiety. In some embodiments,

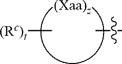

is a universal antibody binding moiety which can bind to antibodies having different Fab regions. In some embodiments,

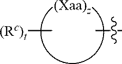

is a universal antibody binding moiety that can bind to a Fc region. In some embodiments, an antibody binding moiety, e.g., a universal antibody binding moiety having the structure of

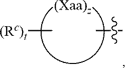

can bind to a Fc region bound to an Fc receptor. In some embodiments, an antibody binding moiety, e.g., of an antibody binding moiety having the structure of

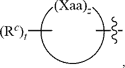

has the structure of

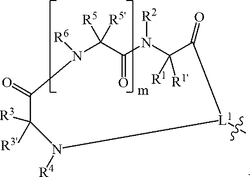

In some embodiments,

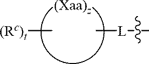

has the structure of

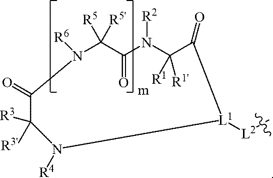

In certain embodiments, the present disclosure provides a compound of formula II:

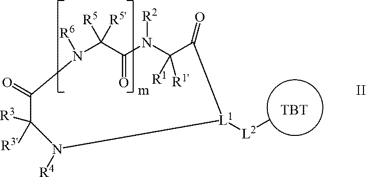

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂— or —Cy¹—, wherein each —Cy¹— is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety that connects

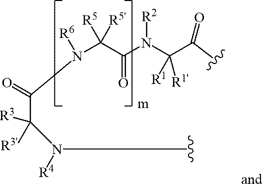

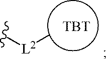

L² is a covalent bond or a C₁₋₃₀ optionally substituted bivalent straight or branched saturated or unsaturated hydrocarbon chain wherein 1-10 methylene units of the chain are independently and optionally replaced with —S—, —N(R)—, —O—, —C(O)—, —OC(O)—, —C(O)O—, —C(O)N(R)—, —N(R)C(O)—, —S(O)—,—S(O)₂—

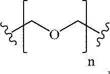

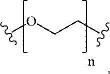

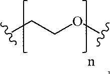

or —Cy¹—, wherein each —Cy¹—is independently a 5-6 membered heteroarylenyl with 1-4 heteroatoms independently selected from nitrogen, oxygen or sulfur;
TBT is a target binding moiety; and
each of m and n is independently 1, 2, 3, 4, 5, 6, 7, 8, 9, or 10.

In some embodiments, an antibody binding moiety is or comprises a peptide moiety. In some embodiments, the present disclosure provides a compound having the structure of formula I-b:

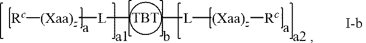

or a salt thereof, wherein:
each Xaa is independently a residue of an amino acid or an amino acid analog;
each z is independently 1-50;
each L is independently a linker moiety;
TBT is a target binding moiety,
each R^{c} is independently —L^{a}—R′;
each of a1 and a2 is independently 0 or 1, wherein at least one of a1 and a2 is not 0;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or—C(O)O—;
each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, al is 1. In some embodiments, a2 is 1. In some embodiments, b is 1. In some embodiments, a compound of formula I-b has the structure of

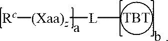

In some embodiments, a compound of formula I-b has the structure of

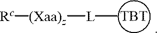

In some embodiments, a compound of formula I-b has the structure of

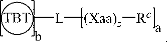

In some embodiments, a compound of formula I-b has the structure of

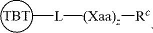

In some embodiments, each residue, e.g., each Xaa in formula I-a, I-b, etc., is independently a residue of amino acid having the structure of formula A-I. In some embodiments, each Xaa independently has the structure of —N(R^{a1})—L^{a1}—C(R^{a2})(R^{a3})—L^{a2}—CO—. In some embodiments, two or more side chains of the amino acid residues, e.g., in compounds of formula I-a, (e.g., R^{a2} or R^{a3} of one amino acid residue with R^{a2} or R^{a3} of another amino acid residue) are optionally take together to form a bridge (e.g., various compounds in Table 1), e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-b through a side chain of an amino acid residue (e.g., Xaa in formula I-a). In some embodiments, such a side chain comprises an amino group or a carboxylic acid group.

In some embodiments, R^{c}-(Xaa)z- is an antibody binding moiety (R^{c}-(Xaa)z-H binds to an antibody). In some embodiments, R^{c}-(Xaa)z- is a universal antibody binding moiety. In some embodiments, R^{c}-(Xaa)z- is a universal antibody binding moiety which can bind to antibodies having different Fab regions. In some embodiments, R^{c}-(Xaa)z- is a universal antibody binding moiety that can bind to a Fc region. In some embodiments, an antibody binding moiety, e.g., a universal antibody binding moiety having the structure of R^{c}-(Xaa)z-, can bind to a Fc region which binds to an Fc receptor. In some embodiments, R^{c}-(Xaa)z- has the structure of

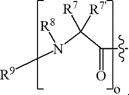

In some embodiments, R^{c}—(Xaa)z—L— has the structure of

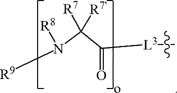

In certain embodiments, the present disclosure provides a compound of formula III:

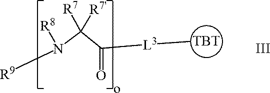

or a pharmaceutically acceptable salt thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
R⁹ is hydrogen, optionally substituted C₁₋₃ aliphatic, or —C(O)—(optionally substituted C₁₋₃ aliphatic);
L³ is a bivalent linker moiety that connects

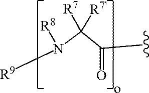

with TBT;
TBT is a target binding moiety; and
o is 1, 2, 3, 4, 5, 6, 7, 8, 9, or 10.

### 2. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March’s Advanced Organic Chemistry”, 5^{th} Ed., Ed.: Smith, M.B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise specified, compounds described herein may be provided and/or utilized in a salt form, particularly a pharmaceutically acceptable salt form.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y’s stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains - an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi- specific antibodies (e.g., Zybodies®, additional bi- or multi- specific antibodies described in Ulrich Brinkmann & Roland E. Kontermann (2017) The making of bispecific antibodies, mAbs, 9:2, 182-212, doi: 10.1080/19420862.2016.1268307, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; KALBITOR®s; CovX-Bodies; and CrossMabs. In some embodiments, antibodies may have enhanced Fc domains. In some embodiments, antibodies may comprise one or more unnatural amino acid residues. In some embodiments, an antibody may lack a covalent modification (e.g., attachment of a glycan) that it would have if produced naturally. In some embodiments, an antibody is an afucosylated antibody. In some embodiments, an antibody is conjugated with another entity. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, polyethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including various forms of such atoms, such as oxidized forms (e.g., of nitrogen, sulfur, phosphorus, or silicon), quaternized form of a basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl) etc.). In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR°, —SC(S)SR°; —(CH₂)₀₋₄SC(O)R°; —(CH₂)₀₋₄C(O)NR°₂; —C(S)NR°₂; —C(S)SR°; —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —Si(R°)₃; —OSi(R°)₃; —B(R°)₂; —OB(R°)₂; —OB(OR°)₂; —P(R°)₂; —P(OR°)₂; —P(R°)(OR°); —OP(R°)₂; —OP(OR°)₂; —OP(R°)(OR°); —P(O)(R°)₂; —P(O)(OR°)₂; —OP(O)(R°)₂; —OP(O)(OR°)₂; —OP(O)(OR°)(SR°); —SP(O)(R°)₂; —SP(O)(OR°)₂; —N(R°)P(O)(R°)₂; —N(R°)P(O)(OR°)₂; —P(R°)₂[B(R°)₃]; —P(OR°)₂[B(R°)₃]; —OP(R°)₂[B(R°)₃]; —OP(OR°)₂[B(R°)₃]; —(C₁₋₄ straight or branched alkylene)O—N(R°)₂; or —(C₁₋₄ straight or branched alkylene)C(O)O—N(R°)₂, wherein each R° may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂—(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R°, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R° (or the ring formed by taking two independent occurrences of R° together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or -SSR^{•} wherein each R^{•} is unsubstituted or where preceded by “halo” is substituted only with one or more halogens, and is independently selected from C₁₋₄ aliphatic, —CH₂Ph, —O(CH₂)₀₋₄Ph, and a 5-6-membered saturated, partially unsaturated, or aryl ring having 0-4 heteroatoms independently selected from nitrogen, oxygen, and sulfur. Suitable divalent substituents on a saturated carbon atom of R° include =O and =S.

Suitable divalent substituents, e.g., on a suitable carbon atom, are independently the following: ═O, ═S, ═NNR^{*}₂, ═NNHC(O)R^{*}, ═NNHC(O)OR*, ═NNHS(O)₂R*, ═NR*, ═NOR*, —O(C(R^{*}₂))₂₋₃O—, or —S(C(R^{*}₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR^{*}₂)₂₋₃O-, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R^{*} are independently halogen, -R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently -R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono-or bicyclic ring having 0-4 heteroatoms independently selected from nitrogen, oxygen, and sulfur.

Suitable substituents on the aliphatic group of R^{†} are independently halogen, -R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i. e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methane sulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate,p-toluenesulfonate, undecanoate, valerate salts, and the like. In some embodiments, a provided compound comprises one or more acidic groups and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′ p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methane sulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1 -ethoxyethyl, 1 -(2-chloroethoxy)ethyl, 2- trimethylsilylethyl, p-chlorophenyl, 2,4-dinitrophenyl, benzyl, benzoyl, p-phenylbenzoyl, 2,6- dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9- fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t- butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-l-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a compound described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure. Unless otherwise stated, salts/salt forms of compounds, agents, moieties, etc., are included.

### 3. Description of Exemplary Embodiments

In some embodiments, the present disclosure provide an agent comprising a target binding moiety as described herein.

In some embodiments, the present disclosure provide an agent comprising:
an antibody binding moiety,
a target binding moiety, and
optionally a linker moiety.

In some embodiments, the present disclosure provide an agent comprising:
an antibody moiety,
a target binding moiety, and
optionally a linker moiety.

In some embodiments, an antibody binding moiety is a uABT. In some embodiments, a target binding moiety can bind to a SARS-CoV-2 spike protein or a fragment thereof. In some embodiments, the present disclosure provides agents that can bind to a SARS-CoV-2 spike protein or a fragment thereof. In some embodiments, an agent is a compound of formula I, I-a, I-b, II or III, or a salt thereof. In some embodiments, the present disclosure provides compounds of formula I, I-a, I-b, II or III, or pharmaceutically acceptable salts thereof. Various embodiments of provided technologies are described herein as examples.

### Antibody Binding, Moieties

Among other things, the present disclosure provides agents, e.g., ARMs, comprising antibody binding moieties. In some embodiments, antibody binding moieties are universal antibody binding moieties which can bind to antibodies having different Fab regions and different specificity. In some embodiments, antibody binding moieties of the present disclosure are universal antibody binding moieties that bind to Fc regions. In some embodiments, binding of universal antibody binding moieties to Fc regions can happen at the same time as binding of Fc receptors, e.g., CD16a, to the same Fc regions (e.g., may at different locations/amino acid residues of the same Fc regions). In some embodiments, upon binding of universal antibody binding moieties, e.g., those in provided agents, compounds, methods, etc., an Fc region can still interact with Fc receptors and perform one or more or all of its immune activities, including recruitment of immune cells (e.g., effector cells such as NK cells), and/or triggering, generating, encouraging, and/or enhancing immune system activities toward target cells, tissues, objects and/or entities, for example, antibody-dependent cell-mediated cytotoxicity (ADCC) and/or ADCP.

Various universal antibody binding moieties can be utilized in accordance with the present disclosure. Certain antibody binding moieties and technologies for identifying and/or assessing universal antibody binding moieties and/or their utilization in ARMs are described in WO/2019/023501 and are incorporated herein by reference. Those skilled in the art appreciates that additional technologies in the art may be suitable for identifying and/or assessing universal antibody binding moieties suitable for ARMs in accordance with the present disclosure. In some embodiments, a universal antibody binding moiety comprises one or more amino acid residues, each independently natural or unnatural. In some embodiments, a universal antibody binding moiety has the structure of

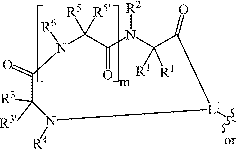

a salt form thereof. In some embodiments, a universal antibody binding moiety has the structure of

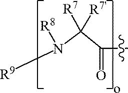

or a salt form thereof. In some embodiments, a universal antibody binding moiety is or comprises a peptide moiety, e.g., a moiety having the structure of R^{c}-(Xaa)z- or a salt form thereof, wherein each of R^{c}, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising -S-S- (which, as in many proteins, can be formed by two -SH groups). In some embodiments, a universal antibody binding moiety is or comprises a cyclic peptide moiety, e.g., a moiety having the structure of

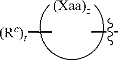

or a salt form thereof. In some embodiments, a universal antibody binding moiety is R^{c}-(Xaa)z- or

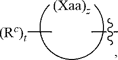

or a salt form thereof, and is or comprises a peptide unit. In some embodiments, -(Xaa)z- is or comprises a peptide unit. In some embodiments, a peptide unit comprises an amino acid residue (e.g., at physiological pH about 7.4, “positively charged amino acid residue”, Xaa^{P}), e.g., a residue of an amino acid of formula A-I that has a positively charged side chain. In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{p}Xaa^{p}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{P}Xaa^{P}Xaa^{A}. In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA. In some embodiments, a peptide unit is or comprises WGRR. In some embodiments, a peptide unit is or comprises RRGW. In some embodiments, a peptide unit is or comprises NKFRGKYK. In some embodiments, a peptide unit is or comprises NRFRGKYK. In some embodiments, a peptide unit is or comprises NARKFYK. In some embodiments, a peptide unit is or comprises NARKFYKG. In some embodiments, a peptide unit is or comprises HWRGWV. In some embodiments, a peptide unit is or comprises KHFRNKD. In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide residue is RHRFNKD. In some embodiments, a peptide unit is TY. In some embodiments, a peptide unit is TYK. In some embodiments, a peptide unit is RTY. In some embodiments, a peptide unit is RTYK. In some embodiments, a peptide unit is or comprises a sequence selected from PAM. In some embodiments, a peptide unit is WHL. In some embodiments, a peptide unit is ELVW. In some embodiments, a peptide unit is or comprises a sequence selected from AWHLGELVW. In some embodiments, a peptide unit is or comprises a sequence selected from DCAWHLGELVWCT, which the two cysteine residues can form a disulfide bond as found in natural proteins. In some embodiments, a peptide unit is or comprises a sequence selected from Fc-III. In some embodiments, a peptide unit is or comprises a sequence selected from DpLpAWHLGELVW. In some embodiments, a peptide unit is or comprises a sequence selected from FcBP-1. In some embodiments, a peptide unit is or comprises a sequence selected from DpLpDCAWHLGELVWCT. In some embodiments, a peptide unit is or comprises a sequence selected from FcBP-2. In some embodiments, a peptide unit is or comprises a sequence selected from CDCAWHLGELVWCTC, wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH.

In some embodiments, -(Xaa)z- is or comprises [X¹]ₚ₁[X²]ₚ₂-X³X⁴X⁵X⁶X⁷X⁸X⁹X¹⁰X¹¹X¹²-[X¹³]ₚ₁₃-[X¹⁴]ₚ₁₄[X¹⁵]ₚ₁₅[X¹⁶]ₚ₁₆, wherein each of X¹, X², X³ X⁴ X⁵, X⁶ X^{7,} X⁸, X⁹ X^{10,} X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises —Cy—. In some embodiments, L^{a} comprises —Cy—, wherein —Cy— is optionally substituted heteroaryl. In some embodiments, —Cy— is

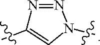

In some embodiments, L^{a} is

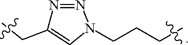

In some embodiments, such an L^{a} can be formed by a —N₃ group of the side chain of one amino acid residue, and the of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—Co—(CH₂)₂—. In some embodiments, —Cy— is optionally substituted phenylene. In some embodiments, —Cy— is optionally substituted 1,2-phenylene. In some embodiments, L^{a} is

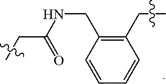

In some embodiments, L^{a} is

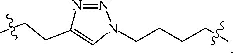

In some embodiments, L^{a} is optionally substituted bivalent C₂-₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises -X³X⁴X⁵X⁶X⁷X⁸X⁹X¹⁰X¹¹X¹²-, wherein: each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.
In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

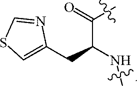

In some embodiments, X⁵ is

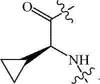

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

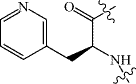

. In some embodiments, X¹² is

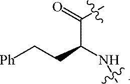

In some embodiments, each of X⁷, X¹⁰, and X¹¹ is independently an amino acid residue with a hydrophobic side chain (“hydrophobic amino acid residue”, XaaH). In some embodiments, X⁷ is Xaa^{H}. In some embodiments, X⁷ is

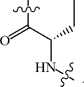

In some embodiments, X⁷ is Val. In some embodiments, X¹⁰ is Xaa^{H}. In some embodiments, X¹⁰ is Met. In some embodiments, X¹⁰ is

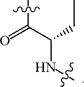

In some embodiments, X¹¹ is Xaa^{H}. In some embodiments, X¹¹ is

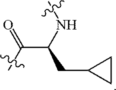

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety.

In some embodiments, -(Xaa)z- is or comprises -X³X⁴X⁵X⁶X⁷X⁸X⁹X¹⁰X¹¹X¹²-, wherein: each of X³, X⁴, X⁵, X⁶, X⁷, X⁸, X⁹, X¹⁰, X¹¹, and X¹² is independently an amino acid residue; at least two amino acid residues are connected through one or more linkages L^{b}; L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂— —S(O)₂N(R′)—C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.
In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

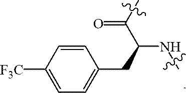

In some embodiments, X¹² is

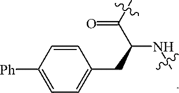

In some embodiments, X¹² is

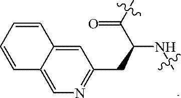

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

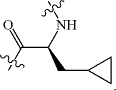

In some embodiments, X¹¹ is Xaa^{H}. In some embodiments, X¹¹ is

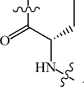

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety. In some embodiments, L^{b} is

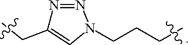

In some embodiments, L^{b} is

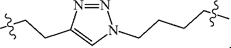

In some embodiments, L^{b} connects two alpha-carbon atoms of two different amino acid residues. In some embodiments, both X⁵ and X¹⁰ are Cys, and the two —SH groups of their side chains form —S—S— (L^{b} is ⁻CH₂—S—S—CH₂—).

In some embodiments, -(Xaa)z- is or comprises -X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.
In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L^{b} is

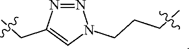

In some embodiments, L^{b} is

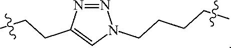

In some embodiments, L^{b} is —CH₂CH₂CO—N(R′)—CH₂CH₂—. In some embodiments, R′ are taken together with an R group on the backbone atom that —N(R′)—CH₂CH₂— is bonded to to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

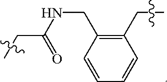

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two -SH groups of their side chains form —S—S— (L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

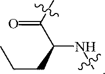

In some embodiments, X¹⁰ is Xaa^{H}. In some embodiments, X¹⁰ is Val. In some embodiments, X¹⁰ is

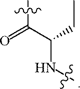

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{L}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)-NHCH₂CH(OH)CH₃. In some embodiments, R^{c} is (S)—NHCH₂CH(OH)CH₃.

In some embodiments, -(Xaa)z- is or comprises -X²X³X⁴X⁵X⁶X⁷X⁸X⁹X¹⁰X¹¹X¹²-, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.
In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

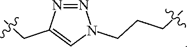

In some embodiments, L^{b} is

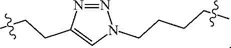

In some embodiments, both X² and X¹² are Cys, and the two —SH groups of their side chains form —S—S— (L^{b} is —CH₂—S—S—CH₂—). In some embodiments, both X⁴ and X¹⁰ are Cys, and the two —SH groups of their side chains form —S—S— (L^{b} is —CH₂—S—S—CH₂—). In some embodiments, X⁴ and X⁹ are connected by L^{b}, wherein L^{b} is

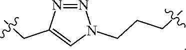

In some embodiments, X⁴ and X⁹ are connected by L^{b}, wherein L^{b} is

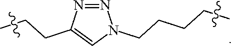

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

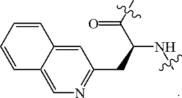

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

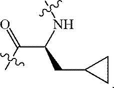

In some embodiments, X⁹ is Xaa^{H}. In some embodiments, X⁹ is Leu. In some embodiments, X⁹ is

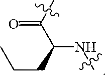

In some embodiments, X¹⁰ is Xaa^{H}. In some embodiments, X¹⁰ is Val. In some embodiments, X⁷ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises -X²X³X⁴X⁵X⁶X⁷X⁸X⁹X¹⁰X¹¹X¹²X¹³X¹⁴X¹⁵X¹⁶-, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}.
In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² are connected to X¹⁶ by L^{b}. In some embodiments, X⁴ are connected to X¹⁴ by L^{b}. In some embodiments, both X² and X¹⁶ are Cys, and the two —SH groups of their side chains form —S—S— (L^{b} is —CH₂—S—S—CH₂—). In some embodiments, both X⁴ and X¹⁴ are Cys, and the two —SH groups of their side chains form —S—S— (L^{b} is —CH₂—S—S—CH₂—). In some embodiments, L^{b} connects two alpha-carbon atoms of two different amino acid residues. In some embodiments, X³ is Asp. In some embodiments, X³ is Glu. In some embodiments, X⁵ is XaaH. In some embodiments, X⁵ is Ala. In some embodiments, X⁶ is Xaa^{A}. In some embodiments, X⁶ is Tyr. In some embodiments, X⁷ is Xaa^{A}. In some embodiments, X⁷ is Xaa^{P}. In some embodiments, X⁷ is His. In some embodiments, X⁸ is Xaa^{H}. In some embodiments, X⁸ is Ala. In some embodiments, X⁹ is Gly. In some embodiments, X¹⁰ is Asp. In some embodiments, X¹⁰ is Glu. In some embodiments, X¹¹ is Xaa^{H}. In some embodiments, X¹¹ is Leu. In some embodiments, X¹² is XaaH. In some embodiments, X¹² is Val. In some embodiments, X¹³ is Xaa^{A}. In some embodiments, X¹³ is Tyr. In some embodiments, X¹⁵ is Xaa^{L}. In some embodiments, X¹⁵ is Thr. In some embodiments, X¹⁵ is Val. In some embodiments, p1 is 1. In some embodiments, In some embodiments, X¹ is Xaa^{N}. In some embodiments, X¹ is Asp. In some embodiments, X¹ is Glu.

As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, Ile, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, an antibody binding moiety, e.g., a universal antibody binding moiety, is or comprises optionally substituted moiety of Table A-1. In some embodiments, an antibody binding moiety, e.g., a universal antibody binding moiety, is selected from able A-1.

TABLE A-1

Exemplary antibody binding moieties

| | |
|---|---|
| 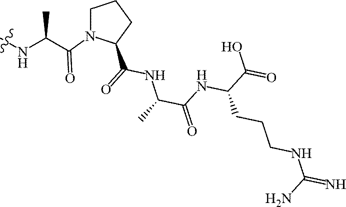 | 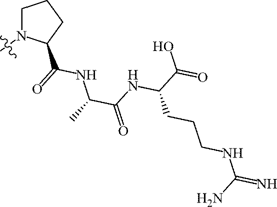 |
| A-1 | A-2 |
| 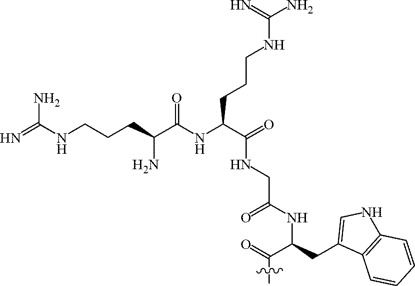 | 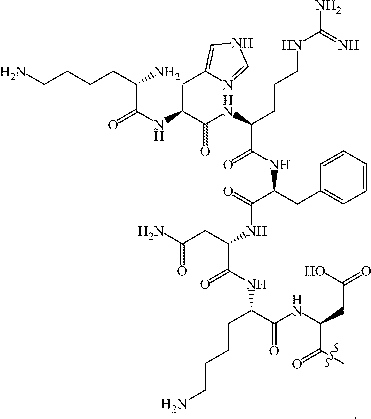 |
| A-3 | A-4 |
| 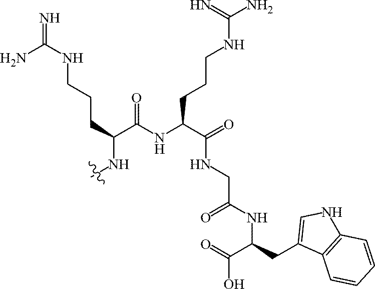 | 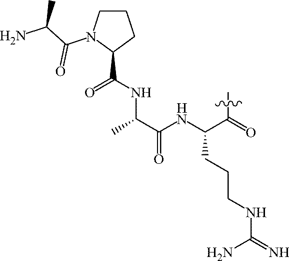 |
| A-5 | A-6 |
| 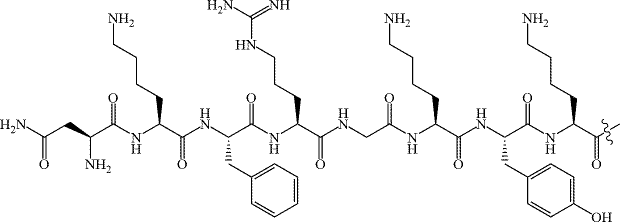 | 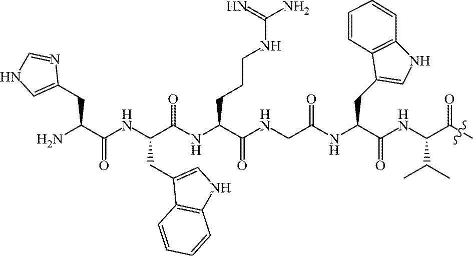 |
| A-7 | A-8 |
| 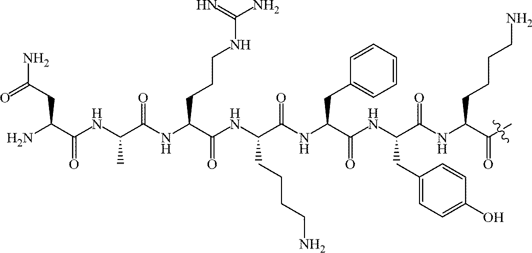 | 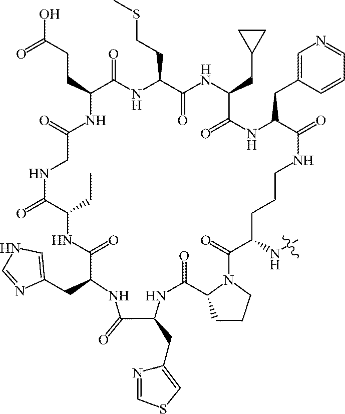 |
| A-9 | A-10 |
| 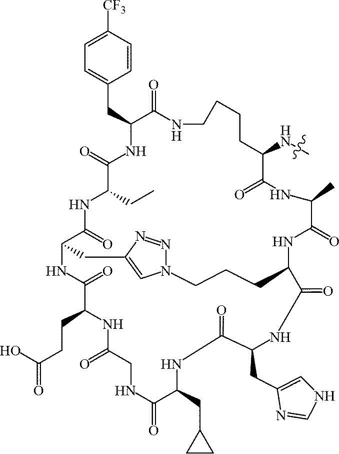 | 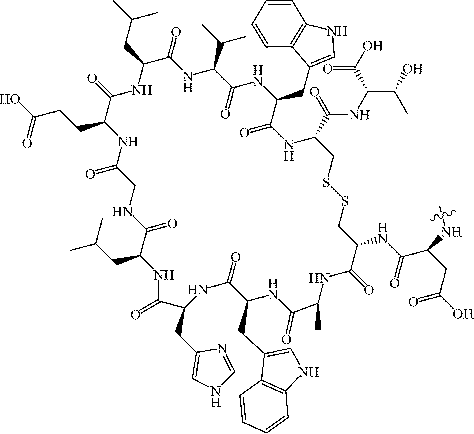 |
| A-11 | A-12 |
| 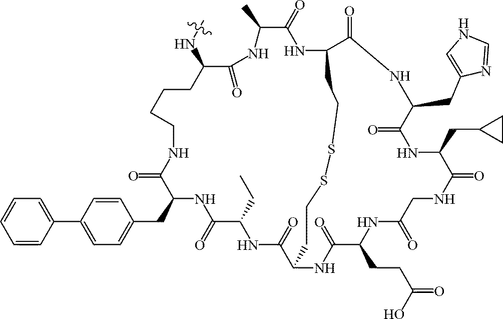 | 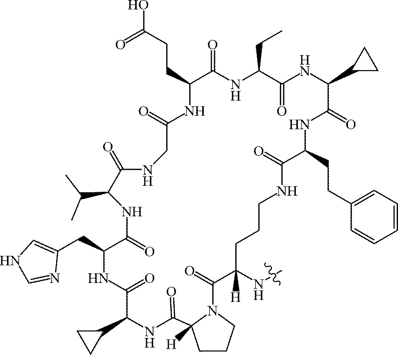 |
| A-13 | A-14 |
| 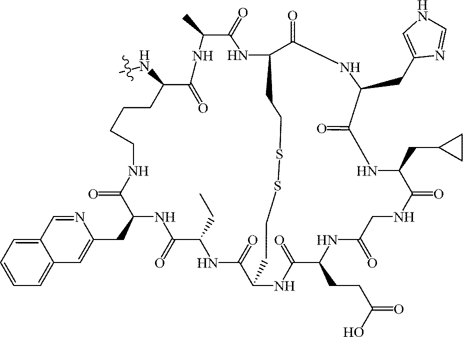 | 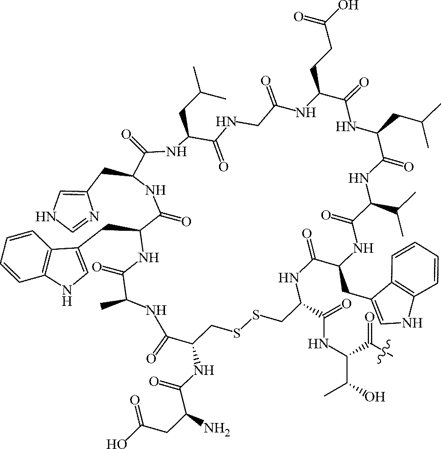 |
| A-15 | A-16 |
| 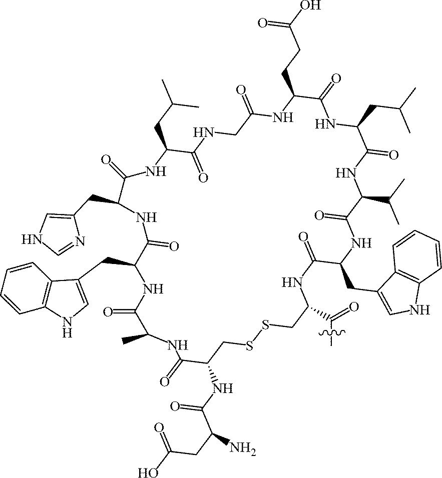 | 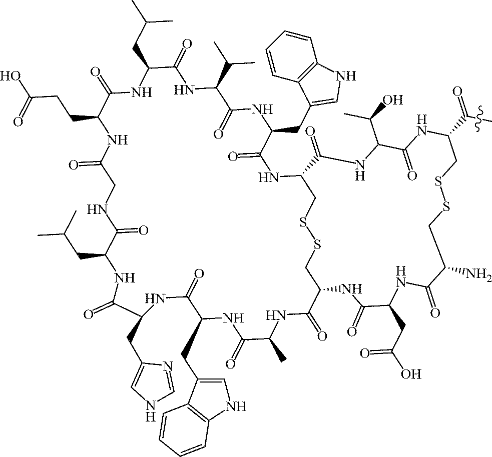 |
| A-17 | A-18 |
| 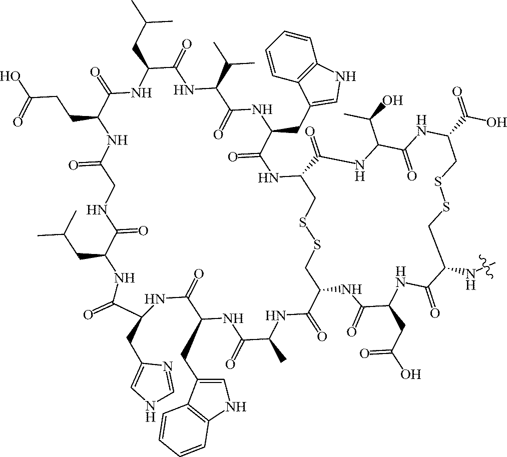 | 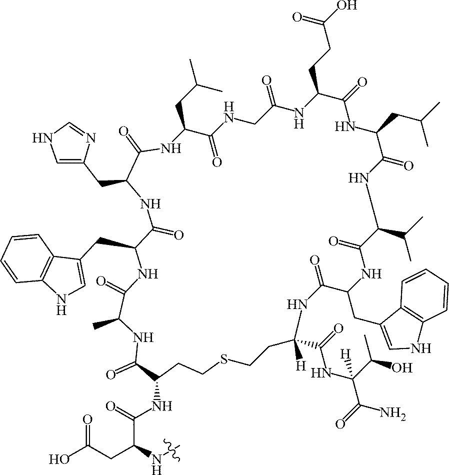 |
| A-19 | A-20 |
| 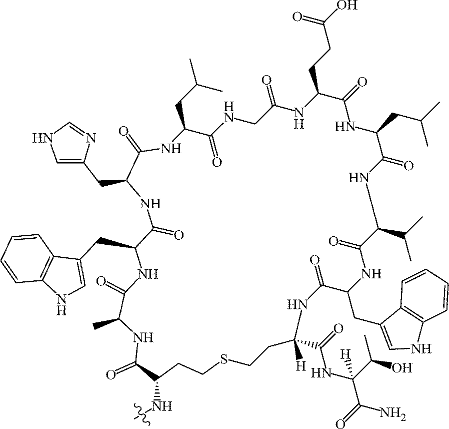 | 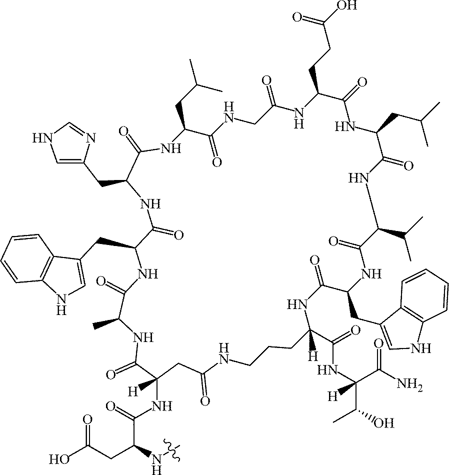 |
| A-21 | A-22 |
| 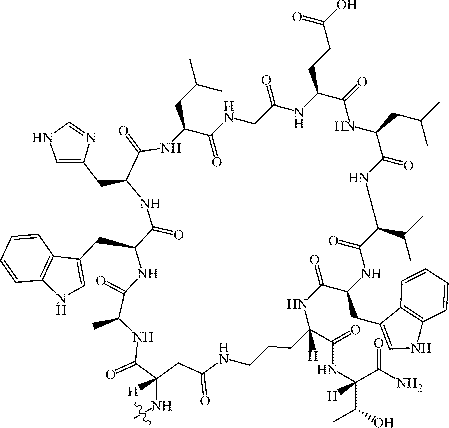 | 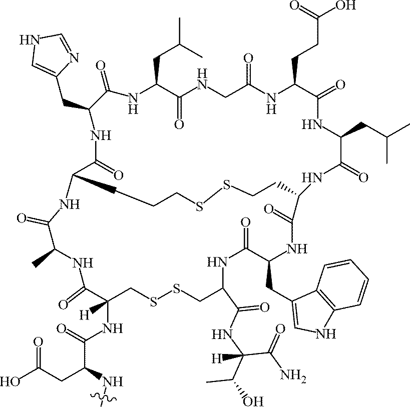 |
| A-23 | A-24 |
| 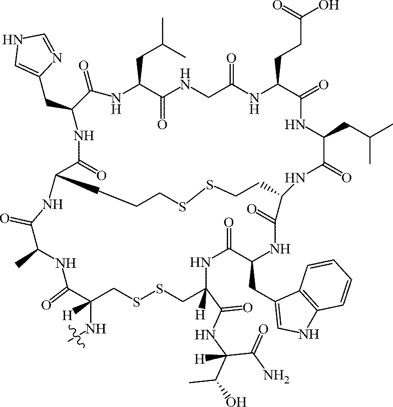 | 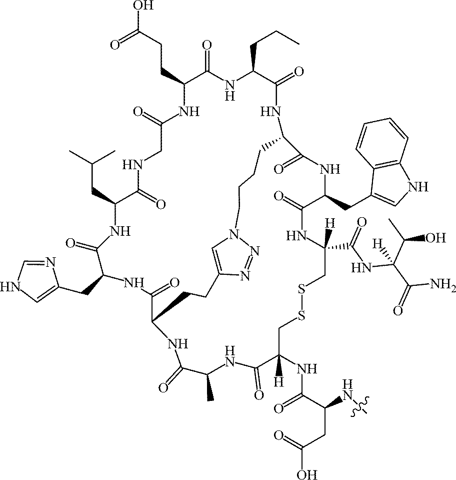 |
| A-25 | A-26 |
| 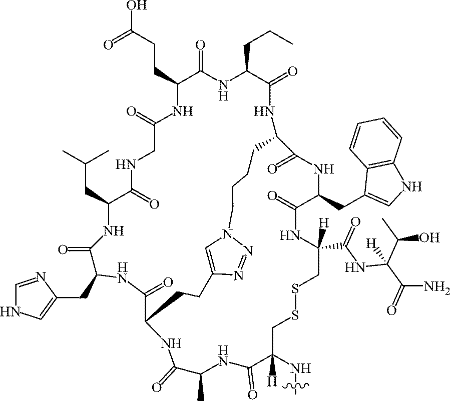 | 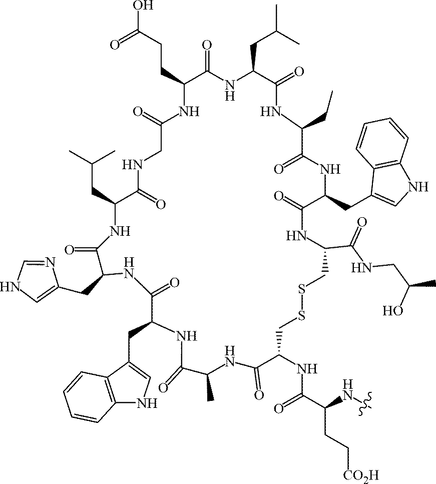 |
| A-27 | A-28 |
| 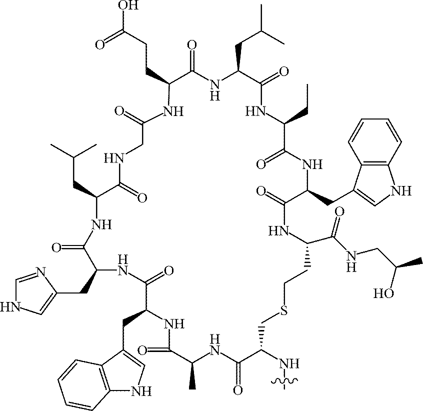 | 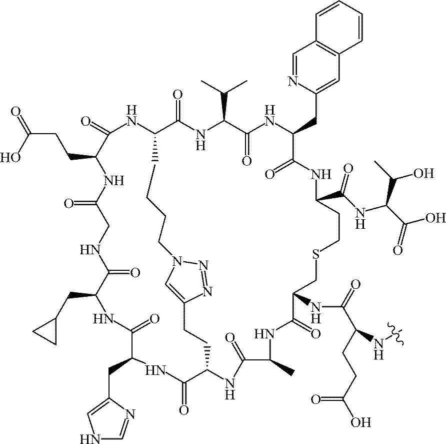 |
| A-29 | A-30 |
| 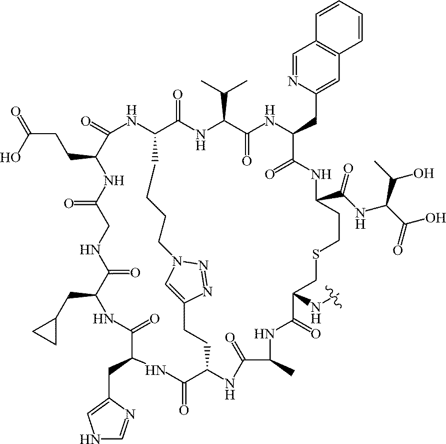 | 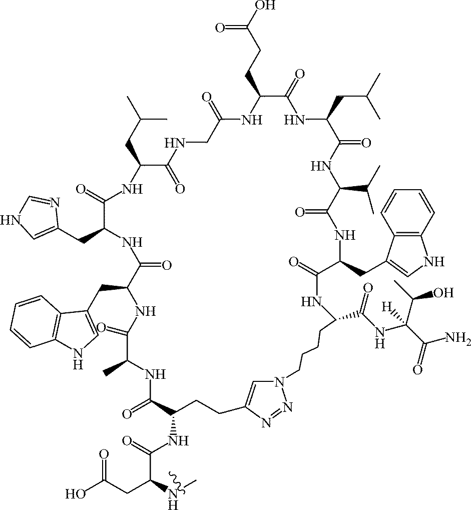 |
| A-31 | A-32 |
| 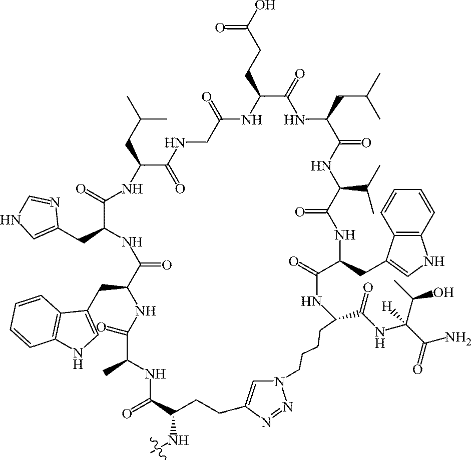 | 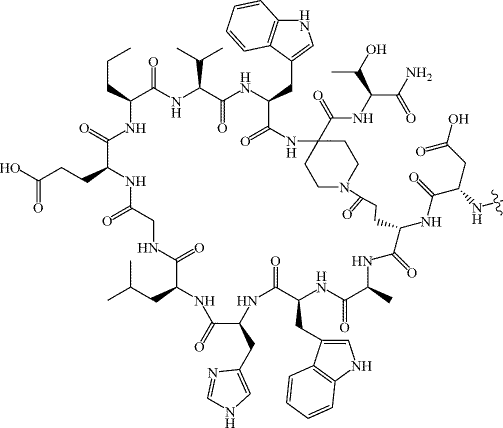 |
| A-33 | A-34 |
| 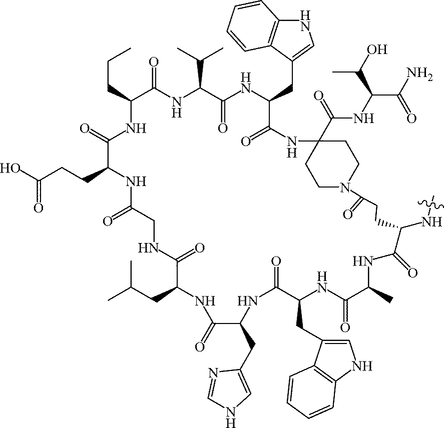 | 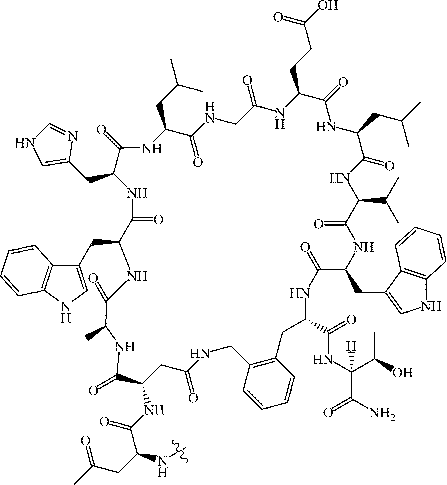 |
| A-35 | A-36 |
| 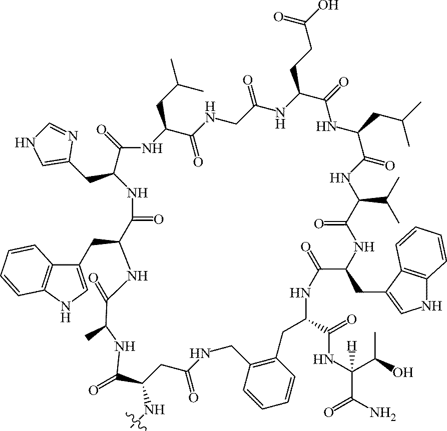 | 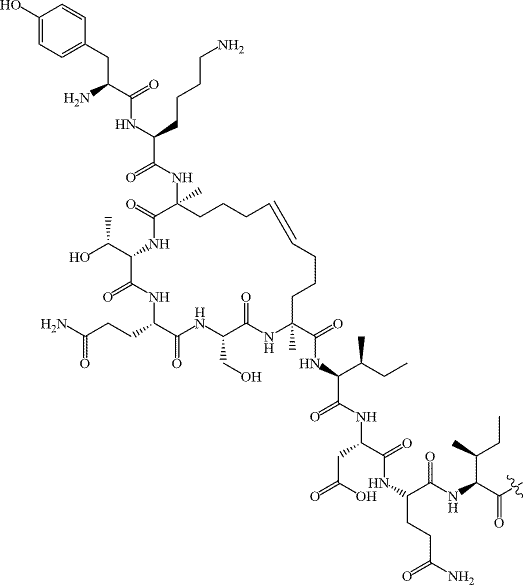 |
| A-37 | A-38 |
| 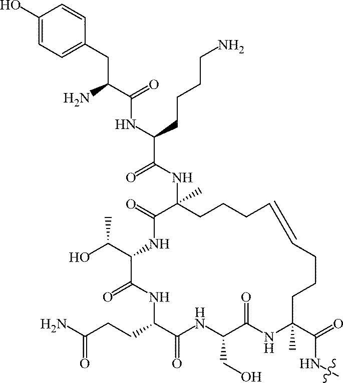 | 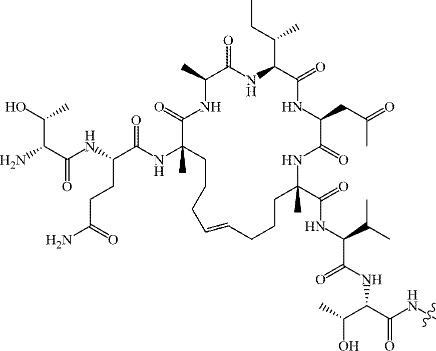 |
| A-39 | A-40 |
| 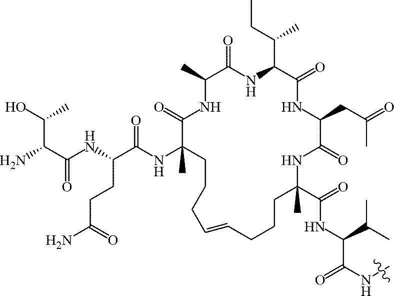 | 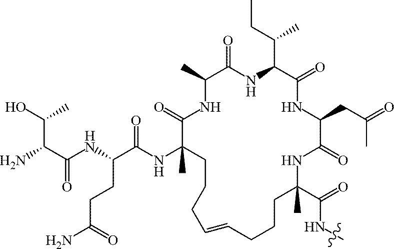 |
| A-41 | A-42 |
| 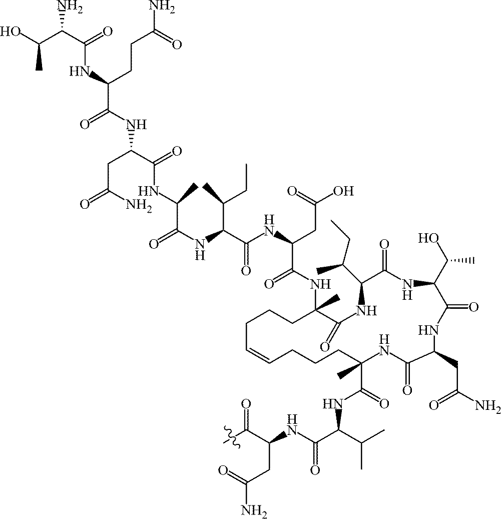 | 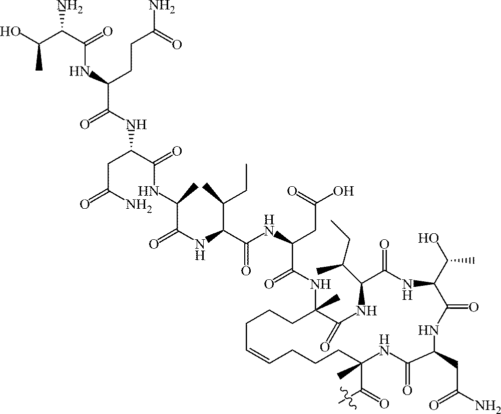 |
| A-43 | A-44 |
| 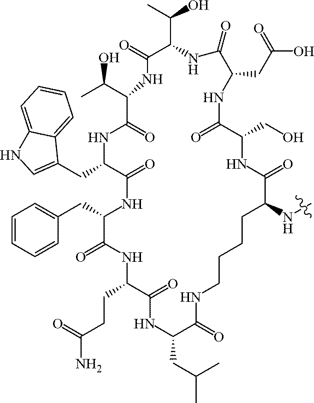 | 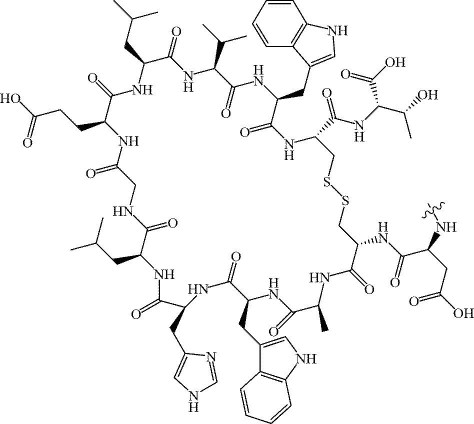 |
| A-45 | A-46 |
| 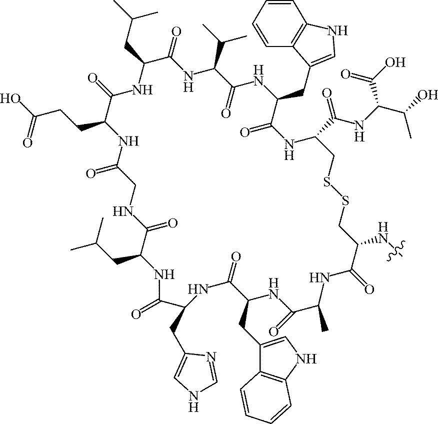 | 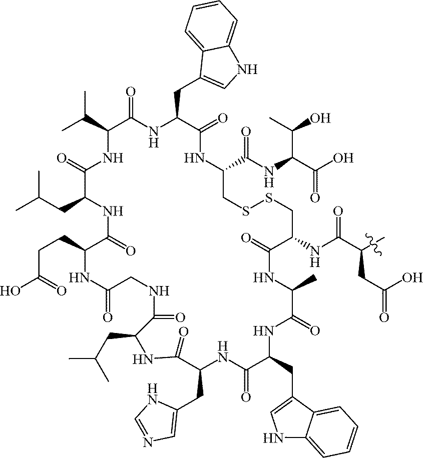 |
| A-47 | A-48 |
| 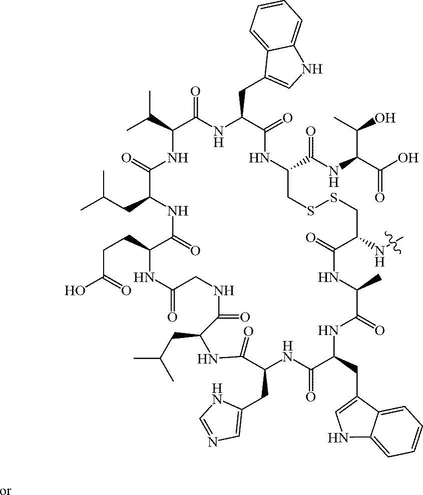 | 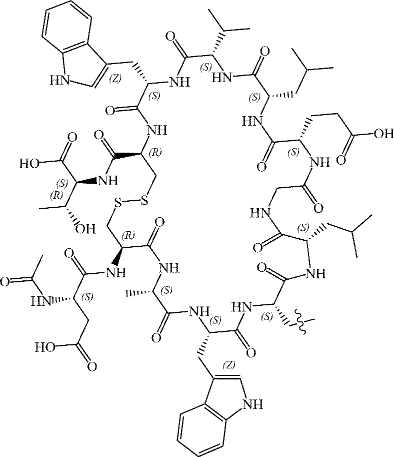 |
| A-49 | A-50 |

In some embodiments, a universal antibody binding moiety is or comprises optionally substituted A-1. In some embodiments, a universal antibody binding moiety is or comprises optionally substituted A-2. In some embodiments, a universal antibody binding moiety is or comprises optionally substituted A-3. In some embodiments, a universal antibody binding moiety is or comprises optionally substituted A-4. In some embodiments, a universal antibody binding moiety is or comprises optionally substituted A-5. In some embodiments, a universal antibody binding moiety is or comprises optionally substituted A-6. In some embodiments, a universal antibody binding moiety is or comprises optionally substituted A-7. In some embodiments, a universal antibody binding moiety is or comprises optionally substituted A-8. In some embodiments, a universal antibody binding moiety is or comprises optionally substituted A-9. In some embodiments, a universal antibody binding moiety is or comprises optionally substituted A-10. In some embodiments, a universal antibody binding moiety is or comprises optionally substituted A-11. In some embodiments, a universal antibody binding moiety is or comprises optionally substituted A-12. In some embodiments, a universal antibody binding moiety is or comprises optionally substituted A-13. In some embodiments, a universal antibody binding moiety is or comprises optionally substituted A-14. In some embodiments, a universal antibody binding moiety is or comprises optionally substituted A-15. In some embodiments, a universal antibody binding moiety is or comprises optionally substituted A-16. In some embodiments, a universal antibody binding moiety is or comprises optionally substituted A-17. In some embodiments, a universal antibody binding moiety is or comprises optionally substituted A-18. In some embodiments, a universal antibody binding moiety is or comprises optionally substituted A-19. In some embodiments, a universal antibody binding moiety is or comprises optionally substituted A-20. In some embodiments, a universal antibody binding moiety is or comprises optionally substituted A-21. In some embodiments, a universal antibody binding moiety is or comprises optionally substituted A-22. In some embodiments, a universal antibody binding moiety is or comprises optionally substituted A-23. In some embodiments, a universal antibody binding moiety is or comprises optionally substituted A-24. In some embodiments, a universal antibody binding moiety is or comprises optionally substituted A-25. In some embodiments, a universal antibody binding moiety is or comprises optionally substituted A-26. In some embodiments, a universal antibody binding moiety is or comprises optionally substituted A-27. In some embodiments, a universal antibody binding moiety is or comprises optionally substituted A-28. In some embodiments, a universal antibody binding moiety is or comprises optionally substituted A-29. In some embodiments, a universal antibody binding moiety is or comprises optionally substituted A-30. In some embodiments, a universal antibody binding moiety is or comprises optionally substituted A-31. In some embodiments, a universal antibody binding moiety is or comprises optionally substituted A-32. In some embodiments, a universal antibody binding moiety is or comprises optionally substituted A-33. In some embodiments, a universal antibody binding moiety is or comprises optionally substituted A-34. In some embodiments, a universal antibody binding moiety is or comprises optionally substituted A-35. In some embodiments, a universal antibody binding moiety is or comprises optionally substituted A-36. In some embodiments, a universal antibody binding moiety is or comprises optionally substituted A-37. In some embodiments, a universal antibody binding moiety is or comprises optionally substituted A-38. In some embodiments, a universal antibody binding moiety is or comprises optionally substituted A-39. In some embodiments, a universal antibody binding moiety is or comprises optionally substituted A-40. In some embodiments, a universal antibody binding moiety is or comprises optionally substituted A-41. In some embodiments, a universal antibody binding moiety is or comprises optionally substituted A-42. In some embodiments, a universal antibody binding moiety is or comprises optionally substituted A-43. In some embodiments, a universal antibody binding moiety is or comprises optionally substituted A-44. In some embodiments, a universal antibody binding moiety is or comprises optionally substituted A-45. In some embodiments, a universal antibody binding moiety is or comprises optionally substituted A-46. In some embodiments, a universal antibody binding moiety is or comprises optionally substituted A-47. In some embodiments, a universal antibody binding moiety is or comprises optionally substituted A-48. In some embodiments, a universal antibody binding moiety is or comprises optionally substituted A-49. In some embodiments, a universal antibody binding moiety is or comprises optionally substituted A-50.

In some embodiments, a universal antibody binding moiety is A-1. In some embodiments, a universal antibody binding moiety is A-2. In some embodiments, a universal antibody binding moiety is A-3. In some embodiments, a universal antibody binding moiety is A-4. In some embodiments, a universal antibody binding moiety is A-5. In some embodiments, a universal antibody binding moiety is A-6. In some embodiments, a universal antibody binding moiety is A-7. In some embodiments, a universal antibody binding moiety is A-8. In some embodiments, a universal antibody binding moiety is A-9. In some embodiments, a universal antibody binding moiety is A-10. In some embodiments, a universal antibody binding moiety is A-11. In some embodiments, a universal antibody binding moiety is A-12. In some embodiments, a universal antibody binding moiety is A-13. In some embodiments, a universal antibody binding moiety is A-14. In some embodiments, a universal antibody binding moiety is A-15. In some embodiments, a universal antibody binding moiety is A-16. In some embodiments, a universal antibody binding moiety is A-17. In some embodiments, a universal antibody binding moiety is A-18. In some embodiments, a universal antibody binding moiety is A-19. In some embodiments, a universal antibody binding moiety is A-20. In some embodiments, a universal antibody binding moiety is A-21. In some embodiments, a universal antibody binding moiety is A-22. In some embodiments, a universal antibody binding moiety is A-23. In some embodiments, a universal antibody binding moiety is A-24. In some embodiments, a universal antibody binding moiety is A-25. In some embodiments, a universal antibody binding moiety is A-26. In some embodiments, a universal antibody binding moiety is A-27. In some embodiments, a universal antibody binding moiety is A-28. In some embodiments, a universal antibody binding moiety is A-29. In some embodiments, a universal antibody binding moiety is A-30. In some embodiments, a universal antibody binding moiety is A-31. In some embodiments, a universal antibody binding moiety is A-32. In some embodiments, a universal antibody binding moiety is A-33. In some embodiments, a universal antibody binding moiety is A-34. In some embodiments, a universal antibody binding moiety is A-35. In some embodiments, a universal antibody binding moiety is A-36. In some embodiments, a universal antibody binding moiety is A-37. In some embodiments, a universal antibody binding moiety is A-38. In some embodiments, a universal antibody binding moiety is A-39. In some embodiments, a universal antibody binding moiety is A-40. In some embodiments, a universal antibody binding moiety is A-41. In some embodiments, a universal antibody binding moiety is A-42. In some embodiments, a universal antibody binding moiety is A-43. In some embodiments, a universal antibody binding moiety is A-44. In some embodiments, a universal antibody binding moiety is A-45. In some embodiments, a universal antibody binding moiety is A-46. In some embodiments, a universal antibody binding moiety is A-47. In some embodiments, a universal antibody binding moiety is A-48. In some embodiments, a universal antibody binding moiety is A-49. In some embodiments, a universal antibody binding moiety is A-50.

In some embodiments, a universal antibody binding moiety comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, a universal antibody binding moiety comprises a peptide unit, and is connected to a target binding moiety optionally through a linker moiety through the C-terminus of the peptide unit. In some embodiments, a universal antibody binding moiety comprises a peptide unit, and is connected to a target binding moiety optionally through a linker moiety through the N-terminus of the peptide unit. In some embodiments, a universal antibody binding moiety comprises a peptide unit, and is connected to a target binding moiety optionally through a linker moiety through a side chain of the peptide unit.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in US 9,745,339, US 201/30131321, etc. In some embodiments, an antibody binding moiety is of such a structure that its corresponding compound is a compound described in US 9,745,339 or US 2013/0131321, the compounds of each of which are independently incorporated herein by reference. In some embodiments, ABT is of such a structure that H-ABT is a compound described in US 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, an antibody binding moiety, e.g., an ABT is or comprises optionally substituted

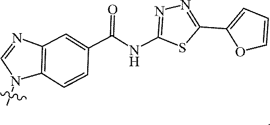

In some embodiments, an ABT is or comprises

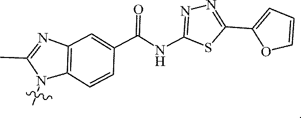

In some embodiments, an ABT is or comprises optionally substituted

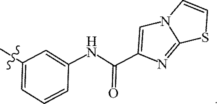

In some embodiments, an ABT is or comprises

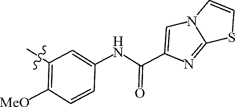

In some embodiments, an ABT is or comprises optionally substituted

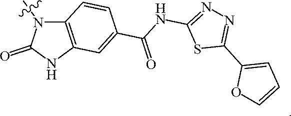

Insome embodiments, an ABT is or comprises

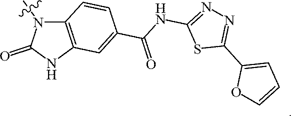

In some embodiments, an ABT is or comprises optionally substituted

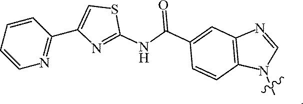

In some embodiments, an ABT is or comprises

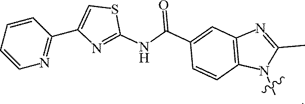

In some embodiments, an antibody binding moiety is a triazine moiety, e.g., one described in US 2009/0286693. In some embodiments, an antibody binding moiety is of such a structure that its corresponding compound is a compound described in US 2009/0286693, the compounds of which are independently incorporated herein by reference. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, an antibody binding moiety is a triazine moiety, e.g., one described in Teng, et al., A strategy for the generation of biomimetic ligands for affinity chromatography. Combinatorial synthesis and biological evaluation of an IgG binding ligand, J. Mol. Recognit. 1999;12:67-75 (“Teng”). In some embodiments, an antibody binding moiety is of such a structure that its corresponding compound is a compound described in Teng, the compounds of which are independently incorporated herein by reference. In some embodiments, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, an antibody binding moiety is a triazine moiety, e.g., one described in Uttamchandani, et al., Microarrays of Tagged Combinatorial Triazine Libraries in the Discovery of Small-Molecule Ligands of Human IgG, J. Comb. Chem. 2004 Nov-Dec;6(6): 862-8 (“Uttamchandani”). In some embodiments, an antibody binding moiety is of such a structure that its corresponding compound is a compound described in Uttamchandani, the compounds of which are independently incorporated herein by reference. In some embodiments, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG. In some embodiments, an antibody binding moiety is described in Choe, W., Durgannavar, T. A., & Chung, S. J. (2016). Fc-binding ligands of immunoglobulin G: An overview of high affinity proteins and peptides. Materials, 9(12). https://doi.org/10.3390/ma9120994.

In some embodiments, an antibody binding moiety can bind to a nucleotide-binding site. In some embodiments, an antibody binding moiety is a small molecule moiety that can bind to a nucleotide-binding site. In some embodiments, a small molecule is tryptamine. In some embodiments, ABT is of such a structure that H-ABT is tryptamine. Certain useful technologies were described in Mustafaoglu, et al., Antibody Purification via Affinity Membrane Chromatography Method Utilizing Nucleotide Binding Site Targeting With A Small Molecule, Analyst. 2016 November 28; 141(24): 6571-6582.

Many technologies are available for identifying and/or assessing and/or characterizing antibody binding moieties, including universal antibody binding moieties, and/or their utilization in ARMs, e.g., those described in WO/2019/023501, the technologies of which are incorporated herein by reference. In some embodiments, an antibody binding moiety is a moiety (e.g., small molecule moiety, peptide moiety, nucleic acid moiety, etc.) that can selectively bind to IgG, and when used in an ARM can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be recruited by antibody binding moieties described in the present disclosure. In some embodiments, such antibodies include antibodies administered to a subject, e.g., for therapeutic purposes. In some embodiments, antibodies recruited by antibody binding moieties comprise antibodies toward different antigens. In some embodiments, antibodies recruited by antibody binding moieties comprise antibodies whose antigens are not present on the surface or cell membrane of target cells (e.g., target cells such as cells infected by SARS-CoV-2). In some embodiments, antibodies recruited by antibody binding moieties comprise antibodies which are not targeting antigens present on surface or cell membrane of targets (e.g., target cells such as cells infected by SARS-CoV-2). In some embodiments, antigens on surface of target cells may interfere with the structure, conformation, and/or one or more properties and/or activities of recruited antibodies which bind such antigens. In some embodiments, as appreciated by those skilled in the art, provided technologies comprise universal antibody binding moieties which recruit antibodies of diverse specificities, and no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of recruited antibodies are toward the same antigen, protein, lipid, carbohydrate, etc. Among other things, one advantage of the present disclosure is that provided technologies comprising universal antibody binding moieties can utilize diverse pools of antibodies such as those present in serum. In some embodiments, universal antibody binding moieties of the present disclosure (e.g., those in ARMs) are contacted with a plurality of antibodies, wherein no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of the plurality of antibodies are toward the same antigen, protein, lipid, carbohydrate, etc. In some embodiments, recruited antibodies are those in IVIG. In some embodiments, IVIG may be administered prior to, concurrently with or subsequently to an agent or composition. Among other things, antibodies of various types of immunoglobulin structures may be recruited. In some embodiments, one or more subclasses of IgG are recruited. In some embodiments, recruited antibodies comprise IgG1. In some embodiments, recruited antibodies comprise IgG2. In some embodiments, recruited antibodies comprise IgG3. In some embodiments, recruited antibodies comprise IgG4. In some embodiments, recruited antibodies are or comprise IgG1 and IgG2. In some embodiments, recruited antibodies are or comprise IgG1, IgG2 and IgG4. In some embodiments, recruited antibodies are or comprise IgG1, IgG2, IgG3 and IgG4. Recruited antibodies may interact various types of receptors, e.g., those expressed by various types of immune cells. In some embodiments, recruited antibodies can effectively interact various types of Fc receptors and provide desired immune activities. In some embodiments, recruited antibodies can recruit immune cells. In some embodiments, recruited antibodies can effectively interact with hFcyRIIIA. In some embodiments, recruited antibodies can effectively interact with hFcyRIIIA on macrophages. In some embodiments, macrophages are recruited to provide ADCC and/or ADCP activities toward a virus, e.g., a SARS-CoV-2 virus, and/or cells infected thereby. In some embodiments, NK cells are recruited to provide immune activities. In some embodiments, recruited antibodies can effectively interact with hFcyRIIA. In some embodiments, recruited antibodies can effectively interact with hFcyRIIA on dendritic cells. In some embodiments, antibody moieties in agents of the present disclosure comprise one or more properties, structures and/or activities of recruited antibodies described herein.

### SARS-CoV-2

It is reported that SARS-CoV-2 may belong to lineage B betacoronavirus and can cause severe respiratory problems. Coughing, fever, difficulties in breathing and/or shortage of breath are reported to be among the common symptoms. Infection by SARS-CoV-2 is reported to lead to COVID-19. SARS-CoV-2 has caused a large number of confirmed cases and deaths globally.

Reportedly, SARS-CoV-2 can utilize human angiotensin-converting enzyme 2 (ACE2) as a receptor to infect human cells. There have been reports that SARS-CoV-2 spike (S) protein S2 subunit plays an important role in mediating virus fusion with and entry into the host cell, in which a heptad repeat 1 (HR1) and heptad repeat 2 (HR2) can interact to form six-helical bundle (6-HB), in some cases, reportedly bringing viral and cellular membranes in close proximity for fusion.

Genetic variations have been reported for SARS-CoV-2. In some embodiments, provided technologies can target one or more or all SARS-CoV-2 variants (e.g., by targeting specific or universal elements).

### Target Binding, Moieties

In some embodiments, the present disclosure provides agents that can bind to a SARS-CoV-2 virus or a cell infected thereby. Among other things, agents of the present disclosure comprise target binding moieties that can bind to a SARS-CoV-2 spike protein or a fragment thereof. In some embodiments, the present disclosure provides agents that can bind to a SARS-CoV-2 spike protein or a fragment thereof. In some embodiments, target binding moieties are or comprise peptide moieties.

In some embodiments, a target binding moiety is or comprises a peptide agent. In some embodiments, a target binding moiety is a peptide moiety. In some embodiments, a peptide moiety can either be linier or cyclic. In some embodiments, a target binding moiety is or comprises a peptide moiety comprising a cyclic structure.

In some embodiments, a provided agent has the structure of R^{CN}-(Xaa)y-R^{CC} or a salt thereof. In some embodiments, a provided target binding moiety is a moiety of R^{CN}-(Xaa)y-R^{CC} or a salt thereof (e.g., removing one or more —H to form a monovalent, bivalent or polyvalent moiety). In some embodiments, a target binding moiety is or comprises -(Xaa)y- as described herein. In some embodiments, as described herein a target binding moiety may be connected to the rest of the molecule, an antibody moiety, or an antibody binding moiety through a N-terminus, C-terminus or middle residue. In some embodiments, -(Xaa)y- is or comprises: -(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xa a^{T10})y10-(Xaa^{T11})y11-(Xaa^{T12})y12-, or a salt form thereof.

In some embodiments, each of Xaa^{T0}, Xaa^{T1}, Xaa^{T2}, Xaa^{T3}, Xaa^{T4}, Xaa^{T5}, Xaa^{T6}, Xaa^{T7}, Xaa^{T8}, Xaa^{T9}, Xaa^{T10}, Xaa^{T11}, and Xaa^{T12} is independently a residue of an amino acid having the structure of formula A-I or a salt thereof. In some embodiments, each of Xaa^{T0}, Xaa^{T1}, Xaa^{T2}, Xaa^{T3}, Xaa^{T4}, Xaa^{T5}, Xaa^{T6}, Xaa^{T7}, Xaa^{T8}, Xaa^{T9}, Xaa^{T10}, Xaa^{T11}, and Xaa^{T12} is independently —N(R^{a1})—L^{a1}—C(R^{a2})(R^{a3})—L^{a2}—CO— or a salt thereof.

In some embodiments, y0 is 0. In some embodiments, y0 is 1-20. In some embodiments, y0 is 1, 2, 3, 4, 5, 6, 7, 8, 9, 10, 11, 12, 13, 14, 15, 16, 17, 18, 19 or 20.

In some embodiments, y1 is 0. In some embodiments, y1 is 1. In some embodiments, y1 is 2. In some embodiments, when y1 is 0, a moiety, e.g., a target binding moiety, comprising -(Xaa)y-, is not connected to another moiety through its N-terminus.

In some embodiments, -(Xaa^{T1})y1- is or comprises a dipeptide residue or an amino acid residue that is suitable for forming a turn. Various suitable structures are available and can be utilized in accordance with the present disclosure.

In some embodiments, -(Xaa^{T1})y1- is or comprises a residue of L-proline, D-proline, a proline derivative, L-serine, D-serine, glycine, L-pseudoproline, or D-psuedoproline.

In some embodiments, -(Xaa^{T1})y1- is or comprises a residue of an amino acid having the structure of formula A-I or a salt thereof. In some embodiments, -(Xaa^{T1})y1- is or comprises a residue of an amino acid having the structure of —N(R^{a1})—L^{a1}—C(R^{a2})(R^{a3})—L^{a2}—CO— or a salt thereof. In some embodiments, y1 is 1.

In some embodiments, R^{a1} and R^{a2} are taken together with their intervening atoms to form an optionally substituted, 3-10 membered, monocyclic, bicyclic or polycyclic ring having, in addition to the intervening atoms, 0-5 heteroatoms independently selected from oxygen, nitrogen, sulfur, phosphorus and silicon. In some embodiments, R^{a1} and R^{a2} are taken together with their intervening atoms to form an optionally substituted, 3-6 membered, monocyclic, bicyclic or polycyclic ring having, in addition to the intervening atoms, 0-1 heteroatoms independently selected from oxygen, nitrogen, and sulfur. In some embodiments, a formed ring is monocyclic. In some embodiments, a formed ring is bicyclic. In some embodiments, a formed ring is polycyclic. In some embodiments, a formed ring is saturated. In some embodiments, a formed ring is partially unsaturated. In some embodiments, a formed ring is substituted. In some embodiments, a formed ring is unsubstituted. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered.

In some embodiments, L^{a1} is a covalent bond. In some embodiments, L^{a2} is a covalent bond.

In some embodiments, R^{a3} is —H. In some embodiments, R^{a3} is optionally substituted C₁₋₄ aliphatic. In some embodiments, R^{a3} is methyl. In some embodiments, R^{a3} is substituted methyl. In some embodiments, R^{a3} is benzyl. In some embodiments, R^{a2} and R^{a3} are bonded is of S configuration. In some embodiments, wherein the carbon to which R^{a2} and R^{a3} are bonded is of R configuration.

In some embodiments, -(Xaa^{T1})y1- is or comprises a L-proline residue. In some embodiments, -(Xaa^{T1})y1- is or comprises a residue of

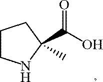

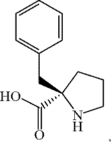

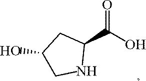

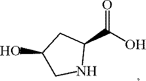

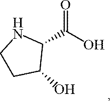

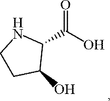

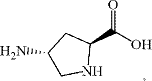

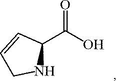

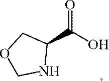

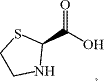

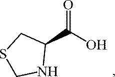

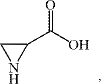

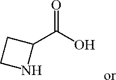

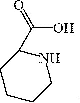

In some embodiments, -(Xaa^{T1})y1- is connected to the rest of a molecule through its N-end and optionally through a linker. In some embodiments, -(Xaa^{T1})y1- is connected to an antibody moiety or an antibody binding moiety through its N-end and optionally through a linker.

In some embodiments, -Xaa^{T2}- comprises a hydrophobic, neutral or negatively charged (e.g., at physiological pH, around 7, etc.) side chain. In some embodiments, -Xaa^{T2}- is or comprises a residue of an amino acid having the structure of formula A-I or a salt thereof, wherein R^{a2} is hydrophobic, neutral or negatively charged. In some embodiments, -Xaa^{T2}- has the structure of —N(Ra¹)—L^{a1}—C(R^{a2})(R^{a3})—L^{a2}—CO— or a salt form thereof, wherein R^{a2} is hydrophobic, neutral or negatively charged. In some embodiments, Xaa^{T2} comprises a hydrophobic side chain. In some embodiments, a hydrophobic side chain is of sufficient volume to interact with a pocket.

In some embodiments, R^{a2} is —L^{a}—R′, wherein L^{a} is an optionally substituted bivalent group selected from C₃-C₁₀ aliphatic or C₃-C₁₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L^{a} is an optionally substituted bivalent group selected from C₃-C₁₀ aliphatic or C₃-C₁₀ heteroaliphatic having 1-5 heteroatoms, wherein one or more methylene units of the group are optionally and independently replaced with —C(R′)₂—, —O—, —S—, —N(R′)—, —C(O)—, —C(S)—, —C(NR′)—, —S(O)—, or —S(O)₂—. In some embodiments, L^{a} is an optionally substituted bivalent C₃ alkylene group wherein one or more methylene units are optionally and independently replaced with —O— or —S—. In some embodiments, L^{a} is —CH₂—CH₂—CH₂—, —CH₂—O—CH₂—, or —CH₂—S—CH₂—. In some embodiments, L^{a} is —CH₂—O—CH₂—.

In some embodiments, R′ is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is phenyl. In some embodiments, R′ is substituted phenyl, wherein each substituent is independently selected from —OH, halogen, and C₁₋₄ optionally substituted with one or more halogen or —OH.

In some embodiments, wherein R^{a1} is —H. In some embodiments, R^{a1} is optionally substituted C₁₋₄ aliphatic. In some embodiments, R^{a1} is optionally substituted C₁₋₄ alkyl.

In some embodiments, L^{a1} is a covalent bond.

In some embodiments, R^{a3} is —H. In some embodiments, R^{a3} is optionally substituted C₁₋₄ aliphatic. In some embodiments, R^{a3} is optionally substituted C₁₋₄ alkyl.

In some embodiments, a carbon to which R^{a2} and R^{a3} are bonded is of S configuration. In some embodiments, a carbon to which R^{a2} and R^{a3} are bonded is of R configuration.

In some embodiments, L^{a2} is a covalent bond.

In some embodiments, -Xaa^{T2}- is a residue of Leu, Ile, Phe, Tyr, Trp, Arg, or Citruline. In some embodiments, -Xaa^{T2}- is

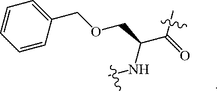

In some embodiments, -Xaa^{T2}- is

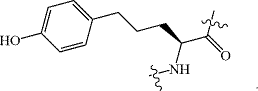

In some embodiments, y3 is 0. In some embodiments, y3 is 1-10. In some embodiments, y3 is 1, 2, 3, 4, 5, 6, 7, 8, 9, or 10. In some embodiments, y3 is 1. In some embodiments, y3 is 2. In some embodiments, y3 is 3. In some embodiments, y3 is 4. In some embodiments, y3 is 5. In some embodiments, y3 is 6. In some embodiments, y3 is 7. In some embodiments, y3 is 8. In some embodiments, y3 is 9. In some embodiments, y3 is 10.

In some embodiments, -(Xaa^{T3})y3- is or comprises TF. In some embodiments, -(Xaa^{T3})y3-is or comprises TFLL. In some embodiments, -(Xaa^{T3})y3- is or comprises TFLLKY.

As described herein, each of Xaa^{T4} and Xaa^{T9} is independently a residue of an amino acid or an amino acid analog, wherein Xaa^{T4} is optionally connected to Xaa^{T9} through a linker. In some embodiments, a linker is L^{a} and is bonded to a backbone atom of Xaa^{T4} and a backbone atom of Xaa^{T9}. In some embodiments, a linker is L^{a} and is bonded to a backbone carbon atom of Xaa^{T4} and a backbone carbon atom of Xaa^{T9}. In some embodiments, a linker is L^{a} and is bonded to an alpha-carbon atom of Xaa^{T4} and an alpha-carbon atom of Xaa^{T9}. In some embodiments, side chains of Xaa^{T4} and Xaa^{T9} are covalently connected through L^{a}. In some embodiments, L^{a} is or comprises —CH₂—CH₂—, —O—, —S— or —S—S—. In some embodiments, L^{a} is or comprises —CH₂—CH₂—. In some embodiments, L^{a} is or comprises —O—. In some embodiments, L^{a} is or comprises —S—. In some embodiments, L^{a} is or comprises —S—S—.

In some embodiments, Xaa^{T4} is Cys. In some embodiments, Xaa^{T9} is Cys.

In some embodiments, y5 is 0. In some embodiments, y5 is 1-10. In some embodiments, y5 is 1, 2, 3, 4, 5, 6, 7, 8, 9, or 10. In some embodiments, y5 is 1. In some embodiments, y5 is 2. In some embodiments, y5 is 3. In some embodiments, y5 is 4. In some embodiments, y5 is 5. In some embodiments, y5 is 6. In some embodiments, y5 is 7. In some embodiments, y5 is 8. In some embodiments, y5 is 9. In some embodiments, y5 is 10.

In some embodiments, -(Xaa^{T5})y5- is or comprises LKY. In some embodiments, -(Xaa^{T5})y5-is or comprises -LKYXaa^{T5}-. In some embodiments, -(Xaa^{T5})y5- is or comprises LKYN. In some embodiments, -(Xaa^{T5})y5- is or comprises YNK.

In some embodiments, -(Xaa^{T5})y5- is connected to the rest of a molecule optionally through a linker. In some embodiments, -(Xaa^{T5})y5- is connected to an antibody moiety or an antibody binding moiety optionally through a linker. In some embodiments, -(Xaa^{T5})y5- is connected to the rest of the molecule through a linker that is bound to the the Xaa^{T5} that is bonded to -(Xaa^{T6})y6-. In some embodiments, -(Xaa^{T5})y5- is connected to the rest of a molecule or an antibody moiety or an antibody binding moiety optionally through a linker through a side chain of a Xaa^{T5}. In some embodiments, -(Xaa^{T5})y5- is connected to the rest of a molecule or an antibody moiety or an antibody binding moiety optionally through a linker through a side change of a lysine residue.

In some embodiments, y6 is 0. In some embodiments, y6 is 1. In some embodiments, y6 is 2.

In some embodiments, -(Xaa^{T6})y6- is or comprises a dipeptide residue or an amino acid residue that is suitable for forming a turn, e.g., those described for -(Xaa^{T1})y1-. In some embodiments, -(Xaa^{T6})y6- is or comprises a residue of L-proline, D-proline, a proline derivative, L-serine, D-serine, glycine, L-pseudoproline, or D-psuedoproline. In some embodiments, -(Xaa^{T6})y6- is or comprises a residue of an amino acid having the structure of formula A-I or a salt thereof, wherein R^{a1} and R^{a2} are taken together with their intervening atoms to form an optionally substituted, 3-10 membered, monocyclic, bicyclic or polycyclic ring having, in addition to the intervening atoms, 0-5 heteroatoms independently selected from oxygen, nitrogen, sulfur, phosphorus and silicon. In some embodiments, -(Xaa^{T6})y6- is or comprises a residue having the structure of —N(R^{a1})—L^{a1}—C(R^{a2})(R^{a3})—L^{a2}—CO— or a salt from thereof, wherein R^{a1} and R^{a2} are taken together with their intervening atoms to form an optionally substituted, 3-10 membered, monocyclic, bicyclic or polycyclic ring having, in addition to the intervening atoms, 0-5 heteroatoms independently selected from oxygen, nitrogen, sulfur, phosphorus and silicon. In some embodiments, R^{a1} and R^{a2} are taken together with their intervening atoms to form an optionally substituted, 3-6 membered, monocyclic, bicyclic or polycyclic ring having, in addition to the intervening atoms, 0-1 heteroatoms independently selected from oxygen, nitrogen, and sulfur.

In some embodiments, a formed ring is monocyclic. In some embodiments, a formed ring is bicyclic. In some embodiments, a formed ring is polycyclic. In some embodiments, a formed ring is saturated. In some embodiments, a formed ring is partially unsaturated. In some embodiments, a formed ring is substituted. In some embodiments, a formed ring is unsubstituted. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered.

In some embodiments, L^{a1} is a covalent bond. In some embodiments, L^{a2} is a covalent bond.

In some embodiments, R^{a3} is —H. In some embodiments, R^{a3} is optionally substituted C₁₋₄ aliphatic. In some embodiments, R^{a3} is methyl. In some embodiments, R^{a3} is substituted methyl. In some embodiments, R^{a3} is benzyl. In some embodiments, R^{a2} and R^{a3} are bonded is of S configuration. In some embodiments, wherein the carbon to which R^{a2} and R^{a3} are bonded is of R configuration.

In some embodiments, -(Xaa^{T6})y6- is or comprises a D-Ser residue.

In some embodiments, -(Xaa^{T6})y6- is or comprises a residue of

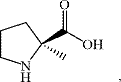

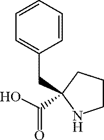 [SMILES: O=C(O)[C@]1(Cc2ccccc2)CCCN1]

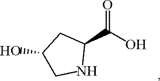

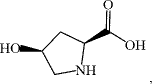

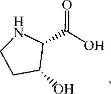

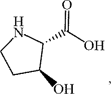

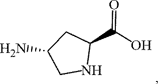

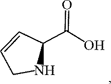

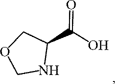

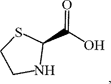

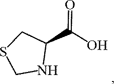

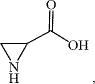

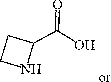

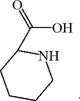

In some embodiments, -(Xaa^{T6})y6- is or comprises a residue that comprises or is further substituted with a negatively charged group (e.g., at physiological pH, around 7, etc.). In some embodiments, a negatively charged group is or comprises —COOH. In some embodiments, is or comprises a residue that comprises or is further substituted with —L^{a}—COOH. In some embodiments, —L^{a}—COOH is part of a side chain or a substituent of a ring (e.g., the ring in a proline residue or an analog thereof).

In some embodiments, L^{a} is an optionally substituted bivalent group selected from C₁-C₁₀ aliphatic or C₁-C₁₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is —O—CH₂—.

In some embodiments, -(Xaa^{T6})y6- is or comprises a residue having the structure of

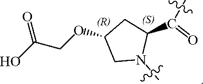

In some embodiments, when -(Xaa^{T6})y6- comprises a negatively charged group, y7 is 0.

In some embodiments, y7 is 0. In some embodiments, y7 is 1.

As described herein, Xaa^{T7} is a negatively-charged residue of an amino acid or an amino acid analog. In some embodiments, Xaa^{T7} comprises —COOH. In some embodiments, Xaa^{T7} is D or E. In some embodiments, it is D. In some embodiments, it is E.

In some embodiments, y8 is 0. In some embodiments, y8 is 1-10. In some embodiments, y8 is 1, 2, 3, 4, 5, 6, 7, 8, 9, or 10. In some embodiments, y8 is 1. In some embodiments, y8 is 2. In some embodiments, y8 is 3. In some embodiments, y8 is 4. In some embodiments, y8 is 5. In some embodiments, y8 is 6. In some embodiments, y8 is 7. In some embodiments, y8 is 8. In some embodiments, y8 is 9. In some embodiments, y8 is 10.

In some embodiments, -(Xaa^{T8})y8- is or comprises GTI. In some embodiments, -(Xaa^{T8})y8-is or comprises GTI-Xaa^{T8}-. In some embodiments, -(Xaa^{T8})y8- is or comprises GTI-Xaa^{T8}-DA. In some embodiments, -(Xaa^{T8})y8- is or comprises G-Xaa^{T8}-IT- Xaa^{T8}-. In some embodiments, -(Xaa^{T8})y8- is or comprises -G-Xaa^{T8}-IT-Xaa^{T8}-, wherein each Xaa^{T8} is independently an alpha amino acid residue. In some embodiments, -(Xaa^{T8})y8- is or comprises GTITDA.

In some embodiments, Xaa^{T9} is Cys. In some embodiments, Xaa^{T4} and Xaa^{T9} are independently Cys and form a disulfide bond —S—S—.

In some embodiments, y10 is 0. In some embodiments, y10 is 1-10. In some embodiments, y10 is 1, 2, 3, 4, 5, 6, 7, 8, 9, or 10. In some embodiments, y10 is 1. In some embodiments, y10 is 2. In some embodiments, y10 is 3. In some embodiments, y10 is 4. In some embodiments, y10 is 5. In some embodiments, y10 is 6. In some embodiments, y10 is 7. In some embodiments, y10 is 8. In some embodiments, y10 is 9. In some embodiments, y10 is 10.

In some embodiments, -(Xaa^{T10})ylO- is or comprises DAV. In some embodiments, ^{_}(Xaa^{T10})y10- is or comprises A. In some embodiments, -(Xaa^{T2})y2- is or comprises AVAD.

In some embodiments, y1 1 is 1. In some embodiments, y1 1 is 2. In some embodiments, y1 1 is 3. In some embodiments, y11 is 4. In some embodiments, y11 is 5.

In some embodiments, -(Xaa^{T11})y11- is or comprises a hydrophobic or negatively charged residue. In some embodiments, -(Xaa^{T11})y11- is or comprises L-Ala, D-Ala, Aib, Gly, or negatively charged residue. In some embodiments, -(Xaa^{T11})y11- is or comprises a hydrophobic residue. In some embodiments, -(Xaa^{T11})y11- is or comprises L-Aib. In some embodiments, -(Xaa^{T11})y11- is Aib. In some embodiments, -(Xaa^{T11})y11- is or comprises —Ala—Aib-. In some embodiments, -(Xaa^{T11})y11- is —Ala—Aib—.

In some embodiments, y12 is 0. In some embodiments, y12 is 1, 2, 3, 4, 5, 6, 7, 8, 9, 10, 11, 12, 13, 14, 15, 16, 17, 18, 19, or 20.

In some embodiment (Xaa)1 is or comprises YAibYY. In some embodiments (Xaa)2 is Glutamine and forms a cyclic peptide with (Xaa)4 which is a Lysine. In some embodiment (Xaa)7 is Lys which forms a cyclic peptide with (Xaa)10 which is a Glutamine.

In some embodiments, a moiety, e.g., a target binding moiety comprising -(Xaa)y is bonded to the rest of a molecule, an antibody moiety or an antibody binding moiety through its C-terminus, optionally through a linker. In some embodiments, -(Xaa^{T11})y11- or -(Xaa^{T12})y12- is bonded to the rest of a molecule, an antibody moiety or an antibody binding moiety, optionally through a linker.

In some embodiments, an agent, a target binding moiety, or -(Xaa)y- is or comprises a sequence selected from, or a sequence designed based on a sequence selected from:

| | |
|---|---|
| Number | Sequence |
| P1 | FKLPLGIN(K)ITNFRAILTAFS(L)\| |
| P2 | PTT(K)FMLKYDENGTITDAVDC |
| P3 | VLYNSTP(S)FSTFKCYGVSATK |
| P4 | PALNCYWPLN(K)DYGFYTTSG1 |
| P5 | RDVSDP(I)TDSVRDPKTSEILD |
| P6 | YQDVNCTDVS(P)TAIHADQLTP |
| P7 | SNNTIAIPTNFS(L)ISITTEVM |
| P8 | QYGSFCT(A)QLNRALSGIAA(V)EQ |
| P9 | GIGVT(A)QNVLYENQKQIANQF |
| P10 | IQK(E)EIDRLNEVAKNLNESU |

Those skilled in the art reading the present disclosure will appreciate that target binding moieties for a protein of similar structure as a SARS-CoV-2 spike protein, e.g., a corresponding protein of SARS-CoV (may be referred to as SARS-Cov-1), may be utilized in accordance with the present disclosure, either directly or with further modifications.

In some embodiments, a an agent, a target binding moiety, or -(Xaa)y- is or comprises, or is or comprises a sequence designed based on, ACE2 or a fragment thereof (e.g., aa24-45 or a fragment thereof) or a corresponding sequence.

In some embodiments, an agent, a target binding moiety, or -(Xaa)y- is or comprises, or is or comprises a sequence designed based on, aa24-45, or a fragment thereof, of ACE2 or a corresponding sequence.

In some embodiments, an agent, a target binding moiety, or -(Xaa)y- is or comprises, or is or comprises a sequence designed based on, IEEQAKTFLDKFNHEAEDLFYQS or a fragment thereof. In some embodiments, -(Xaa)y- is or comprises IEEQAKTFLDKFNHEAEDLFYQS.

In some embodiments, an agent, a target binding moiety, or -(Xaa)y- is or comprises, or is or comprises a sequence designed based on, an HR domain of a spike protein of SARS-CoV-2. In some embodiments, an agent, a target binding moiety, or -(Xaa)y- is or comprises, or is or comprises a sequence designed based on, an HR1 domain of a spike protein of SARS-CoV-2. In some embodiments, an agent, a target binding moiety, or -(Xaa)y- is or comprises, or is or comprises a sequence designed based on, an HR2 domain of a spike protein of SARS-CoV-2. In some embodiments, an agent, a target binding moiety, or -(Xaa)y- is or comprises, or is or comprises a sequence designed based on, DISGINASVVNIQKEIDRLNEVAKNLNESLIDLQEL, or a fragment thereof.

In some embodiments, an agent, a target binding moiety, or -(Xaa)y- binds to a spike protein. In some embodiments, an agent, a target binding moiety, or -(Xaa)y- binds to S1 domain of a spike protein. In some embodiments, an agent, a target binding moiety, or -(Xaa)y- binds to S2 domain of a spike protein. In some embodiments, an agent, a target binding moiety, or -(Xaa)y- binds to HR1 region of a S2 domain of a spike protein. In some embodiments, an agent, a target binding moiety, or -(Xaa)y- binds to HR2 region of a S2 domain of a spike protein. In some embodiments, an agent, a target binding moiety, or -(Xaa)y- binds to S½ domain of a spike protein.

In some embodiments, agents bind to spike proteins (e.g., at S1 and/or S2 domains), blocking viruses from binding ACE2 receptor and infecting human cells. In some embodiments, agents recruit immune cells to attack, inhibit, kill or remove viruses and/or virus-infected cells (e.g., macrophages, NK cells, etc.), in some embodiments, through interactions with FcyRII-III receptors. In some embodiments, agents recruit dendritic cells, and in some embodiments, induce, promote, encourage, enhance, or trigger an immune system to adapt to proteins. In some embodiments, long-term immunity is provided. In some embodiments, immune memory cells (e.g., T-cells and/or B-cells) are generated to instill long-term immunity. In some embodiments, agents recruit IgG1 and IgG2 (e.g., those in human blood stream). In some embodiments, agents recruit IgG1, IgG2 and IgG4 (e.g., those in human blood stream). In some embodiments, agents recruit IgG1, IgG2, IgG3 and IgG4 (e.g., those in human blood stream). In some embodiments, agents comprise IgG1 and IgG2 (e.g., in antibody moieties). In some embodiments, agents comprise IgG1, IgG2 and IgG4. In some embodiments, agents comprise IgG1, IgG2, IgG3 and IgG4.

In some embodiments, an agent, a target binding moiety, or -(Xaa)y- is or comprises a stapled peptide moiety wherein at least two amino acid residues are modified for stapling and stapled together. In some embodiments, a staple is a (i, i+7) staple, wherein i is the position of the first residue connected by the staple, and i+7 is the position of the second residue connected by the staple.

In some embodiments, a provide agent or a target binding moiety, e.g., of or comprising -(Xaa)y-, is selective for SARS-CoV-2 or a protein or a fragment thereof. In some embodiments, a provided agent or target binding moiety can target two or more types of virus, e.g., through interactions with proteins having similar sequences and/or structures. In some embodiments, provided agents and/or compositions thereof can effectively target two or more or all coronaviruses. In some embodiments, provided agents and/or target binding moieties can effectively target two or more or all coronaviruses that infect humans. In some embodiments, provided agents and/or compositions thereof can effectively target two or more or all coronaviruses that share similar sequences/structures of spike proteins or fragments thereof (e.g., portions outside of viruses, portions interacting with human receptors, portions involved in infection humans, etc.). In some embodiments, provided agents and/or target binding moieties target SARS-CoV. In some embodiments, provided agents and/or target binding moieties target MERS-CoV. In some embodiments, provided agents and/or target binding moieties can target SARS-CoV, SARS-CoV-2 and/or MERS-CoV. In some embodiments, provided agents and/or target binding moieties can target SARS-CoV and SARS-CoV-2. In some embodiments, provided agents and/or target binding moieties can target SARS-CoV, SARS-CoV-2 and MERS-CoV. Among other things, the present disclosure provides technologies for inducing, promoting, encouraging, enhancing, triggering, or generating an immune response toward one or two or all of SARS-CoV, SARS-CoV-2 and MERS-CoV. In some embodiments, an immune response is or comprises ADCC, ADCP and/or long-term immunity as described herein. In some embodiments, the present disclosure provides technologies for inhibiting, killing or removing SARS-CoV, SARS-CoV-2 and/or MERS-CoV viruses. In some embodiments, the present disclosure provides technologies for inhibiting, killing or removing cells infected by SARS-CoV, SARS-CoV-2 and/or MERS-CoV viruses. In some embodiments, the present disclosure provides technologies for preventing or treating conditions, disorders or diseases associated with SARS-CoV, SARS-CoV-2 and/or MERS-CoV. In some embodiments, the present disclosure provides technologies for preventing or treating conditions, disorders or diseases associated with SARS-CoV (e.g., severe acute respiratory syndrome). In some embodiments, the present disclosure provides technologies for preventing or treating conditions, disorders or diseases associated with SARS-CoV-2 (e.g., COVID-19). In some embodiments, the present disclosure provides technologies for preventing or treating conditions, disorders or diseases associated with MERS-CoV (e.g., Middle East respiratory syndrome). In some embodiments, the present disclosure provides a method for disrupting, reducing or preventing an infection by SARS-CoV, SARS-CoV-2 and/or MERS-CoV viruses. In some embodiments, provided technologies are useful for inducing, promoting, encouraging, enhancing, triggering, or generating an immune response toward, and/or for inhibiting, killing or removing, and/or for inhibiting, killing or removing cells infected by, and/or for preventing or treating conditions, disorders or diseases associated with, and/or for disrupting, reducing or preventing an infection by, SARS-CoV, SARS-CoV-2 and MERS-CoV viruses. In some embodiments, provided technologies comprise contacting viruses with an effective amount of an agent or composition as described herein. In some embodiments, provided technologies comprise administering to a subject susceptible to or suffering from viral infections and/or conditions, disorders or diseases associated with viral infections an effective amount of an agent or composition as described herein.

In some embodiments, R^{CN} is R—C(O)—. In some embodiments, R is optionally substituted C₁₋₆ aliphatic. In some embodiments, R is methyl.

In some embodiments, R^{CC} is —N(R′)₂. In some embodiments, R^{CC} is —NH₂.

In some embodiments, a provided agent, -(Xaa)y- and or -(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12- comprises one or more of -(Xaa^{T1})y1-, -Xaa^{T2}-, -(Xaa^{T6})y6-, and -(Xaa^{T11})y11-, each of which is independently as described herein. In some embodiments, it is or comprises -(Xaa^{T1})y1-. In some embodiments, it is or comprises -Xaa^{T2}-. In some embodiments, it is or comprises -(Xaa^{T6})y6-. In some embodiments, it is or comprises -(Xaa^{T11})y11-. In some embodiments, it is or comprises -(Xaa^{T1})y1- and -Xaa^{T2}-. In some embodiments, it is or comprises -Xaa^{T2}- and -(Xaa^{T6})y6-. In some embodiments, it is or comprises -(Xaa^{T1})y1- and -(Xaa^{T6})y6-. In some embodiments, it is or comprises -(Xaa^{T1})y1-, -Xaa^{T2}-, and -(Xaa^{T6})y6-. In some embodiments, it is or comprises -(Xaa^{T1})y1-, -Xaa^{T2}-, -(Xaa^{T6})y6-, and -(Xaa^{T11})y11-.

. In some embodiments, a provided agent, -(Xaa)y- and or -(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12- comprises

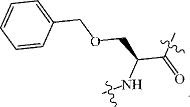

In some embodiments, a provided agent, -(Xaa)y- and or-(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12- comprises

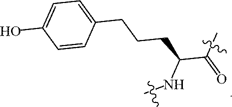

In some embodiments, a provided agent, -(Xaa)y- and or -(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12- comprises

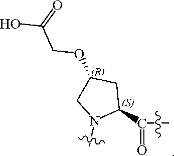

In some embodiments, it is or comprises

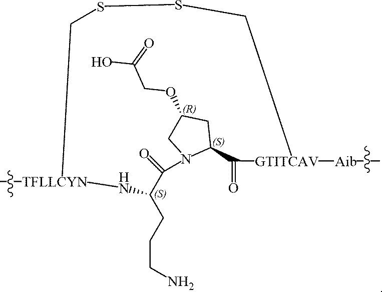

In some embodiments, it is or comprises

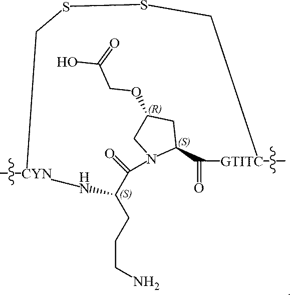

In some embodiments, it is orcomprises

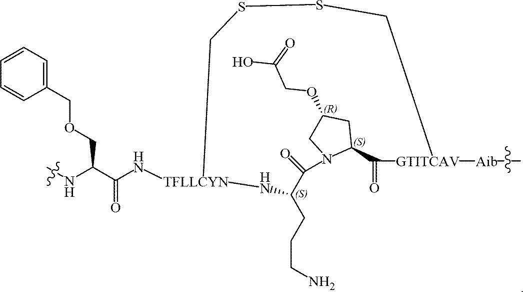

In some embodiments, a Lys residue is bonded to the rest of an agent, e.g., a linker. In some embodiments, it is or comprises D-Ser. In some embodiments, it is or comprises D-Ser-acidic amino acid residue-. In some embodiments, it is or comprises D-Ser-E. In some embodiments, it is or comprises

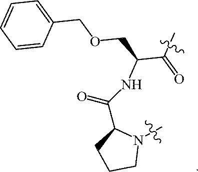

In some embodiments, it is or comprises

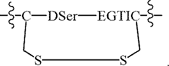

In some embodiments, it is or comprises

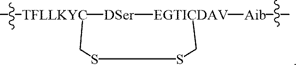

In some embodiments, it is or comprises

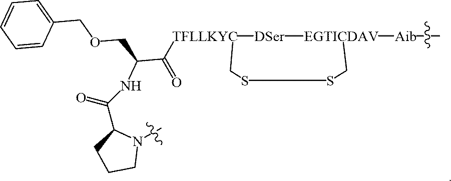

In some embodiments, it is or comprises

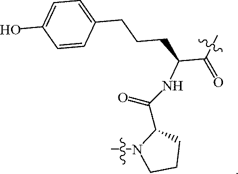

In some embodiments, it is or comprises

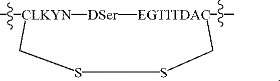

In some embodiments, it is or comprises

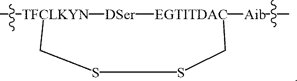

In some embodiments, it is or comprises

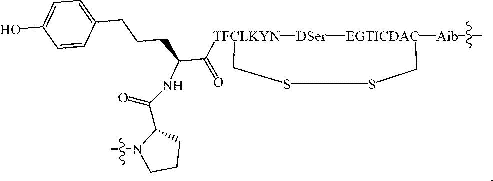

In some embodiments, a Pro residue is bonded to the rest of an agent, e.g., a linker. In some embodiments, it is or comprises

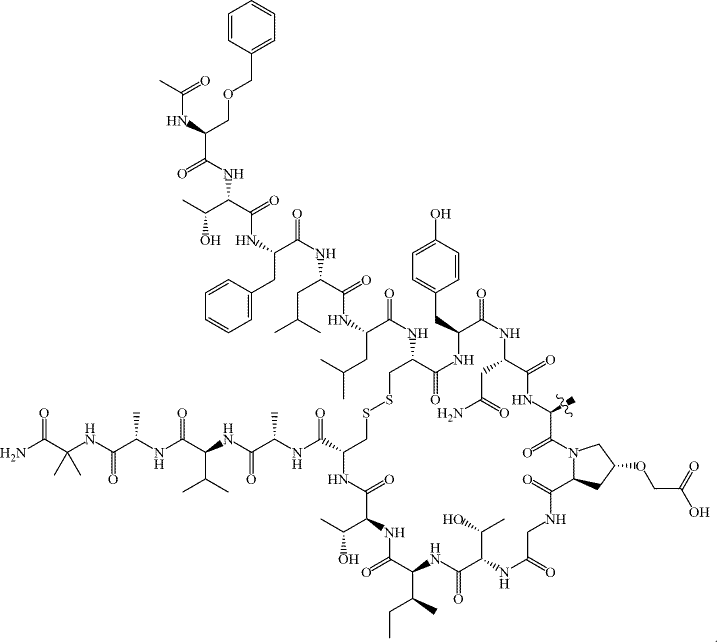

In some embodiments, it is orcomprises

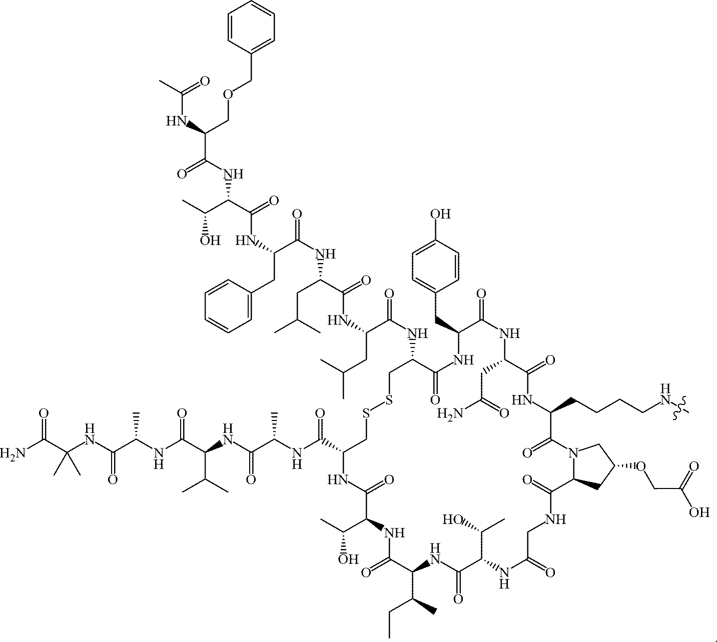

In some embodiments, it is or comprises

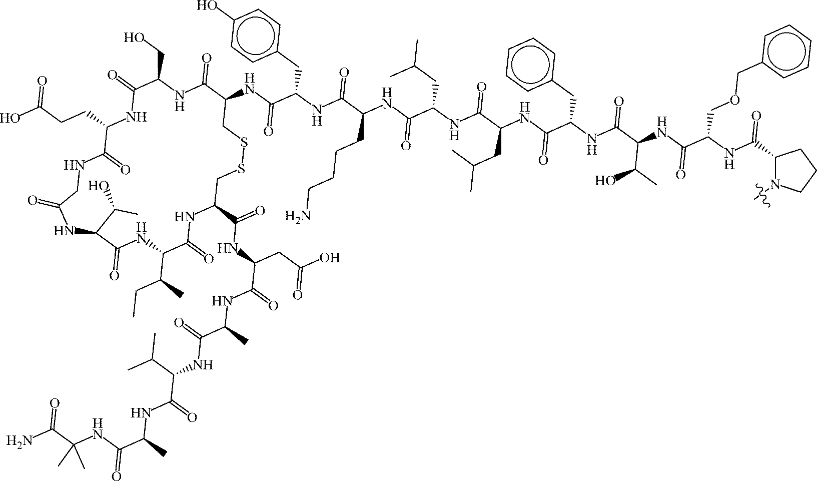

In some embodiments, it is or comprises

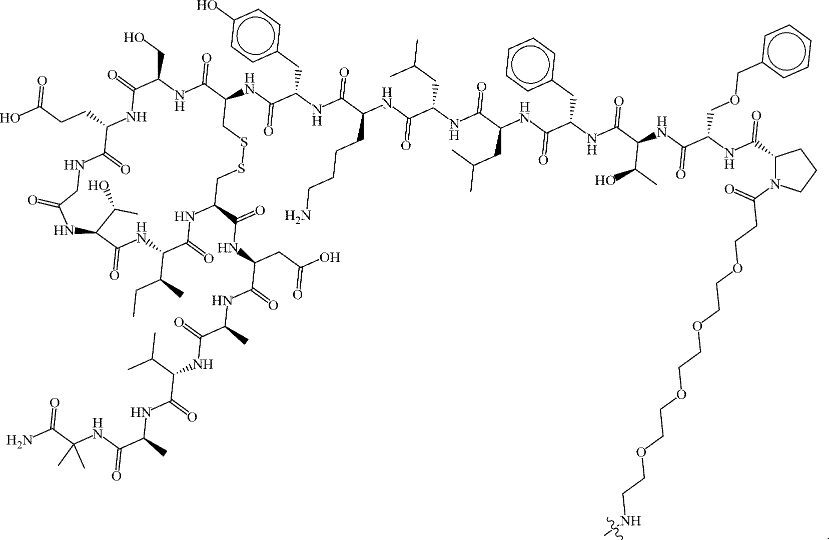

In some embodiments, it comprises one or more staples. In some embodiments, a staple comprises an amide group. In some embodiments, a staple is formed through amidation. In some embodiments, it is or comprises

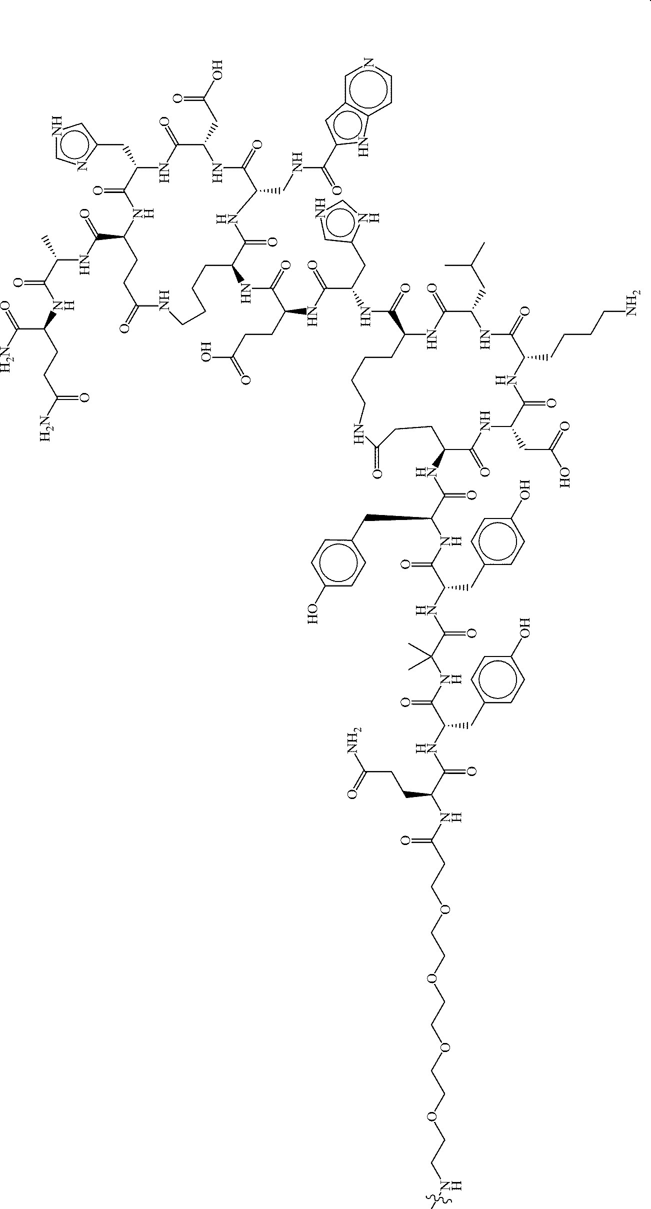

In some embodiments, it is or comprises

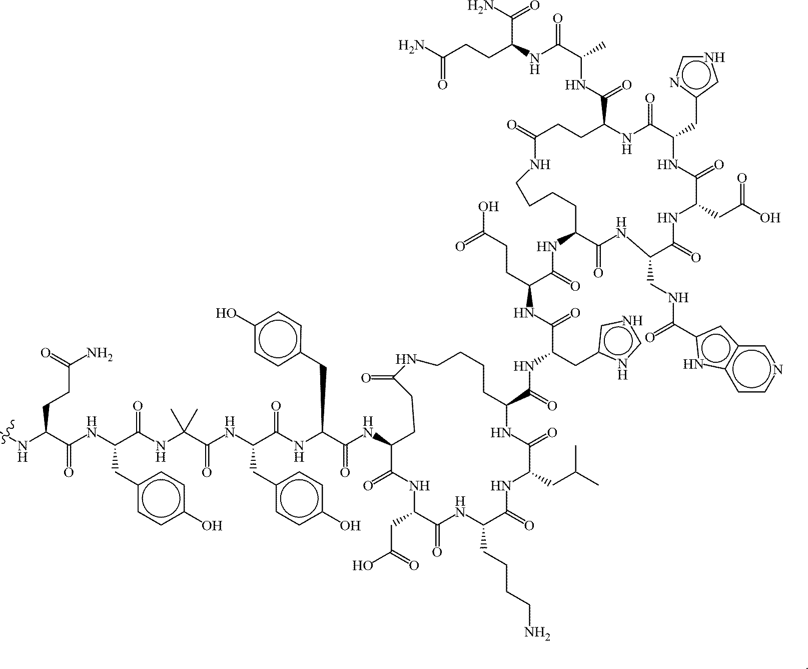

In some embodiments, a staple comprises a double bond. In some embodiments, it is or comprises

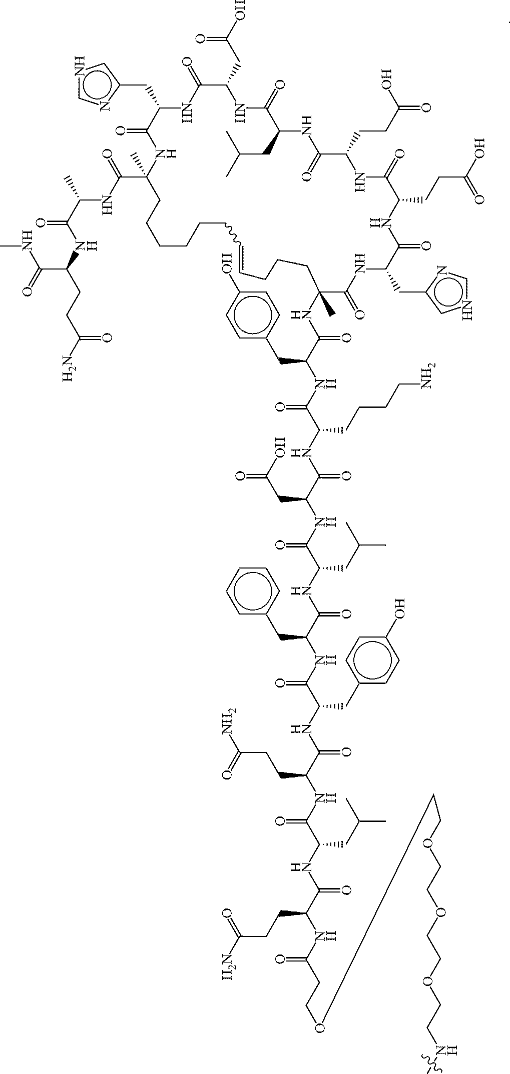

In some embodiments, it is or comprises

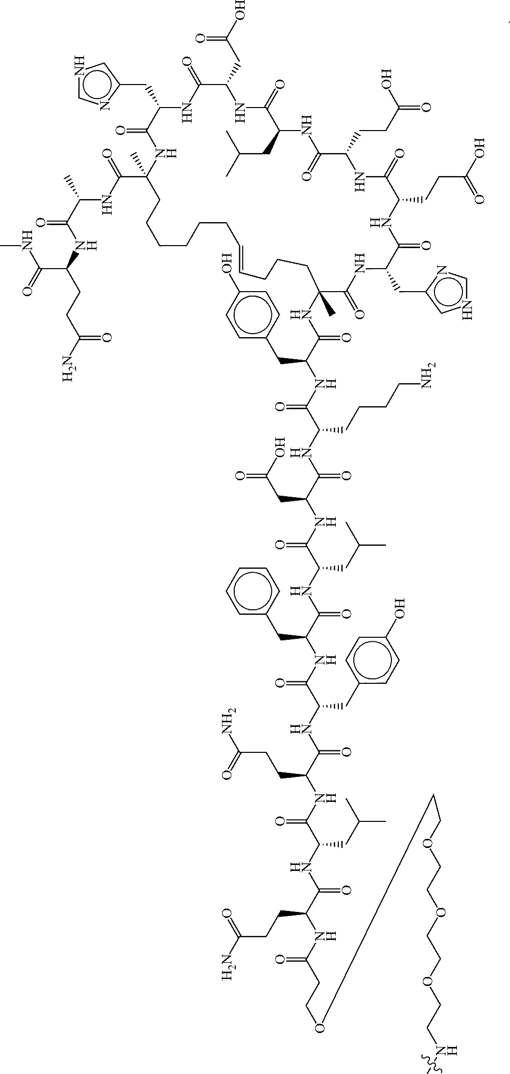

In some embodiments, it is or comprises

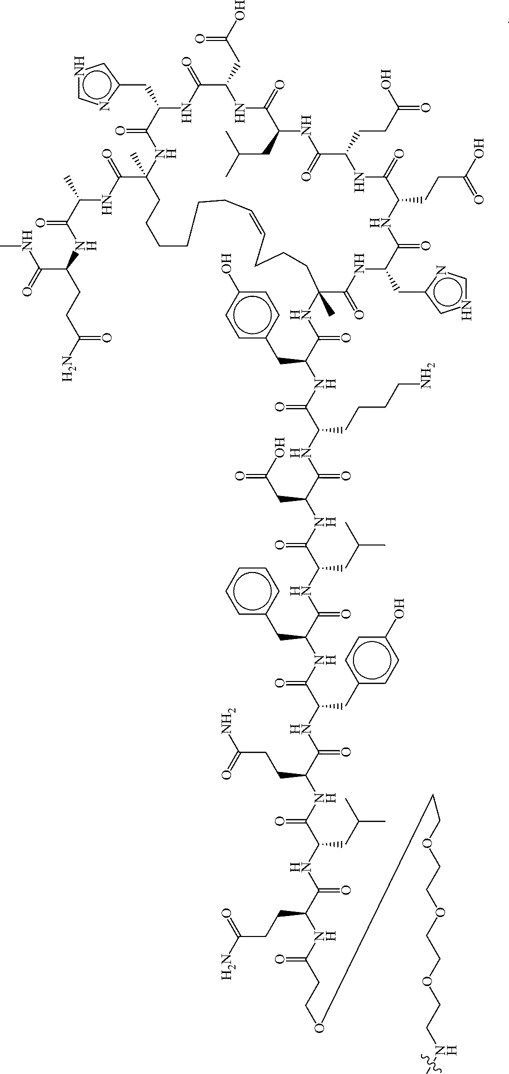

In some embodiments, it is or comprises

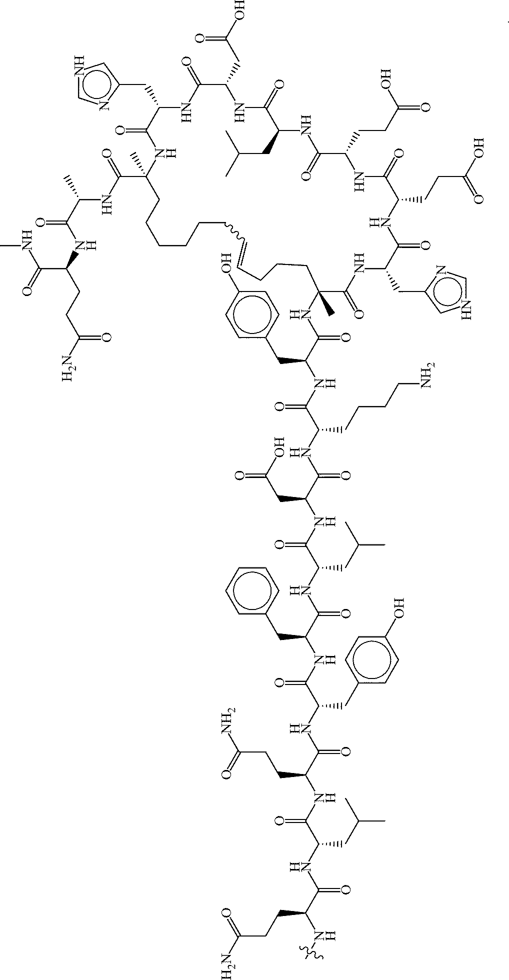

In some embodiments, it is or comprises

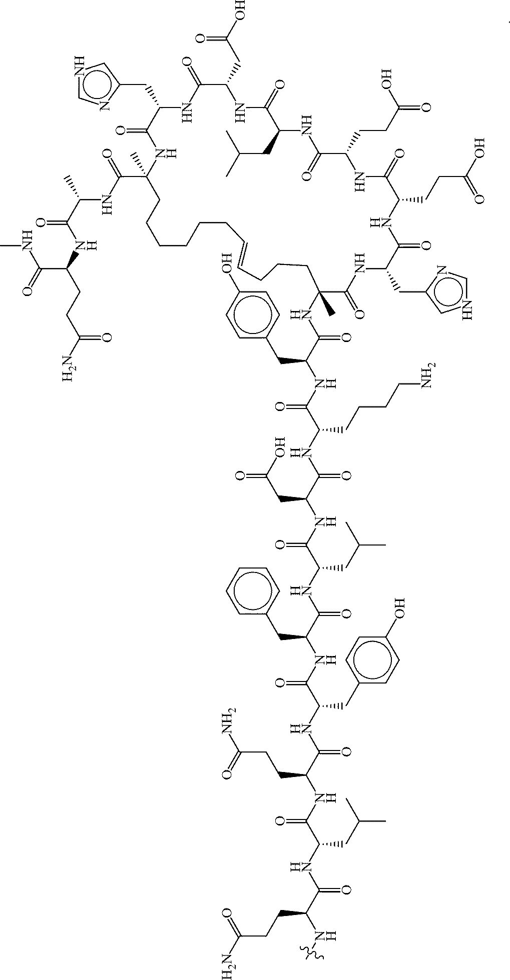

In some embodiments, it is or comprises

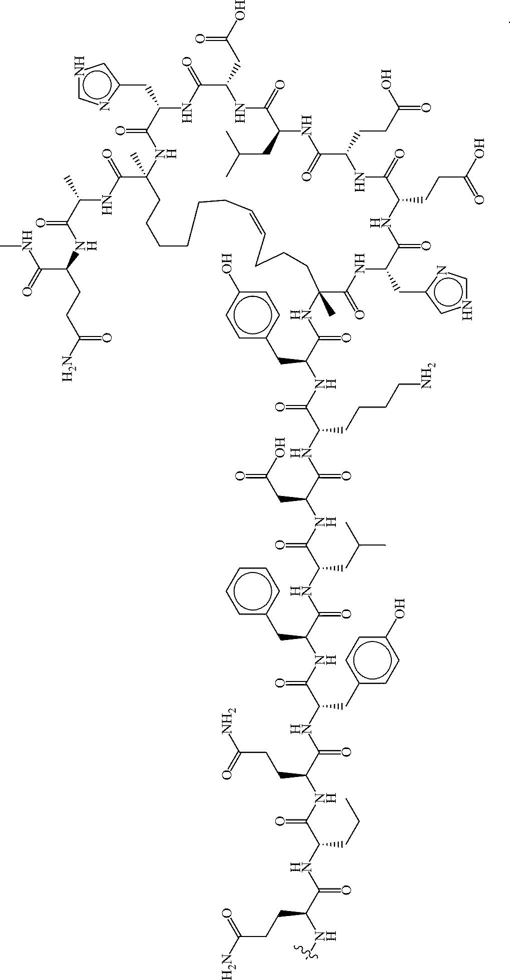

In some embodiments, a provided agent has the structure of

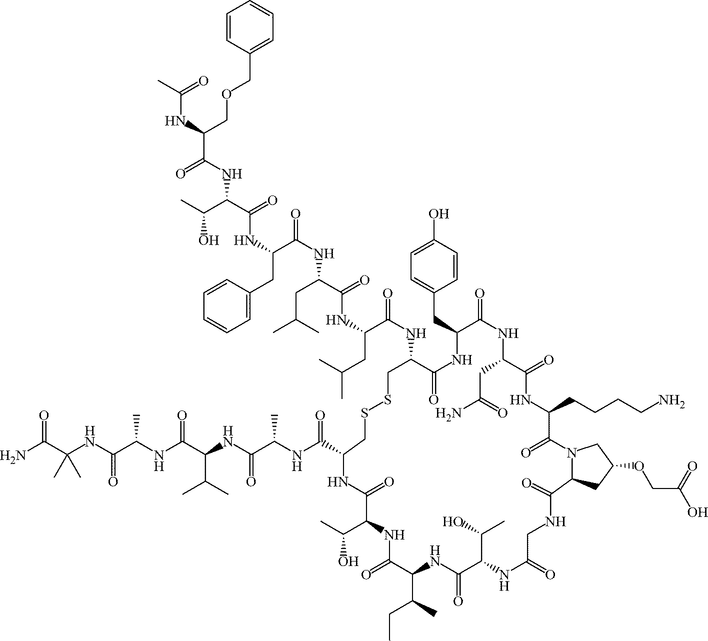 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCN)NC(=O)[C@H](CC(N)=O)NC(=O)[C@H](Cc2ccc(O)cc2)NC(=O)[C@@H](NC(=O)[C@H](CC(C)C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O]

or a salt thereof. In some embodiments, a provided agent has the structure of

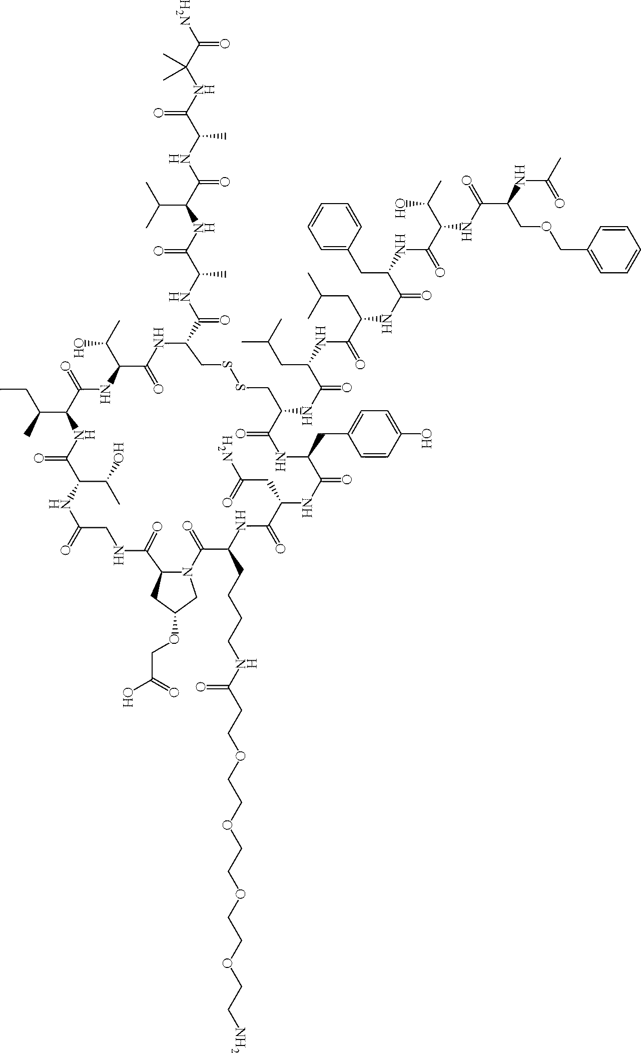 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCNC(=O)CCOCCOCCOCCOCCN)NC(=O)[C@H](CC(N)=O)NC(=O)[C@H](Cc2ccc(O)cc2)NC(=O)[C@@H](NC(=O)[C@H](CC(C)C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O]

or a salt thereof. In some embodiments, a provided agent has the structure of

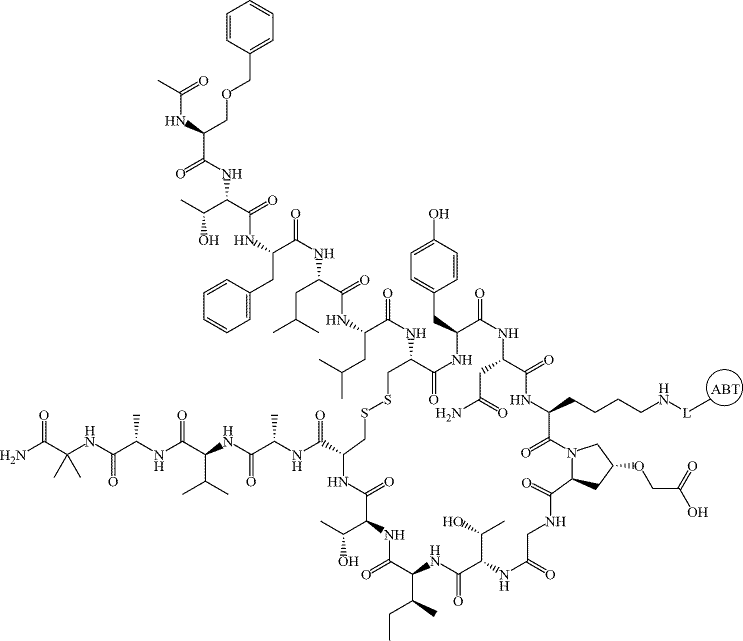

or a salt thereof. In some embodiments, a provided agent has the structure of

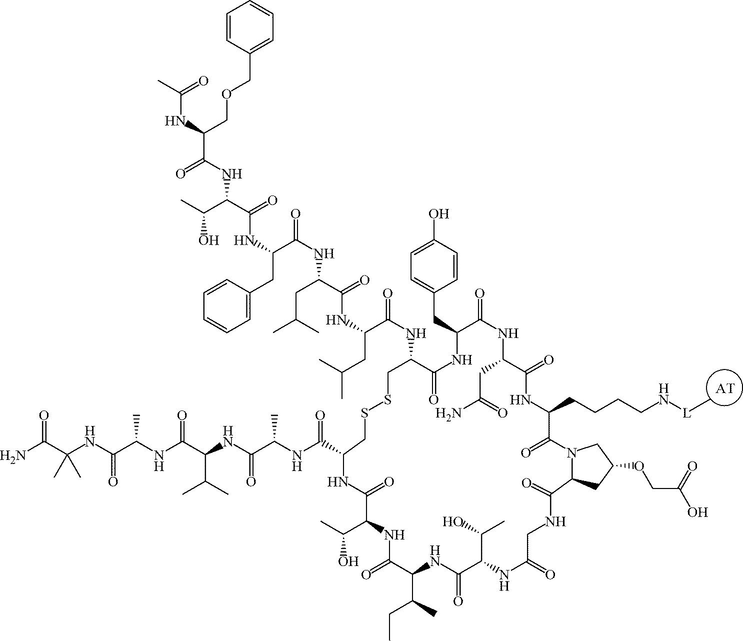

or a salt thereof. In some embodiments, a provided agent has the structure of

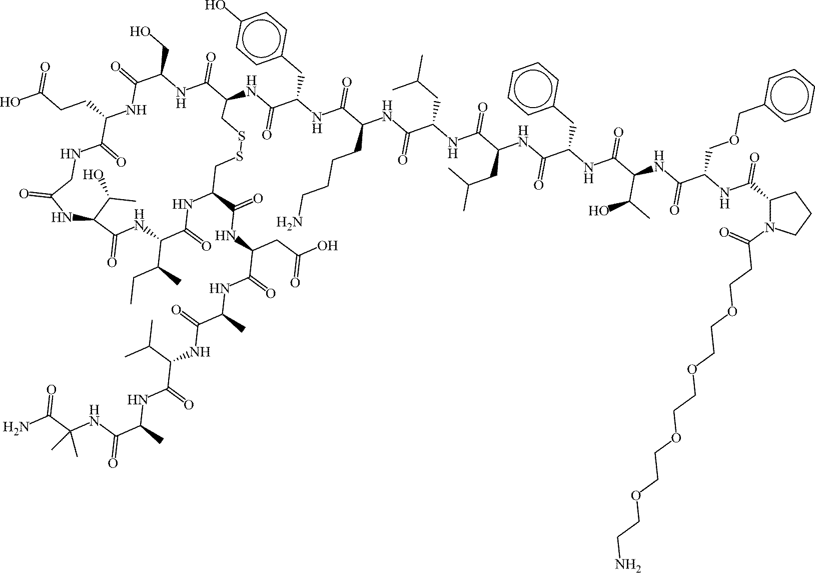

or a salt thereof. In some embodiments, a provided agent has the structure of

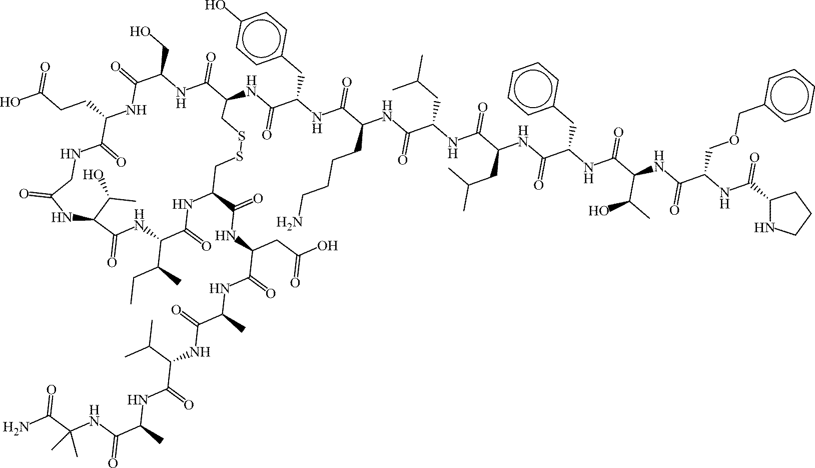

or a salt thereof. In some embodiments, a provided agent has the structure of

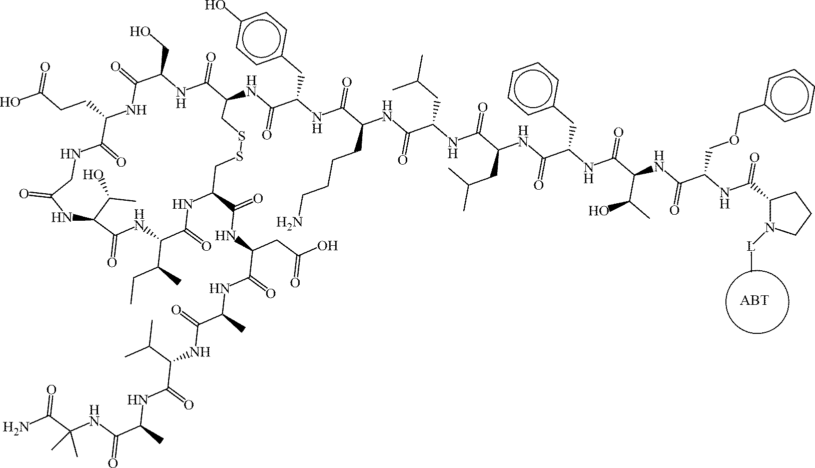

or a salt thereof. In some embodiments, a provided agent has the structure of

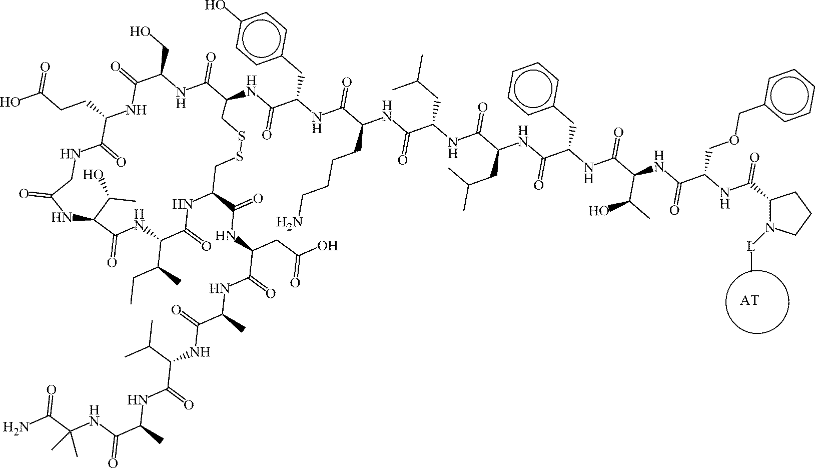

or a salt thereof. In some embodiments, a provided agent has the structure of

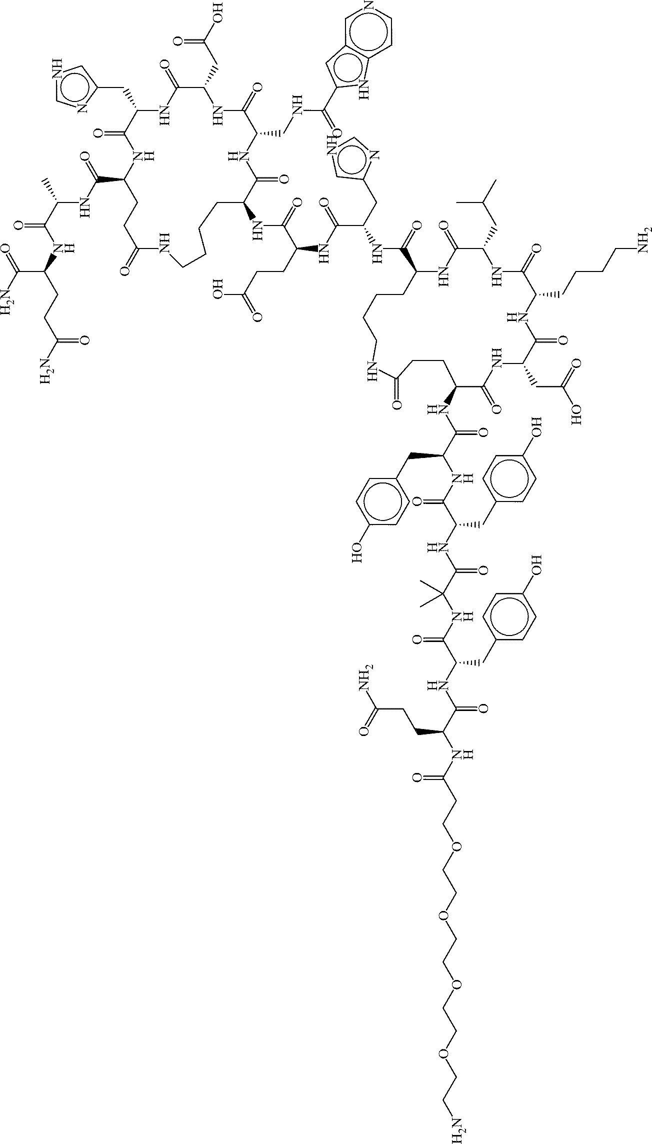

or a salt thereof. In some embodiments, a provided agent has the structure of

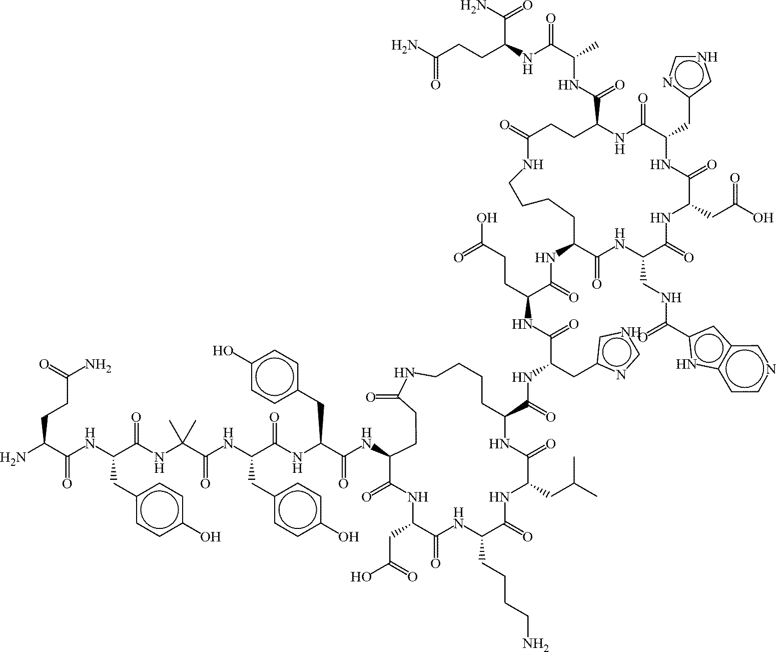

or a salt thereof. In some embodiments, a provided agent has the structure of

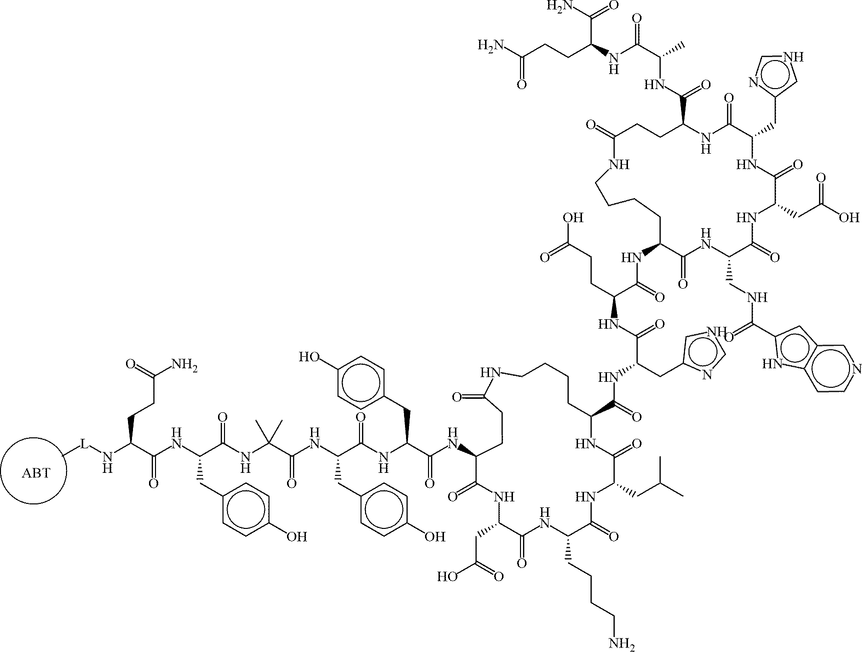

or a salt thereof. In some embodiments, a provided agent has the structure of

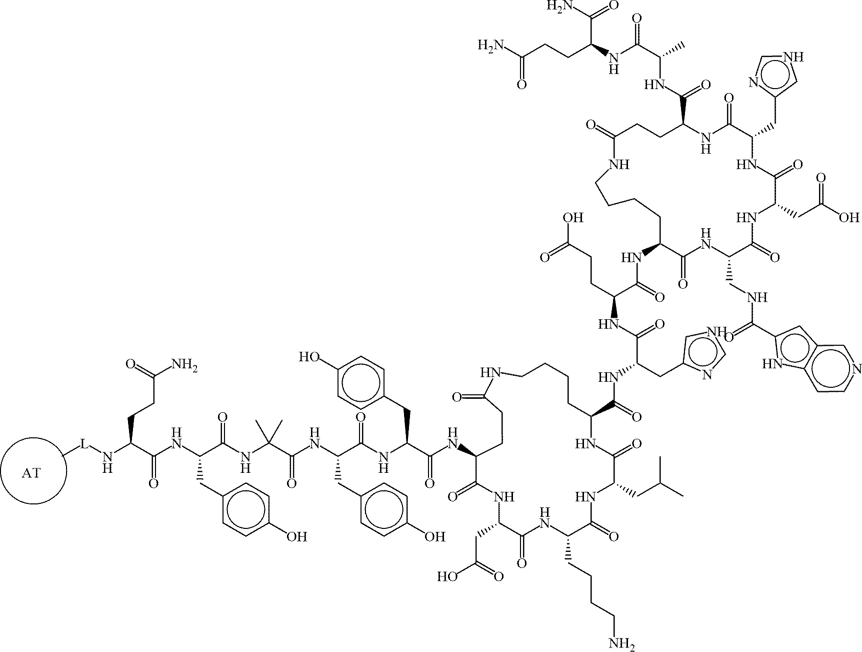

or a saltthereof. In some embodiments, a provided agent has the structure of

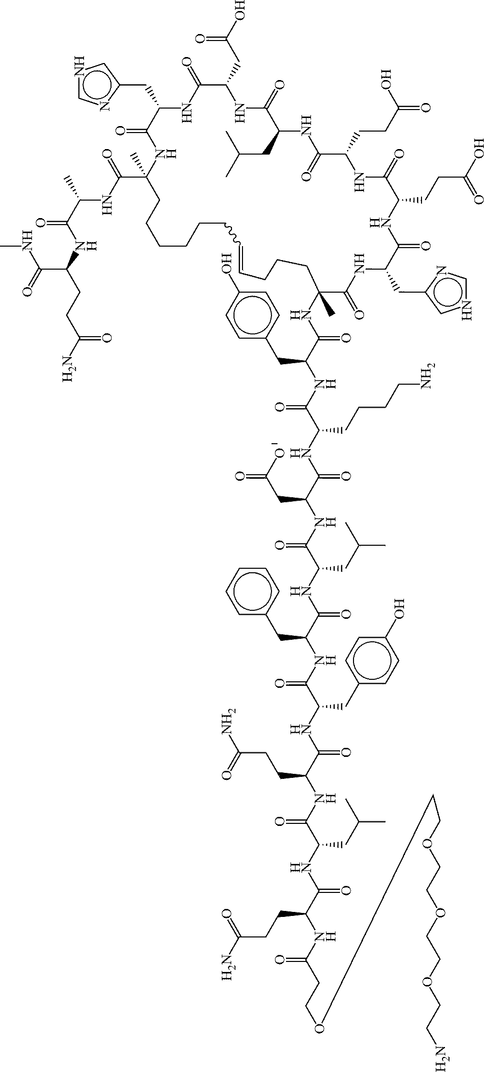

or a salt thereof. In some embodiments, a provided agent has the structure of

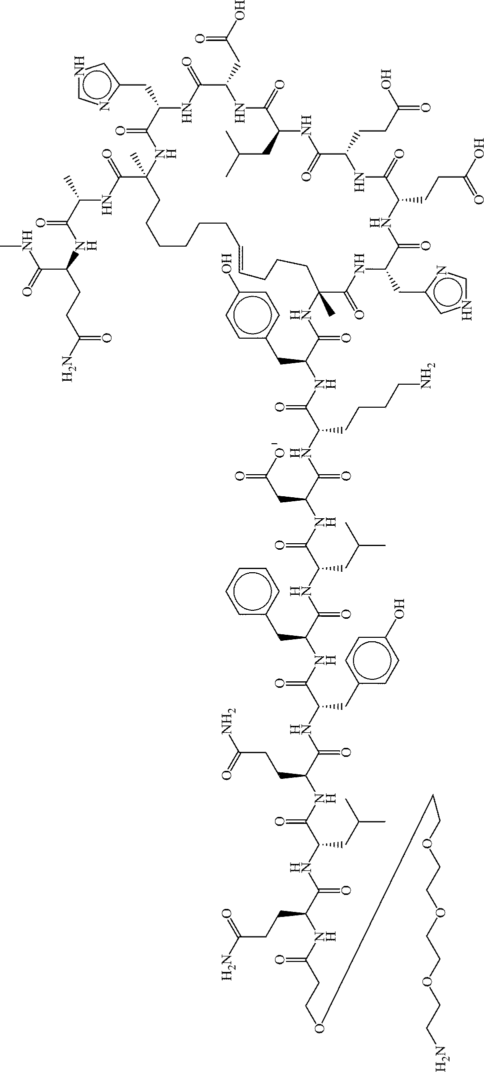

or a salt thereof. In some embodiments, a provided agent has the structure of

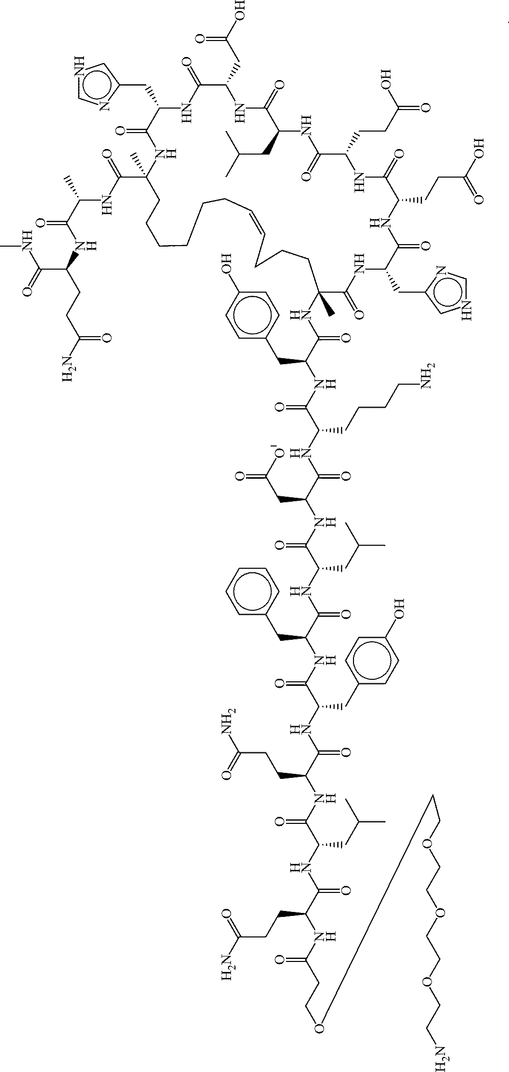

or a salt thereof. In some embodiments, a provided agent has the structure of

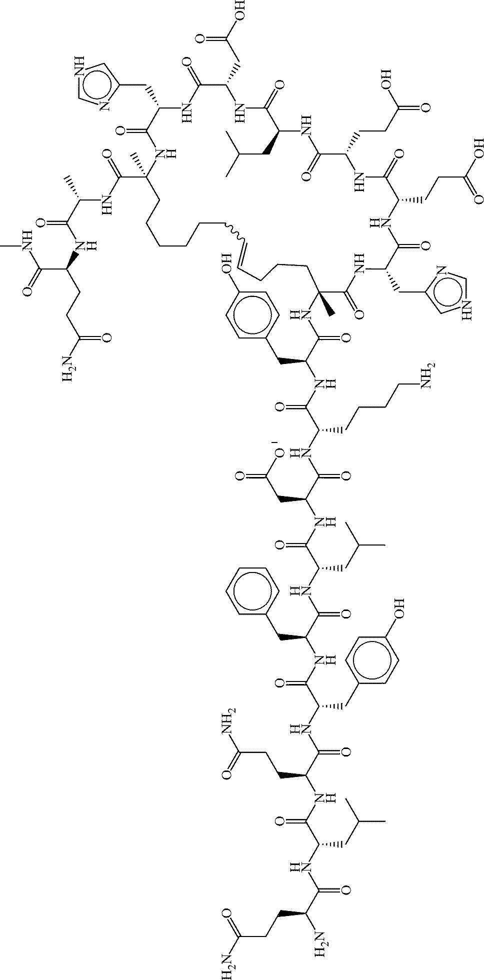

or a salt thereof. In some embodiments, a provided agent has the structure of

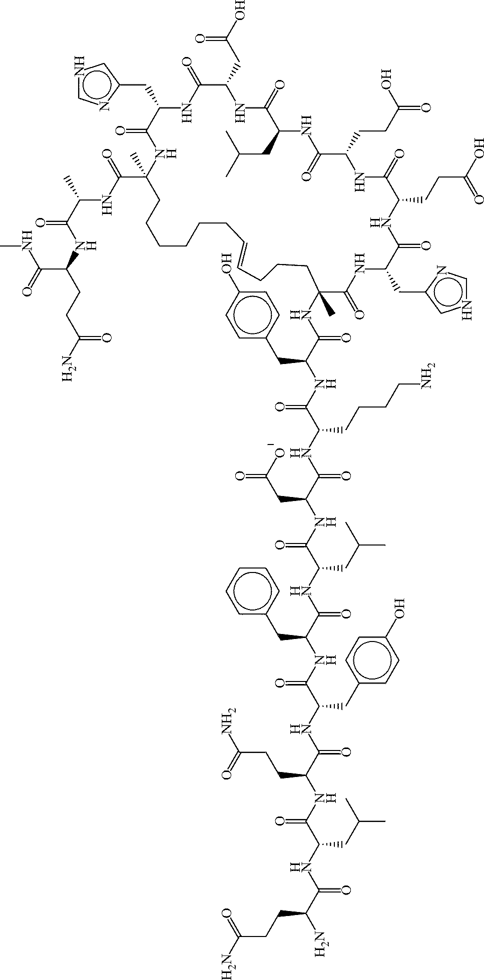

or a salt thereof. In some embodiments, a provided agent has the structure of

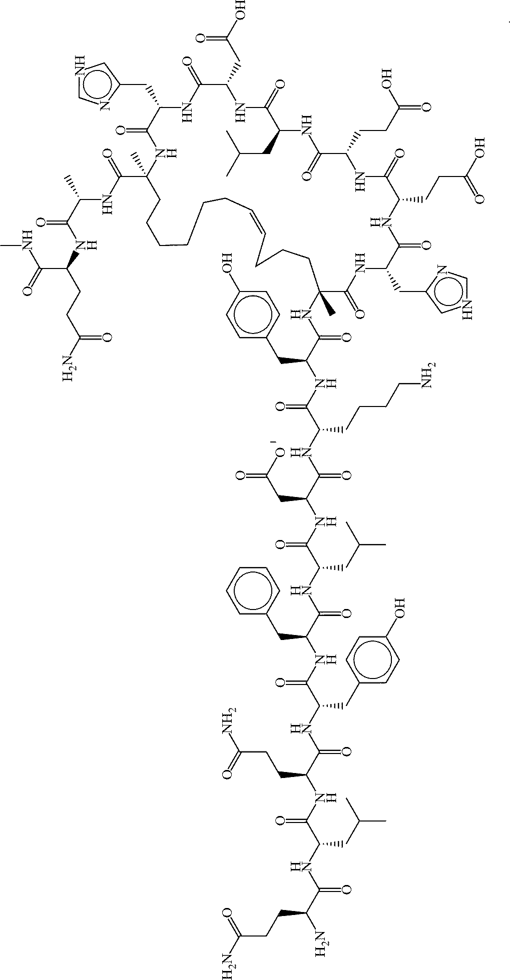

or a salt thereof. In some embodiments, a provided agent has the structure of

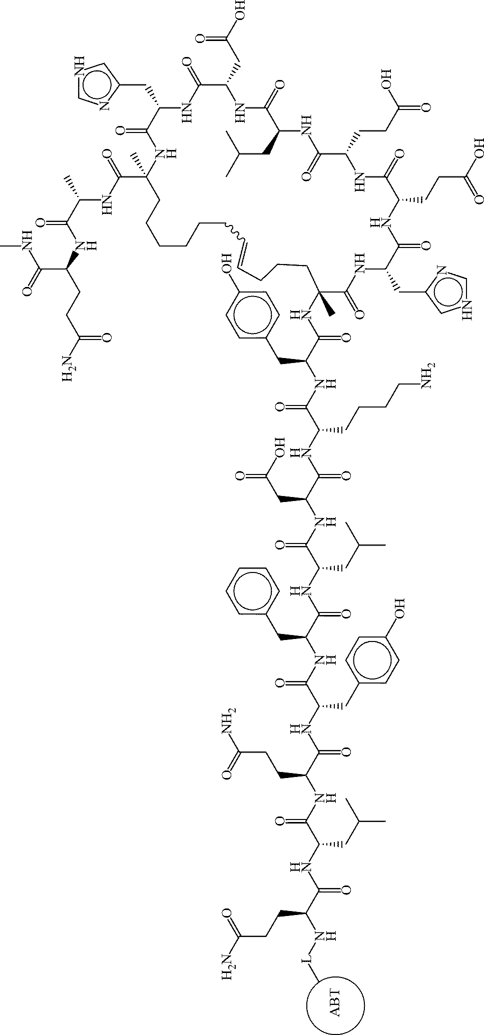

or a salt thereof. In some embodiments, a provided agent has the structure of

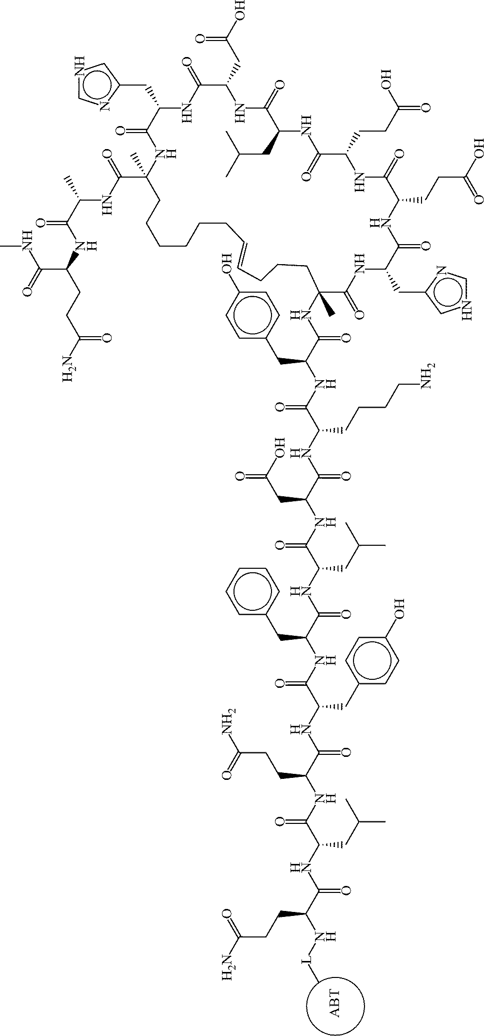

or a salt thereof. In some embodiments, a provided agent has the structure of

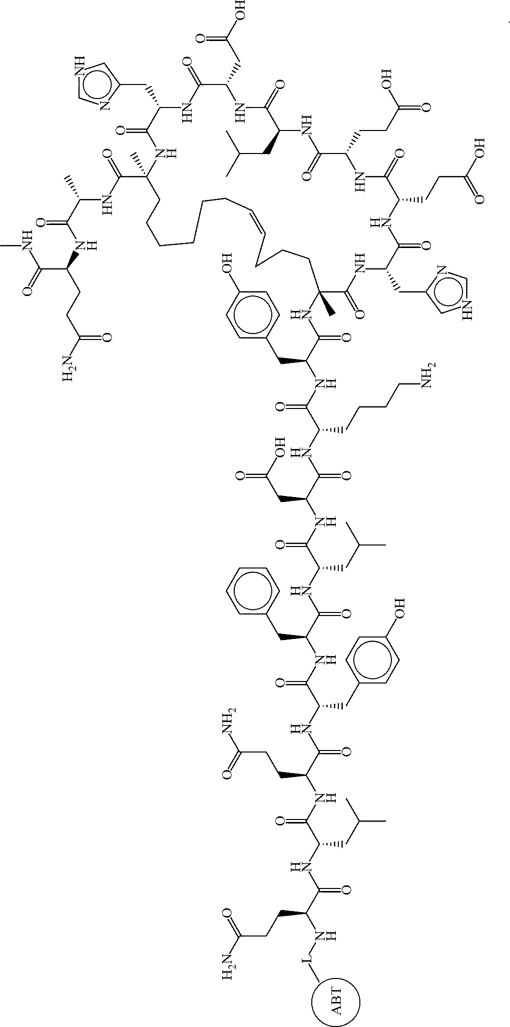

or a salt thereof. In some embodiments, a provided agent has the structure of

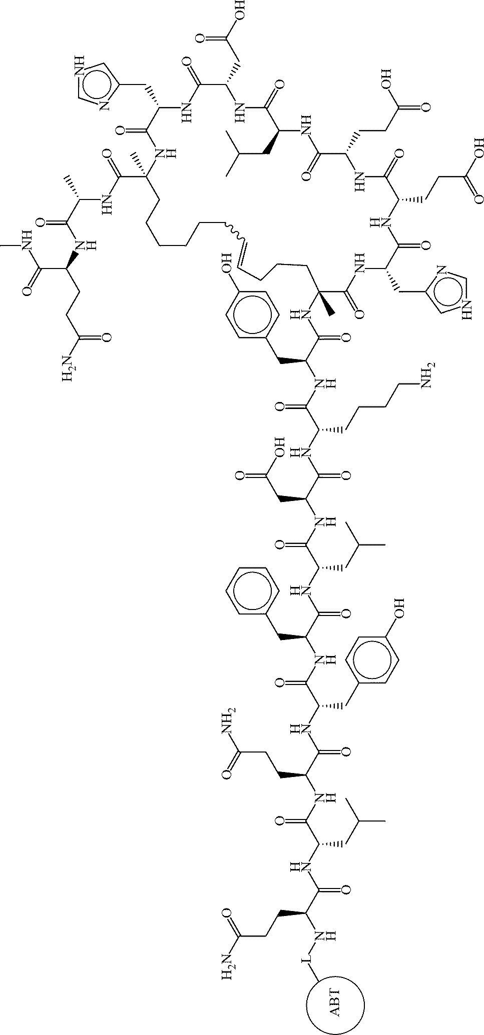

or a salt thereof. In some embodiments, a provided agent has the structure of

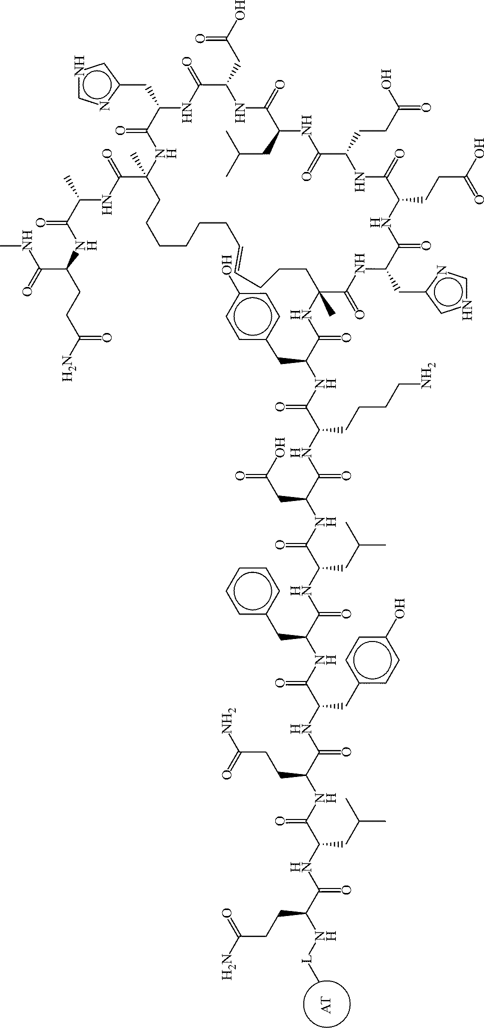

or a salt thereof. In some embodiments, a provided agent has the structure of

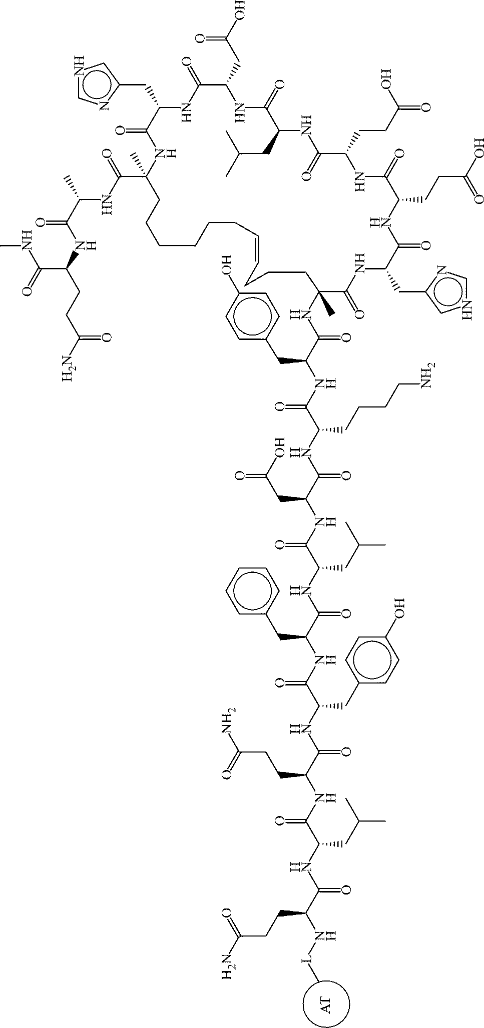

or a salt thereof.

In some embodiments, a provided agent has the structure of

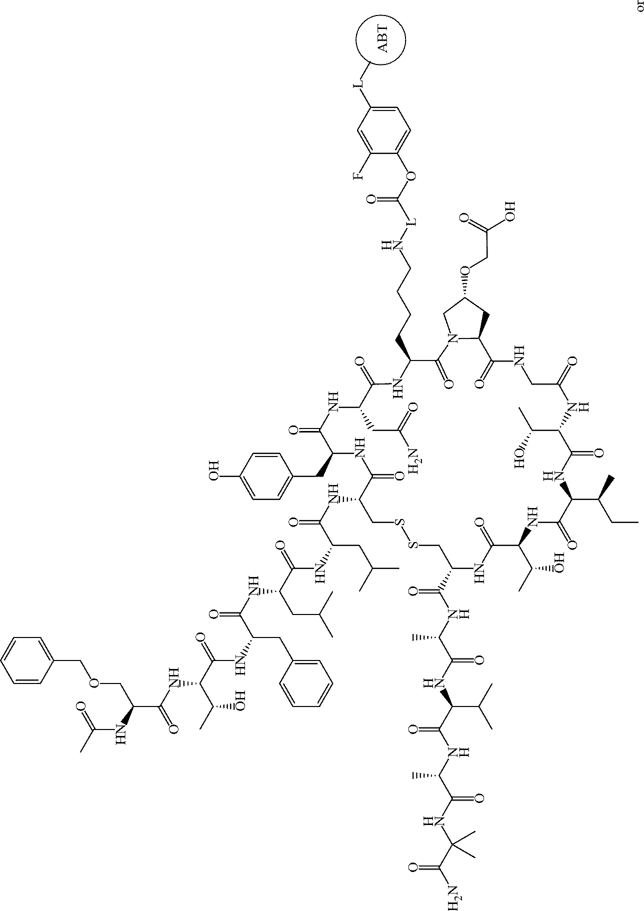

a salt thereof (as in various structures of the present disclosure, each variable is independently as described herein). In some embodiments, a provided agent has the structure of

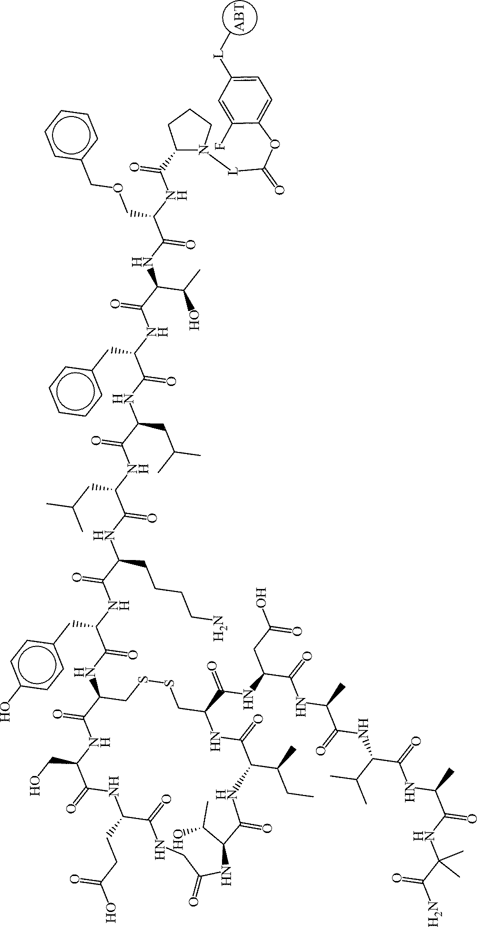

or a salt thereof. In some embodiments, a provided agent has the structure of

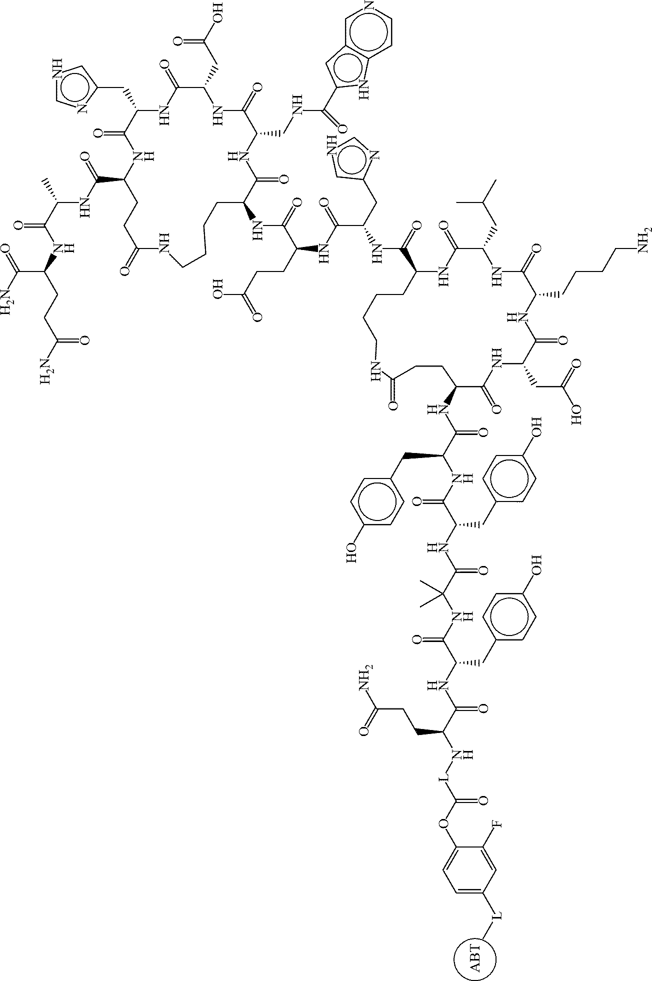

or a salt thereof. In some embodiments, a provided agent has the structure of

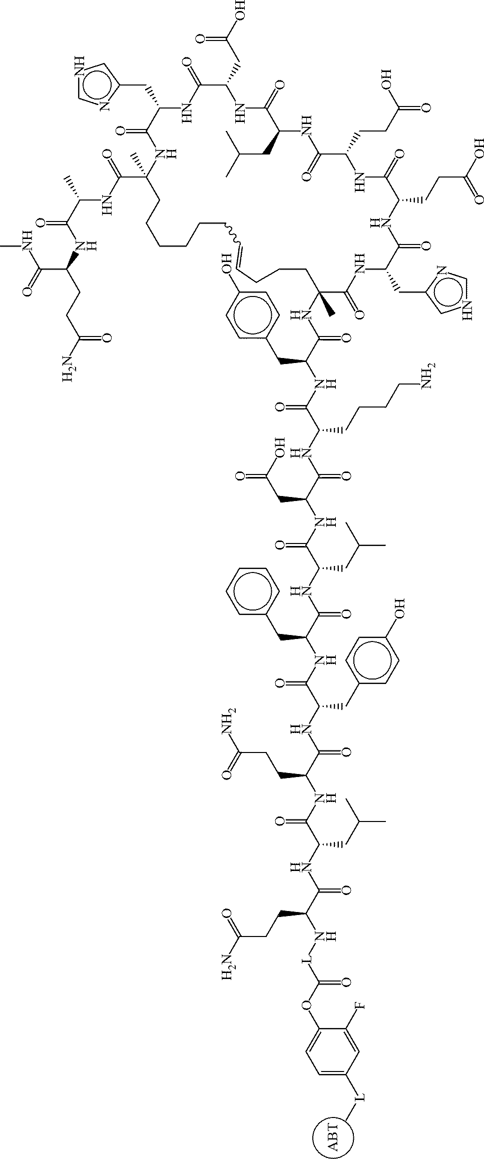

or a salt thereof. In some embodiments, a provided agent has the structure of

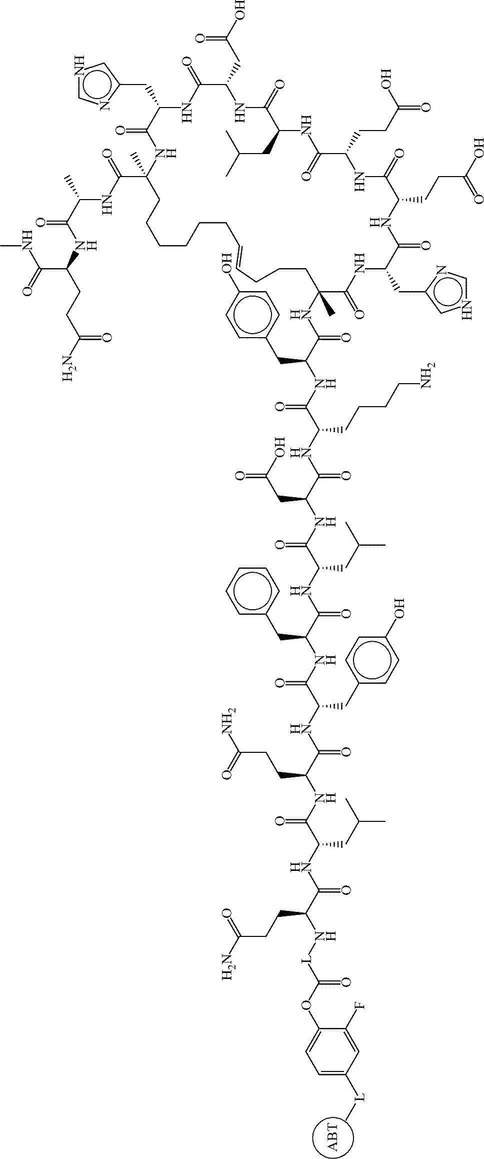

or a salt thereof. In some embodiments, a provided agent has the structure of

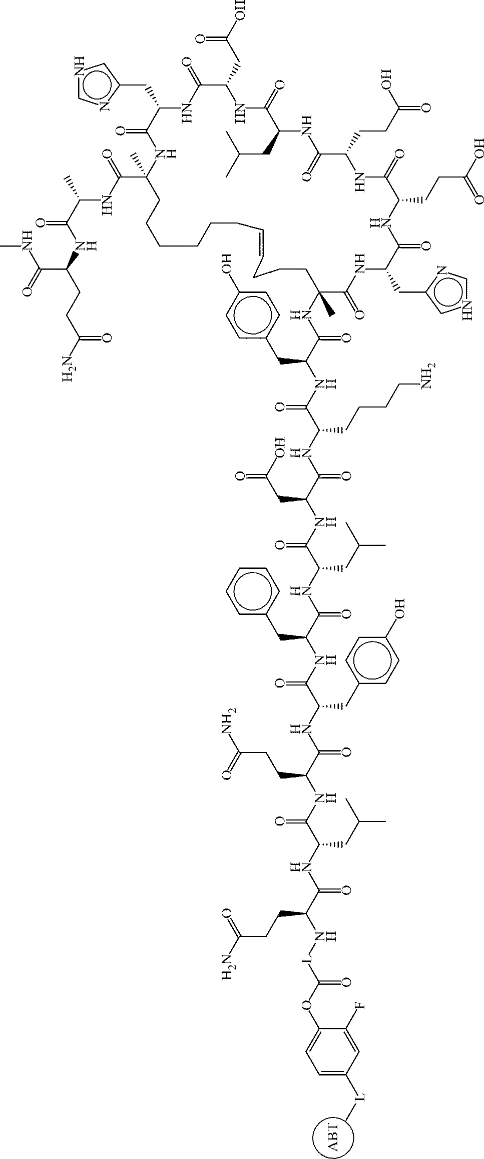

or a salt thereof.

In some embodiments, a provided agent has the structure of:

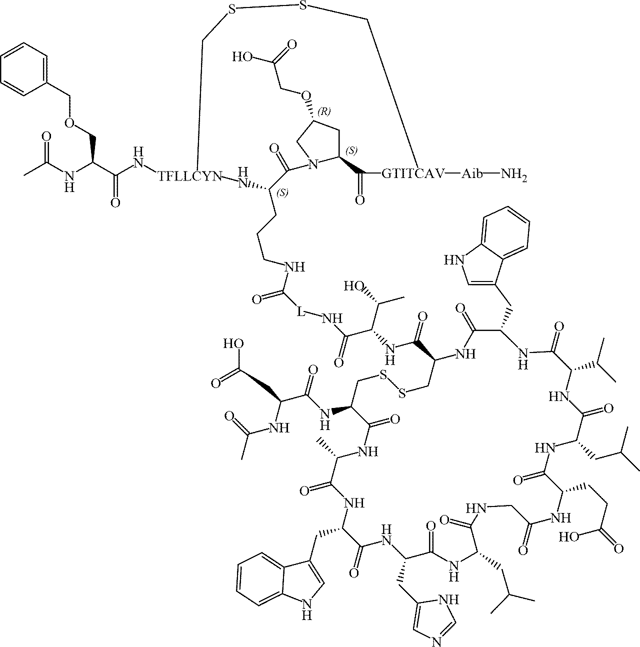

or a salt thereof.

In some embodiments, a provided agent has the structure of:

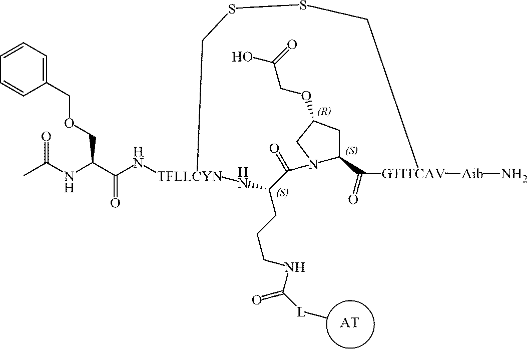

or a salt thereof.

In some embodiments, a provided agent has the structure of:

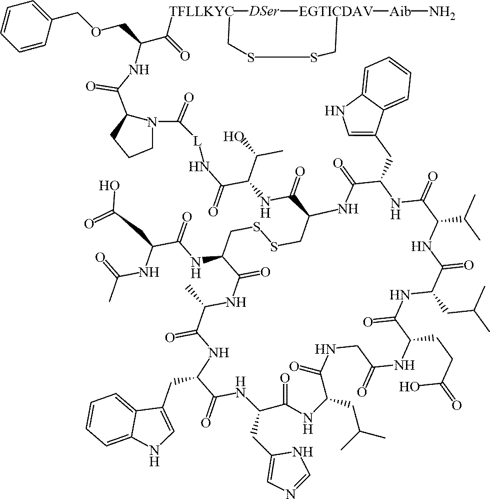

or a salt thereof.

In some embodiments, a provided agent has the structure of:

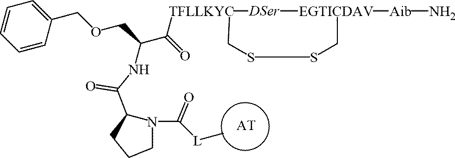

or a salt thereof.

In some embodiments, a provided agent has the structure of:

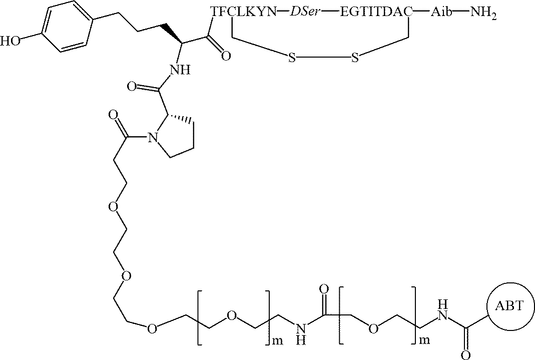

or a salt thereof.

In some embodiments, a provided agent has the structure of:

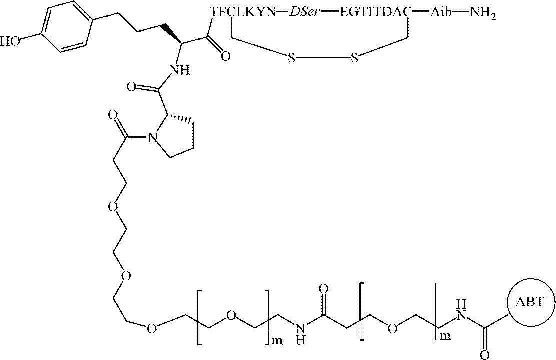

or a salt thereof.

In some embodiments, a provided agent has the structure of:

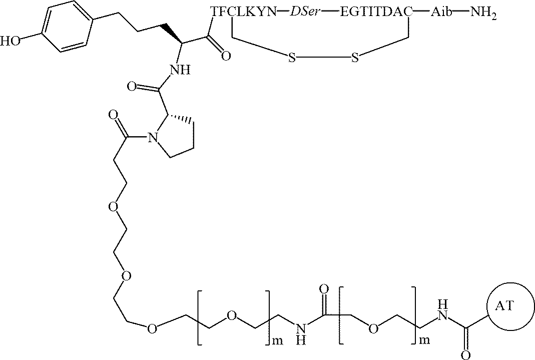

or a salt thereof.

In some embodiments, a provided agent has the structure of:

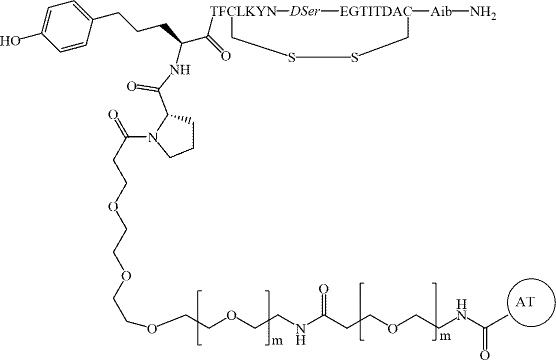

or a salt thereof.

In some embodiments, a provided agent has the structure of:

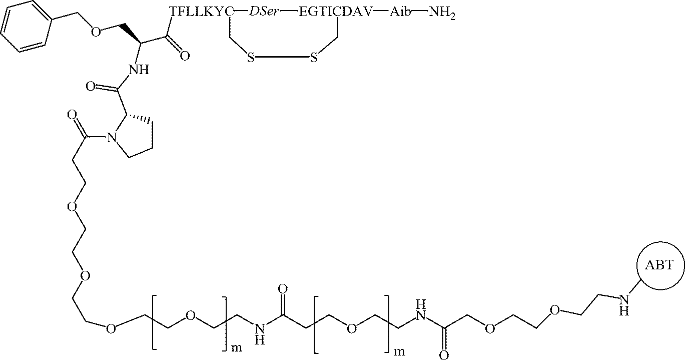

or a salt thereof.

In some embodiments, a provided agent has the structure of:

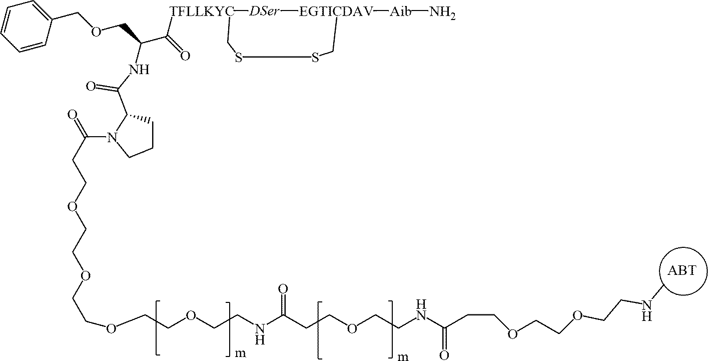

or a salt thereof.

In some embodiments, a provided agent has the structure of:

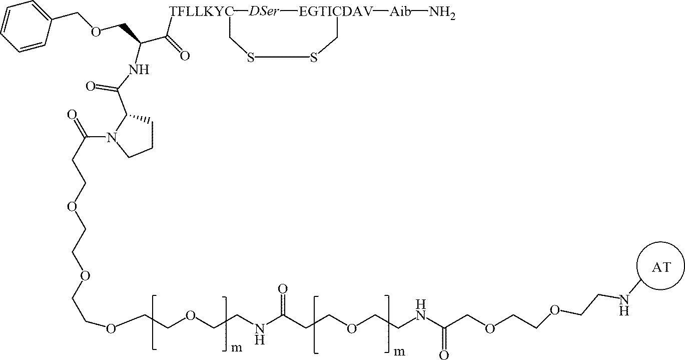

or a salt thereof.

In some embodiments, a provided agent has the structure of:

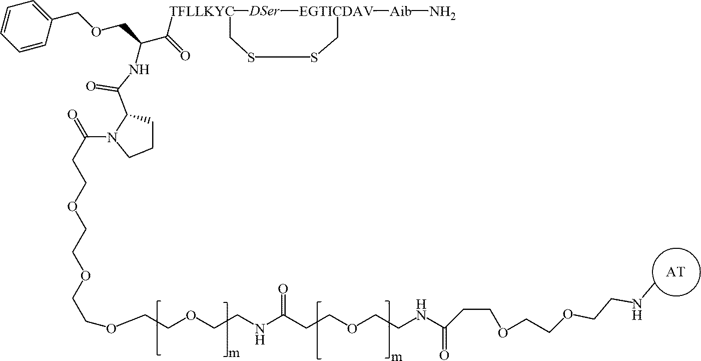

or a salt thereof.

In some embodiments, a provided agent has the structure of:

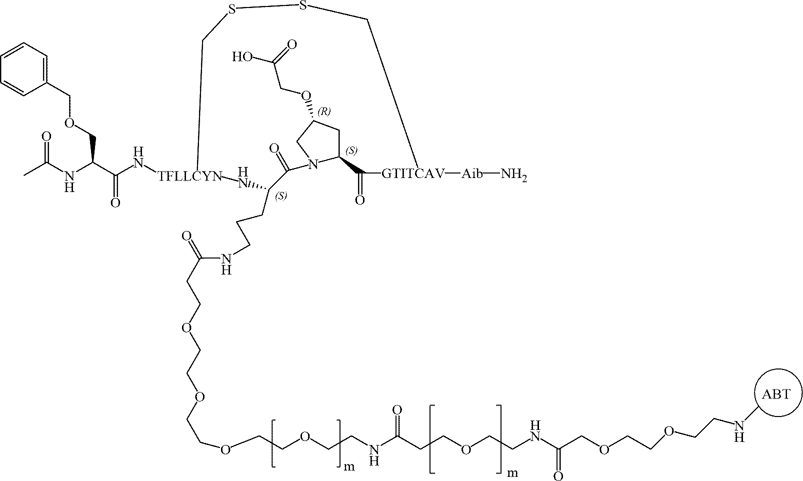

or a salt thereof.

In some embodiments, a provided agent has the structure of:

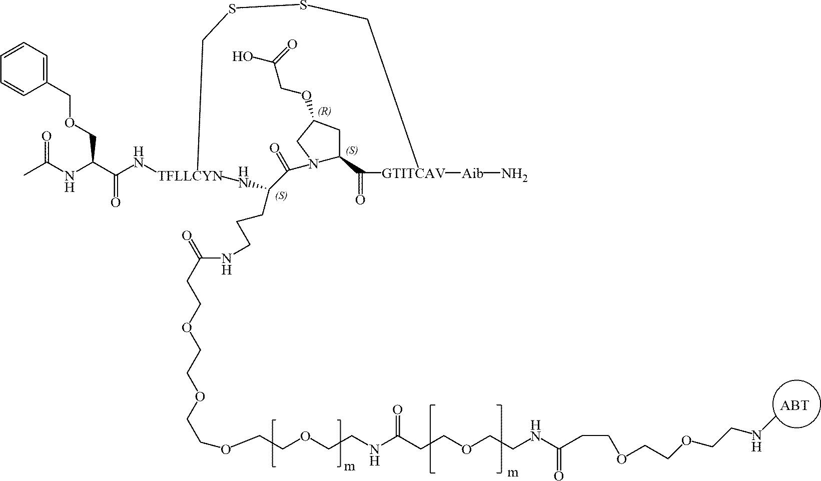

or a salt thereof.

In some embodiments, a provided agent has the structure of:

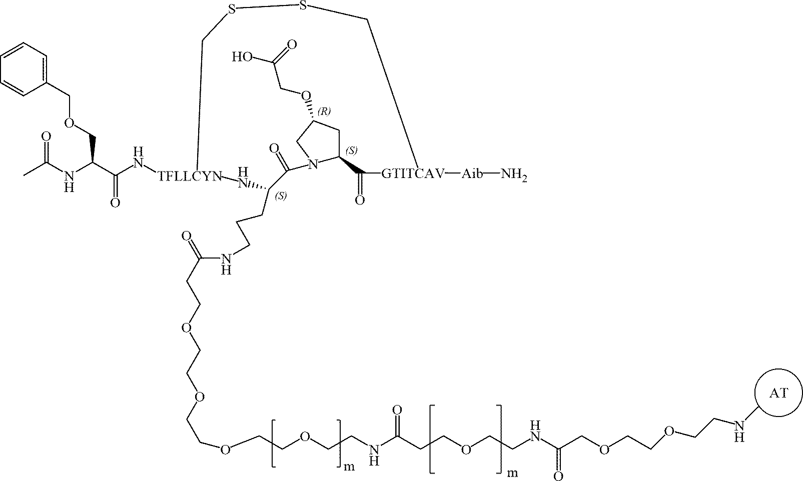

or a salt thereof.

In some embodiments, a provided agent has the structure of:

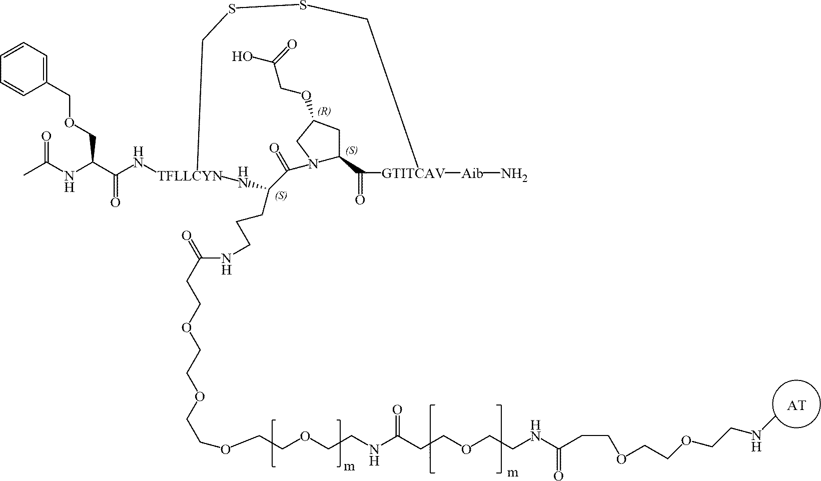

or a salt thereof.

In some embodiments, a provided agent has the structure of:

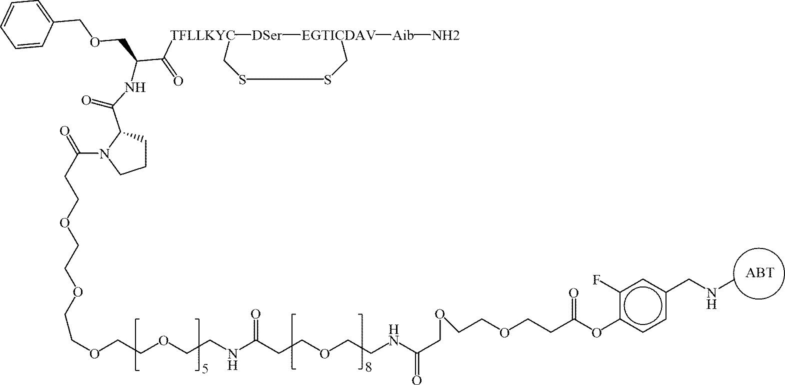

or a salt thereof.

In some embodiments, a provided agent has the structure of:

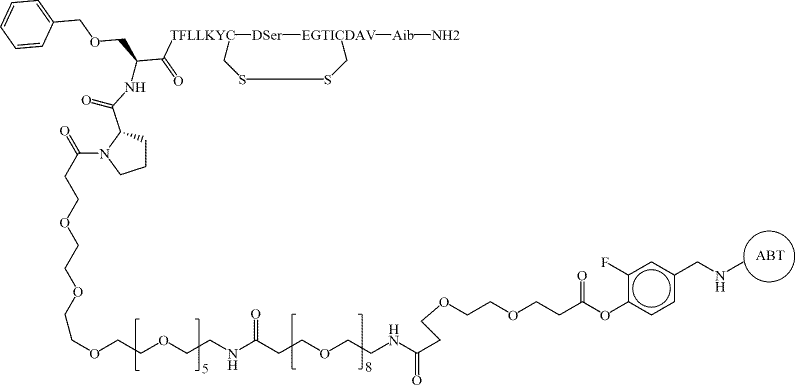

or a salt thereof.

In some embodiments, a provided agent has the structure of:

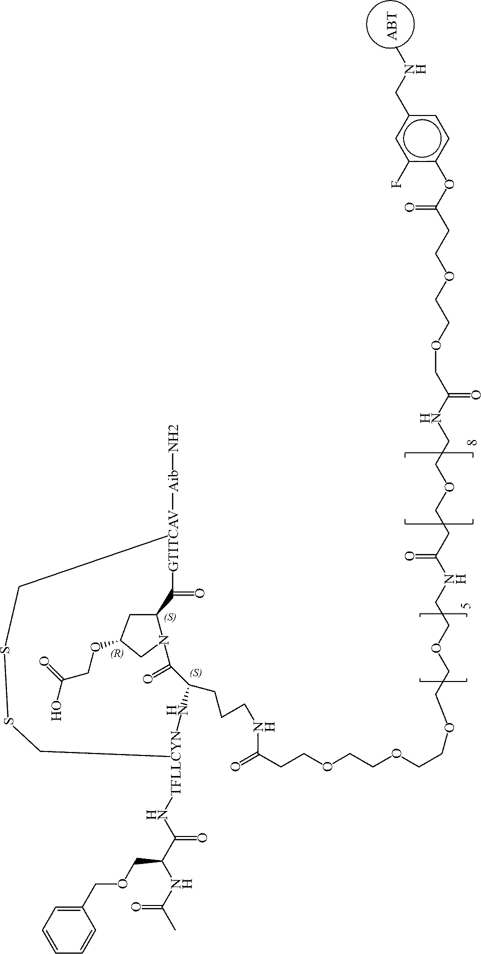

or a salt thereof.

In some embodiments, a provided agent has the structure of:

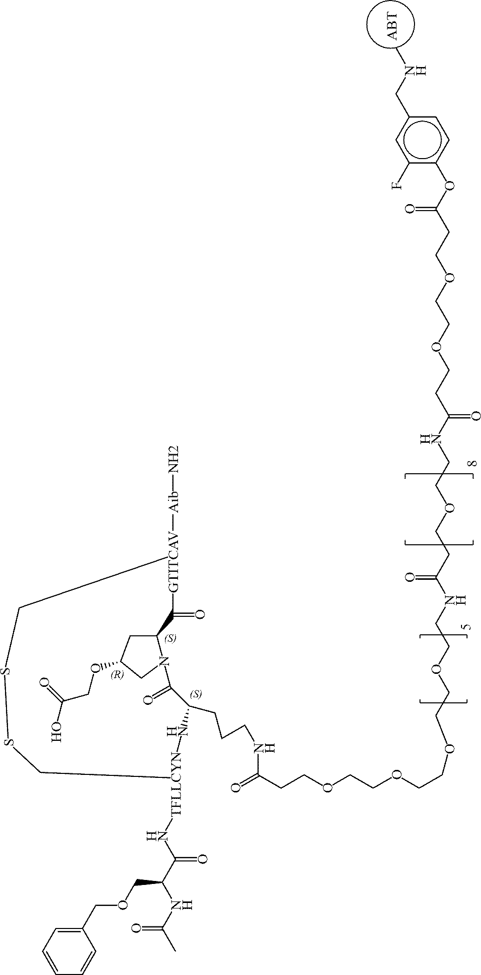

or a salt thereof.

In some embodiments, a provided agent has the structure of:

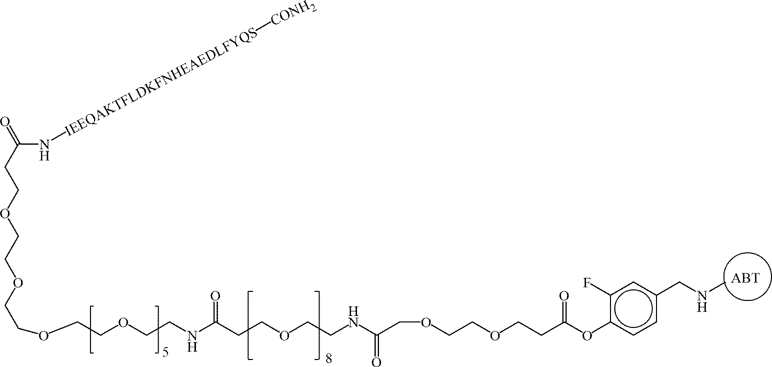

or a salt thereof.

In some embodiments, a provided agent has the structure of:

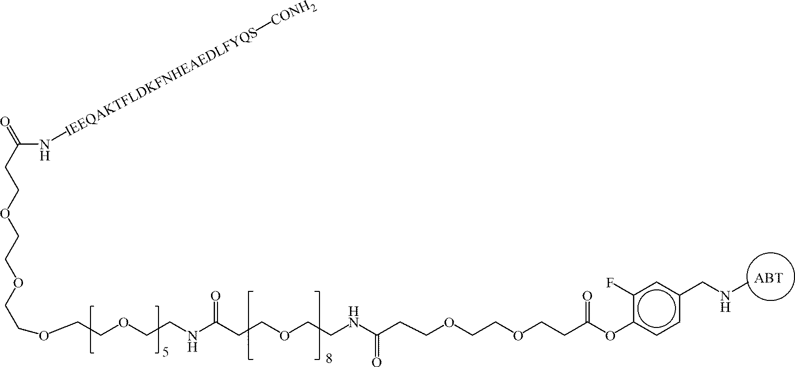

or a salt thereof.

In some embodiments, a provided agent has the structure of:

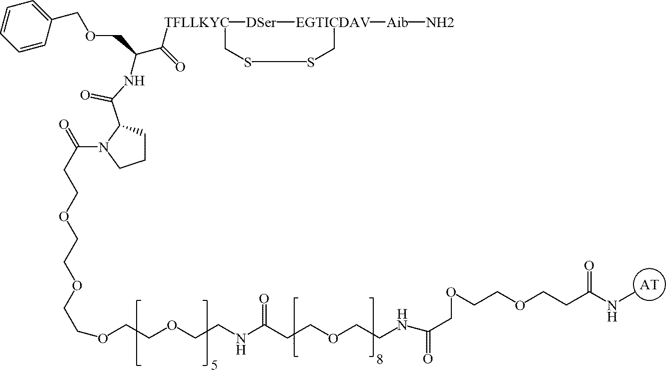

or a salt thereof.

In some embodiments, a provided agent has the structure of:

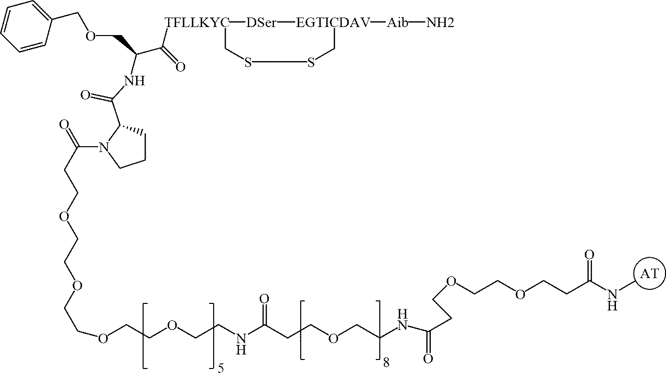

or a salt thereof.

In some embodiments, a provided agent has the structure of:

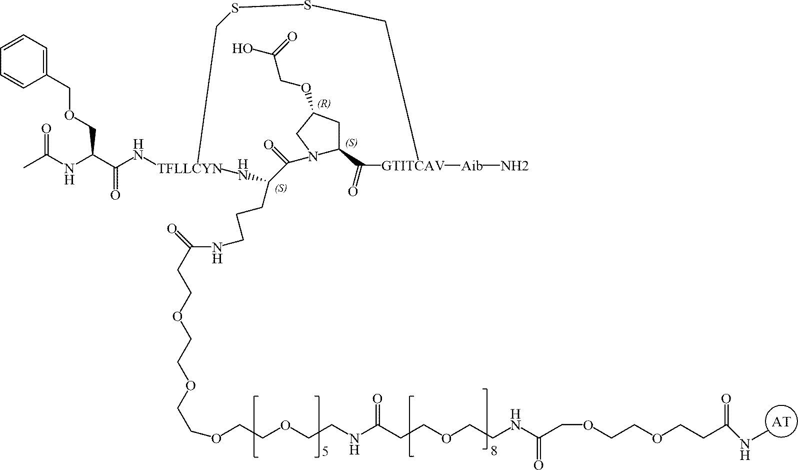

or a salt thereof.

In some embodiments, a provided agent has the structure of:

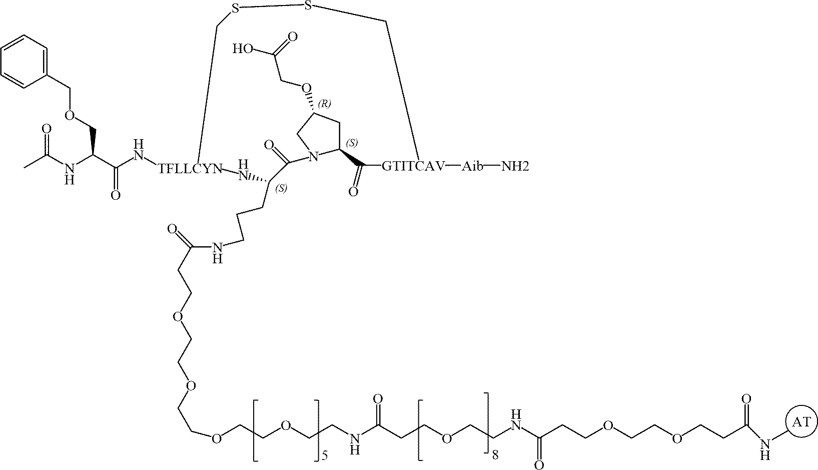

or a salt thereof.

In some embodiments, a provided agent has the structure of:

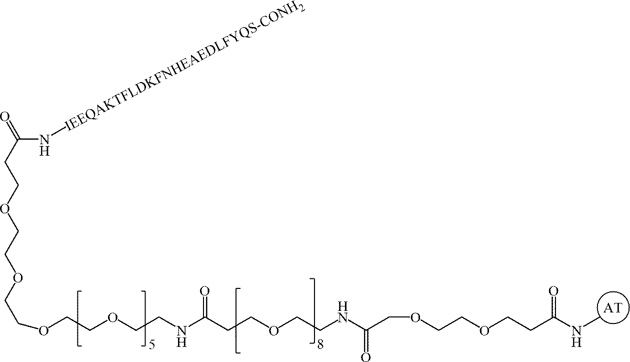

or a salt thereof.

In some embodiments, a provided agent has the structure of:

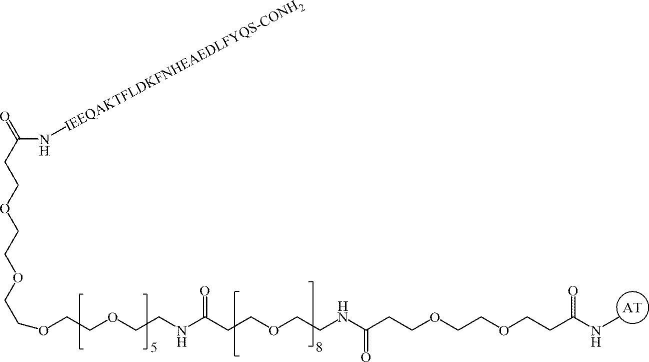

or a salt thereof.

In some embodiments, a peptide unit, e.g., a target binding moiety, comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises —Cy—. In some embodiments, L^{a} comprises —Cy—, wherein —Cy— is optionally substituted heteroaryl. In some embodiments, —Cy— is

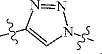

In some embodiments, L^{a} is

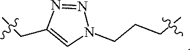

In some embodiments, such an L^{a} can be formed by a —N₃ group of the side chain of one amino acid residue, and the -≡- of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, —Cy— is optionally substituted phenylene. In some embodiments, —Cy— is optionally substituted 1,2-phenylene. In some embodiments, L^{a} is

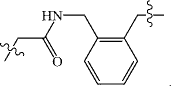

In some embodiments, L^{a} is

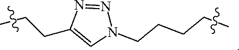

In some embodiments, L^{a} is optionally substituted bivalent C₂-₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, a target binding moiety comprises a peptide unit, and an antibody binding moiety is connected to a backbone atom of the peptide unit optionally via a linker. In some embodiments, a target binding moiety comprises a peptide unit, and an antibody binding moiety is connected to an atom of a side chain, e.g., through an atom or group in the side chain, of an amino acid residue of the peptide unit optionally via a linker. For example, in some embodiments, an antibody binding moiety is connected through a —SH,—OH, —COOH, or —NH₂ of a side chain.

### Amino Acids

In some embodiments, provided compounds and agents may comprise one or more amino acid moieties, e.g., in universal antibody binding moieties, linker moieties, etc. Amino acid moieties can either be those of natural amino acids or unnatural amino acids. In some embodiments, an amino acid has the structure of formula A-I:

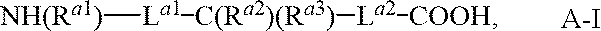

or a salt thereof, wherein each variable is independent as described in the present disclosure. In some embodiments, an amino acid residue, e.g., of an amino acid having the structure of formula A-I, has the structure of —N(R^{a1})—L^{a1}—C(R^{a2})(R^{a3})—L^{a2}—CO—. In some embodiments, each amino acid residue in a peptide independently has the structure of —N(R^{a1})—L^{a1}—C(R^{a2})(R^{a3})—L^{a2}—CO—.

In some embodiments, L^{al} is a covalent bond. In some embodiments, a compound of formula A-I is of the structure NH(R^{a1})—C(R^{a2})(R^{a3})—L^{a2}—COOH. In some embodiments, L^{a2} is —CH₂SCH₂—.

In some embodiments, L^{a2} is a covalent bond. In some embodiments, a compound of formula A-I is of the structure NH(R^{a1})—L^{a1}—C(R^{a2})(R^{a3})—COOH. In some embodiments, an amino acid residue has the structure of —N(R^{a1})—L^{a1}—C(R^{a2})(R^{a3})—CO—. In some embodiments, L^{a1} is —CH₂CH₂S—. In some embodiments, L^{a1} is —CH₂CH₂S—, wherein the CH₂ is bonded to NH(R^{a1}).

In some embodiments, L^{a1} is a covalent bond and L^{a2} is a covalent bond. In some embodiments, a compound of formula A-I is of the structure NH(R^{a1})—C(R^{a2})(R^{a3})—COOH. In some embodiments, a compound of formula A-I is of the structure NH(R^{a1})—CH(R^{a2})—COOH. In some embodiments, a compound of formula A-I is of the structure NH(R^{a1})—CH(R^{a3})—COOH. In some embodiments, a compound of formula A-I is of the structure NH₂—CH(R^{a2})—COOH. In some embodiments, a compound of formula A-I is of the structure NHz—CH(R^{a3})—COOH. In some embodiments, an amino acid residue has the structure of —N(R^{a1})—C(R^{a2})(R^{a3})—CO—. In some embodiments, an amino acid residue has the structure of —N(R^{a1})—CH(R^{a2})—CO—. In some embodiments, an amino acid residue has the structure of —N(R^{a1})—CH(R^{a3})—CO—. In some embodiments, an amino acid residue has the structure of —NH—CH(R^{a2})—CO—. In some embodiments, an amino acid residue has the structure of —NH—CH(R^{a3})—CO—.

In some embodiments, L^{a} is a covalent bond. In some embodiments, L^{a} is optionally substituted C₁₋₆ bivalent aliphatic. In some embodiments, L^{a} is optionally substituted C₁₋₆ alkylene. In some embodiments, L^{a} is —CH₂—. In some embodiments, L^{a} is —CH₂CH₂—. In some embodiments, L^{a} is —CH₂CH₂CH₂—.

In some embodiments, R′ is R. In some embodiments, R^{a1} is R, wherein R is as described in the present disclosure. In some embodiments, R^{a1} is R, wherein R methyl. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen. In some embodiments, R^{a2} is —L^{a}—R and R^{a3} is —H. In some embodiments, R^{a3} is —L^{a}—R and R^{a2} is —H. In some embodiments, R^{a2} is —CH₂—R and R^{a3} is —H. In some embodiments, R^{a3} is —CH₂—R and R^{a2} is —H. In some embodiments, R^{a2} is R and R^{a3} is —H. In some embodiments, R^{a3} is R and R^{a2} is -H.

In some embodiments, R^{a2} is —L^{a}—R, wherein R is as described in the present disclosure. In some embodiments, R^{a2} is —L^{a}—R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is —L^{a}—R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is —L^{a}—R, wherein R is as described in the present disclosure. In some embodiments, R^{a3} is —L^{a}—R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is —L^{a}—R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

In some embodiments, R is an optionally substituted C₁₋₆ aliphatic. In some embodiments, R is an optionally substituted C₁₋₆ alkyl. In some embodiments, R is —CH₃. In some embodiments, R is optionally substituted pentyl. In some embodiments, R is n-pentyl.

In some embodiments, R is a cyclic group. In some embodiments, R is an optionally substituted C₃₋₃₀ cycloaliphatic group. In some embodiments, R is cyclopropyl.

In some embodiments, R is an optionally substituted aromatic group, and an amino acid residue of an amino acid of formula A-I is a Xaa^{A}. In some embodiments, R^{a2} or R^{a3} is —CH₂—R, wherein R is an optionally substituted aryl or heteroaryl group. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

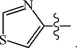

In some embodiments, R is optionally substituted pyridinyl. In some embodiments, R is 1- pyridinyl. In some embodiments, R is 2-pyridinyl. In some embodiments, R is 3- pyridinyl. In some embodiments, R is

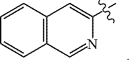

In some embodiments, R′ is-COOH. In some embodiments, a compound of and an amino acid residue of an amino acid of formula A-I is a Xaa^{N}.

In some embodiments, R′ is-NHz. In some embodiments, a compound of an amino acid residue of an amino acid of formula A-I is a Xaa^{P}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is n-propyl. In some embodiments, R is butyl. In some embodiments, R is n-butyl. In some embodiments, R is pentyl. In some embodiments, R is n-pentyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid. In some embodiments, a compound of formula A-I is a natural amino acid. In some embodiments, a compound of formula A-I is an unnatural amino acid.

In some embodiments, an amino acid comprises a hydrophobic side chain. In some embodiments, an amino acid with a hydrophobic side chain is A, V, I, L, M, F, Y or W. In some embodiments, an amino acid with a hydrophobic side chain is A, V, I, L, M, or F. In some embodiments, an amino acid with a hydrophobic side chain is A, V, I, L, or M. In some embodiments, an amino acid with a hydrophobic side chain is A, V, I, or L. In some embodiments, a hydrophobic side chain is R wherein R is C_{1-1O} aliphatic. In some embodiments, R is C_{1-1O} alkyl. In some embodiments, R is methyl. In some embodiments, R is ethyl. In some embodiments, R is propyl. In some embodiments, R is butyl. In some embodiments, R is pentyl. In some embodiments, R is n-pentyl. In some embodiments, an amino acid with a hydrophobic side chain is NH₂CH(CH₂CH₂CH₂CH₂CH₃)COOH. In some embodiments, an amino acid with a hydrophobic side chain is (,S)-NH₂CH(CH₂CH₂CH₂CH₂CH₃)COOH. In some embodiments, an amino acid with a hydrophobic side chain is (R)-NHzCH(CHzCHzCHzCHzCH₃)COOH. In some embodiments, a hydrophobic side chain is —CH₂R wherein R is optionally substituted phenyl. In some embodiments, R is phenyl. In some embodiments, R is phenyl substituted with one or more hydrocarbon group. In some embodiments, R is 4-phenylphenyl. In some embodiments, an amino acid with a hydrophobic side chain is NH₂CH(CH₂-4-phenylphenyl)COOH. In some embodiments, an amino acid with a hydrophobic side chain is (S)—NH₂CH(CH₂—4-phenylphenyl)COOH. In some embodiments, an amino acid with a hydrophobic side chain is (R)-NH₂CH(CH₂-4-phenylphenyl)COOH.

In some embodiments, an amino acid comprises a positively charged side chain (e.g., at physiological pH) as described herein. In some embodiments, such an amino acid comprises a basic nitrogen in its side chain. In some embodiments, such an amino acid is Arg, His or Lys. In some embodiments, such an amino acid is Arg. In some embodiments, such an amino acid is His. In some embodiments, such an amino acid is Lys.

In some embodiments, an amino acid comprises a negatively charged side chain (e.g., at physiological pH) as described herein. In some embodiments, such an amino acid comprises a —COOH in its side chain. In some embodiments, such an amino acid is Asp. In some embodiments, such an amino acid is Glu.

In some embodiments, an amino acid comprises a side chain comprising an aromatic group as described herein. In some embodiments, such an amino acid is Phe, Tyr, Trp, or His. In some embodiments, such an amino acid is Phe. In some embodiments, such an amino acid is Tyr. In some embodiments, such an amino acid is Trp. In some embodiments, such an amino acid is His. In some embodiments, such an amino acid is NH₂—CH(CH₂—4-phenylphenyl)-COOH. In some embodiments, such an amino acid is (S)—NH₂—CH(CH₂—4-phenylphenyl)-COOH. In some embodiments, such an amino acid is (R)—NH₂—CH(CH₂—4-phenylphenyl)-COOH.

In some embodiments, an amino acid is an amino acid residue corresponding to a residue described for Xaa, Xaa^{T0}, Xaa^{T1}, Xaa^{T2}, Xaa^{T3}, Xaa^{T4}, Xaa^{T5}, Xaa^{T6}, Xaa^{T7}, Xaa^{T8}, Xaa^{T9}, Xaa^{T10}, Xaa^{T11}, or Xaa^{T12}.

### Target

In some embodiments, the present disclosure provides technologies for selectively directing agents comprising target binding moieties (e.g. ARM compounds) and/or antibodies (and optionally immune cells recruited by antibodies, e.g., NK cells) to desired target sites comprising one or more targets. As those skilled in the art will appreciate, provided technologies are useful for various types of targets, particularly those comprising components of SARS-CoV-2, e.g. SARS-CoV-2 viruses, cells infected thereby, cells expressing a SARS-CoV-2 spike protein or a fragment thereof, etc.

In some embodiments, targets are damaged or defective tissues. In some embodiments, a target is a damaged tissue. In some embodiments, a target is a defective tissue. In some embodiments, a target is associated with a disease, disorder or condition, e.g., COVID-19. In some embodiments, targets are or comprise diseased cells. In some embodiments, targets are or comprise cells infected by SARS-CoV-2 viruses. In some embodiments, a target is a foreign object. In some embodiments, a target is or comprises an infectious agent, e.g., a SARS-CoV-2 virus. In some embodiments, a target is or comprises viruses, e.g. SARS-CoV-2 viruses. In some embodiments, targets comprise or express a SARS-CoV-2 spike protein or a fragment thereof.

### Linker Moieties

In some embodiments, antibody binding moieties are optionally connected to target binding moieties through linker moieties. Linker moieties of various types and/or for various purposes, e.g., those utilized in antibody-drug conjugates, etc., may be utilized in accordance with the present disclosure.

Linker moieties can be either bivalent or polyvalent. In some embodiments, a linker moiety is bivalent. In some embodiments, a linker is polyvalent and connecting more than two moieties.

In some embodiments, a linker moiety is L. In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C═C—, —Cy—, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20. In some embodiments, each amino acid residue is independently a residue of an amino acid having the structure of formula A-I or a salt thereof. In some embodiments, each amino acid residue independently has the structure of —N(R^{a1})—L^{a1}—C(R^{a2})(R^{a3})—L^{a2}—CO— or a salt form thereof.

In some embodiments, L is bivalent. In some embodiments, L is a bivalent or optionally substituted, linear or branched group selected from C₁₋₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C═C—,—Cy—, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂— —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid or —[(—O—C(R′)₂—C(R′)₂₋).]-.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅₋₁₀₀ heteroarylaliphatic group having 1-20 hetereoatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})—L^{a1}—C(R^{a2})(R^{a3})—L^{a2}—CO— or a salt form thereof.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂— is —CHR′—. In some embodiments, R′ is —(CH₂)₂C(O)NH(CH₂)nCOOH. In some embodiments, R′ is —(CH₂)₂COOH. In some embodiments, R′ is —COOH.

In some embodiments, a linker moiety is or comprises one or more ring moieties, e.g., one or more methylene units of L are replaced with —Cy—. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

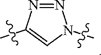

In someembodiments, a methylene unit of L is replaced with

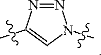

In some embodiments, —Cy— is

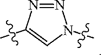

In some embodiments, a linker moiety (e.g., L) is or comprises —Cy—. In some embodiments, a methylene unit of L is replaced with —Cy—. In some embodiments, —Cy— is

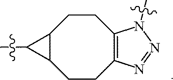

In some embodiments, —Cy— is

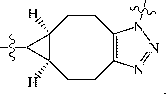

In some embodiments, —Cy— is

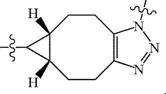

In some embodiments, a linker moiety, e.g., L, in a provided agent, e.g., a compound in Table 1, comprises

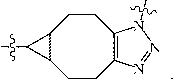

In some embodiments,

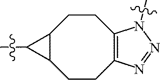

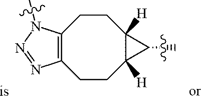

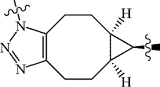

in the structure. In some embodiments,

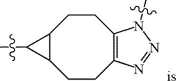

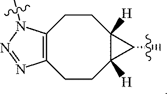

In some embodiments,

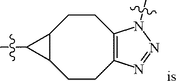

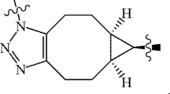

In some embodiments, a linker moiety is as described in Table 1. Additional linker moiety, for example, include those described for L². In some embodiments, L is L¹ ad present disclosure. In some embodiments, L is L² as described in the present disclosure. In some embodiments, L is L³ as described in the present disclosure. In some embodiments, L is L^{b} as described in the present disclosure.

In some embodiments, L is

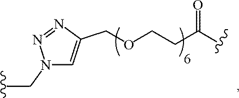

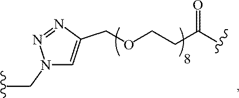

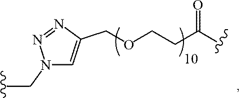

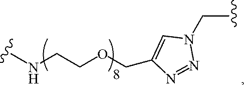

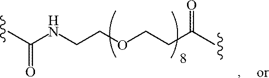

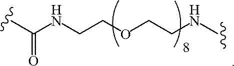

In some embodiments, a linker comprises an amino acid sequence comprising one or more amino acid residues. In some embodiments, a linker is or comprises

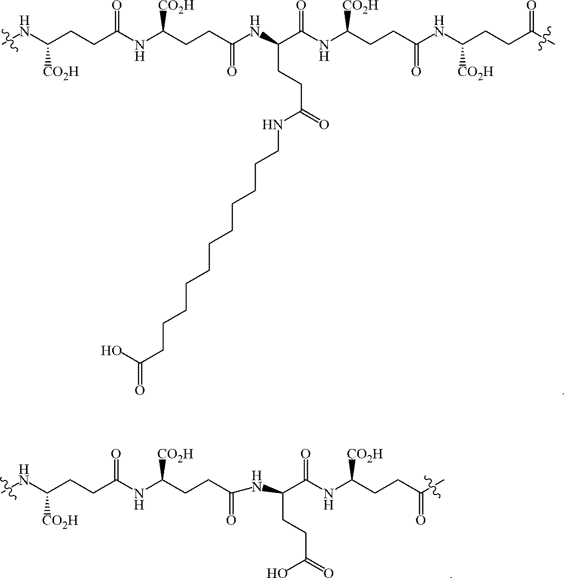

In some embodiments, a linker is or comprises

In some embodiments, a linker is or comprises a moiety, or a fragment thereof, that between two cyclic peptide moieties of a provided compound, e.g., in Table 1.

In some embodiments, a linker comprises one or more —(CH₂)n—O—, wherein each n is independently 1-50. In some embodiments, a linker comprises one or more — [(CH₂)n—O]m—, wherein each n is independently 1-50, and m is 1-100. In some embodiments, a linker comprises one or more —(O)C—[(CH₂)nO]m(CH₂)nNH—, wherein each n is independently 1-50, and each m is independently 1-100. In some embodiments, a linker comprises one or more —(CH₂)₂—O—.

In some embodiments, n is 1-10. In some embodiments, n is 1-5. In some embodiments, n is 1. In some embodiments, each n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, m is 1-50. In some embodiments, m is 1-20. In some embodiments, m is 1, 2, 3, 4, 5, 6, 7, 8, 9, 10, 11, 12, 13, 14, 15, 16, 17, 18, 19, or 20. In some embodiments, m is m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20.

In some embodiments, a linker comprises a reactive group. In some embodiments, a linker comprises a reactive group, wherein upon contact with an antibody, the reactive group reacts with a group of the antibody and conjugates a target binding moiety, or a moiety comprising -(Xaa)y-, to the antibody optionally through a linker. In some embodiments, a reactive group is or comprises

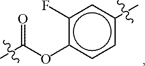

wherein —C(O)— is connected to a target binding moiety, or a moiety comprising -(Xaa)y-, optionally through a linker. In some embodiments, a reactive group is or comprises

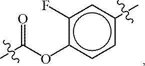

wherein —C(O)— is connected to a target binding moiety, or a moiety comprising -(Xaa)y-, optionally through a linker and the other end is connected to an antibody binding moiety optionally through another linker. Among other things, agents comprising such linkers (and optionally antibody binding moieties) are useful for preparing agents comprising antibody moieties.

In some embodiments, a linker moiety, e.g., L, is or comprises —C(O)—[(CH₂CH₂O)]m—CH₂CH₂NH—C(O)—[(CH₂CH₂O)]m—CH₂CH₂NH—. In some embodiments, a linker moiety is or comprises —C(O)—[(CH₂CH₂O)]₃—[(CH₂CH₂O)]m—CH₂CH₂NH—C(O)—[(CH₂CH₂O)]m—CH₂CH₂NH—. In some embodiments, a linker moiety is or comprises —C(O)—[(CH₂CH₂O)]m—CH₂CH₂NH—C(O)—[(CH₂CH₂O)]m—CH₂CH₂NH—C(O)—CH₂O—CH₂CH₂O—CH₂ CH₂NH—. In some embodiments, a linker moiety is or comprises —C(O)—[(CH₂CH₂O)]₃—[(CH₂CH₂O)]m—CH₂CH₂NH—C(O)—[(CH₂CH₂O)]m—CH₂CH₂NH—C(O)—CH₂O—CH₂CH₂O—CH₂CH₂NH—. In some embodiments, a linker moiety is or comprises —C(O)—[(CH₂CH₂O)]m—CH₂CH₂NH—C(O)—[(CH₂CH₂O)]m—CH₂CH₂NH—C(O)—CH₂CH₂O—CH₂CH₂O—CH₂CH₂NH—. In some embodiments, a linker moiety is or comprises —C(O)—[(CH₂CH₂O)]₃—[(CH₂CH₂O)]m—CH₂CH₂NH—C(O)—[(CH₂CH₂O)]m—CH₂CH₂NH—C(O)—CH₂CH ₂O—CH₂CH₂O—CH₂CH₂NH—. In some embodiments, a linker moiety is or comprises —C(O)—[(CH₂CH₂O)]m—CH₂CH₂NH—C(O)—[(CH₂CH₂O)]m—CH₂CH₂NH—C(O)—[(CH₂CH₂O)]m—CH₂C H₂—C(O)—. In some embodiments, a linker moiety is or comprises —C(O)—[(CH₂CH₂O)]m—CH₂CH₂NH—C(O)—[(CH₂CH₂O)]m—CH₂CH₂NH—C(O)—CH₂CH₂O—CH₂CH₂O—CH₂CH₂—C(O)—. In some embodiments, a linker moiety is or comprises —C(O)—[(CH₂CH₂O)]₃—[(CH₂CH₂O)]m—CH₂CH₂NH—C(O)—[(CH₂CH₂O)]m—CH₂CH₂NH—C(O)—CH₂CH ₂O—CH₂CH₂O—CH₂CH₂—C(O)—. In some embodiments, a linker moiety is or comprises —C(O)—[(CH₂CH₂O)]₅—CH₂CH₂NH—C(O)—[(CH₂CH₂O)]₈—CH₂CH₂NH—C(O)—CH₂CH₂O—CH₂CH₂O—C H₂CH₂—C(O)—. In some embodiments, a linker moiety is or comprises —C(O)—[(CH₂CH₂O)]₈—CH₂CH₂NH—C(O)—[(CH₂CH₂O)]₈—CH₂CH₂NH—C(O)—CH₂CH₂O—CH₂CH₂O—C H₂CH₂—C(O)—. In some embodiments, a linker moiety is or comprises —C(O)—[(CH₂CH₂O)]m—CH₂CH₂NH—C(O)—[(CH₂CH₂O)]m—CH₂CH₂NH—C(O)—CH₂CH₂O—CH₂CH₂O—CH₂CH₂—R^{RG}— wherein R^{RG} is

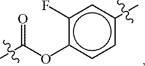

wherein the —C(O)O— of R^{RG} is bonded to —CH₂CH₂—.

In some embodiments, a linker moiety is or comprises —C(O)—[(CH₂CH₂O)]₃—[(CH₂CH₂O)]m—CH₂CH₂NH—C(O)—[(CH₂CH₂O)]m—CH₂CH₂NH—C(O)—CH₂CH ₂O—CH₂CH₂O—CH₂CH₂—R^{RG}—, wherein R^{RG} is

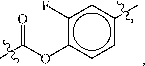

wherein the —C(O)O— of R^{RG} is bonded to —CH₂CH₂—. In some embodiments, a linker moiety is or comprises —C(O)—[(CH₂CH₂O)]₈CH₂CH₂NH—C(O)—[(CH₂CH₂O)]₈—CH₂CH₂NH—C(O)—CH₂CH₂O—CH₂CH₂O—CH₂ CH₂—R^{RG}— wherein R^{RG} is

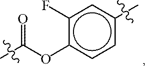

wherein the —C(O)O— of R^{RG} is bonded to —CH₂CH₂—.

In some embodiments, an antibody reacting moiety is or comprises a reactive group as described herein and optionally an antibody binding moiety. In some embodiments, an antibody reacting moiety is or comprises a reactive group as described herein and an antibody binding moiety.

As appreciated by those skilled in the art, provided technologies can be utilized for many applications (e.g., detection, diagnosis, therapeutic, etc.), particularly those that may utilize or benefit from interactions with SARS-CoV-2 or components thereof (e.g., a protein such as a spike protein or a fragment thereof). In some embodiments, provided agents may be conjugated with or incorporated into other useful agents (e.g., detection, diagnosis and/or therapeutic agents (e.g., drug agents). In some embodiments, the present disclosure provides various conjugates comprising a provided agent. In some embodiments, the present disclosure provides various conjugates comprising a provided peptide. In some embodiments, a provided agent or peptide comprises -(Xaa)y- as described herein. In some embodiments, moieties of provided agents or peptides are or comprises -(Xaa)y- as described herein. In some embodiments, moieties of provided agents or peptides are or comprises target binding moieties as described herein. In some embodiments, a provided agent or peptide is or comprises an agent of formula T-I or a salt thereof. In some embodiments, provided agents, peptides, moieties, etc. can bind to SARS-CoV-2 or components thereof (e.g., a protein such as a spike protein or a fragment thereof). In some embodiments, a provided agent, in addition to a moiety comprising -(Xaa)y- (e.g., a moiety derived from an agent of formula T-I, a target binding moiety, etc. (e.g., one that can bind to SARS-CoV-2 or components thereof (e.g., a protein such as a spike protein or a fragment thereof))), further comprises a detectable moiety. Various detectable moieties can be utilized in accordance with the present disclosure. In some embodiments, a detectable moiety can be detected directly. In some embodiments, a detectable moiety is or comprises a fluorescence moiety. In some embodiments, a detectable moiety can be detected indirectly. In some embodiments, a detectable moiety is or comprises a biotin or a derivative thereof. In some embodiments, a detectable moiety is or comprises an antibody or a fragment thereof. In some embodiments, a detectable moiety is linked to the rest of a molecule (e.g., a target binding moiety, a moiety derived from a structure of formula T-1 (e.g., by removing one or more —H to provide one or more connection sites) optionally through a linker (e.g., L) as described herein. In some embodiments, provided agent in addition to a moiety comprising -(Xaa)y- (e.g., a moiety derived from an agent of formula T-I, a target binding moiety (e.g., one that can bind to SARS-CoV-2), further comprises a reactive group (optionally connected through a linker, e.g., L. as described herein) which can serve as a handle so that other useful moieties, e.g., detectable moiety, drug moieties, etc. can be connect through reactions at the handle. For example, in some embodiments, a reactive group is azide or alkyne, which among other things can be connected through other moieties via click reactions.

In some embodiments, the present disclosure provides an agent having the structure of

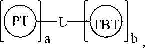

wherein PT is independently a partner moiety, and each other variable is independently as described herein. In some embodiments, the present disclosure provides an agent having the structure of

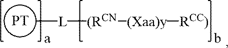

wherein each PT is independently a partner moiety, and each other variable is independently as described herein. In some embodiments, PT is a detection agent. In some embodiments, PT is diagnostic agent. In some embodiments, PT is a therapeutic agent. In some embodiments, PT is an antibody agent. In some embodiments, PT is an antibody-binding agent. In some embodiments, PT is a detectable moiety. In some embodiments, PT is or comprises

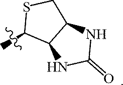

In some embodiments, PT is or comprises

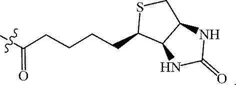

In some embodiments, the present disclosure provides methods for detecting SARS-CoV-2 or a component thereof (e.g., a spike protein or a fragment thereof) in a sample, comprising contacting the sample with a provided agent or a composition thereof. In some embodiments, the present disclosure provides methods for diagnosing a condition, disorder or disease associated with SARS-CoV-2 utilizing a provided agent or a composition thereof.

In some embodiments, a provided agent has the structure of

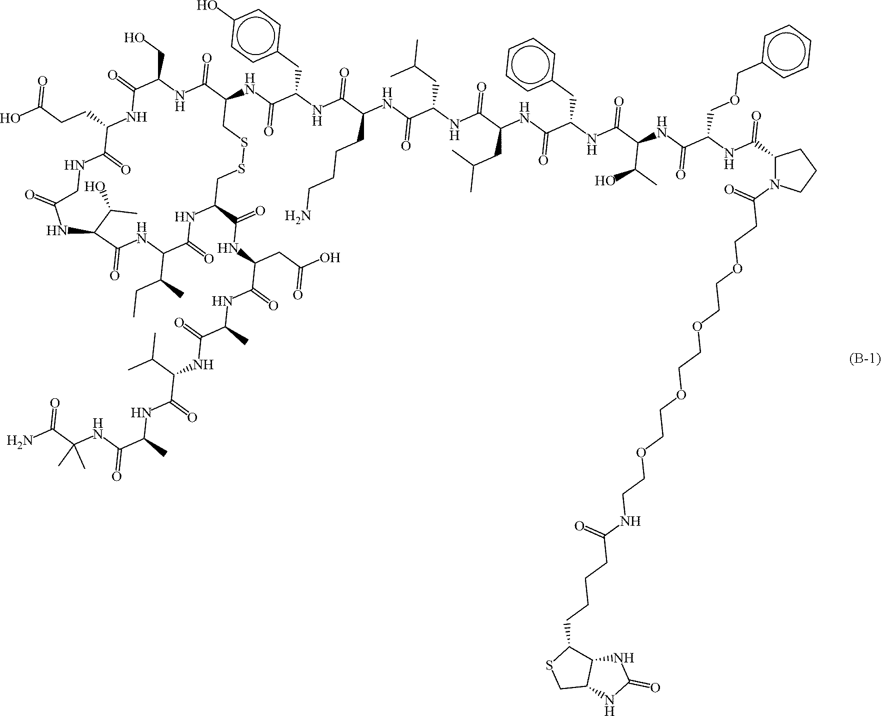

or a salt thereof. In some embodiments, a provided agent has the structure of

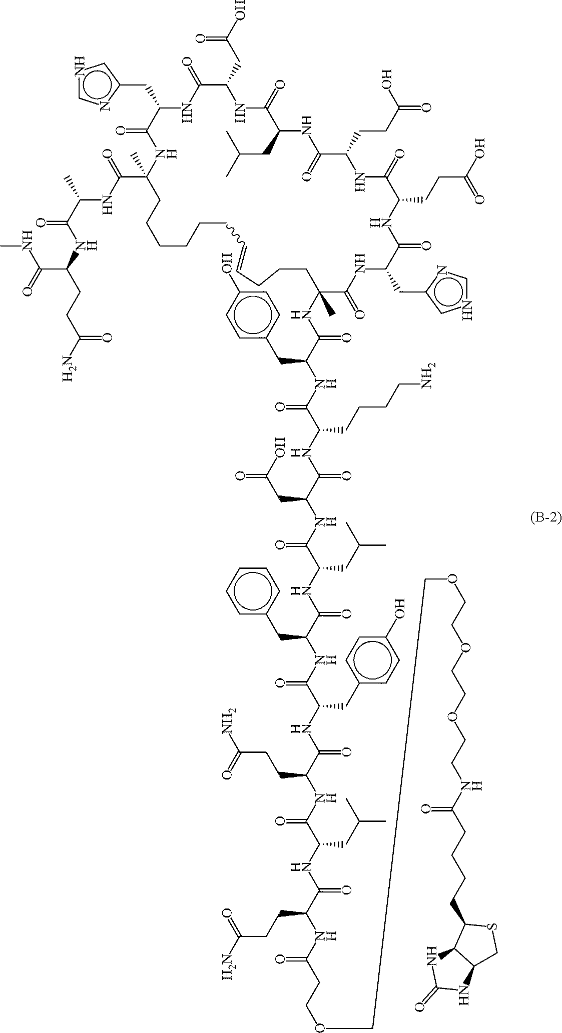

or a salt thereof. In some embodiments, a provided agent has the structure of

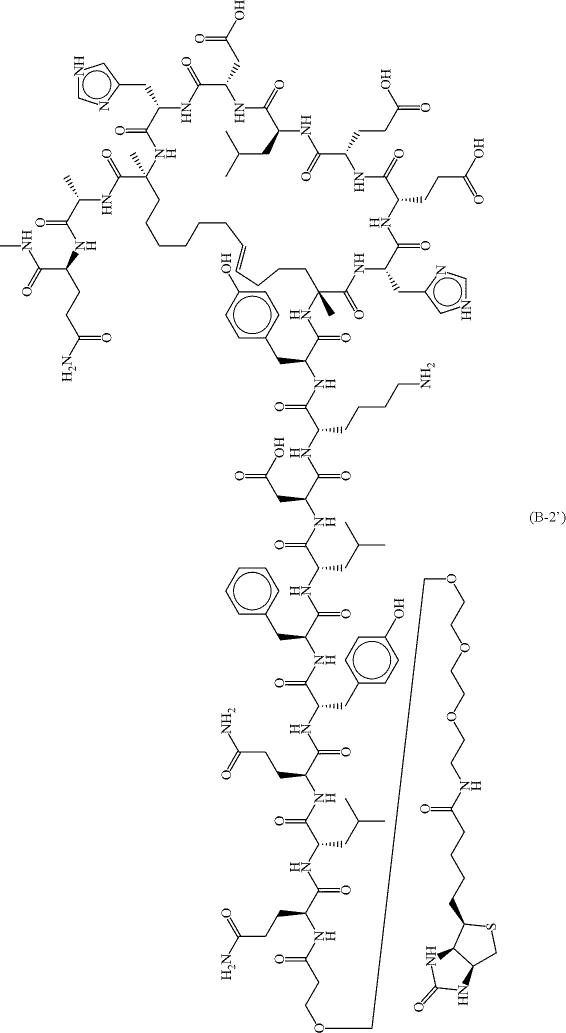

or a salt thereof. In some embodiments, a provided agent has the structure of

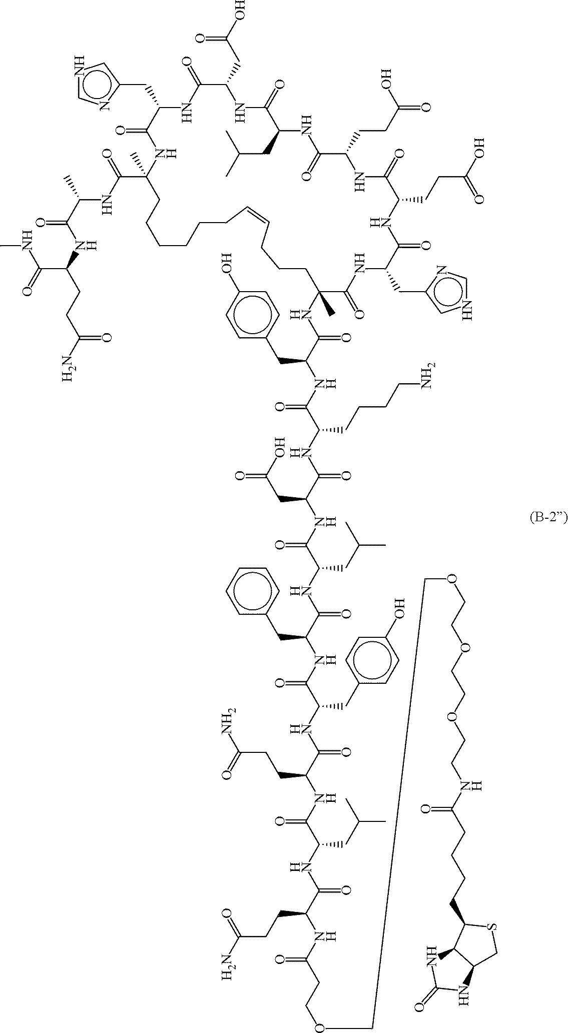

or a salt thereof. In some embodiments, a provided agent has the structure of

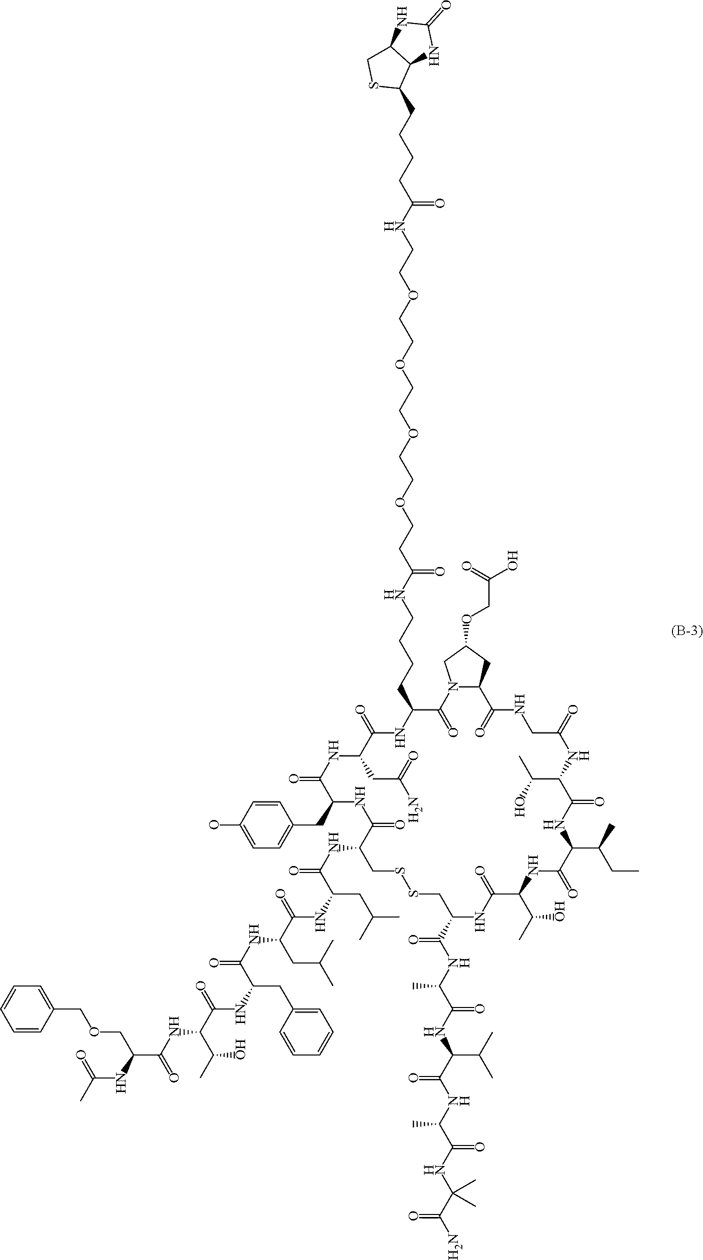 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCNC(=O)CCOCCOCCOCCOCCNC(=O)CCCC[C@H]2SC[C@H]3NC(=O)N[C@H]32)NC(=O)[C@H](CC(N)=O)NC(=O)[C@H](Cc2ccc(O)cc2)NC(=O)[C@@H](NC(=O)[C@H](CC(C)C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O]

or a salt thereof. In some embodiments, a provided agent has the structure of

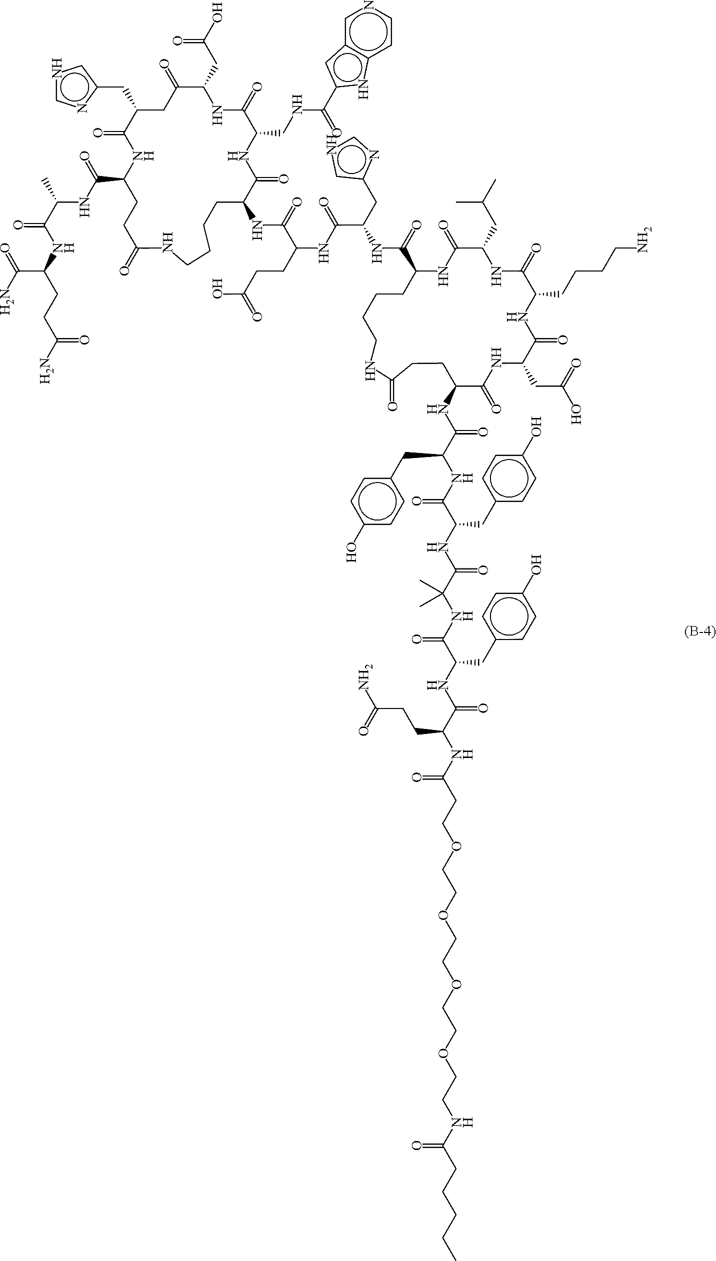

or a salt thereof.

### Certain Embodiments of Variables

As examples, exemplary embodiments of variables are described throughout the present disclosure. As appreciated by those skilled in the art, embodiments for different variables may be optionally combined.

As defined above and described herein, ABT is an antibody binding moiety as described herein. In some embodiments, an ABT is an ABT of a compound selected from those depicted in Table 1, below. In some embodiments, an ABT is a moiety selected from Table A-1. In some embodiments, an ABT is a moiety described in Table 1.

In some embodiments, L is a bivalent or multivalent linker moiety linking one or more antibody binding moieties with one or more target binding moieties. In some embodiments, L is a bivalent linker moiety that connects ABT with TBT. In some embodiments, L is a multivalent linker moiety that connects ABT with TBT.

In some embodiments, L is a linker moiety of a compound selected from those depicted in Table 1, below.

As defined above and described herein, TBT is a target binding moiety as described herein.

In some embodiments, TBT is a target binding moiety of a compound selected from those depicted in Table 1, below. In some embodiments, a TBT is a moiety selected from Table T-1. In some embodiments, an TBT is a moiety described in Table 1.

As defined above and described herein, each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: R¹ and R¹ are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; R³ and R³′ are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; an R⁵ group and the R⁵ group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; or two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or —Cy¹—, wherein each —Cy¹— is independently a 5-6 membered heteroarylenyl with 1-4 heteroatoms independently selected from nitrogen, oxygen or sulfur.

In some embodiments, R¹ is hydrogen. In some embodiments, R¹ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R¹ is an optionally substituted 8-10 membered bicyclic heteroaromatic ring having 1-5 heteroatoms independently selected from nitrogen, oxygen, or sulfur.

In some embodiments, R¹ is

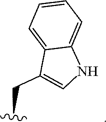

In some embodiments, R¹ is

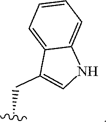

In some embodiments, R¹ is

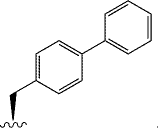

In some embodiments, R¹ is

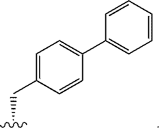

In some embodiments, R¹ is

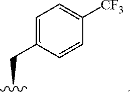

In some embodiments, R¹ is

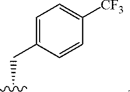

In some embodiments, R¹ is

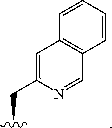

In some embodiments, R¹ is

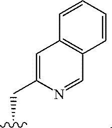

In some embodiments, R¹ is

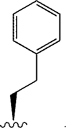

In some embodiments, R¹ is

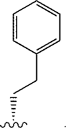

In some embodiments, R¹ is

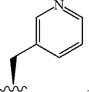

In some embodiments, R¹ is

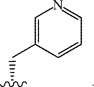

In some embodiments, R¹ is

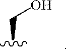

In some embodiments, R¹ is

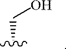

In some embodiments, R¹ is

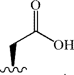

In some embodiments, R¹ is

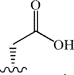

In some embodiments, R¹ is

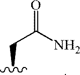

In some embodiments, R¹ is

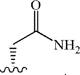

In some embodiments, R¹ is

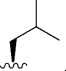

In some embodiments, R¹ is

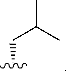

In some embodiments, R¹ and R¹ are optionally taken together with their intervening carbon atom to form a 3-8 membered saturated or partially unsaturated spirocyclic carbocyclic ring. In some embodiments, R¹ and R¹ are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R¹ is selected from those depicted in Table 1, below.

In some embodiments, R is R¹ as described in the present disclosure. In some embodiments, R^{a2} is R¹ as described in the present disclosure. In some embodiments, R^{a3} is R¹ as described in the present disclosure.

In some embodiments, R³ is hydrogen. In some embodiments, R³ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R³ is an optionally substituted C₁₋₆ aliphatic group. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, or sulfur. In some embodiments, R³ is an optionally substituted 5-6 membered monocyclic heteroaromatic ring having 1-4 heteroatoms independently selected from nitrogen, oxygen, or sulfur. In some embodiments, R³ is an optionally substituted 8-10 membered bicyclic heteroaromatic ring having 1-5 heteroatoms independently selected from nitrogen, oxygen, or sulfur.

In some embodiments, R³ is methyl. In some embodiments, R³ is

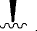

In some embodiments, R³ is

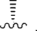

In some embodiments, R³ is

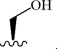

In some embodiments, R³ is

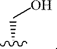

In someembodiments, R³ is

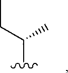

wherein the site of attachment has (S) stereochemistry. In some embodiments, R³ is

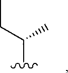

wherein the site of attachment has (R) stereochemistry. In some embodiments, R³ is

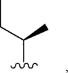

wherein the site of attachment has (S) stereochemistry. In some embodiments, R³ is

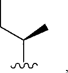

wherein the site of attachment has (R) stereochemistry.

In some embodiments, R³ is

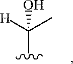

wherein the site of attachment has (S) stereochemistry. In some embodiments, R³ is

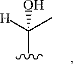

wherein the site of attachment has (R) stereochemistry.

In some embodiments, R³ and R³′ are optionally taken together with their intervening carbon atom to form a 3-8 membered saturated or partially unsaturated spirocyclic carbocyclic ring. In some embodiments, R³ and R³′ are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R³ is selected from those depicted in Table 1, below.

In some embodiments, R is R² as described in the present disclosure. In some embodiments, R^{a2} is R² as described in the present disclosure. In some embodiments, R^{a3} is R² as described in the present disclosure.

In some embodiments, R⁵ is hydrogen. In some embodiments, R⁵ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁵ is an optionally substituted C₁₋₆ aliphatic group. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 4-8 membered saturated or partially unsaturated monocyclic heterocyclic ring having 1-2 heteroatoms independently selected from nitrogen, oxygen, or sulfur. In some embodiments, R⁵ is an optionally substituted 5-6 membered monocyclic heteroaromatic ring having 1-4 heteroatoms independently selected from nitrogen, oxygen, or sulfur. In some embodiments, R⁵ is an optionally substituted 8-10 membered bicyclic heteroaromatic ring having 1-5 heteroatoms independently selected from nitrogen, oxygen, or sulfur.

In some embodiments, R⁵ is methyl. In some embodiments, R⁵ is

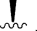

In some embodiments, R⁵ is

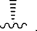

In some embodiments, R⁵ is

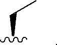

In some embodiments, R⁵ is

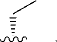

In some embodiments, R⁵ is

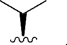

In some embodiments, R⁵ is

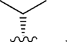

In some embodiments, R⁵ is

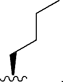

In some embodiments, R⁵ is

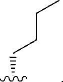

In some embodiments, R⁵ is

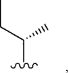

wherein the site of attachment has (S) stereochemistry. In some embodiments, R⁵ is

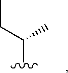

wherein the site of attachment has (R) stereochemistry. In some embodiments, R⁵ is

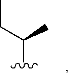

wherein the site of attachment has (S) stereochemistry. In some embodiments, R⁵ is

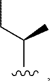

wherein the site of attachment has (R) stereochemistry. In some embodiments, R⁵ is

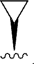

In some embodiments, R⁵ is

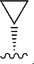

In some embodiments, R⁵ is

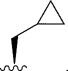

In some embodiments, R⁵ is

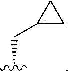

In some embodiments, R⁵ is

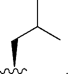

In some embodiments, R⁵ is

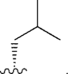

In some embodiments, R⁵ is

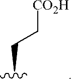

In some embodiments, R⁵ is

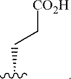

In some embodiments, R⁵ is

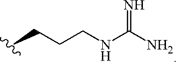

In some embodiments, R⁵ is

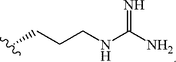

In some embodiments, R⁵ is

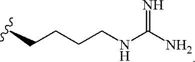

In some embodiments, R⁵ is

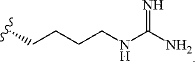

In some embodiments, R⁵ is

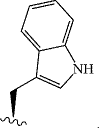

In some embodiments, R⁵ is

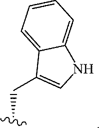

In some embodiments, R⁵ is

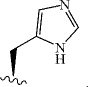

In some embodiments, R⁵ is

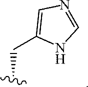

In some embodiments, R⁵ is

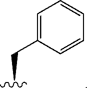

In some embodiments, R⁵ is

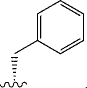

In some embodiments, R⁵ is

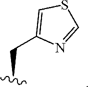

In some embodiments, R⁵ is

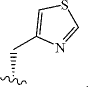

In some embodiments, R⁵ is

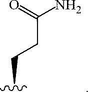

In some embodiments, R⁴ is5

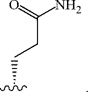

In some embodiments, R⁵ is

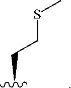

In some embodiments, R⁵ is

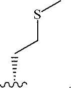

In some embodiments, R⁵ is

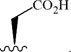

In some embodiments, R⁵ is

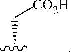

In some embodiments, R⁴ is

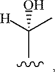

wherein the site of attachment has (S) stereochemistry. In some embodiments, R⁴ is

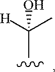

wherein the site of attachment has (R) stereochemistry.

In some embodiments, R⁵ and the R⁵ group attached to the same carbon atom are optionally taken together with their intervening carbon atom to form a 3-8 membered saturated or partially unsaturated spirocyclic carbocyclic ring. In some embodiments, R⁵ and the R⁵ group attached to the same carbon atom are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, two R⁵ groups are taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or —Cy¹—, wherein each —Cy¹— is independently a 5-6 membered heteroarylenyl with 1-4 heteroatoms independently selected from nitrogen, oxygen or sulfur.

In some embodiments, two R⁵ groups are taken together with their intervening atoms to form

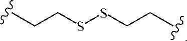

In some embodiments, two R⁵ groups are taken together with their intervening atoms to form

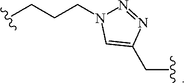

In some embodiments, two R⁵ groups are taken together with their intervening atoms to form

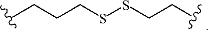

In some embodiments, two R⁵ groups are taken together with their intervening atoms to form

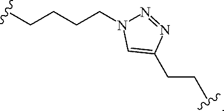

In some embodiments, R⁵ is selected from those depicted in Table 1, below.

In some embodiments, R is R⁵ as described in the present disclosure. In some embodiments, R^{a2} is R⁵ as described in the present disclosure. In some embodiments, R^{a3} is R⁵ as described in the present disclosure.

As defined above and described herein, each of R¹, R³ and R⁵ is independently hydrogen or C₁₋₃ aliphatic.

In some embodiments, R¹ is hydrogen. In some embodiments, R¹ is C₁₋₃ aliphatic.

In some embodiments, R¹ is methyl. In some embodiments, R¹ is ethyl. In some embodiments, R¹ is n-propyl. In some embodiments, R¹ is isopropyl. In some embodiments, R¹ is cyclopropyl.

In some embodiments, R¹ is selected from those depicted in Table 1, below.

In some embodiments, R^{3′} is hydrogen. In some embodiments, R^{3′} is C₁₋₃ aliphatic.

In some embodiments, R^{3′} is methyl. In some embodiments, R^{3′} is ethyl. In some embodiments, R^{3′} is n-propyl. In some embodiments, R^{3′} is isopropyl. In some embodiments, R^{3′} is cyclopropyl.

In some embodiments, R^{3′} is selected from those depicted in Table 1, below.

In some embodiments, R^{5′} is hydrogen. In some embodiments, R^{5′} is C₁₋₃ aliphatic.

In some embodiments, R^{5′} is methyl. In some embodiments, R^{5′} is ethyl. In some embodiments, R^{5′} is n-propyl. In some embodiments, R^{5′} is isopropyl. In some embodiments, R^{5′} is cyclopropyl.

In some embodiments, R^{5′} is selected from those depicted in Table 1, below.

As defined above and described herein, each of R², R⁴ and R⁶ is independently hydrogen, or C₁₋₄ aliphatic, or: R² and R¹ are optionally taken together with their intervening atoms to form a 4-8 membered saturated or partially unsaturated monocyclic heterocyclic ring having 1-2 heteroatoms independently selected from nitrogen, oxygen, or sulfur; R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R² is hydrogen. In some embodiments, R² is C₁₋₄ aliphatic. In some embodiments, R² is methyl. In some embodiments, R² is ethyl. In some embodiments, R² is n-propyl. In some embodiments, R² is isopropyl. In some embodiments, R² is n-butyl. In some embodiments, R² is isobutyl. In some embodiments, R² is tert-butyl.

In some embodiments, R² and R¹ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R² and R¹ are taken together with their intervening atoms to form

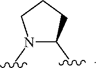

In some embodiments, R² and R¹ are taken together with their intervening atoms to form

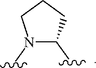

In some embodiments, R² is selected from those depicted in Table 1, below.

In some embodiments, R⁴ is hydrogen. In some embodiments, R⁴ is C₁₋₄ aliphatic. In some embodiments, R⁴ is methyl. In some embodiments, R⁴ is ethyl. In some embodiments, R⁴ is n-propyl. In some embodiments, R⁴ is isopropyl. In some embodiments, R⁴ is n-butyl. In some embodiments, R⁴ is isobutyl. In some embodiments, R⁴ is tert-butyl.

In some embodiments, R⁴ and R³ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁴ and R³ are taken together with their intervening atoms to form

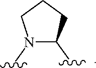

In some embodiments, R⁴ and R³ are taken together with their intervening atoms to form

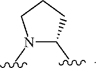

In some embodiments, R⁴ is selected from those depicted in Table 1, below.

In some embodiments, R⁶ is hydrogen. In some embodiments, R⁶ is C₁₋₄ aliphatic. In some embodiments, R⁶ is methyl. In some embodiments, R⁶ is ethyl. In some embodiments, R⁶ is n-propyl. In some embodiments, R⁶ is isopropyl. In some embodiments, R⁶ is n-butyl. In some embodiments, R⁶ is isobutyl. In some embodiments, R⁶ is tert-butyl.

In some embodiments, an R⁶ group and its adjacent R⁵ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁶ group and its adjacent R⁵ group are taken together with their intervening atoms to form

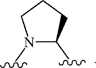

In some embodiments, an R⁶ group and its adjacent R⁵ group are taken together with their intervening atoms to form

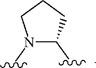

In some embodiments, R⁶ is selected from those depicted in Table 1, below.

In some embodiments, R is R^{1′} as described in the present disclosure. In some embodiments, R^{a2} is R^{1′} as described in the present disclosure. In some embodiments, R^{a3} is R^{1′} as described in the present disclosure. In some embodiments, R is R^{3′} as described in the present disclosure. In some embodiments, R^{a2} is R^{3′} as described in the present disclosure. In some embodiments, R^{a3} is R^{3′} as described in the present disclosure. In some embodiments, R is R² as described in the present disclosure. In some embodiments, R^{a2} is R² as described in the present disclosure. In some embodiments, R^{a3} is R² as described in the present disclosure. In some embodiments, R is R⁴ as described in the present disclosure. In some embodiments, R^{a2} is R⁴ as described in the present disclosure. In some embodiments, R^{a3} is R⁴ as described in the present disclosure. In some embodiments, R is R⁶ as described in the present disclosure. In some embodiments, R^{a2} is R⁶ as described in the present disclosure. In some embodiments, R^{a3} is R⁶ as described in the present disclosure.

As defined above and described herein, L¹ is a trivalent linker moiety that connects

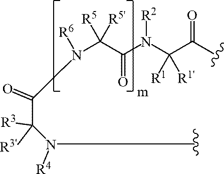

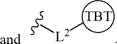

In some embodiments, L¹ is

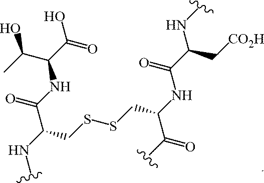

In some embodiments, L¹ is

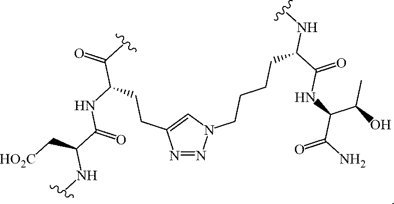

In some embodiments, L¹ is

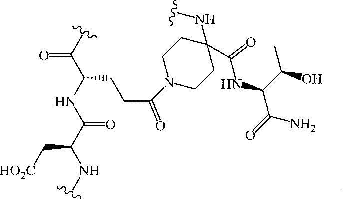

In some embodiments, L¹ is

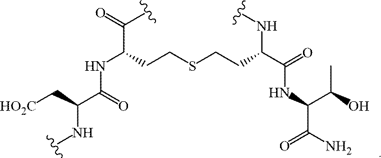

In some embodiments, L¹ is

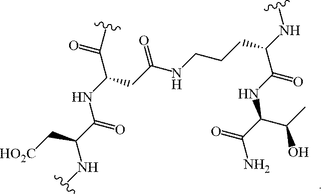

In some embodiments, L¹ is

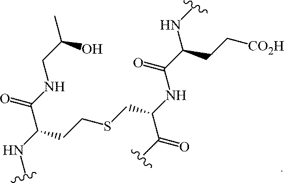

In someembodiments, L¹ is

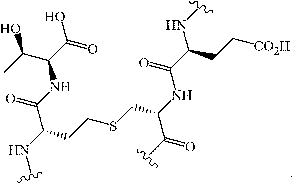

In some embodiments, L¹ is

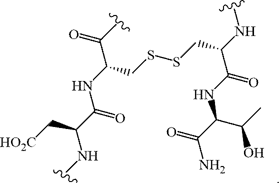

In some embodiments, L¹ is

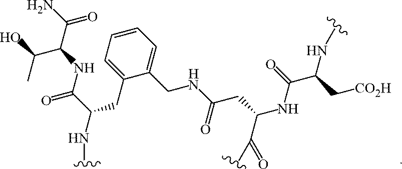

In some embodiments, L¹ is

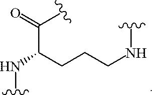

In some embodiments, L¹ is

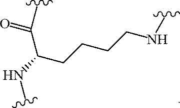

In some embodiments,

L¹ is

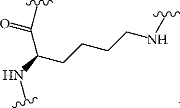

In some embodiments, L¹ is

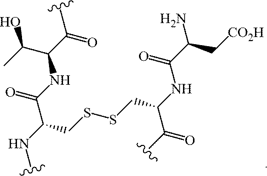

In some embodiments, L¹ is

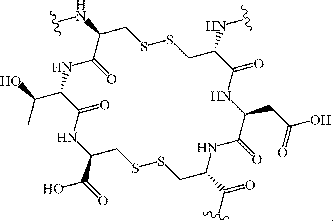

In some embodiments, L¹ is

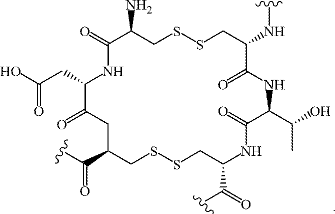

In some embodiments, L¹ is

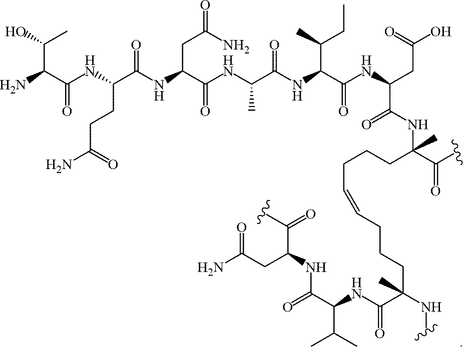

In someembodiments, L¹ is

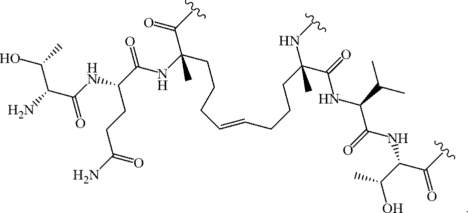

In some embodiments, L¹ is

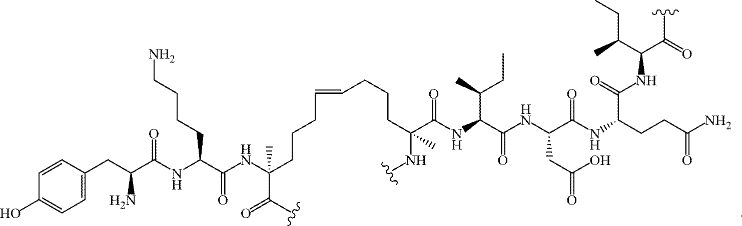

In some embodiments, L¹ is

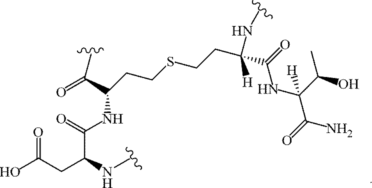

In some embodiments, L¹ is

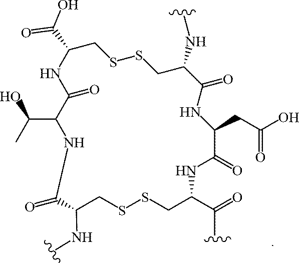

In some embodiments, L¹ is

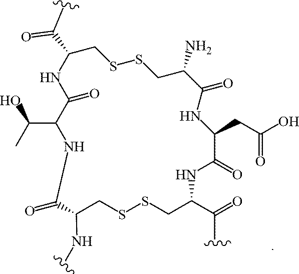

In some embodiments, L¹ is

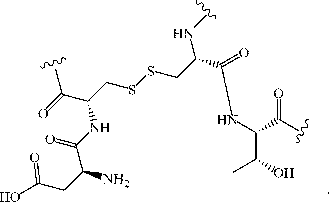

In some embodiments, L¹ is selected from those depicted in Table 1, below.

As defined above and described herein, L² is a covalent bond or a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—,—N(R)C(O)—, —S(O)—, —S(O)₂—,

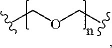

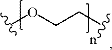

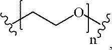

or —Cy¹—, wherein each —Cy¹— is independently a 5-6 membered heteroarylenyl with 1-4 heteroatoms independently selected from nitrogen, oxygen or sulfur.

In some embodiments, L² is a covalent bond. In some embodiments, L² is a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

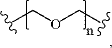

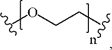

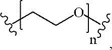

or —Cy¹—, wherein each —Cy¹— is independently a 5-6 membered heteroarylenyl with 1-4 heteroatoms independently selected from nitrogen, oxygen or sulfur.

In some embodiments, L² is

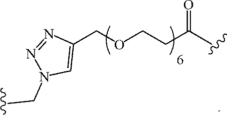

In some embodiments, L² is

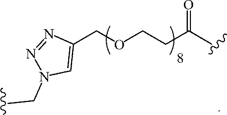

In some embodiments, L² is

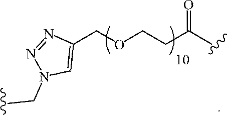

In some embodiments, L² is

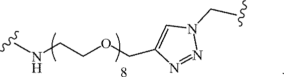

In some embodiments, L² is

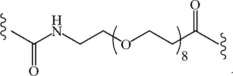

In some embodiments, L² is

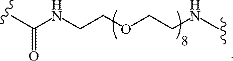

In some embodiments, L² is selected from those depicted in Table 1, below.

In some embodiments, L is L² as described in the present disclosure.

As defined above and described herein, TBT is a target binding moiety.

In some embodiments, TBT is a target binding moiety.

In some embodiments, TBT is

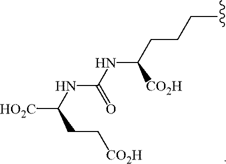

In some embodiments, TBT is

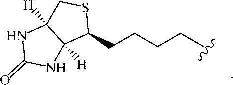

In some embodiments, TBT is selected from those depicted in Table 1, below.

As defined above and described herein, each of m and n is independently 1, 2, 3, 4, 5, 6, 7, 8, 9, or 10.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, m is selected from those depicted in Table 1, below.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, n is selected from those depicted in Table 1, below.

As defined above and described herein, each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: an R⁷ group and the R^{7′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur.

In some embodiments, R⁷ is hydrogen. In some embodiments, R⁷ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, or sulfur. In some embodiments, R⁷ is an optionally substituted 5-6 membered monocyclic heteroaromatic ring having 1-4 heteroatoms independently selected from nitrogen, oxygen, or sulfur. In some embodiments, R⁷ is an optionally substituted 8-10 membered bicyclic heteroaromatic ring having 1-5 heteroatoms independently selected from nitrogen, oxygen, or sulfur.

In some embodiments, R⁷ is methyl. In some embodiments, R⁷ is

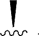

In some embodiments, R⁷ is

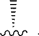

In some embodiments, R⁷ is

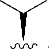

In some embodiments, R⁷ is

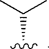

In some embodiments, R⁷ is

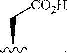

In some embodiments, R⁷ is

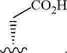

In some embodiments, R⁷ is

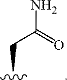

In some embodiments, R⁷ is

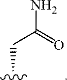

In some embodiments, R⁷ is

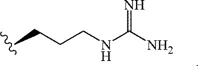

In some embodiments, R⁷ is

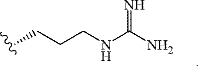

In some embodiments, R⁷ is

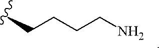

In some embodiments, R⁷ is

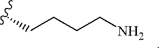

In some embodiments, R⁷ is

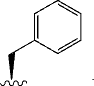

In some embodiments, R⁷ is

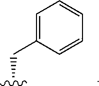

In someembodiments, R⁷ is

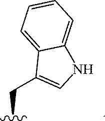

In some embodiments, R⁷ is

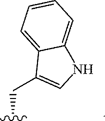

In some embodiments, R⁷ is

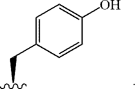

In some embodiments, R⁷ is

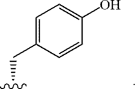

In some embodiments, R⁷ is

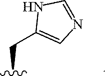

In some embodiments, R⁷ is

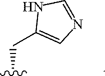

In some embodiments, an R⁷ group and the R^{7′} group attached to the same carbon atom are taken together with their intervening carbon atom to form a 3-8 membered saturated or partially unsaturated spirocyclic carbocyclic ring. In some embodiments, an R⁷ group and the R^{7′} group attached to the same carbon atom are taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R⁷ is selected from those depicted in Table 1, below.

As defined above and described herein, each of R^{7′} is independently hydrogen or C₁₋₃ aliphatic.

In some embodiments, R^{7′} is hydrogen. In some embodiments, R^{7′} is methyl. In some embodiments, R^{7′} is ethyl. In some embodiments, R^{7′} is n-propyl. In some embodiments, R^{7′} is isopropyl.

In some embodiments, R^{7′} is selected from those depicted in Table 1, below.

As defined above and described herein, each of R⁸ is independently hydrogen, or C₁₋₄ aliphatic, or: an R⁸ group and its adjacent R⁷ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁸ is hydrogen. In some embodiments, R⁸ is C₁₋₄ aliphatic. In some embodiments, R⁸ is methyl. In some embodiments, R⁸ is ethyl. In some embodiments, R⁸ is n-propyl. In some embodiments, R⁸ is isopropyl. In some embodiments, R⁸ is n-butyl. In some embodiments, R⁸ is isobutyl. In some embodiments, R⁸ is tert-butyl.

In some embodiments, an R⁸ group and its adjacent R⁷ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁸ group and its adjacent R⁷ group are taken together with their intervening atoms to form

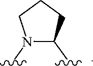

In some embodiments, an R⁸ group and its adjacent R⁷ group are taken together with their intervening atoms to form

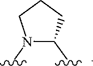

In some embodiments, R⁸ is selected from those depicted in Table 1, below.

As defined above and described herein, R⁹ is hydrogen, C₁₋₃ aliphatic, or —C(O)C₁₋₃ aliphatic.

In some embodiments, R⁹ is hydrogen. In some embodiments, R⁹ is C₁₋₃ aliphatic. In some embodiments, R⁹ is —C(O)C₁—₃ aliphatic.

In some embodiments, R⁹ is methyl. In some embodiments, R⁹ is ethyl. In some embodiments, R⁹ is n-propyl. In some embodiments, R⁹ is isopropyl. In some embodiments, R⁹ is cyclopropyl.

In some embodiments, R⁹ is —C(O)Me. In some embodiments, R⁹ is —C(O)Et. In some embodiments, R⁹ is —C(O)CH₂CH₂CH₃. In some embodiments, R⁹ is —C(O)CH(CH₃)₂. In some embodiments, R⁹ is —C(O)cyclopropyl.

In some embodiments, R⁹ is selected from those depicted in Table 1, below.

In some embodiments, R is R⁷ as described in the present disclosure. In some embodiments, R^{a2} is R⁷ as described in the present disclosure. In some embodiments, R^{a3} is R⁷ as described in the present disclosure. In some embodiments, R is R^{7′} as described in the present disclosure. In some embodiments, R^{a2} is R^{7′} as described in the present disclosure. In some embodiments, R^{a3} is R^{7′} as described in the present disclosure. In some embodiments, R is R⁸ as described in the present disclosure. In some embodiments, R^{a2} is R⁸ as described in the present disclosure. In some embodiments, R^{a3} is R⁸ as described in the present disclosure. In some embodiments, R is R^{8′} as described in the present disclosure. In some embodiments, R^{a2} is R^{8′} as described in the present disclosure. In some embodiments, R^{a3} is R^{8′} as described in the present disclosure. In some embodiments, R is R⁹ as described in the present disclosure. In some embodiments, R^{a2} is R⁹ as described in the present disclosure. In some embodiments, R^{a3} is R⁹ as described in the present disclosure.

As defined above and described herein, L³ is a bivalent linker moiety that connects

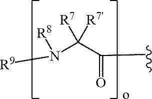

with TBT.

In some embodiments, L³ is a bivalent linker moiety that connects

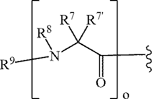

with TBT.

In some embodiments, L³ is

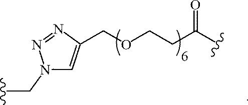

In some embodiments, L³ is

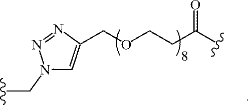

In some embodiments, L³ is

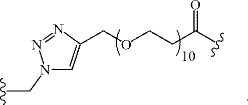

In some embodiments, L³ is

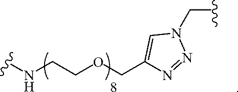

In some embodiments, L³ is

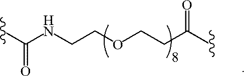

In some embodiments, L³ is

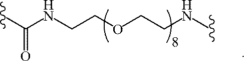

In some embodiments, L³ is selected from those depicted in Table 1, below.

In some embodiments, L is L³ as described in the present disclosure.

As defined above and described herein, o is 1, 2, 3, 4, 5, 6, 7, 8, 9, or 10.

In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10.

In some embodiments, o is selected from those depicted in Table 1, below.

In certain embodiments, the present disclosure provides a compound of formula II, wherein L² is

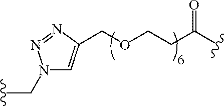

and TBT is

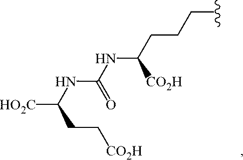

thereby forming a compound of formula II-a:

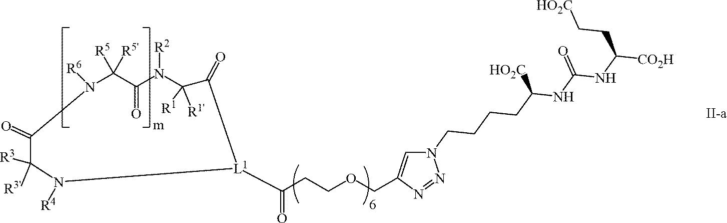

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

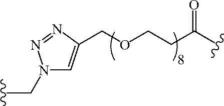

and TBT is

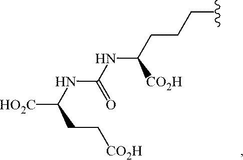

thereby forming a compound of formula II-b:

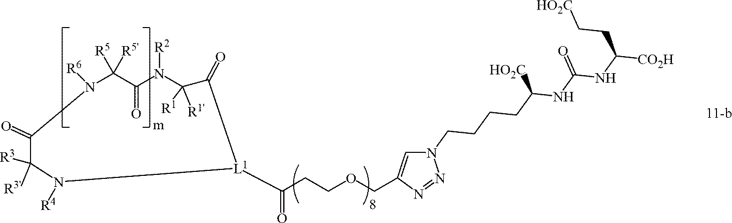

or a pharmaceutically acceptable salt thereof, wherein each of L¹, R¹, R¹, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

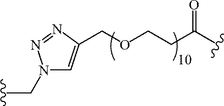

and TBT is

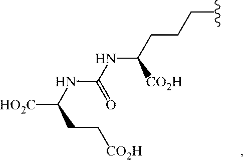

thereby forming a compound of formula II-c:

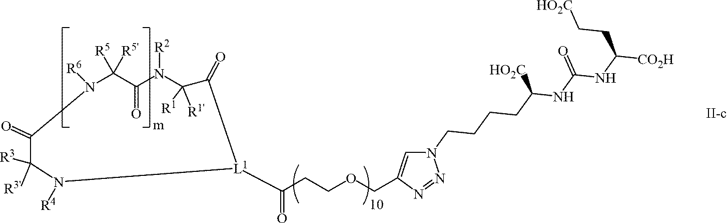

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

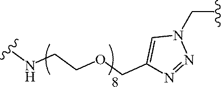

and TBT is

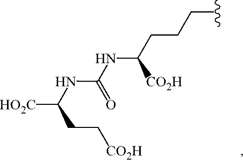

thereby forming a compound of formula II-d:

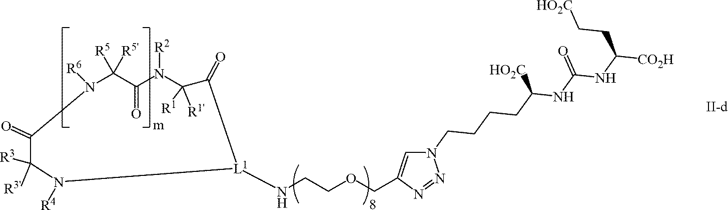

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

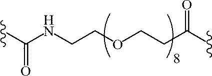

and TBT is

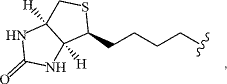

thereby forming a compound of formula II-e:

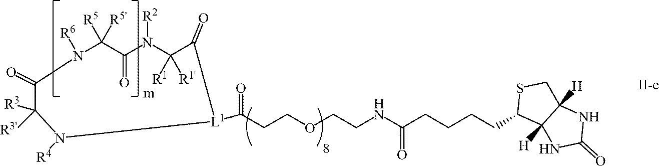

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present disclosure provides a compound of formula II, wherein L² is

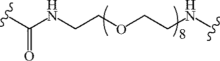

and TBT is

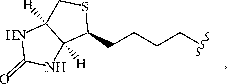

thereby forming a compound of formula II-f:

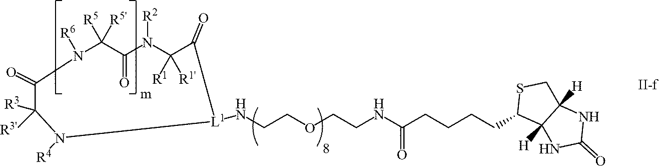

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In some embodiments, R^{a1} is R as described in the present disclosure. In some embodiments, R^{a1} is optionally substituted C₁₋₄ aliphatic. In some embodiments, R^{a1} is optionally substituted C₁₋₄ alkyl. In some embodiments, R^{a1} is methyl.

In some embodiments, L^{a1} is L^{a} as described in the present disclosure. In some embodiments, L^{a1} is a covalent bond.

In some embodiments, L^{a2} is L^{a} as described in the present disclosure. In some embodiments, L^{a2} is a covalent bond.

In some embodiments, L^{T} is L^{a} as described herein. In some embodiments, L^{T} is L as described herein. In some embodiments, L^{T} is a covalent bond. In some embodiments, L^{T} is —CH₂—C(O)—. In some embodiments, L^{T} links a —S— of a side chain (e.g., through —CH₂) with the amino group of an amino acid residue (e.g., through —C(O)—).

In some embodiments, L^{a} is a covalent bond. In some embodiments, L^{a} is an optionally substituted bivalent group selected from C₁-C₁₀ aliphatic or C₁-C₁₀ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent group selected from C₁-C₅ aliphatic or C₁-C₅ heteroaliphatic having 1-5 heteroatoms, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, -, —Cy—, —O—, —S—, —S—S—, —N(R′)—, —C(O)—, —C₂—, —S(O)₂N(R′)—, —C(O)S—, or —C(O)O—. In some embodiments, L^{a} is an optionally substituted bivalent C₁-C₅ aliphatic, wherein one or more methylene units of the group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, -S(0)₂-, —S(O)₂N(R′)—, —C(O)S—, or —C(O)O—. In some embodiments, L^{a} is an optionally substituted bivalent C₁-C₅ aliphatic. In some embodiments, L^{a} is an optionally substituted bivalent C₁-C₅ heteroaliphatic having 1-3 heteroatoms independently selected from nitrogen, oxygen and sulfur.

In some embodiments, R^{a2} is R as described in the present disclosure. In some embodiments, R^{a2} is a side chain of a natural amino acid. In some embodiments, R^{a3} is R as described in the present disclosure. In some embodiments, R^{a3} is a side chain of a natural amino acid. In some embodiments, one of R^{2a} and R^{3a} is hydrogen. In some embodiments, R^{a2} and/or R^{a3} are R, wherein R is optionally substituted C₁₋₈ alphatic or aryl. In some embodiments, R is optionally substituted linear C₂₋₈ alkyl. In some embodiments, R is linear C₂₋₈ alkyl. In some embodiments, R is optionally substituted branched C₂₋₈ alkyl. In some embodiments, R is branched C₂₋₈ alkyl. In some embodiments, R is n-pentyl. In some embodiments, R is optionally substituted phenyl. In some embodiments, R is optionally substituted -CH₂-phenyl. In some embodiments, R is 4-phenylphenyl—CH₂—.

In some embodiments, each —Cy— is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, each —Cy— is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, —Cy— is an optionally substituted ring as described in the present disclosure, for example, for R and Cy^{L}, but is bivalent.

In some embodiments, —Cy— is monocyclic. In some embodiments, —Cy— is bicyclic. In some embodiments, —Cy— is polycyclic. In some embodiments, —Cy— is saturated. In some embodiments, —Cy— is partially unsaturated. In some embodiments, —Cy— is aromatic. In some embodiments, —Cy— comprises a saturated monocyclic moiety. In some embodiments, —Cy— comprises a partially unsaturated monocyclic moiety. In some embodiments, —Cy— comprises an aromatic monocyclic moiety. In some embodiments, —Cy— comprises a combination of a saturated, a partially unsaturated, and/or an aromatic cyclic moiety. In some embodiments, —Cy— is or comprises 3-membered ring. In some embodiments, —Cy— is or comprises 4-membered ring. In some embodiments, —Cy— is or comprises 5-membered ring. In some embodiments, —Cy— is or comprises 6-membered ring. In some embodiments, —Cy— is or comprises 7-membered ring. In some embodiments, —Cy— is or comprises 8-membered ring. In some embodiments, —Cy— is or comprises 9-membered ring. In some embodiments, —Cy— is or comprises 10-membered ring. In some embodiments, —Cy— is or comprises 11-membered ring. In some embodiments, —Cy— is or comprises 12-membered ring. In some embodiments, —Cy— is or comprises 13-membered ring. In some embodiments, —Cy— is or comprises 14-membered ring. In some embodiments, —Cy— is or comprises 15-membered ring. In some embodiments, —Cy— is or comprises 16-membered ring. In some embodiments, —Cy— is or comprises 17-membered ring. In some embodiments, —Cy— is or comprises 18-membered ring. In some embodiments, —Cy— is or comprises 19-membered ring. In some embodiments, —Cy— is or comprises 20-membered ring.

In some embodiments, —Cy— is or comprises an optionally substituted bivalent C₃₋₂₀ cycloaliphatic ring. In some embodiments, —Cy— is or comprises an optionally substituted bivalent, saturated C₃₋₂₀ cycloaliphatic ring. In some embodiments, —Cy— is or comprises an optionally substituted bivalent, partially unsaturated C₃₋₂₀ cycloaliphatic ring. In some embodiments, —Cy—H is optionally substituted cycloaliphatic as described in the present disclosure, for example, cycloaliphatic embodiments for R.

In some embodiments, —Cy— is or comprises an optionally substituted C₆₋₂₀ aryl ring. In some embodiments, —Cy— is or comprises optionally substituted phenylene. In some embodiments, —Cy—is or comprises optionally substituted 1,2-phenylene. In some embodiments, —Cy— is or comprises optionally substituted 1,3-phenylene. In some embodiments, —Cy— is or comprises optionally substituted 1,4-phenylene. In some embodiments, —Cy— is or comprises an optionally substituted bivalent naphthalene ring. In some embodiments, —Cy—H is optionally substituted aryl as described in the present disclosure, for example, aryl embodiments for R.

In some embodiments, —Cy— is or comprises an optionally substituted bivalent 5-20 membered heteroaryl ring having 1-10 heteroatoms independently selected from oxygen, nitrogen, sulfur, phosphorus and silicon. In some embodiments, —Cy— is or comprises an optionally substituted bivalent 5-20 membered heteroaryl ring having 1-10 heteroatoms independently selected from oxygen, nitrogen, and sulfur. In some embodiments, —Cy— is or comprises an optionally substituted bivalent 5-6 membered heteroaryl ring having 1-4 heteroatoms independently selected from oxygen, nitrogen, sulfur. In some embodiments, —Cy— is or comprises an optionally substituted bivalent 5-6 membered heteroaryl ring having 1-3 heteroatoms independently selected from oxygen, nitrogen, sulfur. In some embodiments, —Cy— is or comprises an optionally substituted bivalent 5-6 membered heteroaryl ring having 1-2 heteroatoms independently selected from oxygen, nitrogen, sulfur. In some embodiments, —Cy— is or comprises an optionally substituted bivalent 5-6 membered heteroaryl ring having one heteroatom independently selected from oxygen, nitrogen, sulfur. In some embodiments, —Cy—H is optionally substituted heteroaryl as described in the present disclosure, for example, heteroaryl embodiments for R. In some embodiments, —Cy— is

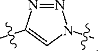

In some embodiments, —Cy— is or comprises an optionally substituted bivalent 3-20 membered heterocyclyl ring having 1-10 heteroatoms independently selected from oxygen, nitrogen, sulfur, phosphorus and silicon. In some embodiments, —Cy— is or comprises an optionally substituted bivalent 3-20 membered heterocyclyl ring having 1-10 heteroatoms independently selected from oxygen, nitrogen, and sulfur. In some embodiments, —Cy— is or comprises an optionally substituted bivalent 3-6 membered heterocyclyl ring having 1-4 heteroatoms independently selected from oxygen, nitrogen, sulfur. In some embodiments, —Cy— is or comprises an optionally substituted bivalent 5-6 membered heterocyclyl ring having 1-4 heteroatoms independently selected from oxygen, nitrogen, sulfur. In some embodiments, —Cy— is or comprises an optionally substituted bivalent 5-6 membered heterocyclyl ring having 1-3 heteroatoms independently selected from oxygen, nitrogen, sulfur. In some embodiments, —Cy— is or comprises an optionally substituted bivalent 5-6 membered heterocyclyl ring having 1-2 heteroatoms independently selected from oxygen, nitrogen, sulfur. In some embodiments, —Cy— is or comprises an optionally substituted bivalent 5-6 membered heterocyclyl ring having one heteroatom independently selected from oxygen, nitrogen, sulfur. In some embodiments, —Cy— is or comprises an optionally substituted saturated bivalent heterocyclyl group. In some embodiments, —Cy— is or comprises an optionally substituted partially unsaturated bivalent heterocyclyl group. In some embodiments, —Cy—H is optionally substituted heterocyclyl as described in the present disclosure, for example, heterocyclyl embodiments for R.

In some embodiments, —Cy— is

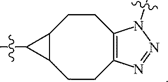

In some embodiments, —Cy— is

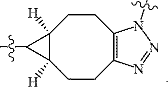

In some embodiments, —Cy— is

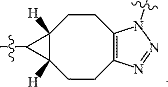

In some embodiments, —Cy—is

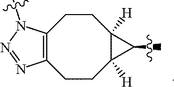

In some embodiments, —Cy— is

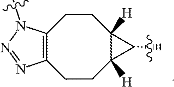

In some embodiments, each Xaa is independently an amino acid residue. In some embodiments, each Xaa is independently an amino acid residue of an amino acid of formula A-I.

In some embodiments, t is 0. In some embodiments, t is 1-50. In some embodiments, t is z as described in the present disclosure.

In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15. In some embodiments, y is 16. In some embodiments, y is 17. In some embodiments, y is 18. In some embodiments, y is 19. In some embodiments, y is 20. In some embodiments, y is greater than 20.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, z is 13. In some embodiments, z is 14. In some embodiments, z is 15. In some embodiments, z is 16. In some embodiments, z is 17. In some embodiments, z is 18. In some embodiments, z is 19. In some embodiments, z is 20. In some embodiments, z is greater than 20.

In some embodiments, R^{c} is R′ as described in the present disclosure. In some embodiments, R^{c} is R as described in the present disclosure. In some embodiments, R^{c} is —N(R′)₂, wherein each R′ is independently as described in the present disclosure. In some embodiments, R^{c} is —NH₂. In some embodiments, R^{c} is R—C(O)—, wherein R is as described in the present disclosure. In some embodiments, R^{c} is —H.

In some embodiments, a is 1. In some embodiments, a is 2-100. In some embodiments, a is 5. In some embodiments, a is 10. In some embodiments, a is 20. In some embodiments, a is 50.

In some embodiments, b is 1. In some embodiments, b is 2-100. In some embodiments, b is 5. In some embodiments, b is 10. In some embodiments, b is 20. In some embodiments, b is 50.

In some embodiments, a1 is 0. In some embodiments, a1 is 1.

In some embodiments, a2 is 0. In some embodiments, a2 is 1.

In some embodiments, L^{b} is L^{a} as described in the present disclosure. In some embodiments, L^{b} comprises —Cy—. In some embodiments, L^{b} comprises a double bond. In some embodiments, L^{b} comprises —S—. In some embodiments, L^{b} comprises —S—S—. In some embodiments, L^{b} comprises —C(O)—N(R′)—.

In some embodiments, R′ is -R, —C(O)R, —C(O)OR, or —S(O)₂R, wherein R is as described in the present disclosure. In some embodiments, R′ is R, wherein R is as described in the present disclosure. In some embodiments, R′ is —C(O)R, wherein R is as described in the present disclosure. In some embodiments, R′ is —C(O)OR, wherein R is as described in the present disclosure. In some embodiments, R′ is —S(O)₂R, wherein R is as described in the present disclosure. In some embodiments, R′ is hydrogen. In some embodiments, R′ is not hydrogen. In some embodiments, R′ is R, wherein R is optionally substituted C₁₋₂₀ aliphatic as described in the present disclosure. In some embodiments, R′ is R, wherein R is optionally substituted C₁₋₂₀ heteroaliphatic as described in the present disclosure. In some embodiments, R′ is R, wherein R is optionally substituted C₆₋₂₀ aryl as described in the present disclosure. In some embodiments, R′ is R, wherein R is optionally substituted C₆₋₂₀ arylaliphatic as described in the present disclosure. In some embodiments, R′ is R, wherein R is optionally substituted C₆₋₂₀ arylheteroaliphatic as described in the present disclosure. In some embodiments, R′ is R, wherein R is optionally substituted 5-20 membered heteroaryl as described in the present disclosure. In some embodiments, R′ is R, wherein R is optionally substituted 3-20 membered heterocyclyl as described in the present disclosure. In some embodiments, two or more R′ are R, and are optionally and independently taken together to form an optionally substituted ring as described in the present disclosure.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon.

In some embodiments, R is hydrogen. In some embodiments, R is not hydrogen. In some embodiments, R is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, R is hydrogen or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, and sulfur, a 4-7 membered saturated or partially unsaturated heterocyclic ring having 1-3 heteroatoms independently selected from nitrogen, oxygen, and sulfur, a 7-10 membered bicyclic saturated or partially unsaturated heterocyclic ring having 1-5 heteroatoms independently selected from nitrogen, oxygen, and sulfur, or an 8-10 membered bicyclic heteroaryl ring having 1-5 heteroatoms independently selected from nitrogen, oxygen, and sulfur.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂CN.

In some embodiments, R is optionally substituted C₃₋₃₀ cycloaliphatic. In some embodiments, R is optionally substituted C₃₋₂₀ cycloaliphatic. In some embodiments, R is optionally substituted C₃₋₁₀ cycloaliphatic. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 3-30 membered saturated or partially unsaturated carbocyclic ring. In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, when R is or comprises a ring structure, e.g., cycloaliphatic, cycloheteroaliphatic, aryl, heteroaryl, etc., the ring structure can be monocyclic, bicyclic or polycyclic. In some embodiments, R is or comprises a monocyclic structure. In some embodiments, R is or comprises a bicyclic structure. In some embodiments, R is or comprises a polycyclic structure.

In some embodiments, R is optionally substituted C₁₋₃₀ heteroaliphatic having 1-10 heteroatoms independently selected from oxygen, nitrogen, sulfur, phosphorus and silicon. In some embodiments, R is optionally substituted C₁₋₂₀ heteroaliphatic having 1-10 heteroatoms. In some embodiments, R is optionally substituted C₁₋₂₀ heteroaliphatic having 1-10 heteroatoms independently selected from oxygen, nitrogen, sulfur, phosphorus or silicon, optionally including one or more oxidized forms of nitrogen, sulfur, phosphorus or selenium. In some embodiments, R is optionally substituted C₁₋₃₀ heteroaliphatic comprising 1-10 groups independently selected from

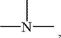

—N═, ═N, —S—, —S(O)—, —S(O)₂—, —O—, ═O,

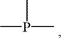

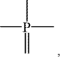

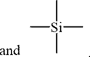

In some embodiments, R is optionally substituted C₆₋₃₀ aryl. In some embodiments, R is optionally substituted phenyl. In some embodiments, R is phenyl. In some embodiments, R is substituted phenyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted 5-30 membered heteroaryl ring having 1-10 heteroatoms independently selected from oxygen, nitrogen, sulfur, phosphorus and silicon. In some embodiments, R is optionally substituted 5-30 membered heteroaryl ring having 1-10 heteroatoms independently selected from oxygen, nitrogen, and sulfur. In some embodiments, R is optionally substituted 5-30 membered heteroaryl ring having 1-5 heteroatoms independently selected from oxygen, nitrogen, sulfur, phosphorus and silicon. In some embodiments, R is optionally substituted 5-30 membered heteroaryl ring having 1-5 heteroatoms independently selected from oxygen, nitrogen, and sulfur.

In some embodiments, R is an optionally substituted 5-6 membered monocyclic heteroaryl ring having 1-4 heteroatoms independently selected from nitrogen, oxygen, and sulfur. In some embodiments, R is a substituted 5-6 membered monocyclic heteroaryl ring having 1-4 heteroatoms independently selected from nitrogen, oxygen, and sulfur. In some embodiments, R is an unsubstituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, and sulfur.

In some embodiments, R is an optionally substituted 5-membered monocyclic heteroaryl ring having one heteroatom selected from nitrogen, oxygen, and sulfur. In some embodiments, R is optionally substituted pyrrolyl, furanyl, or thienyl.

In some embodiments, R is an optionally substituted 5-membered heteroaryl ring having two heteroatoms independently selected from nitrogen, oxygen, and sulfur. In certain embodiments, R is an optionally substituted 5-membered heteroaryl ring having one nitrogen atom, and an additional heteroatom selected from sulfur or oxygen. In some embodiments, R is an optionally substituted 5-membered heteroaryl ring having three heteroatoms independently selected from nitrogen, oxygen, and sulfur. In some embodiments, R is an optionally substituted 5-membered heteroaryl ring having four heteroatoms independently selected from nitrogen, oxygen, and sulfur.

In some embodiments, R is an optionally substituted 6-membered heteroaryl ring having 1-4 nitrogen atoms. In some embodiments, R is an optionally substituted 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In certain embodiments, R is an optionally substituted 6,6-fused heteroaryl ring having 1-4 heteroatoms independently selected from nitrogen, oxygen, and sulfur.

In some embodiments, R is 3-30 membered heterocyclic ring having 1-10 heteroatoms independently selected from oxygen, nitrogen, sulfur, phosphorus and silicon. In some embodiments, R is 3-30 membered heterocyclic ring having 1-10 heteroatoms independently selected from oxygen, nitrogen, and sulfur. In some embodiments, R is 3-30 membered heterocyclic ring having 1-5 heteroatoms independently selected from oxygen, nitrogen, sulfur, phosphorus and silicon. In some embodiments, R is 3-30 membered heterocyclic ring having 1-5 heteroatoms independently selected from oxygen, nitrogen, and sulfur.

In some embodiments, R is an optionally substituted 3-7 membered saturated or partially unsaturated heterocyclic ring having 1-3 heteroatoms independently selected from nitrogen, oxygen, and sulfur. In some embodiments, R is a substituted 3-7 membered saturated or partially unsaturated heterocyclic ring having 1-3 heteroatoms independently selected from nitrogen, oxygen, and sulfur. In some embodiments, R is an unsubstituted 3-7 membered saturated or partially unsaturated heterocyclic ring having 1-3 heteroatoms independently selected from nitrogen, oxygen, and sulfur. In certain embodiments, R is an optionally substituted 5-7 membered partially unsaturated monocyclic ring having 1-3 heteroatoms independently selected from nitrogen, oxygen, and sulfur. In certain embodiments, R is an optionally substituted 5-6 membered partially unsaturated monocyclic ring having 1-3 heteroatoms independently selected from nitrogen, oxygen, and sulfur. In certain embodiments, R is an optionally substituted 5-membered partially unsaturated monocyclic ring having 1-3 heteroatoms independently selected from nitrogen, oxygen, and sulfur. In certain embodiments, R is an optionally substituted 6-membered partially unsaturated monocyclic ring having 1-3 heteroatoms independently selected from nitrogen, oxygen, and sulfur. In certain embodiments, R is an optionally substituted 7-membered partially unsaturated monocyclic ring having 1-3 heteroatoms independently selected from nitrogen, oxygen, and sulfur. In some embodiments, R is optionally substituted 3-membered heterocyclic ring having one heteroatom selected from nitrogen, oxygen or sulfur. In some embodiments, R is optionally substituted 4-membered heterocyclic ring having 1-3 heteroatoms independently selected from nitrogen, oxygen, and sulfur. In some embodiments, R is optionally substituted 5-membered heterocyclic ring having 1-3 heteroatoms independently selected from nitrogen, oxygen, and sulfur. In some embodiments, R is optionally substituted 6-membered heterocyclic ring having 1-3 heteroatoms independently selected from nitrogen, oxygen, and sulfur. In some embodiments, R is optionally substituted 7-membered heterocyclic ring having 1-3 heteroatoms independently selected from nitrogen, oxygen, and sulfur.

In some embodiments, R is an optionally substituted 3-membered saturated or partially unsaturated heterocyclic ring having 1-2 heteroatoms independently selected from nitrogen, oxygen, and sulfur. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 7-membered saturated or partially unsaturated heterocyclic ring having 1-2 heteroatoms independently selected from nitrogen, oxygen, and sulfur.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 7-10 membered bicyclic saturated or partially unsaturated heterocyclic ring having 1-5 heteroatoms independently selected from nitrogen, oxygen, and sulfur. In some embodiments, R is optionally substituted indolinyl. In some embodiments, R is optionally substituted isoindolinyl. In some embodiments, R is optionally substituted 1, 2, 3, 4-tetrahydroquinolinyl. In some embodiments, R is optionally substituted 1, 2, 3, 4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, and sulfur. In some embodiments, R is an optionally substituted 5,6-fused heteroaryl ring having 1-5 heteroatoms independently selected from nitrogen, oxygen, and sulfur.

In some embodiments, R is optionally substituted C₆₋₃₀ arylaliphatic. In some embodiments, R is optionally substituted C₆₋₂₀ arylaliphatic. In some embodiments, R is optionally substituted C₆₋₁₀ arylaliphatic. In some embodiments, an aryl moiety of the arylaliphatic has 6, 10, or 14 aryl carbon atoms. In some embodiments, an aryl moiety of the arylaliphatic has 6 aryl carbon atoms. In some embodiments, an aryl moiety of the arylaliphatic has 10 aryl carbon atoms. In some embodiments, an aryl moiety of the arylaliphatic has 14 aryl carbon atoms. In some embodiments, an aryl moiety is optionally substituted phenyl.

In some embodiments, R is optionally substituted C₆₋₃₀ arylheteroaliphatic having 1-10 heteroatoms independently selected from oxygen, nitrogen, sulfur, phosphorus and silicon. In some embodiments, R is optionally substituted C₆₋₃₀ arylheteroaliphatic having 1-10 heteroatoms independently selected from oxygen, nitrogen, and sulfur. In some embodiments, R is optionally substituted C₆₋₂₀ arylheteroaliphatic having 1-10 heteroatoms independently selected from oxygen, nitrogen, sulfur, phosphorus and silicon. In some embodiments, R is optionally substituted C₆₋₂₀ arylheteroaliphatic having 1-10 heteroatoms independently selected from oxygen, nitrogen, and sulfur. In some embodiments, R is optionally substituted C₆₋₁₀ arylheteroaliphatic having 1-5 heteroatoms independently selected from oxygen, nitrogen, sulfur, phosphorus and silicon. In some embodiments, R is optionally substituted C₆₋₁₀ arylheteroaliphatic having 1-5 heteroatoms independently selected from oxygen, nitrogen, and sulfur.

In some embodiments, two R groups are optionally and independently taken together to form a covalent bond. In some embodiments, —C═O is formed. In some embodiments, —C═C— is formed. In some embodiments,

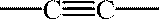

is formed.

In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-10 membered monocyclic, bicyclic or polycyclic ring having, in addition to the atom, 0-5 heteroatoms independently selected from oxygen, nitrogen, sulfur, phosphorus and silicon. In some embodiments, two or more R groups on the same atom are optionally and independently taken together with the atom to form an optionally substituted, 3-6 membered monocyclic, bicyclic or polycyclic ring having, in addition to the atom, 0-3 heteroatoms independently selected from oxygen, nitrogen, sulfur, phosphorus and silicon. In some embodiments, two or more R groups on the same atom are optionally and independently taken together with the atom to form an optionally substituted, 3-5 membered monocyclic, bicyclic or polycyclic ring having, in addition to the atom, 0-3 heteroatoms independently selected from oxygen, nitrogen, sulfur, phosphorus and silicon.

In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-5 heteroatoms independently selected from oxygen, nitrogen, sulfur, phosphorus and silicon. In some embodiments, two or more R groups on two or more atoms are optionally and independently taken together with their intervening atoms to form an optionally substituted, 3-6 membered monocyclic, bicyclic or polycyclic ring having, in addition to the intervening atoms, 0-3 heteroatoms independently selected from oxygen, nitrogen, sulfur, phosphorus and silicon. In some embodiments, two or more R groups on two or more atoms are optionally and independently taken together with their intervening atoms to form an optionally substituted, 3-5 membered monocyclic, bicyclic or polycyclic ring having, in addition to the intervening atoms, 0-3 heteroatoms independently selected from oxygen, nitrogen, sulfur, phosphorus and silicon.

In some embodiments, heteroatoms in R groups, or in the structures formed by two or more R groups taken together, are selected from oxygen, nitrogen, and sulfur. In some embodiments, a formed ring is 3, 4, 5, 6, 7, 8, 9, 10, 11, 12, 13, 14, 15, 16, 17, 18, 19, or 20-membered. In some embodiments, a formed ring is saturated. In some embodiments, a formed ring is partially saturated. In some embodiments, a formed ring is aromatic. In some embodiments, a formed ring comprises a saturated, partially saturated, or aromatic ring moiety. In some embodiments, a formed ring comprises 5, 6, 7, 8, 9, 10, 11, 12, 13, 14, 15, 16, 17, 18, 19, or 20 aromatic ring atoms. In some embodiments, a formed contains no more than 5, 6, 7, 8, 9, 10, 11, 12, 13, 14, 15, 16, 17, 18, 19, or 20 aromatic ring atoms. In some embodiments, aromatic ring atoms are selected from carbon, nitrogen, oxygen and sulfur.

In some embodiments, a ring formed by two or more R groups (or two or more groups selected from R and variables that can be R) taken together is a C₃₋₃₀ cycloaliphatic, C₆₋₃₀ aryl, 5-30 membered heteroaryl having 1-10 heteroatoms independently selected from oxygen, nitrogen, sulfur, phosphorus and silicon, or 3-30 membered heterocyclyl having 1-10 heteroatoms independently selected from oxygen, nitrogen, sulfur, phosphorus and silicon, ring as described for R, but bivalent or multivalent.

Exemplary compounds are set forth in Table 1, below. In some embodiments, a provided agent is or comprise a compound selected from Table 1 or a salt, e.g. pharmaceutically acceptable salt, thereof.

Table 1

Exemplary compounds

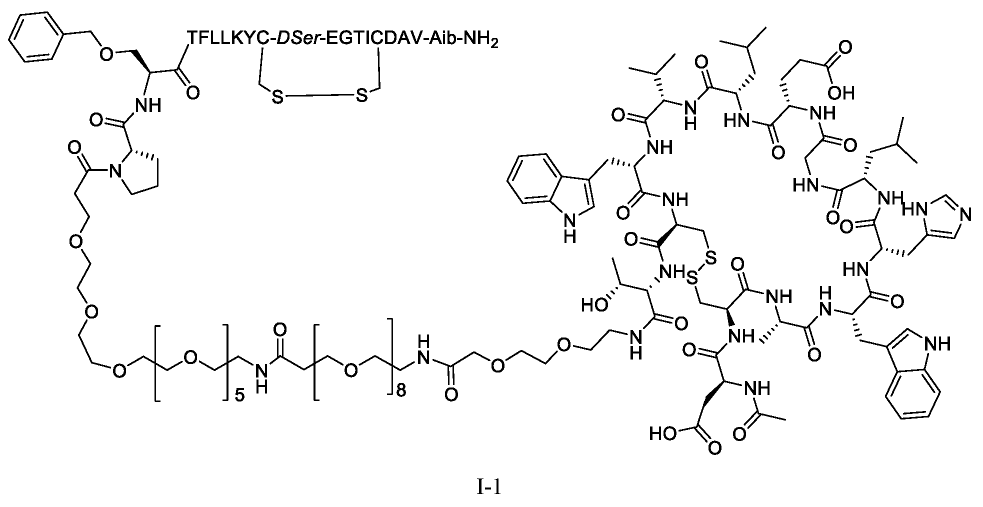

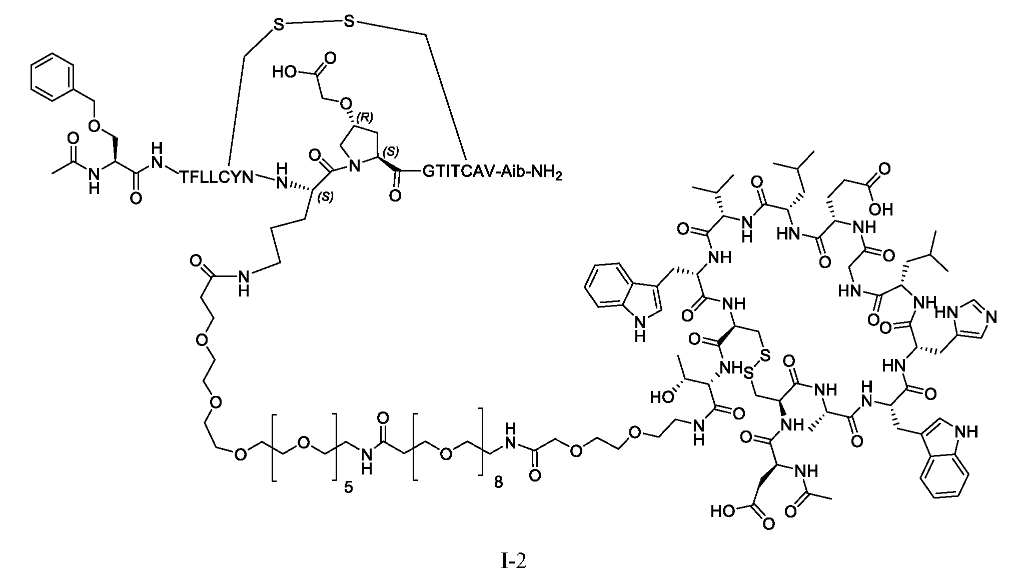

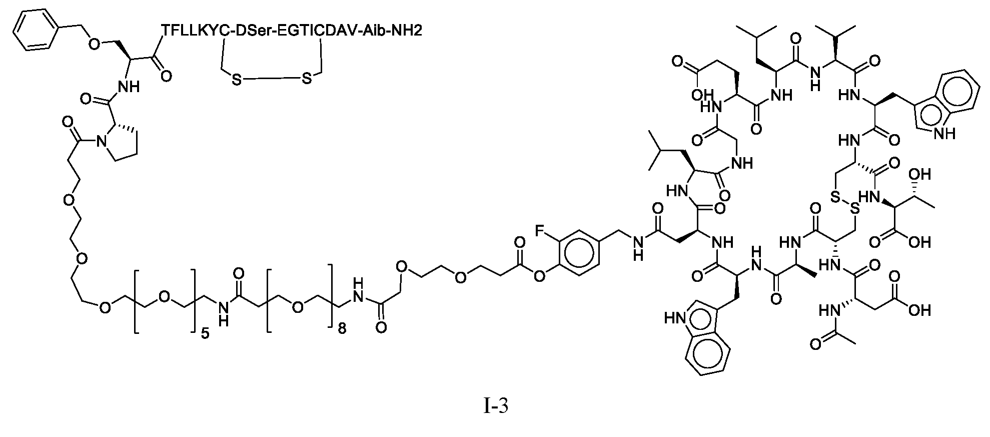

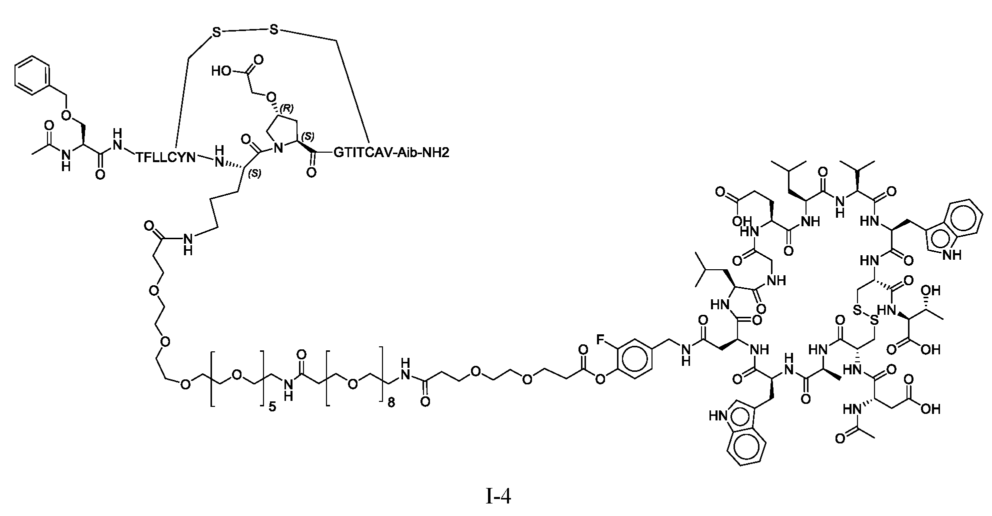

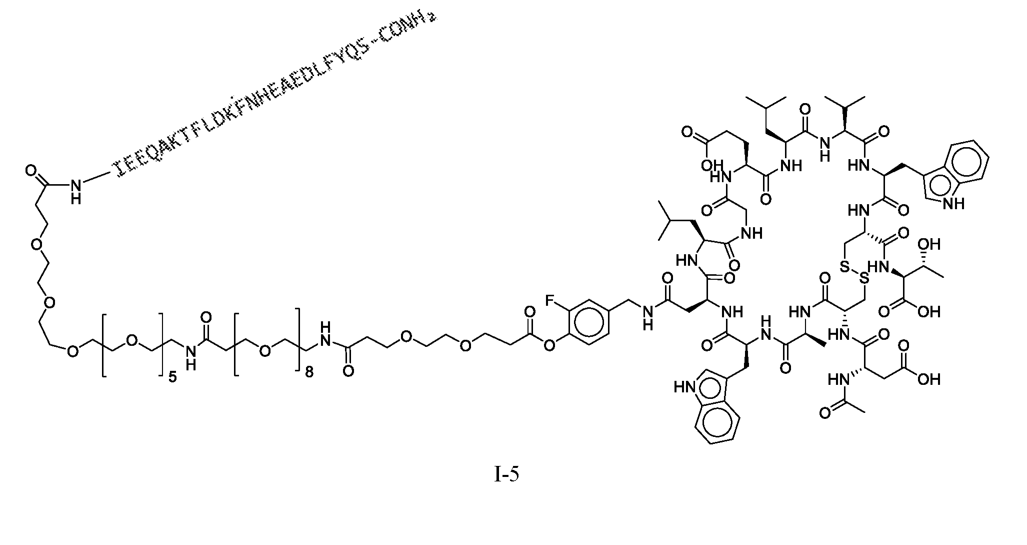

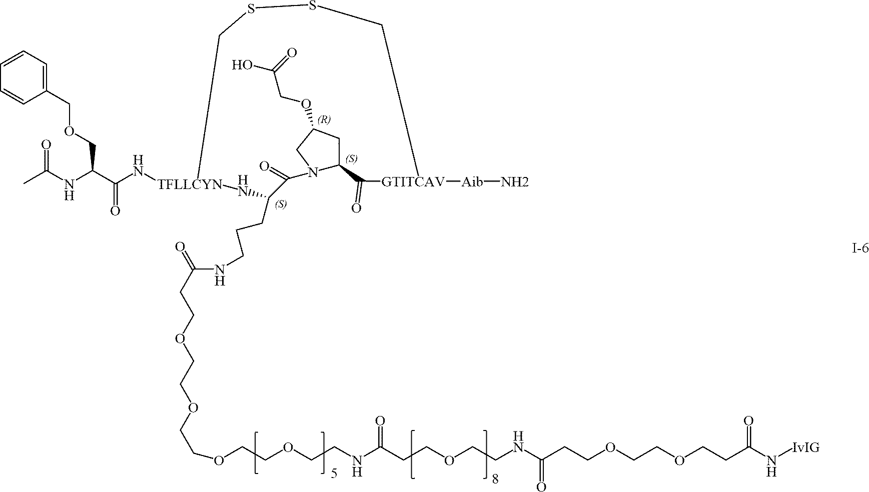

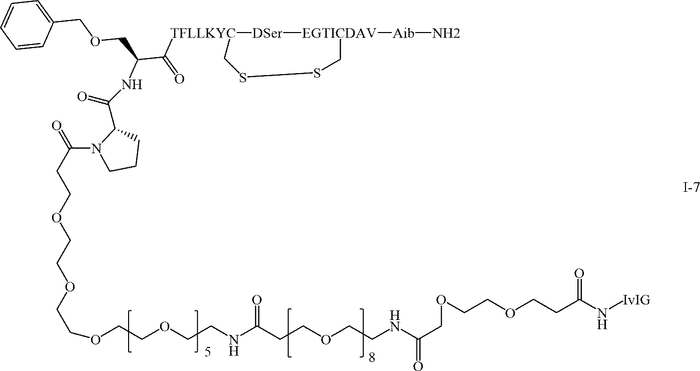 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCNC(=O)CCOCCOCCOCCOCCNC(=O)CCCC[C@H]2SC[C@H]3NC(=O)N[C@H]32)NC(=O)[C@H](CC(N)=O)NC(=O)[C@H](Cc2ccc(O)cc2)NC(=O)[C@@H](NC(=O)[C@H](C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O]

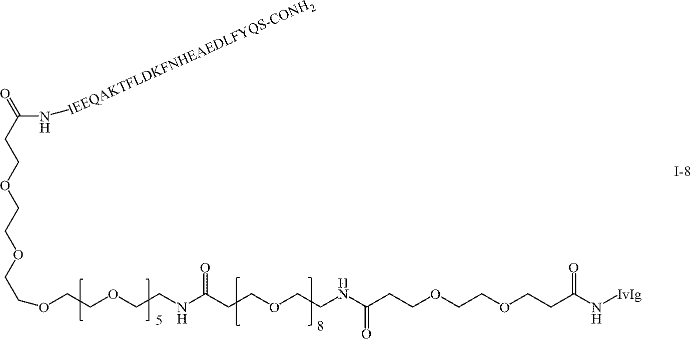

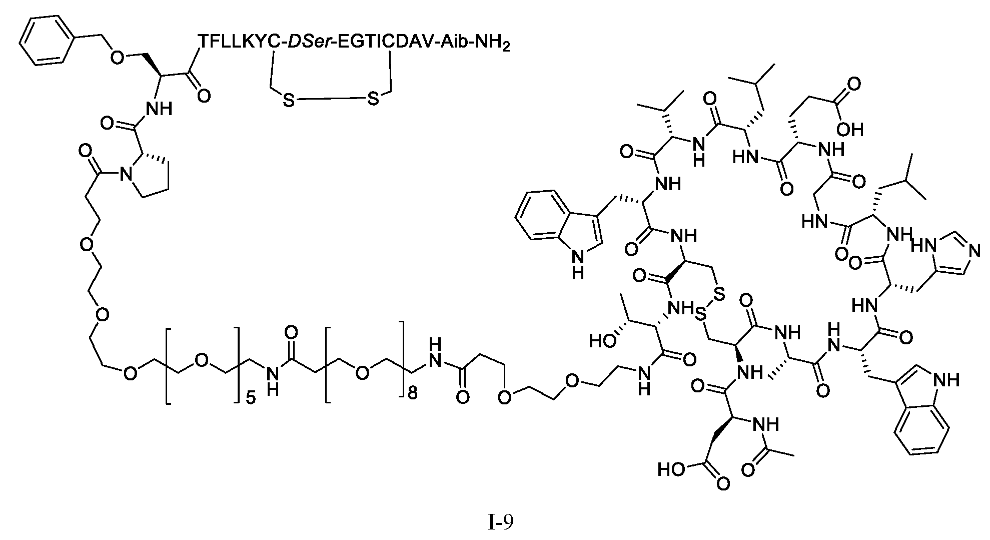

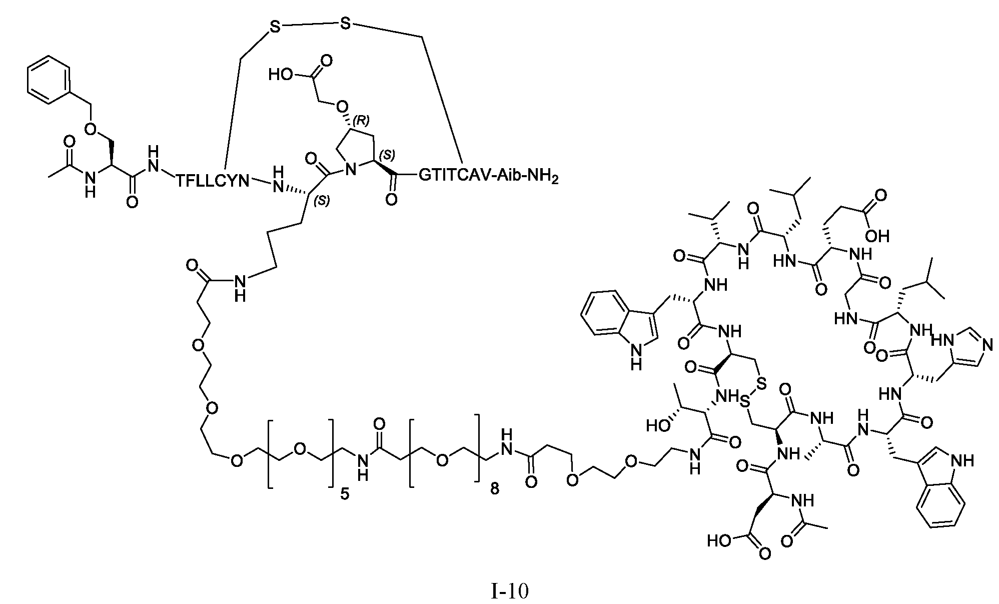

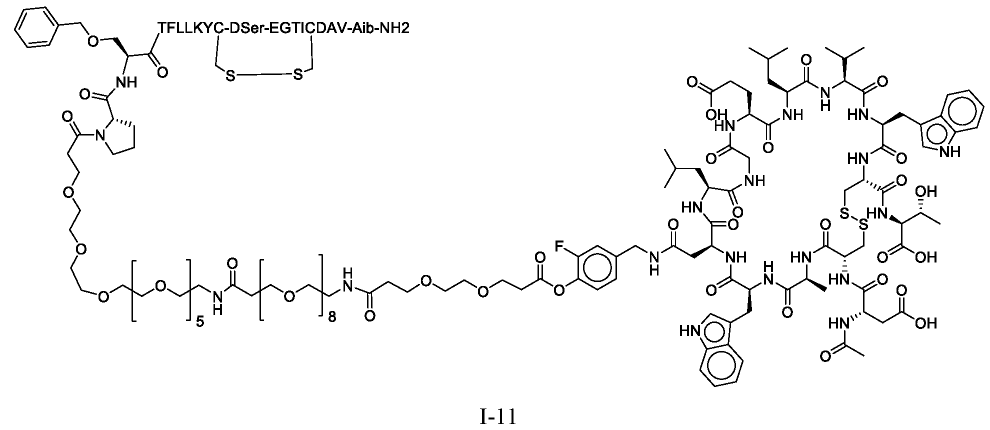

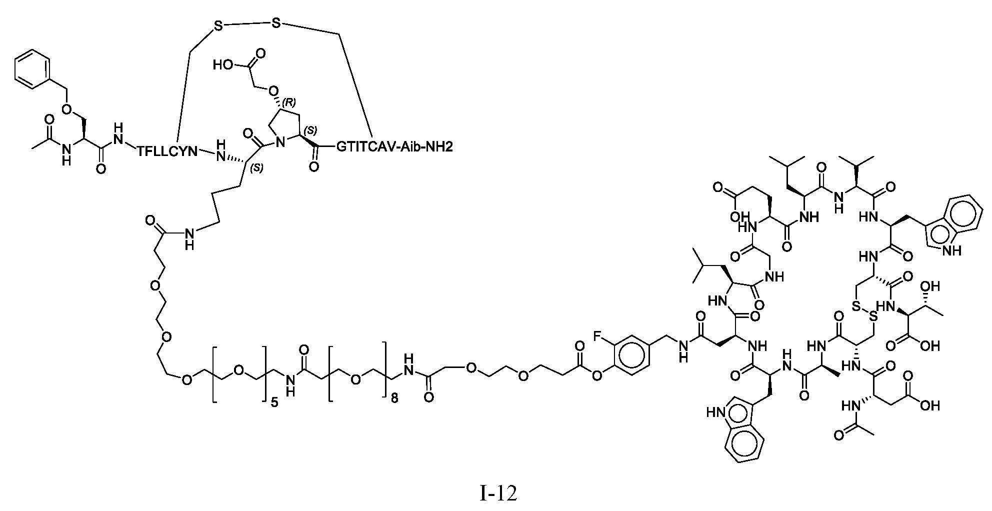

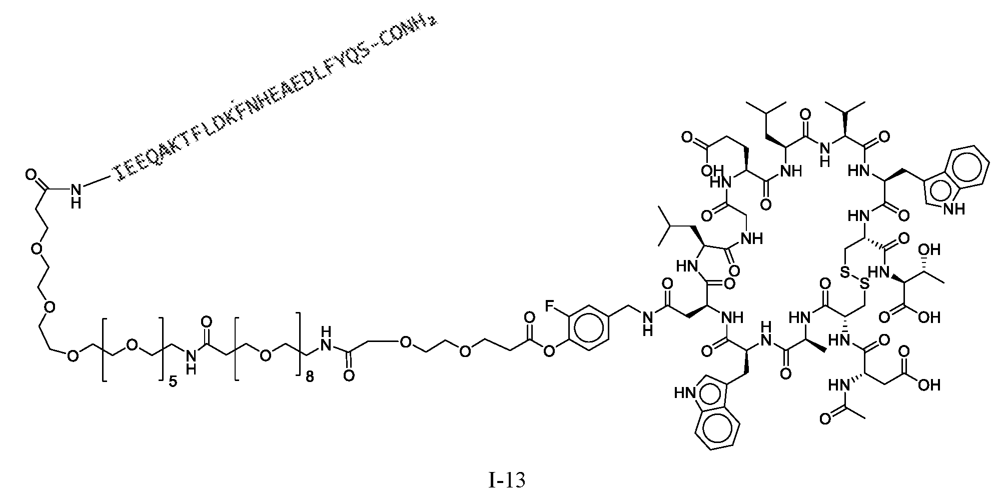

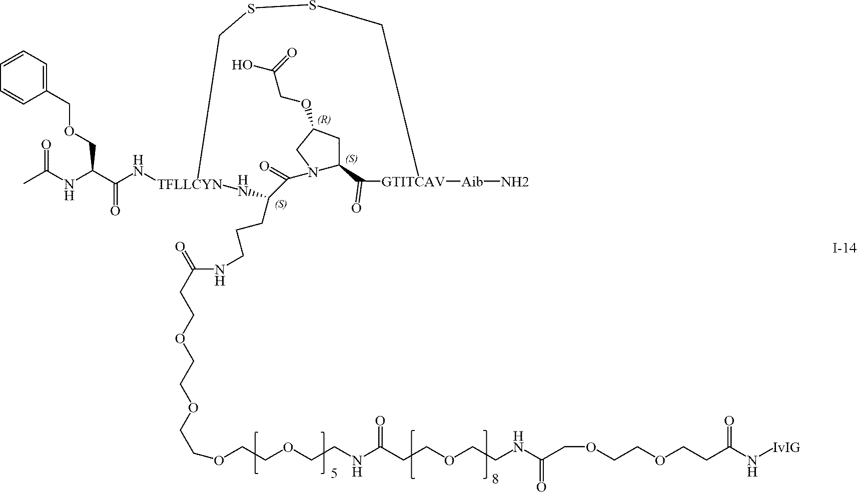

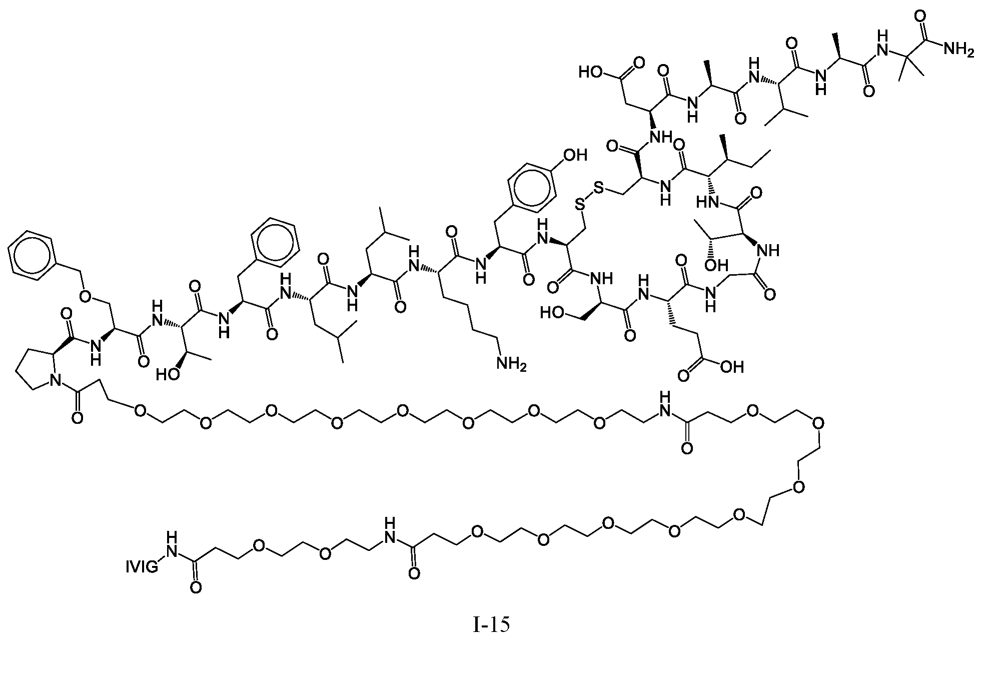

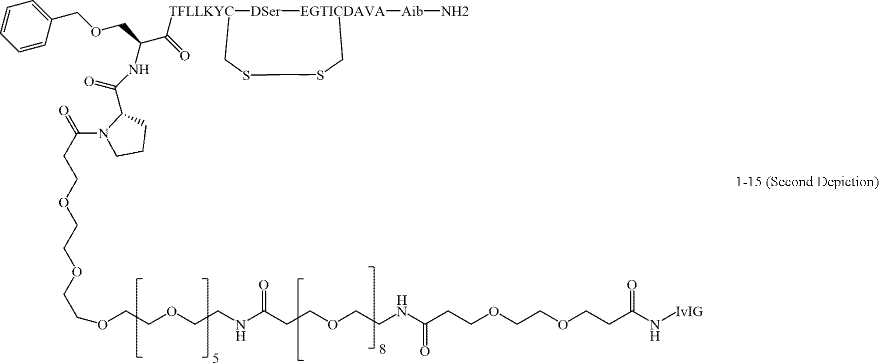

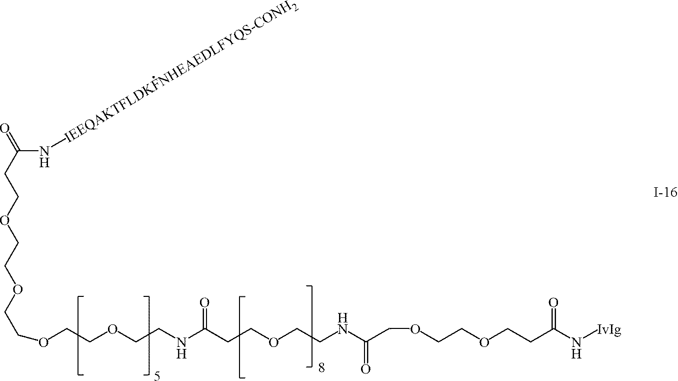

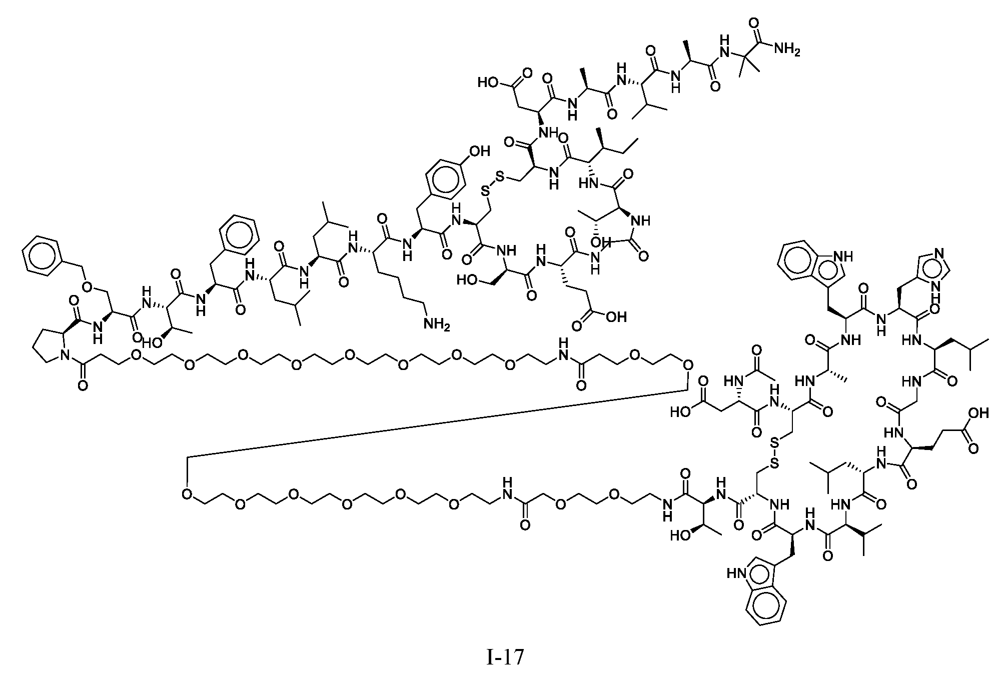

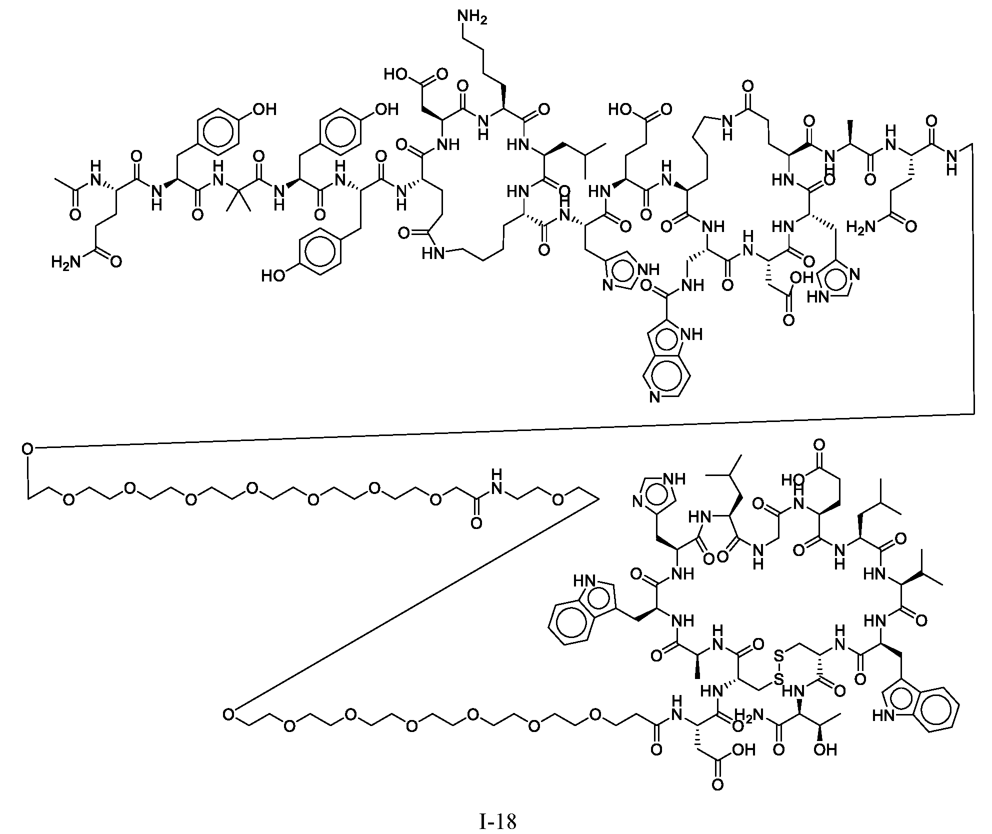

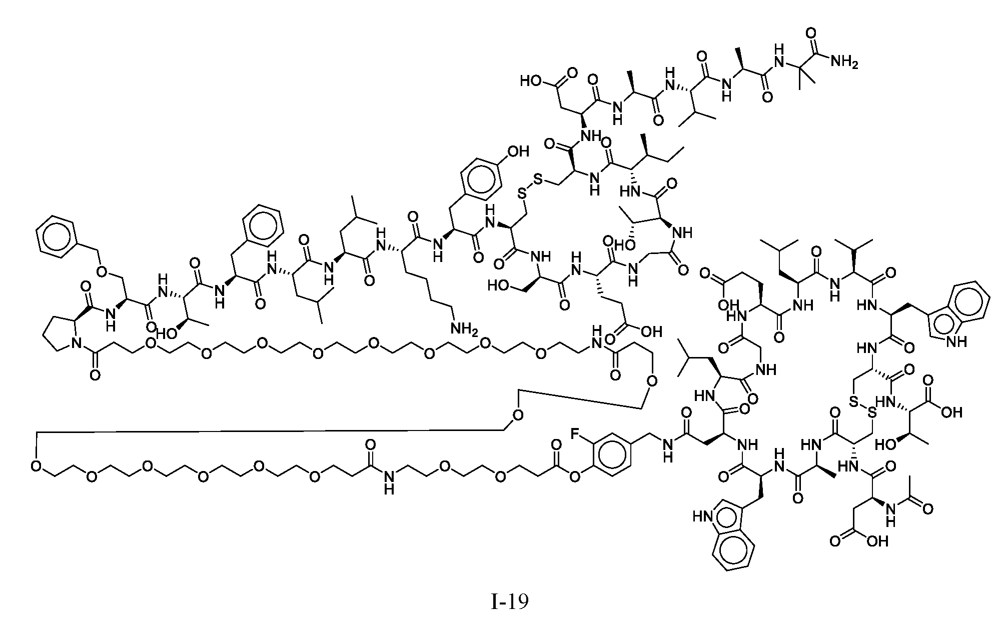

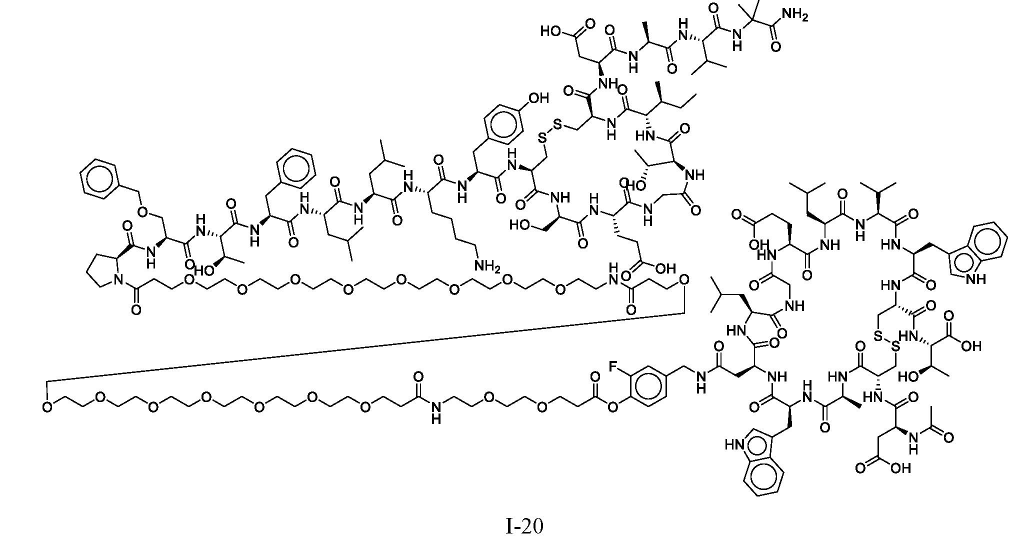

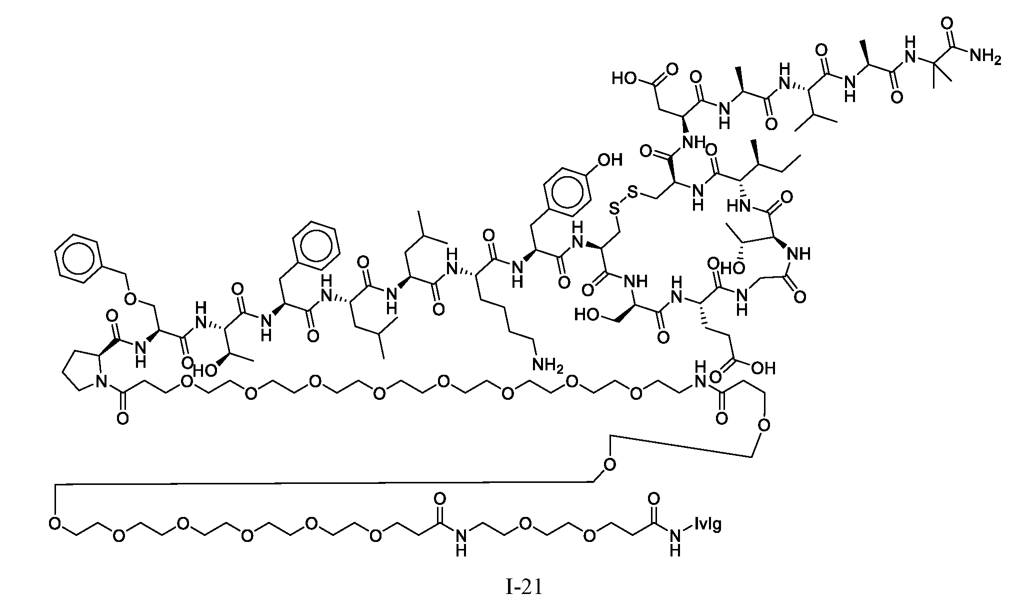

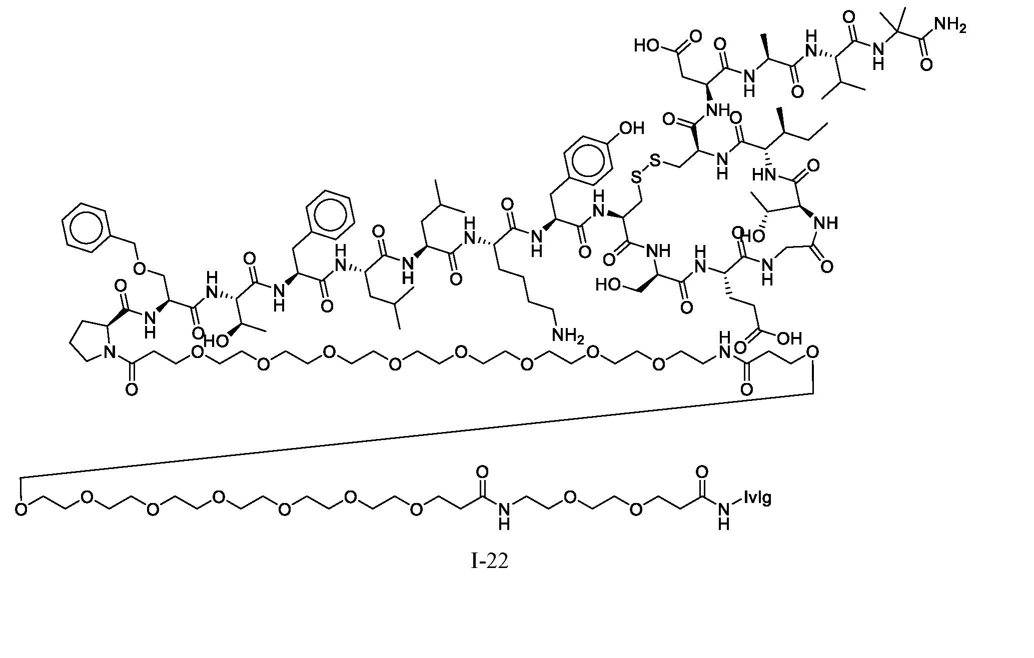

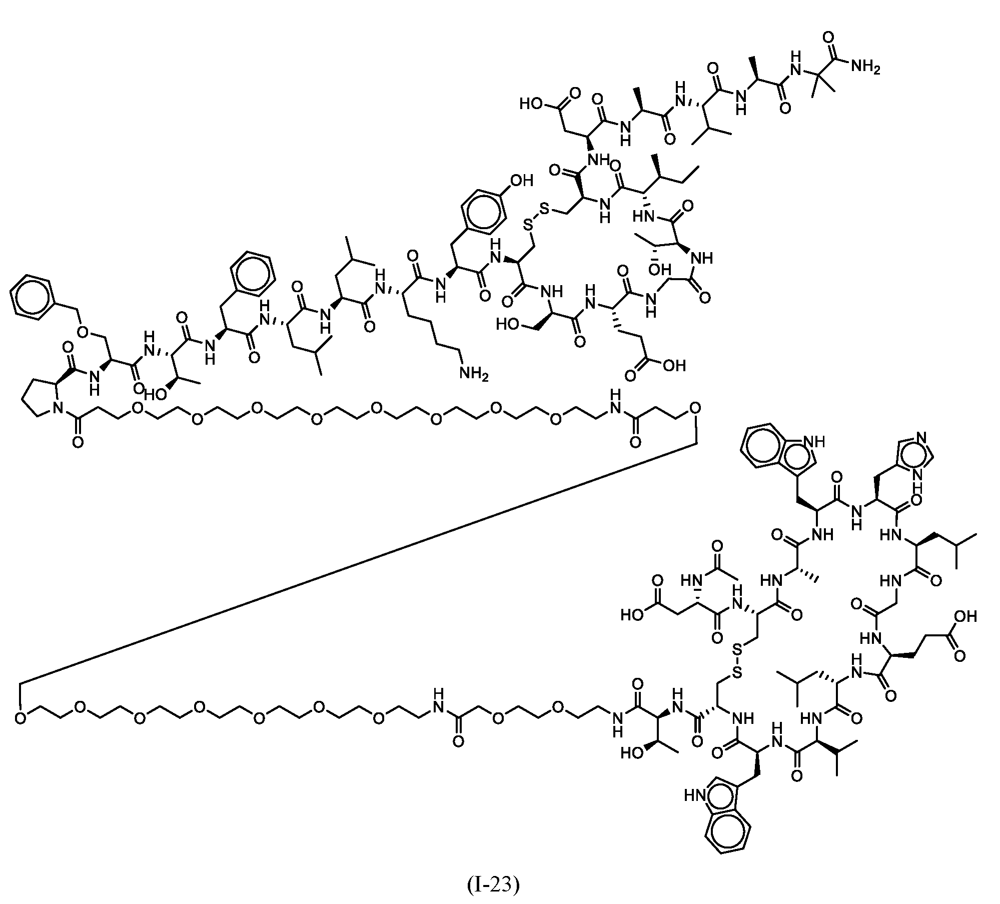

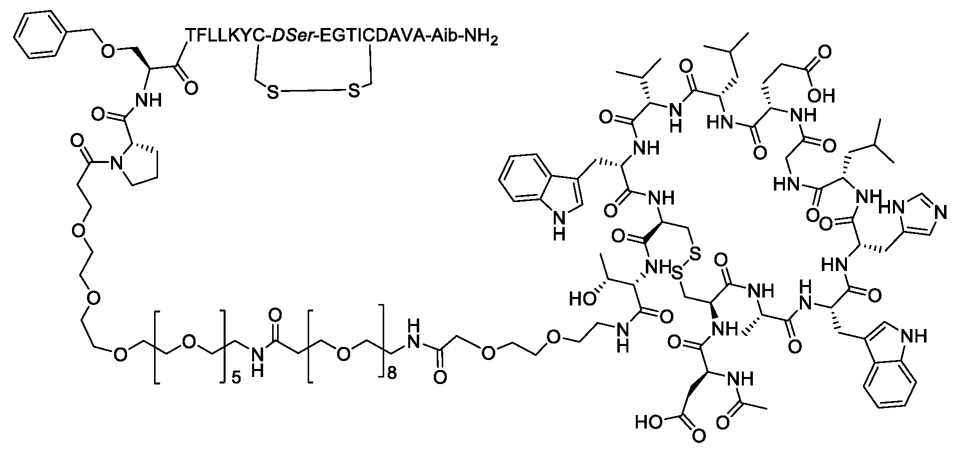

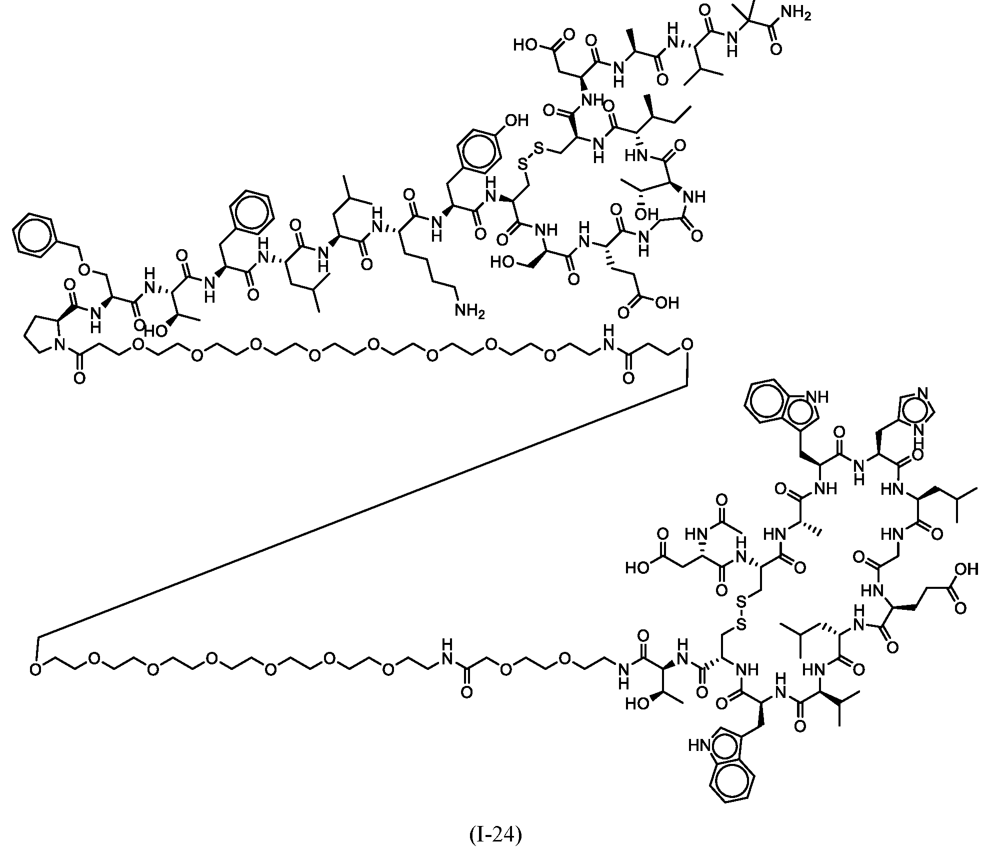

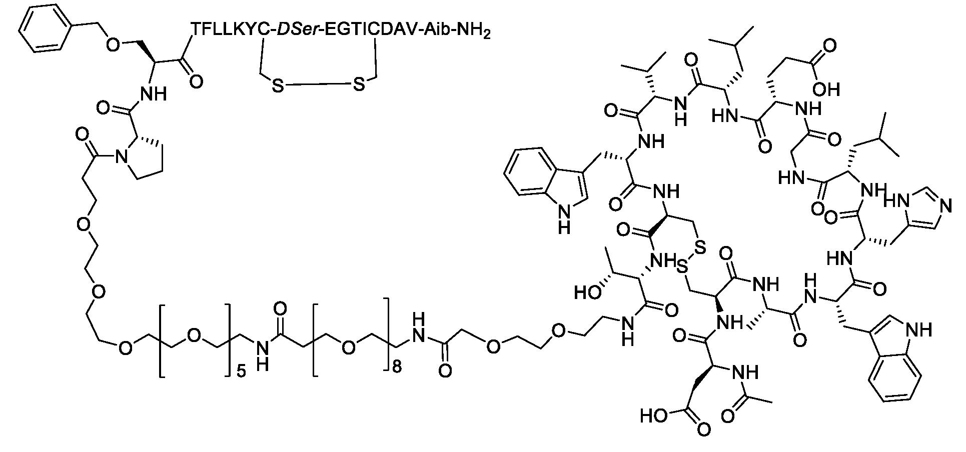

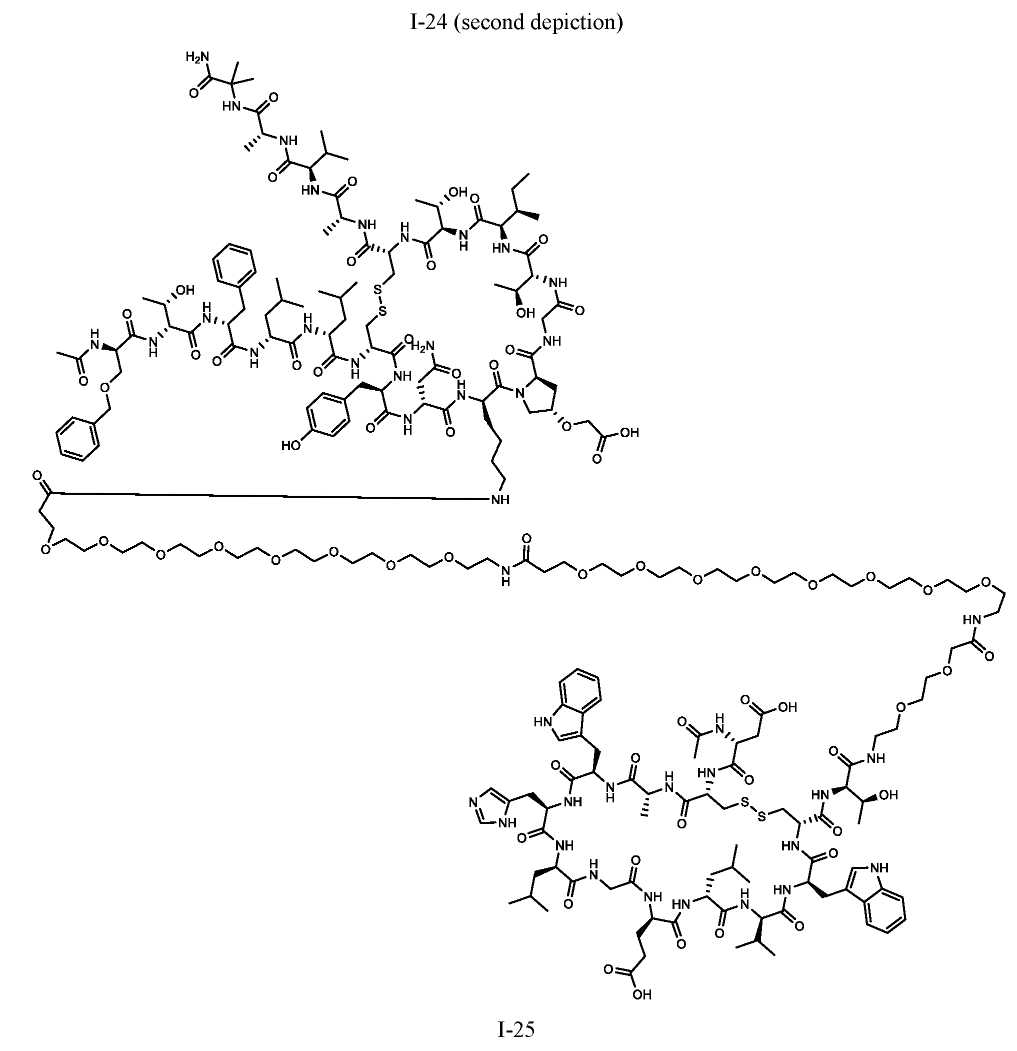

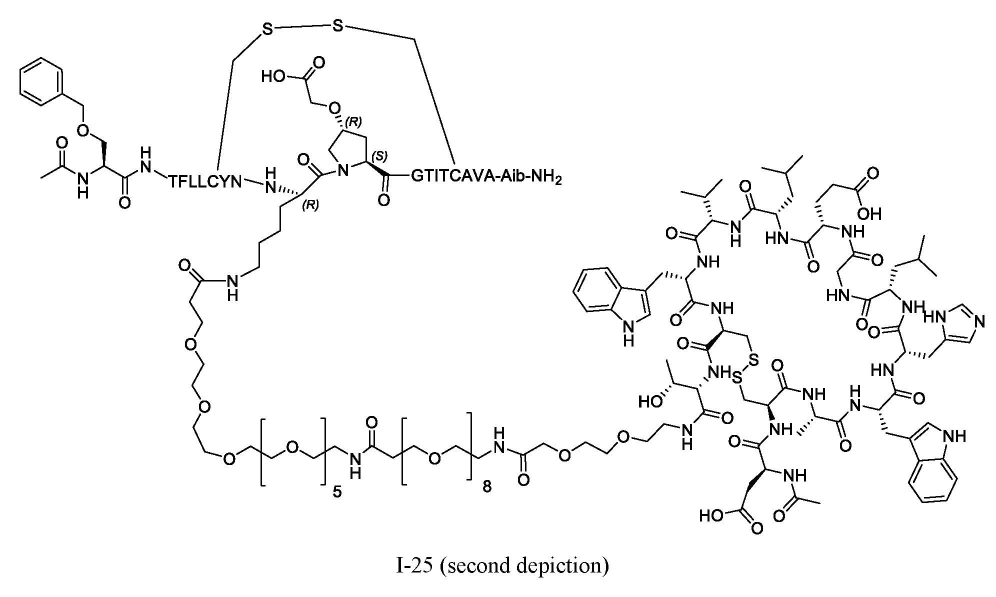

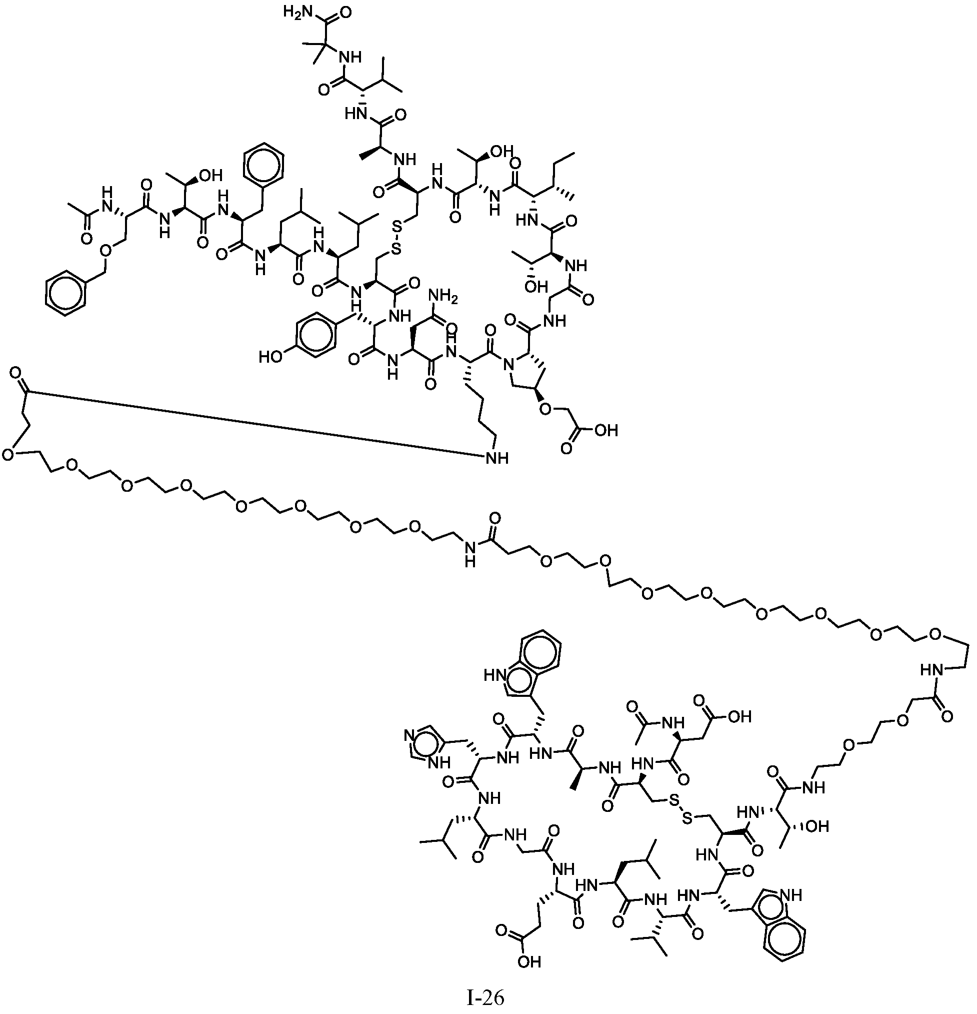

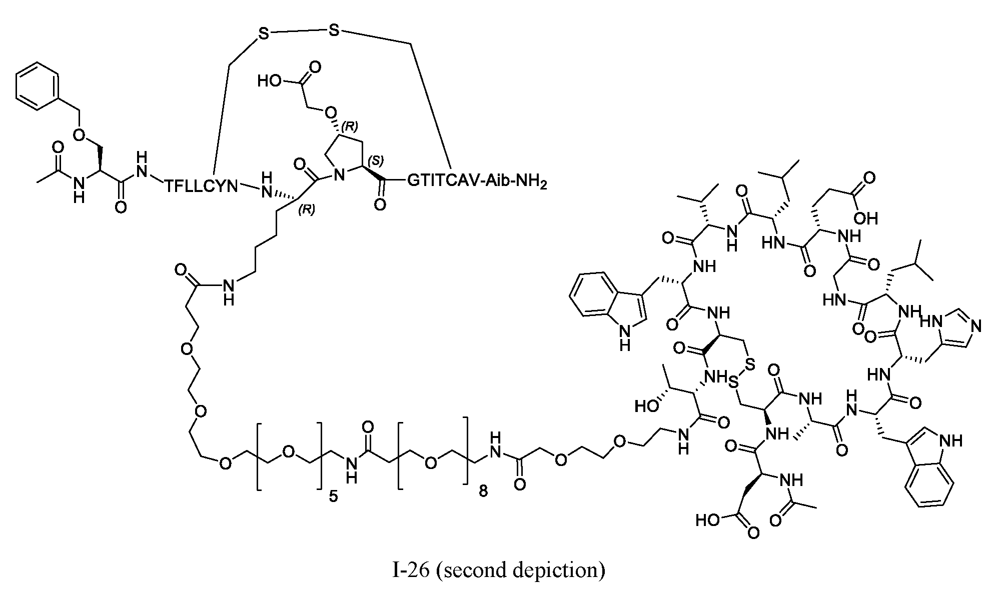

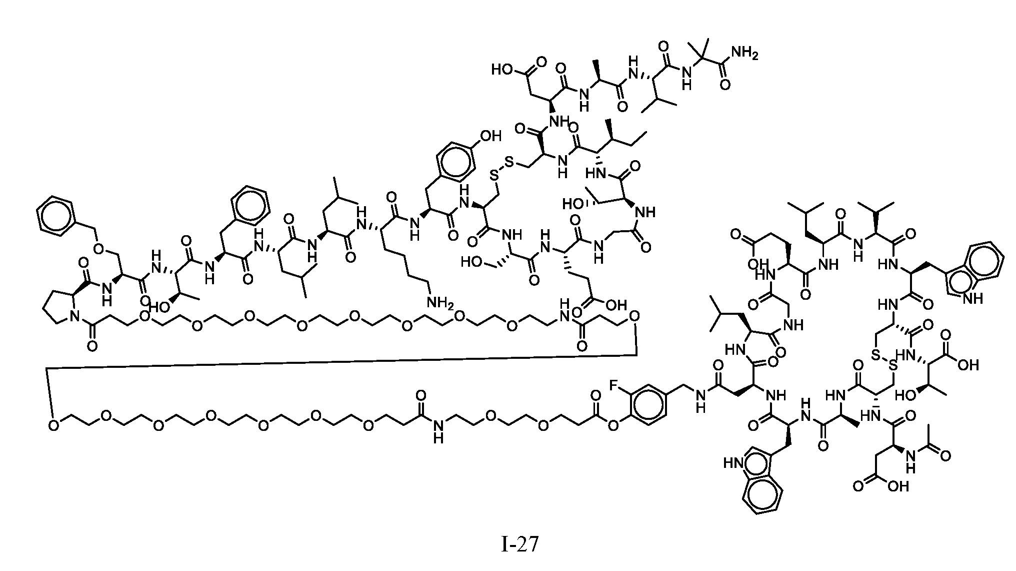

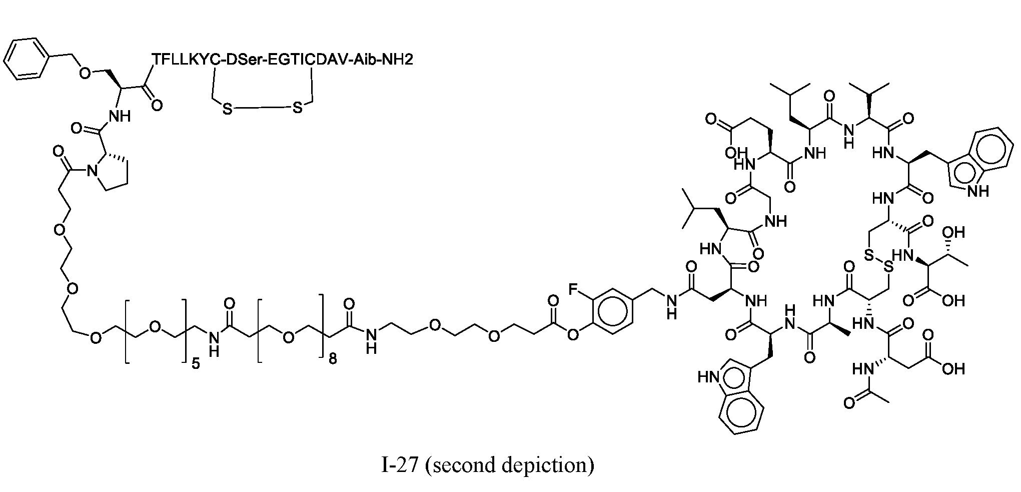

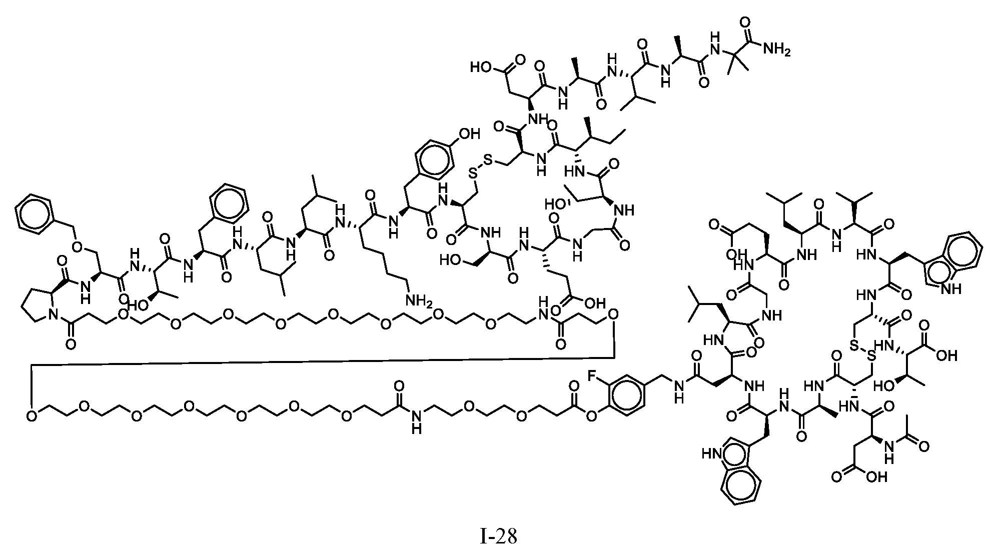

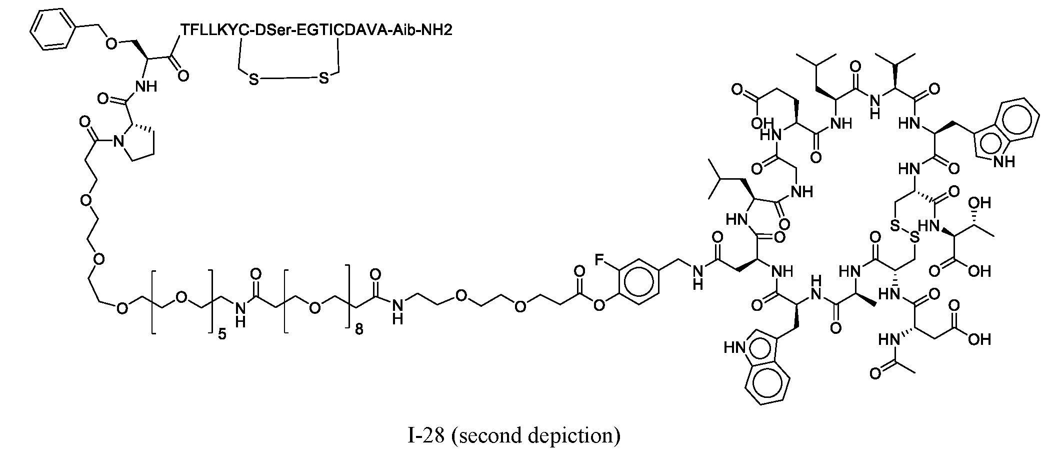

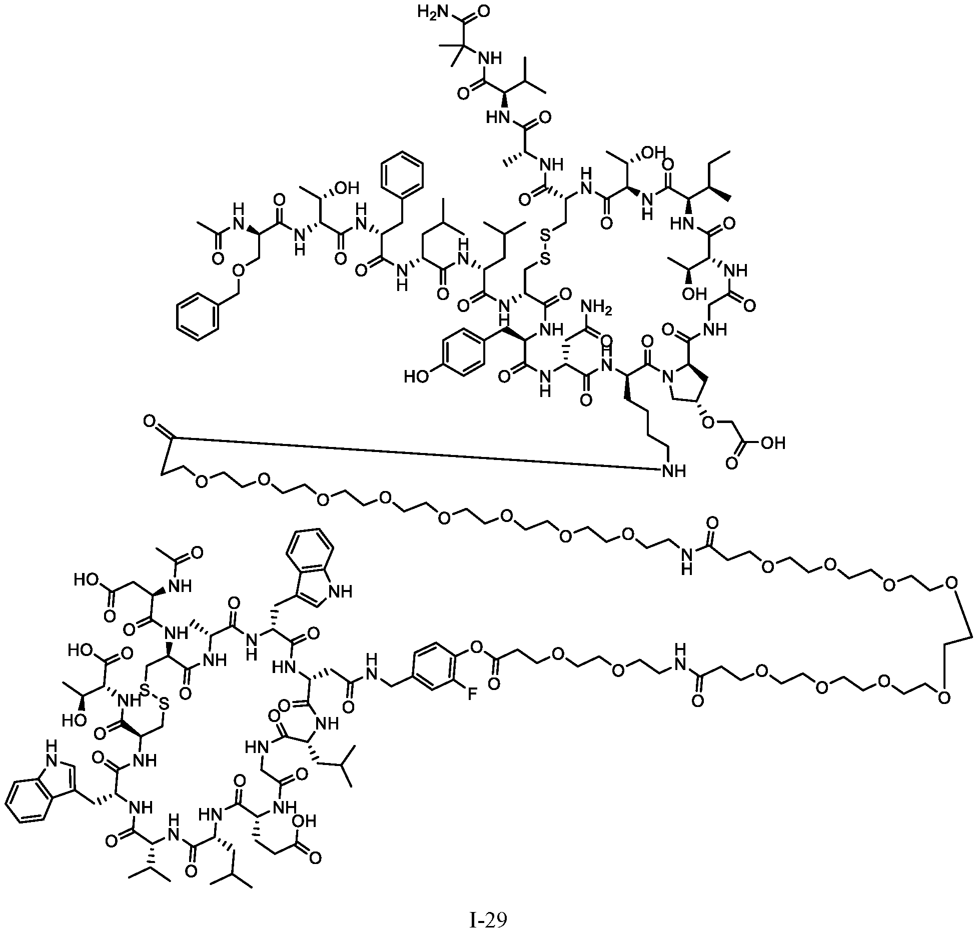

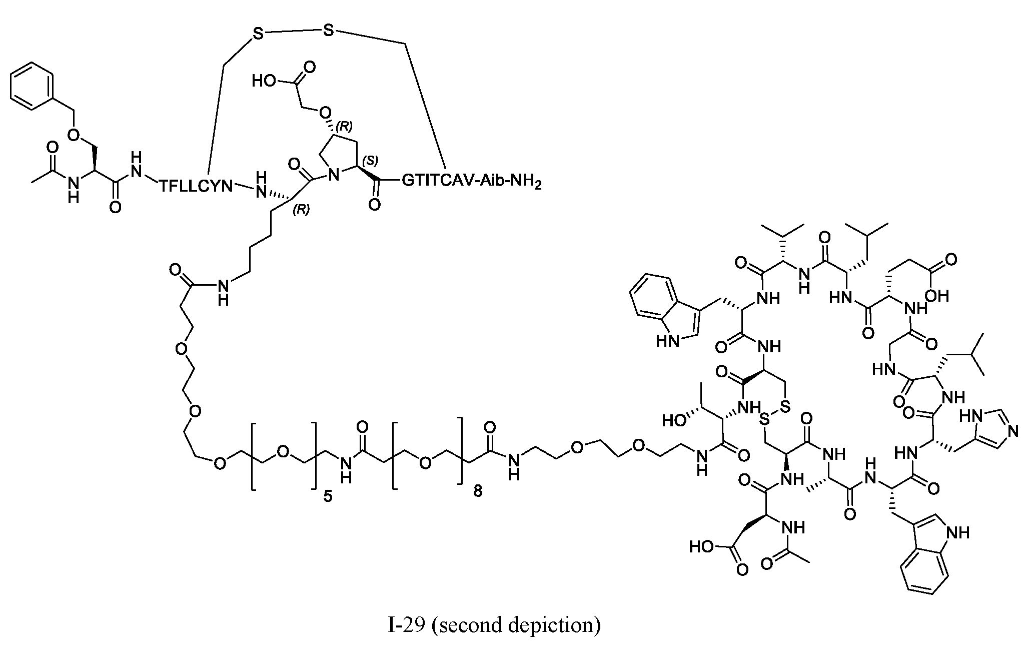

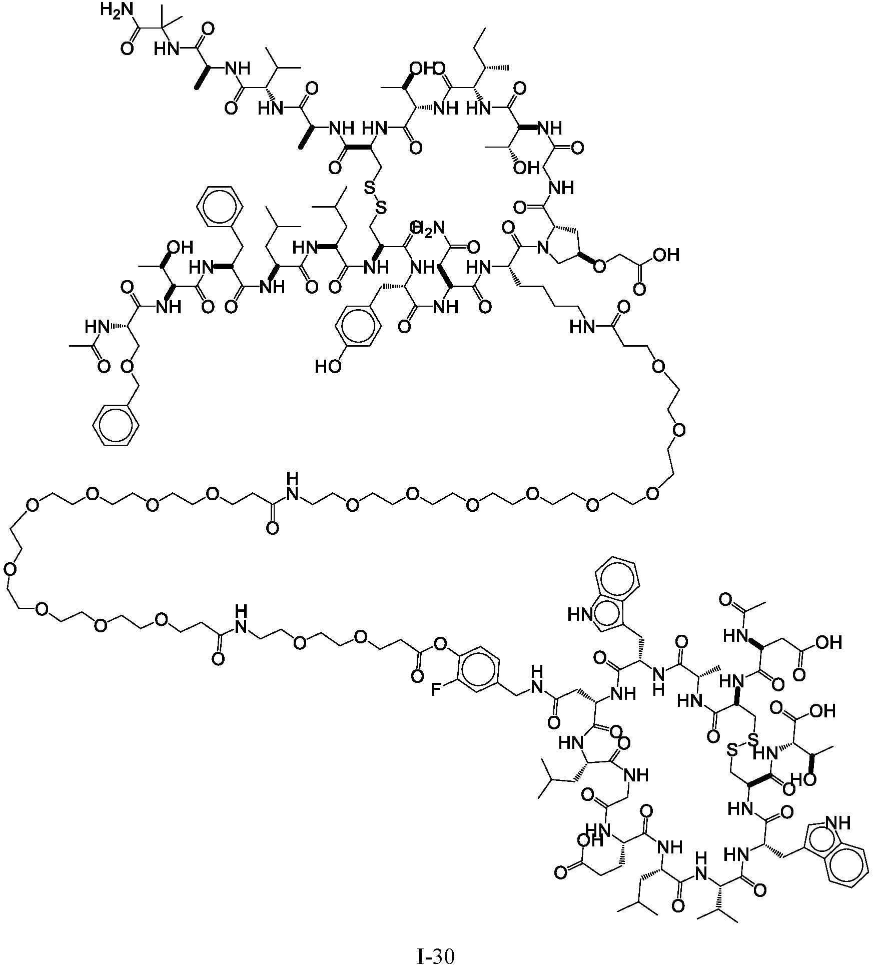

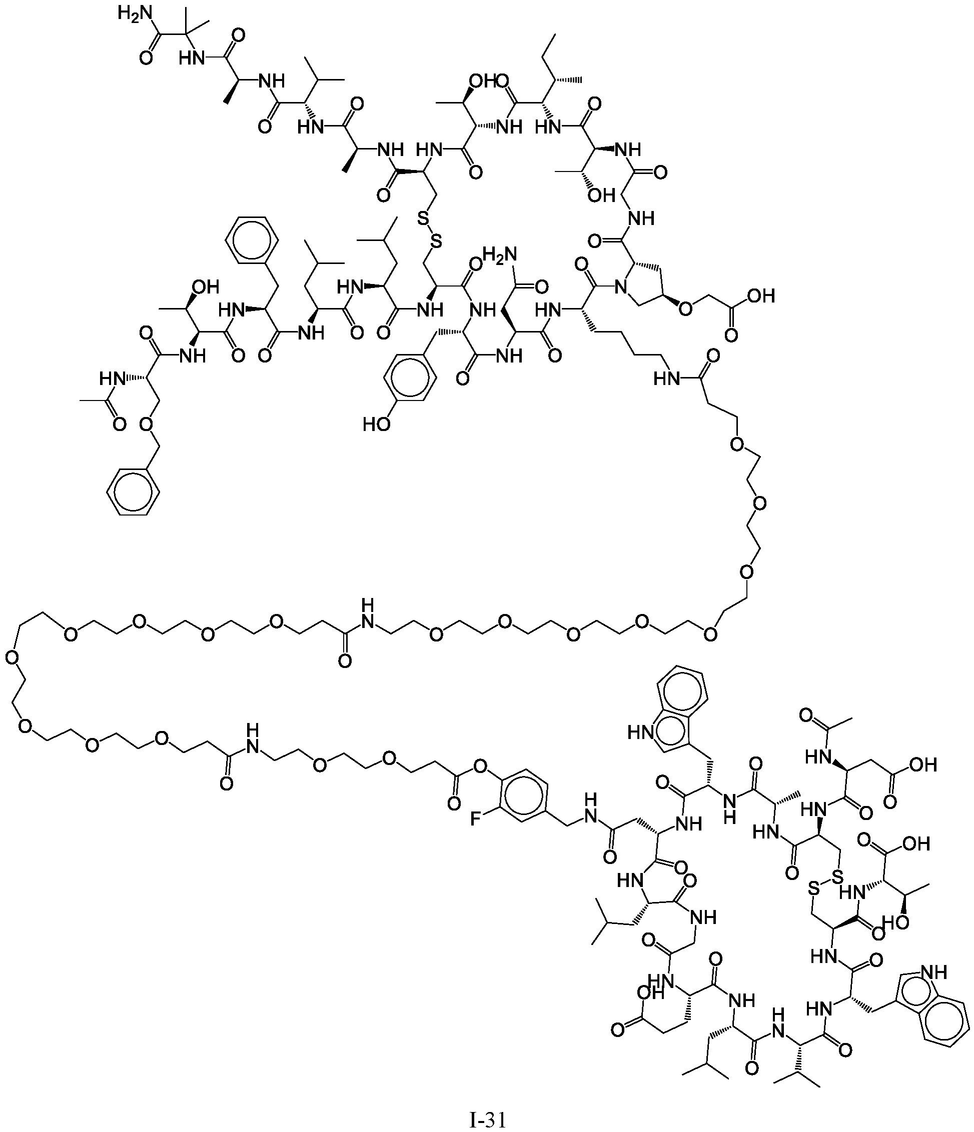

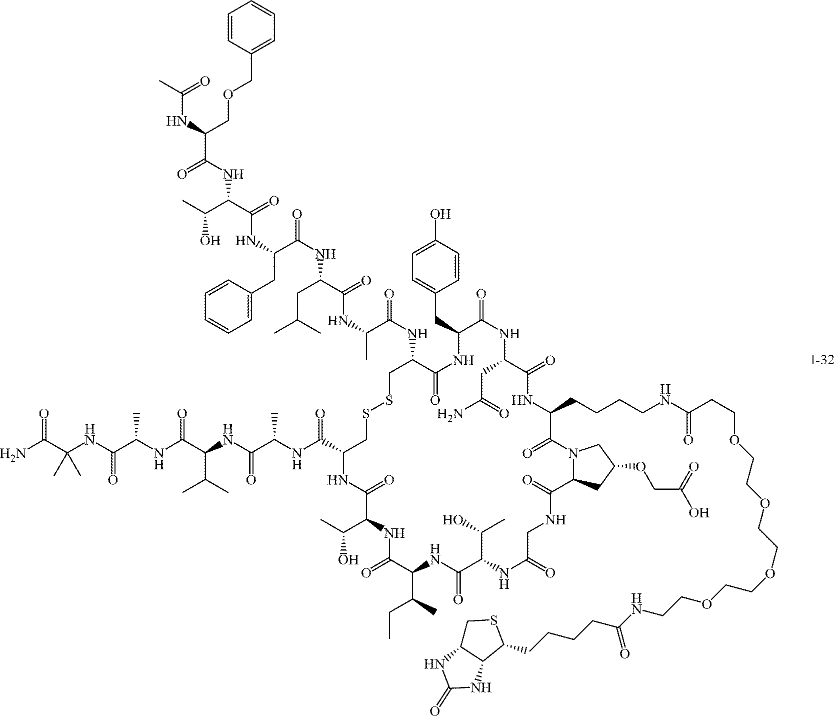

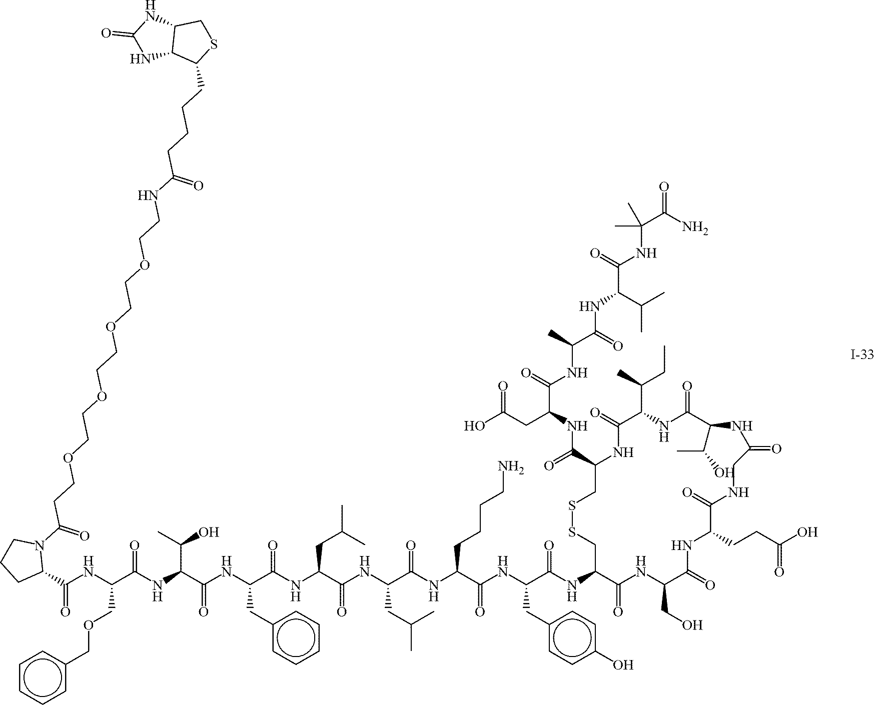

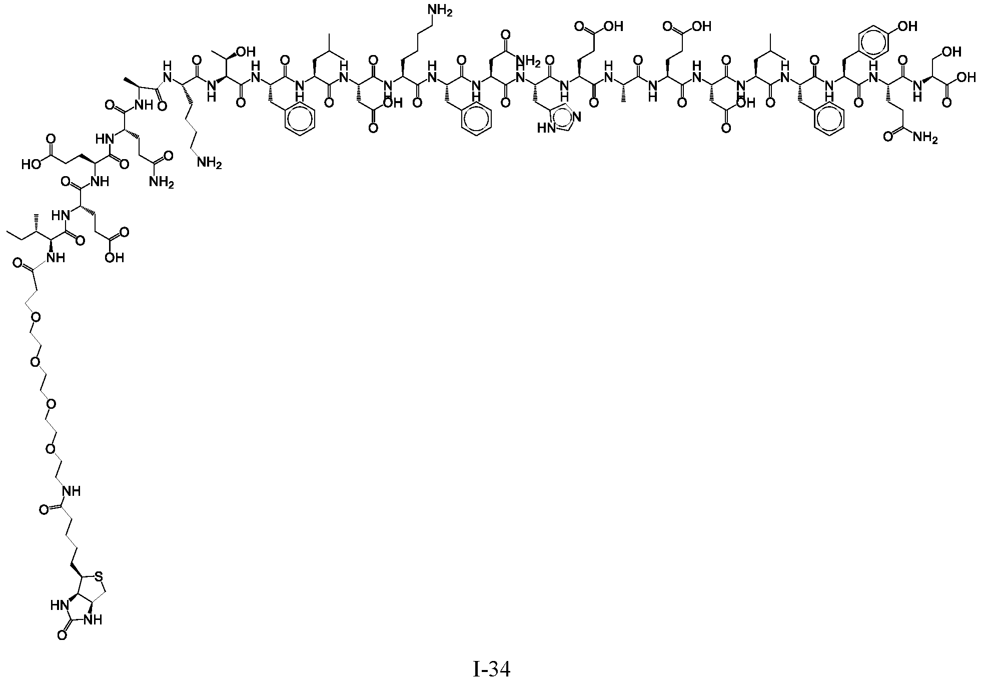

In some embodiments, the present disclosure provides a compound set forth in Table 1, above, or a pharmaceutically acceptable salt thereof.

In some embodiments, provided agents are conjugates of antibodies (e.g., IgG of a subject, pooled IgG, IVIG, etc.) with moieties comprising -(Xaa)y- (e.g., target binding moieties, agents of formula T-1, etc.) optionally through linker moieties (e.g., L). In some embodiments, provided agents are IVIG conjugates with target binding moieties optionally through linker moieties. In some embodiments, the present disclosure provides a plurality of such agents. In some embodiments, the present disclosure provides compositions comprising such agents. In some embodiments, the present disclosure provides compositions comprising a plurality of such agents.

In some embodiments, an antibody moiety is or comprises an IgG moiety (or a fragment thereof). In some embodiments, an antibody moiety is IVIG.

In some embodiments, the present disclosure provides a composition comprising a plurality of agents, wherein each agent independently comprises:
an antibody binding moiety,
a target binding moiety, and
optionally a linker moiety linking an antibody binding moiety and a target binding moiety.

In some embodiments, the present disclosure provides compositions comprising a plurality of such agents.

In some embodiments, each agent of the plurality is independently an agent described herein. In some embodiments, one or more agents of the plurality share the same target binding moiety. In some embodiments, agents of the plurality share the same target moiety. In some embodiments, one or more agents of the plurality share the same linker moiety. In some embodiments, agents of the plurality share the same linker moiety.

In some embodiments, one or more agents of the plurality each independently comprise an IgG moiety. In some embodiments, one or more agents of the plurality each independently comprise an IgG1 moiety. In some embodiments, one or more agents of the plurality each independently comprise an IgG2 moiety. In some embodiments, one or more agents of the plurality can each independently interact hFcyRIIIA. In some embodiments, one or more agents of the plurality can each independently interact hFcyRIIIA on macrophages. In some embodiments, one or more agents of the plurality each independently comprise an antibody moiety that can interact hFcyRIIIA. In some embodiments, one or more agents of the plurality each independently comprise an antibody moiety that can interact hFcyRIIIA on macrophages. In some embodiments, one or more agents of the plurality can each independently interact hFcyRIIA. In some embodiments, one or more agents of the plurality can each independently interact hFcγRIIA on dendritic cells. In some embodiments, one or more agents of the plurality each independently comprise an antibody moiety that can interact hFcyRIIA. In some embodiments, one or more agents of the plurality each independently comprise an antibody moiety that can interact hFcyRIIA on dendritic cells. In some embodiments, agents of the plurality can recruit immune cells. In some embodiments, one or more agents of the plurality each independently comprise an antibody moiety that can recruit an immune cell. In some embodiments, one or more agents of the plurality can recruit immune cells that inhibit, kill or remove a target (e.g., a small molecule, lipid, sugar, nucleic acid, microbe, bacteria, virus, foreign objects, diseased cells, etc.). In some embodiments, a target is a microbe. In some embodiments, a target is a virus. In some embodiments, a target is a SARS-CoV-2 virus. In some embodiments, agents of the plurality recruit immune cells. In some embodiments, agents of the plurality recruit NK cells. In some embodiments, agents of the plurality recruit macrophages. In some embodiments, agents of the plurality recruit dendritic cells.

In some embodiments, an agent induces, promotes, encourages, enhances, triggers, or generates ADCC and/or ADCP. In some embodiments, an agent induces, promotes, encourages, enhances, triggers, or generates ADCC and/or ADCP against a virus, e.g., a SARS-CoV-2 virus. In some embodiments, one or more agents of a plurality can induce, promote, encourage, enhance trigger or generate ADCC and/or ADCP. Those skilled in the art appreciate that technologies of the present disclosure may provide various types of immune activities and/or responses, including those involved in inhibition, killing and/or removal of viruses and cells infected thereby (in some cases, alternative to or in addition to ADCC and/or ADCP). In some embodiments, an agent induces, promotes, encourages, enhances, triggers, or generates long-term immunity (e.g., one or more vaccination effects). In some embodiments, an agent induces, promotes, encourages, enhances, triggers, or generates long-term immunity (e.g., one or more vaccination effects) against SARS-CoV-2. In some embodiments, technologies of the present disclosure provide long-term immunity. In some embodiments, a long-term immunity comprises memory T cells. In some embodiments, a long-term immunity comprises memory B cells. In some embodiments, a long-term immunity comprises memory T or B cells. In some embodiments, technologies of the present disclosure provide memory T and/or B cells against a target. In some embodiments, technologies of the present disclosure provide memory T and/or B cells against SARS-CoV-2. In some embodiments, one or more agents of a plurality can induce, promote, encourage, enhance, trigger or generate ADCC and/or ADCP, e.g., against SARS-CoV-2. In some embodiments, one or more agents of a plurality can induce, promote, encourage, enhance, trigger or generate long-term immunity, e.g., against SARS-CoV-2. In some embodiments, one or more agents of a plurality can provide memory T and/or B cells against SARS-CoV-2 when administered to a subject through one or more immunological processes.

In some embodiments, agents of a plurality comprise enriched levels of one or more types of antibody moieties. In some embodiments, one or more IgG isotypes are enriched in the composition. In some embodiments, IgG1 is enriched. In some embodiments, IgG2 is enriched. In some embodiments, IgG3 is enriched. In some embodiments, IgG4 is enriched. In some embodiments, two or three of IgG1, IgG2, IgG3, and IgG4 are enriched. In some embodiments, IgG1 and IgG2 are enriched. In some embodiments, enrichment is relative to a suitable reference. In some embodiments, a reference is serum of a subject (e.g., to whom an agent or composition is to be administered). In some embodiments, a reference is relevant levels in a population, e.g., a human population. In some embodiments, a reference is IVIG.

In some embodiments, antibody moieties in agents and/or compositions are or comprise structure features of recruited antibodies by antibody binding moieties. In some embodiments, antibody moieties in agents and/or compositions have properties and/or activities of recruited antibodies by antibody binding moieties.

In some embodiments, at least about 50%, 55%, 60%, 65%, 70%, 75%, 80%, 85%, 90%, 95%, 96%, 97%, 98%, or 99% of all agents, or substantially all agents, that comprise an antibody moiety and a target binding moiety in a composition are agents of the plurality.

In some embodiments, provided agents comprising antibody moieties can provide comparable or better safety profiles and/or therapeutic effects compared to serum derived antibodies obtained from subjects infected by SARS-CoV-2, e.g., those who have recovered or are recovering from COVID-19. In some embodiments, provided agents can be prepared from readily available antibodies, e.g., “off-the-shelf” IVIG and target binding moieties, and can be manufactured at much larger scale and/or much lower cost.

### 4. General Methods of Providing the Present Compounds

Compounds of the present disclosure may be prepared or isolated in general by synthetic and/or semi-synthetic methods known to those skilled in the art for analogous compounds and by methods described in detail in the Examples, herein.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March’s Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that provided agents may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March’s Advanced Organic Chemistry”, 5^{th} Ed., Ed.: Smith, M.B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

In some embodiments, the present disclosure provides methods for preparing a composition comprising a plurality of agents, wherein each agent independently comprises:
an antibody binding moiety,
a target binding moiety, and
optionally a linker moiety linking an antibody binding moiety and a target binding moiety; which method comprise:
contacting a plurality of agents each of which independently comprises a reactive group with a plurality of antibody molecules.

In some embodiments, an agent comprising a reactive group comprises an antibody binding moiety, a target binding moiety and optionally a linker. In some embodiments, agents agent comprising a reactive group share the same target binding moiety. In some embodiments, agents agent comprising a reactive group share the same structure. In some embodiments, antibody molecules are of such structures, properties and/or activities to provide antibody moieties in agents described herein. In some embodiments, a plurality of antibody molecules comprise two or more IgG subclasses. In some embodiments, a plurality of antibody molecules comprise IgG1. In some embodiments, a plurality of antibody molecules comprise IgG2. In some embodiments, a plurality of antibody molecules comprise IgG4. In some embodiments, a plurality of antibody molecules comprise IgG1 and IgG2. In some embodiments, a plurality of antibody molecules comprise IgG1, IgG2 and IgG4. In some embodiments, a plurality of antibody molecules comprise IgG1, IgG2, IgG3 and IgG4. In some embodiments, a plurality of antibody molecules are IVIG antibody molecules.

In some embodiments, provided agents comprise a reactive group, e.g.,

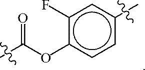

In some embodiments, —C(O)— is connected to a target binding moiety, or a moiety comprising -(Xaa)y-, optionally through a linker and the other end is connected to an antibody binding moiety. In some embodiments,

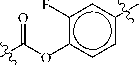

reacts with an amino group of another moiety, e.g., an antibody moiety,forming an amide group with the moiety and releasing a moiety which is or comprises antibody binding moiety. In some embodiments, an amino group is —NH₂ of a lysine side chain. In some embodiments, —C(O)— is connected to a target binding moiety, or a moiety comprising -(Xaa)y-, optionally through a linker and the other end is connected to R′ or an optional substituent.. In some embodiments, provided agents comprise optionally substituted

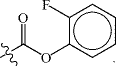

Such reactive groups may be useful for conjugation with detection, diagnosis or therapeutic agents. Those skilled in the art will appreciate that a variety of agents, and many technologies (e.g,, click chemistry, reactions based on functional groups such as amino groups (e.g., amide formation), hydroxyl groups, carboxyl groups, etc.) can be utilized for conjugation in accordance with the present disclosure.

In some embodiments, antibody binding moieties bind to Fc regions of antibodies. In some embodiments, reactions occur at residues at Fc regions. In some embodiments, target binding moieties are conjugated to residues of Fc regions, optionally through linker moieties. In some embodiments, a residue is a Lys residue. In some embodiments, an antibody is or comprises IgG1. In some embodiments, an antibody is or comprises IgG2. In some embodiments, an antibody is or comprises IgG4. In some embodiments, an antibody composition utilized in a method comprises IgG1 and IgG2. In some embodiments, an antibody composition utilized in a method comprises IgG1, IgG2 and IgG4. In some embodiments, an antibody composition utilized in a method comprises IgG1, IgG2, IgG3 and IgG4.

In some embodiments, a product is or comprises IgG1. In some embodiments, a product is or comprises IgG2. In some embodiments, a product is or comprises IgG4. In some embodiments, a product composition comprises IgG1 and IgG2. In some embodiments, a product composition comprises IgG1, IgG2 and IgG4. In some embodiments, a product composition comprises IgG1, IgG2, IgG3 and IgG4.

In some embodiments, provided agents comprising antibody moieties provide one or more or substantially all antibody immune activities, e.g. for recruiting one or more types of immune cells and/or provide short-term and long-term immune activities. In some embodiments, provided agents comprising antibody moieties do not significantly reduce one or more or substantially all relevant antibody immune activities. In some embodiments, provided agents comprising antibody moieties improve one or more or substantially all relevant antibody immune activities (e.g., compared to antibody moieties by themselves). In some embodiments, provided agents provides comparable or better stability compared to antibody moieties by themselves (e.g., residence time in blood). In some embodiments, antibody moieties in provided agents can bind to FcRy of immune cells (e.g., various FcRy of immune effector cells for desired immune activities; typically at comparable or better levels). In some embodiments, antibody moieties in provided agents have comparable Fab/antigen binding capabilities. In some embodiments, antibody moieties in provided agents have comparable Fab/antigen binding capabilities. In some embodiments, antibody moieties in provided agents provide FcRn binding. In some embodiments, antibody moieties in provided agents provide FcRn binding, e.g., for antibody recycle and/or prolonged half-life. In some embodiments, provided technologies are particularly useful for modifying blood-derived IgG products as provided technologies are suitable for and can utilize all IgG subclasses.

In some embodiments, a provided method comprises one of the steps described below. In some embodiments,

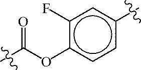

reacts with an amino group of a lysine side chain to form an amide bond with an antibody molecule, and releases

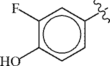

or a salt form thereof.

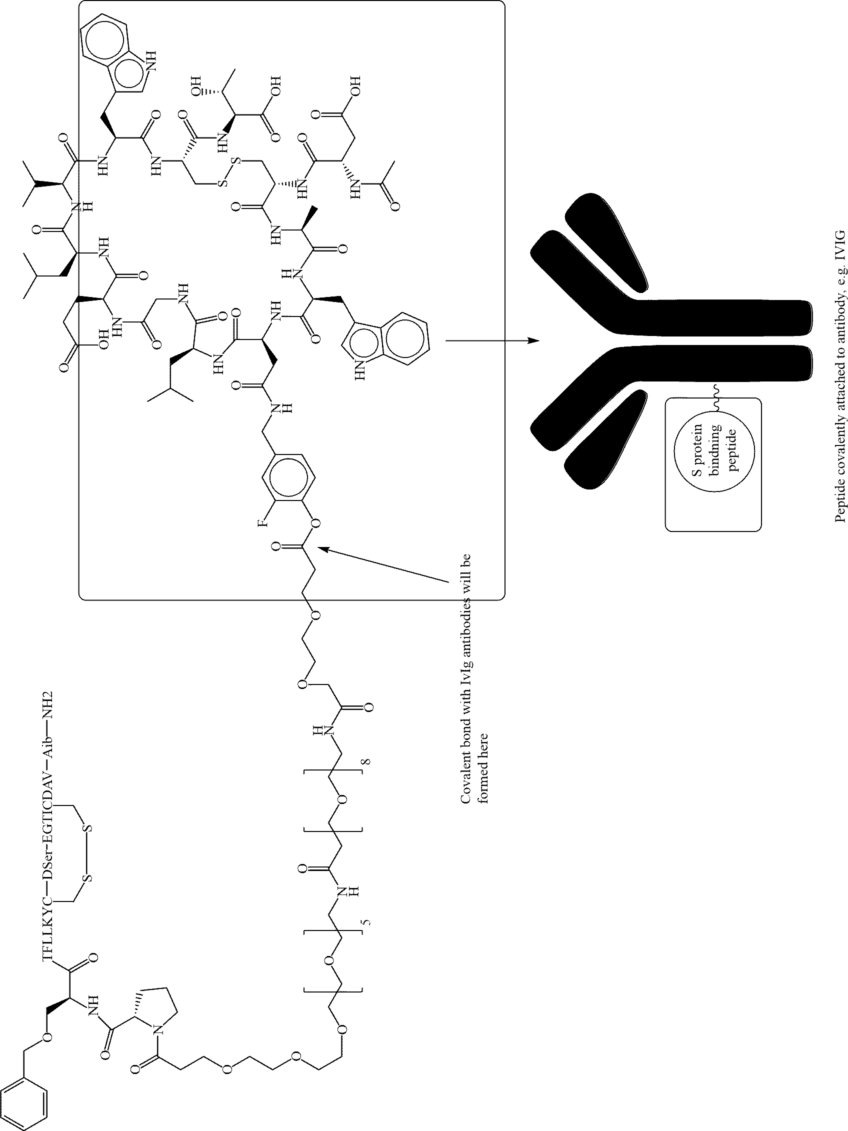

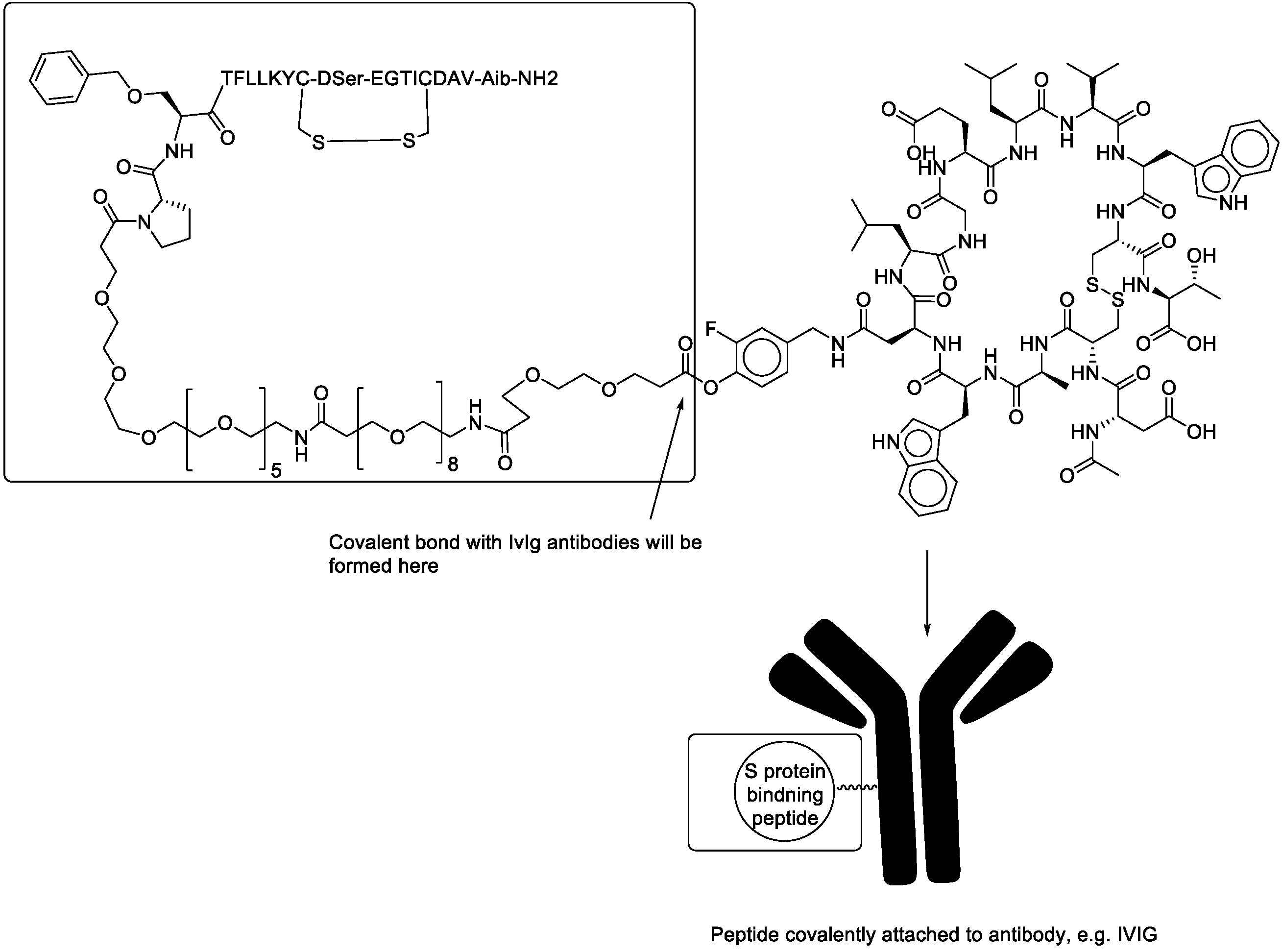

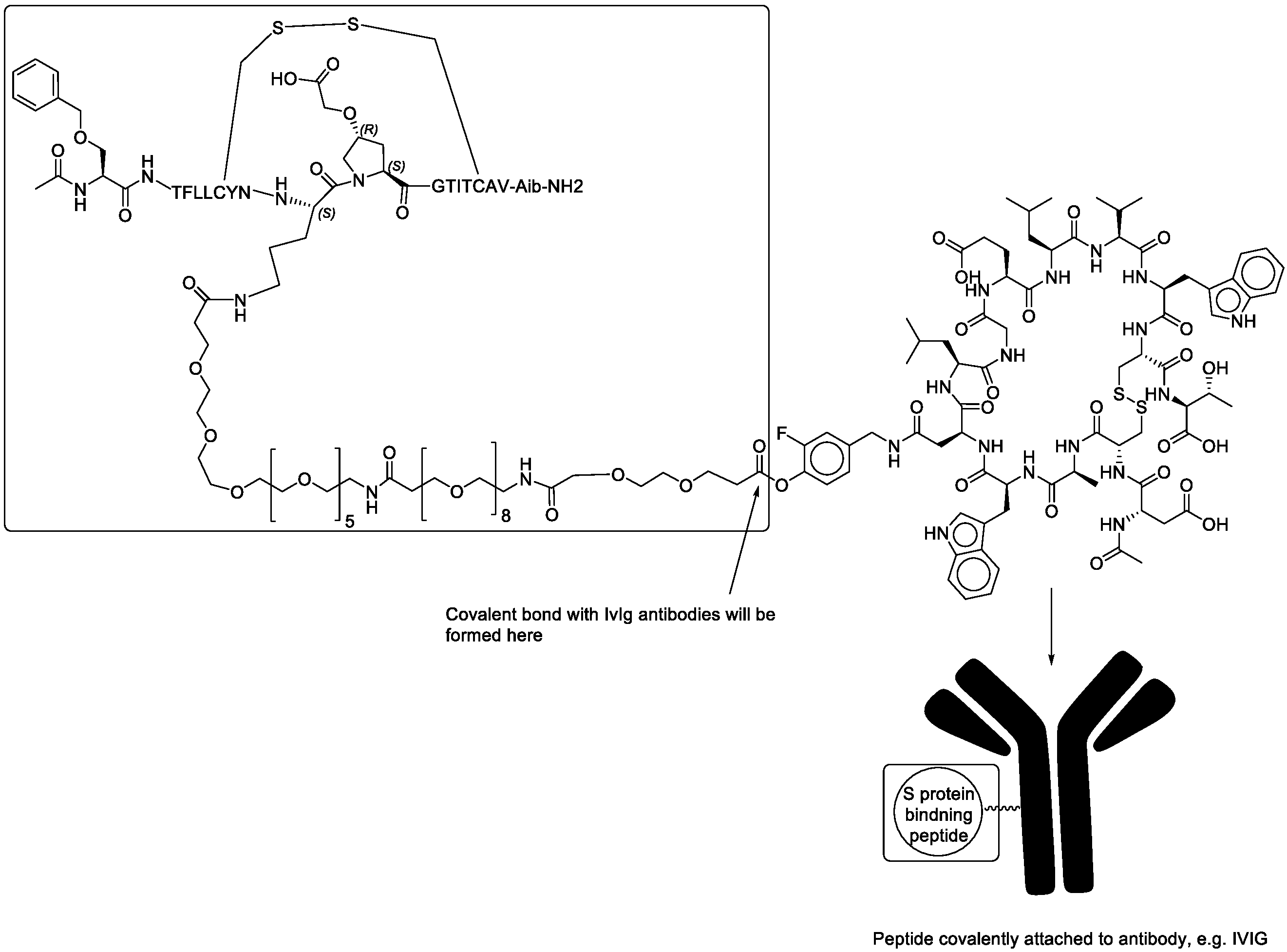

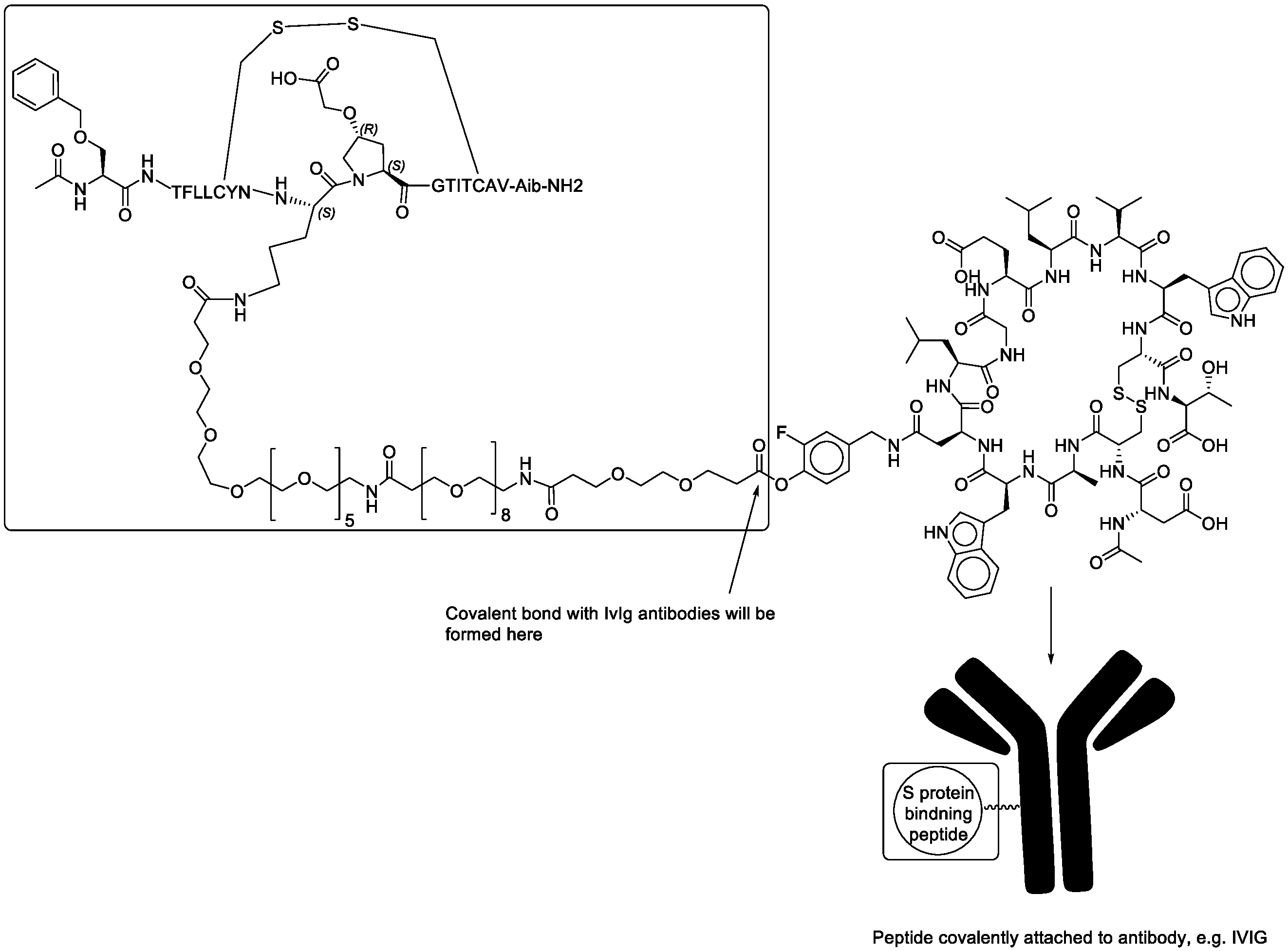

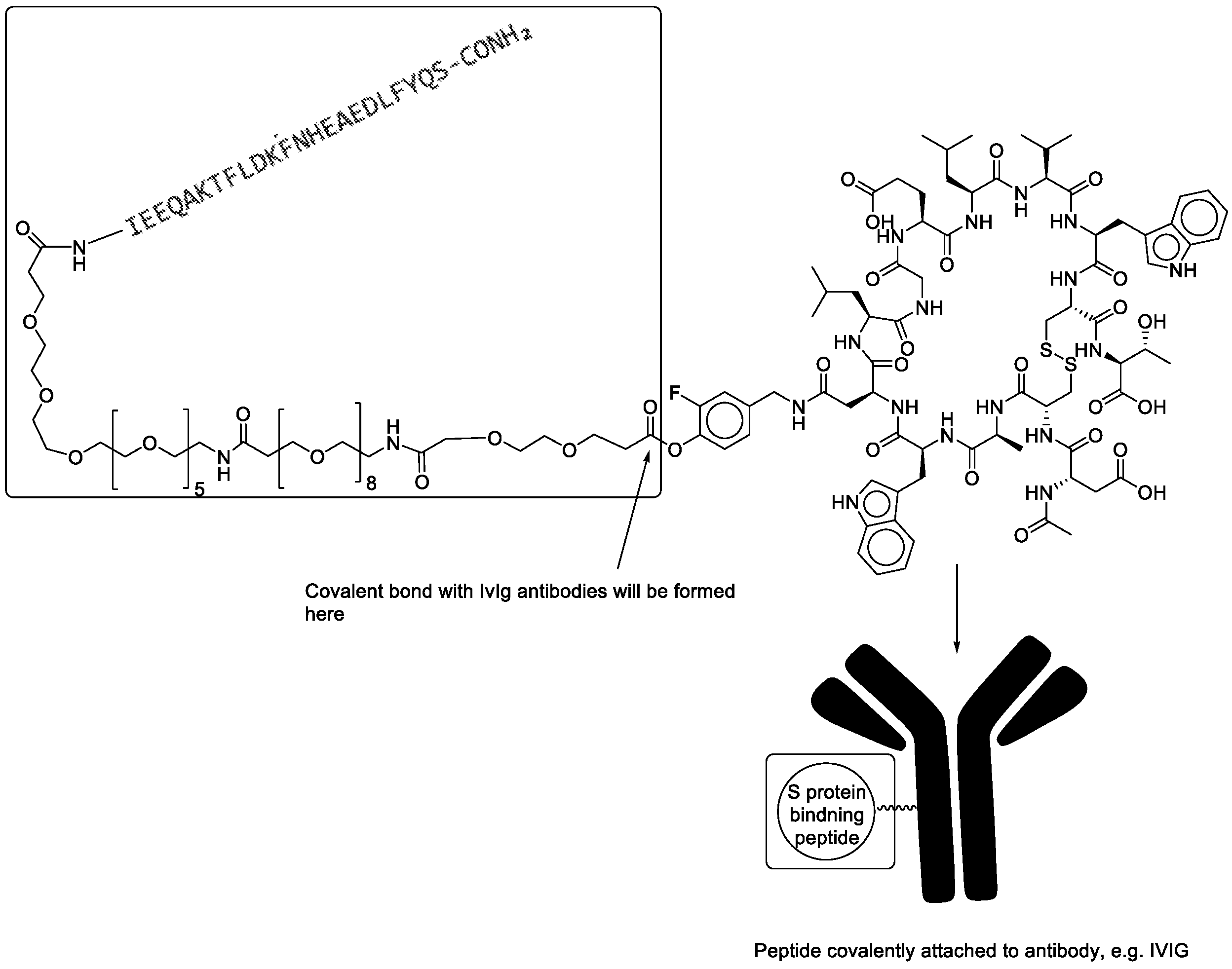

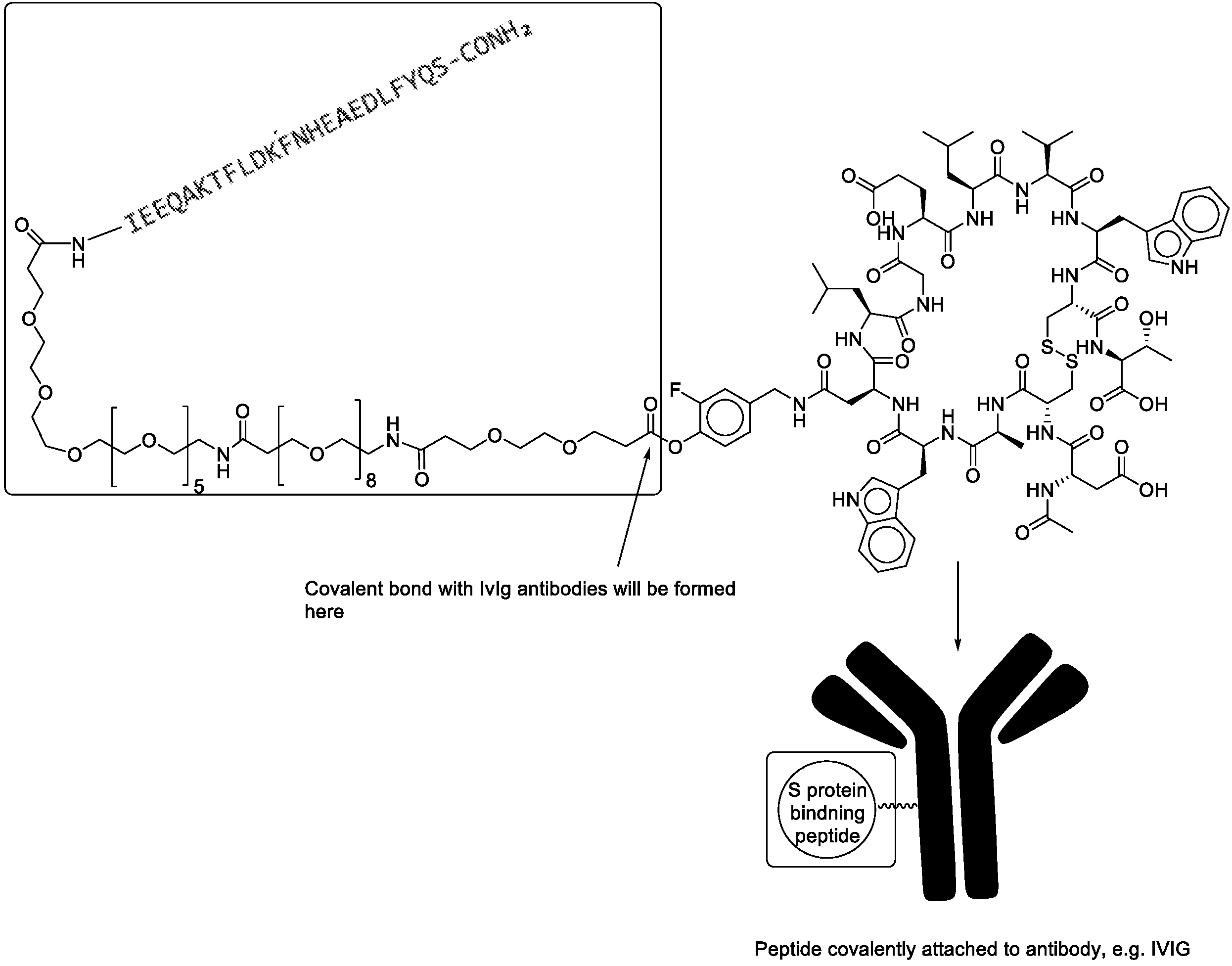

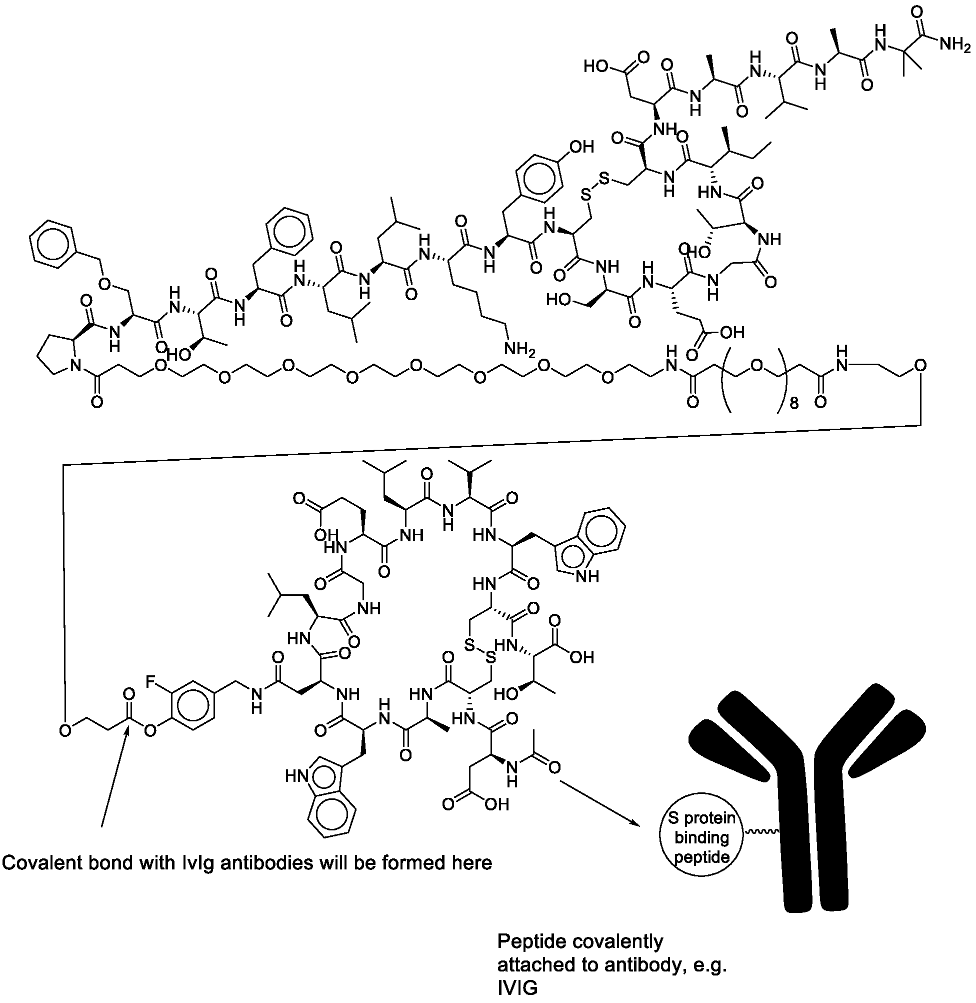

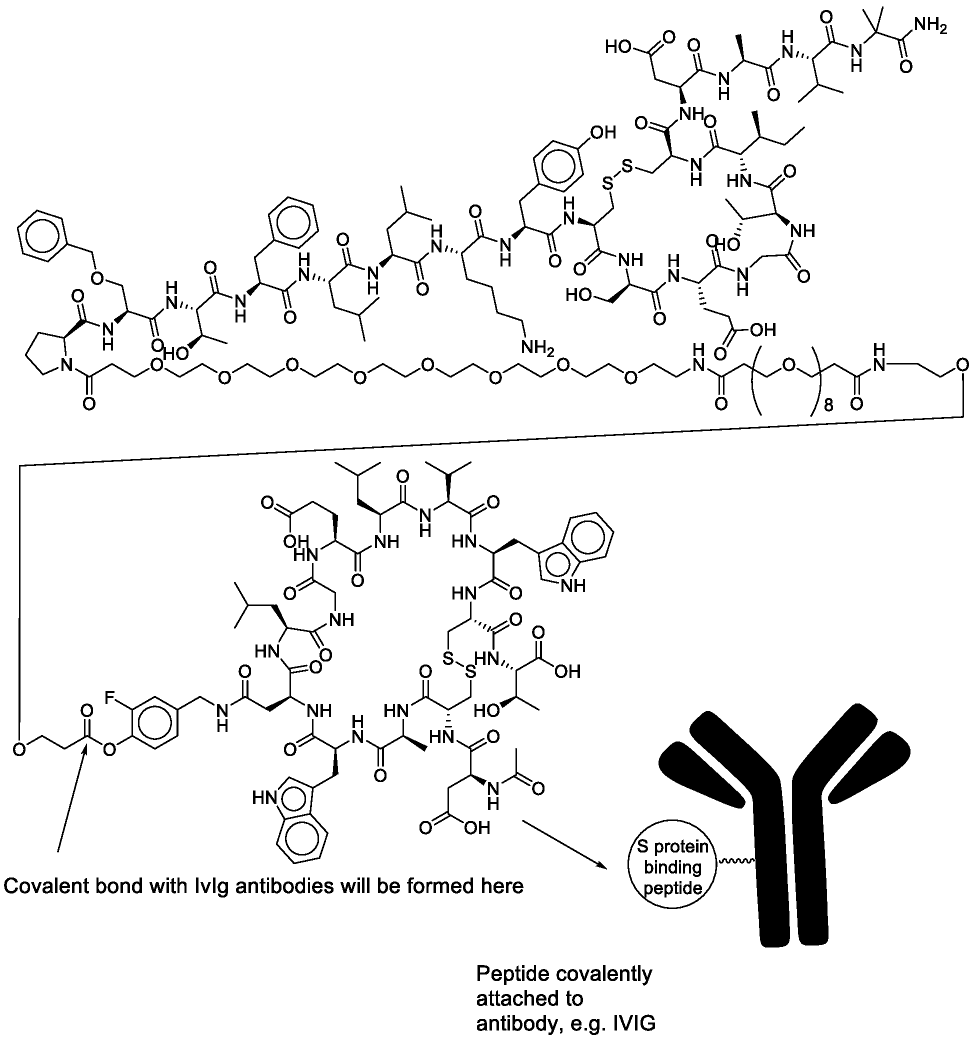

### 5. Uses, Formulation and Administration

### Pharmaceutically Acceptable Compositions

According to another embodiment, present disclosure provides a composition comprising a compound described herein or a pharmaceutically acceptable derivative thereof and a pharmaceutically acceptable carrier, adjuvant, or vehicle. In some embodiments, the present disclosure provides a pharmaceutical composition comprising a compound, e.g., an ARM, of the present disclosure and a pharmaceutically acceptable carrier. In some embodiments, the present disclosure provides a pharmaceutical composition comprising a therapeutically effective amount of a compound, e.g., an ARM, of the present disclosure and a pharmaceutically acceptable carrier. In some embodiments, an amount of a compound in a composition is such that it is effective to direct antibodies selectively to targets, e.g., diseased cells (e.g., SARS-CoV-2 infected cells), and/or induce antibody-directed activities, e.g., cell-mediated immunity such as cytotoxicity. In certain embodiments, an amount of a compound in a composition is such that is effective to direct antibodies selectively to cells expressing a SARS-CoV-2 spike protein or a fragment thereof, and induce antibody-directed activities, e.g., cell-mediated cytotoxicity, in a biological sample or in a subject (e.g., a SARS-CoV-2 infected patient). In certain embodiments, a composition is formulated for administration to a patient in need of such composition. In some embodiments, a composition is formulated for oral administration to a patient.

In some embodiments, a pharmaceutically acceptable carrier, adjuvant, or vehicle is a non-toxic carrier, adjuvant, or vehicle that does not destroy the pharmacological activity of the compound with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of a compound that, upon administration to a recipient, is capable of providing, either directly or indirectly, a compound or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium.

In some embodiments, a bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions may be administered in the form of suppositories for rectal administration. In some embodiments,, these can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, pharmaceutically acceptable compositions may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

Amounts of compounds that may be combined with the carrier materials to produce a composition in a single dosage form will vary depending upon the host treated, the particular mode of administration. In some embodiments, provided compositions are formulated so that a dosage of between 0.01 - 100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Provided Agents and Compositions

In some embodiments, when contacted with its target, provided agents and compounds form complexes with antibodies and Fc receptors, e.g., those of various immune cells. In some embodiments, the present disclosure provides a complex comprising:
an agent comprising:
   an antibody binding moiety,
   a target binding moiety, and
   optionally a linker moiety,
an Fc region, and
an Fc receptor.
In some embodiments, an antibody binding moiety is a universal antibody binding moiety.

In some embodiments, the present disclosure provides a complex comprising:
an agent comprising:
   an antibody moiety,
   a target binding moiety, and
   optionally a linker moiety, and
an Fc receptor.
In some embodiments, an antibody binding moiety is or comprises a Fc region. In some embodiments, an antibody moiety is or comprises IgG1. In some embodiments, an antibody moiety is or comprises IgG2. In some embodiments, an antibody moiety is or comprises IgG3. In some embodiments, an antibody moiety is or comprises IgG4.

In some embodiments, a complex further comprises a target, e.g., a virus or a cell infected thereby. In some embodiments, a complex comprises a SARS-CoV-2 virus. In some embodiments, a complex comprise a cell infected by a SARS-CoV-2 virus. In some embodiments, a complex comprises a cell expressing a SARS-CoV-2 spike protein or a fragment thereof.

In some embodiments, the present disclosure provides a plurality of complexes, each independently comprising:
an agent comprising:
   an antibody binding moiety,
   a target binding moiety, and
   optionally a linker moiety,
an Fc region, and
an Fc receptor,
wherein Fc regions of the complexes are of antibodies and/or fragments thereof toward different antigens or proteins.

In some embodiments, the present disclosure provides a plurality of complexes, each independently comprising:
an agent comprising:
   an antibody moiety, a target binding moiety, and
   optionally a linker moiety, and
an Fc receptor,
wherein Fc regions of the complexes are of antibodies and/or fragments thereof toward different antigens or proteins.

In some embodiments, Fc regions are of Fc regions of antibodies (e.g., antibodies recruited antibodies by agents comprising antibody binding moieties, antibody moieties in provided agents, etc.). In some embodiments, Fc regions of the complexes are of antibodies and/or fragments thereof toward different proteins. In some embodiments, one or more Fc regions are of endogenous antibodies and/or fragments thereof. In some embodiments, an Fc region is an Fc region of IgG1. In some embodiments, an Fc region is an Fc region of IgG2. In some embodiments, an Fc region is an Fc region of IgG3. In some embodiments, an Fc region is an Fc region of IgG4. In some embodiments, the present disclosure provides a plurality of complexes, wherein one or more complexes independently comprise an Fc region of IgG1. In some embodiments, the present disclosure provides a plurality of complexes, wherein one or more complexes independently comprise an Fc region of IgG2. In some embodiments, the present disclosure provides a plurality of complexes, wherein one or more complexes independently comprise an Fc region of IgG3. In some embodiments, the present disclosure provides a plurality of complexes, wherein one or more complexes independently comprise an Fc region of IgG4. In some embodiments, the present disclosure provides a plurality of complexes, wherein one or more complexes independently comprise an Fc region of IgG1, and one or more complexes independently comprise an Fc region of IgG2. In some embodiments, the present disclosure provides a plurality of complexes, wherein one or more complexes independently comprise an Fc region of IgG1, one or more complexes independently comprise an Fc region of IgG2, one or more complexes independently comprise an Fc region of IgG3, and one or more complexes independently comprise an Fc region of IgG4. In some embodiments, one or more complexes independently comprise a SARS-CoV-2 virus, and/or one or more complexes independently comprise a cell infected by SARS-CoV-2.

Without the intention to be bound by any theory, in some embodiments, provided technologies can deliver antibodies (e.g., through recruitment (e.g., antibody binding moieties) or by including antibody moieties) to an entity expressing a SARS-CoV-2 spike protein (unless otherwise indicated, including mutants thereof (e.g., those in viruses and/or infected cells)) or a fragment thereof (e.g., a SARS-CoV-2 virus, a cell infected by a SARS-CoV-2 virus, etc.). In some embodiments, antibodies reduce, inhibit or prevent interaction of SARS-CoV-2 viruses with other cells (e.g., mammalian cells that can be infected), in some embodiments, through disrupting, inhibiting or preventing interactions between SARS-CoV-2 spike proteins and cell proteins, e.g., receptors such as ACE2. In some embodiments, antibodies can induce, recruit, promote, encourage, or enhance one or more immune activities to inhibit, suppress, kill, or remove SARS-CoV-2 viruses and/or celled infected thereby. In some embodiments, antibodies can recruit dendritic cells. In some embodiments, a complex, e.g., a complex comprising a virus (e.g., a SARS-CoV-2 virus), an agent (e.g., an ARM agent comprising an antibody binding moiety, a target binding moiety and a linker as described herein) and an antibody moiety (either recruited antibody by an ARM agent or an antibody moiety in an agent; in some embodiment, such an antibody moiety is or comprises IgG2 which in some instances may have stronger binding to hFcyRIIA) binds hFcyRIIA on dendritic cells, and is internalized. Fragments of a virus, e.g., proteins and/or fragment thereof, are presented to immune cells (e.g., T cells) to provide long term immunity. In some embodiments, a complex comprising a virus-infected cell instead of a virus may similarly provide long term immunity. In some embodiments, provided technologies can provide long-term immunity (e.g., one or more vaccination effects). In some embodiments, provided technologies provide memory T and/or B cells against SARS-CoV-2.

In some embodiments, the present disclosure provides methods for inducing, promoting, encouraging, enhancing, triggering, or generating an immune response toward an infectious entity, e.g., a virus like SARS-CoV-2, comprising administering to a subject infected thereby an agent or a composition as described herein. In some embodiments, an immune response is or comprises ADCC. In some embodiments, an immune response is or comprises ADCP. In some embodiments, an immune response comprises ADCC and ADCP. In some embodiments, an immune response is or comprises long-term immunity. In some embodiments, an immune response is or comprises memory T and/or B cells. In some embodiments, a single dose is administered. In some embodiments, multiple doses are administered. In some embodiments, dosing intervals are about or not less than 1, 2 or 3 weeks, or about or not less than 1, 2, 3, 4, 5, 6, 7, 8, 9, 11 or 12 months, or about or not less than 1, 2, 3, 4, or 5 years. In some embodiments, at least one dosing interval is not less than 1, 2 or 3 weeks, or not less than 1, 2, 3, 4, 5, 6, 7, 8, 9, 11 or 12 months, or not less than 1, 2, 3, 4, or 5 years. In some embodiments, each dosing interval is independently not less than 1, 2 or 3 weeks, or not less than 1, 2, 3, 4, 5, 6, 7, 8, 9, 11 or 12 months, or not less than 1, 2, 3, 4, or 5 years. In some embodiments, a dosing interval is not less than 1 week. In some embodiments, a dosing interval is not less than 2 weeks. In some embodiments, a dosing interval is not less than 3 weeks. In some embodiments, a dosing interval is not less than 4 weeks. In some embodiments, a dosing interval is not less than 1 month. In some embodiments, a dosing interval is not less than 2 months. In some embodiments, a dosing interval is not less than 3 months. In some embodiments, a dosing interval is not less than 6 months. In some embodiments, a dosing interval is not less than or about 1 year. In some embodiments, a dosing interval is not less than or about 2 year. In some embodiments, a dosing interval is not less than or about 3 years. In some embodiments, a dosing interval is not less than or about 4 years. In some embodiments, a dosing interval is not less than or about 5 years.

In some embodiments, recruited antibodies or antibody moieties can induce, promote, encourage, enhance, trigger, or generate long-term immunity (e.g., after the initial ADCC and/or ADCP after an infection, or after 1, 2, 3, 4 or weeks, or after 1, 2, 3, 4, 5, 6, 7, 8, 9, 10, 11, 12 or more months after a last dose of an agent or a composition). In some embodiments, technologies of the present disclosure provide long-term immunity, e.g., toward SARS-CoV-2. In some embodiments, technologies of the present disclosure provide immunity in a period of time (e.g., 1, 2, 3, 4, 5, 6, 7, 8, 9, 10, 11, 12 or more months) after administration of an agent (in some embodiments, if multiple doses are administered as a regimen, after the first, the first several, or the last dose(s)) or a composition of the present disclosure. In some embodiments, a period of time is 6 months or more. In some embodiments, it is 7 months or more. In some embodiments, it is 8 months or more. In some embodiments, it is 9 months or more. In some embodiments, it is 10 months or more. In some embodiments, it is 11 months or more. In some embodiments, it is 1 year or more. In some embodiments, it is 2 years or more. In some embodiments, it is 3 years or more. In some embodiments, it is 4 years or more. In some embodiments, it is 5 years or more. In some embodiments, provided technologies can provide memory T or B cells against a target, e.g., SARS-CoV-2.

In some embodiments, the present disclosure provides a method for inhibiting, killing or removing a virus, e.g., SARS-CoV-2 virus, comprising administering to a subject infected thereby an effective amount of an agent or a composition. As appreciated by those skilled in the art, in some embodiments, an infected subject may not display a symptom (asymptomatic) when an infection is detected. In some embodiments, an agent or composition is administered before an infected subject displays a relevant symptom and/or when symptoms are considered mild (e.g., to prevent virus spreading, to prevent development of symptoms, and/or to prevent worsening of infection and/or overall condition of a subject). In some embodiments, a subject displays one or more symptoms considered medically “mild.” It is reported that common symptoms of SARS-CoV-2 infection/COVID-19 may be fever, tiredness, difficulty breathing, and/or dry cough. It is also reported that some subjects may have aches and pains, nasal congestion, runny nose, sore throat, loss of taste, loss of smell, and/or diarrhea. In some embodiments, symptoms are mild and begin gradually. In some embodiments, some subjects become infected but don’t develop any symptoms and don’t feel unwell. In some embodiments, a subject is seriously ill and develops difficulty breathing. In some embodiments, a subject is hospitalized. In some embodiments, provided agents and/or compositions are administered to subjects without symptoms, with mild symptoms, not hospitalized and/or hospitalized.

In some embodiments, the present disclosure provides a method for preventing and/or treating a condition, disorder or disease associated with an infection, e.g., a SARS-CoV-2 infection, comprising administering to a subject suffering therefrom a provided agent or composition. In some embodiments, the present disclosure provides a method for treating COVID-19, comprising administering to a subject suffering therefrom a provided agent or composition. In some embodiments, the present disclosure provides a method for inhibiting, killing or removing a virus, e.g., a SARS-CoV-2 virus, comprising contacting a virus, e.g., a SARS-CoV-2 virus, with a provided agent or composition. In some embodiments, the present disclosure provides a method for disrupting or reducing an interaction between a cell and a virus, e.g., a SARS-CoV-2 virus, comprising contacting a virus, e.g., a SARS-CoV-2 virus, with a provided agent or composition. In some embodiments, the present disclosure provides a method for disrupting or reducing an infection of a virus, e.g., a SARS-CoV-2 virus, of a cell, comprising contacting a virus, e.g., a SARS-CoV-2 virus, with a provided agent or composition. In some embodiments, the present disclosure provides a method for inhibiting, killing or removing a cell infected by a virus, e.g., a SARS-CoV-2 virus, comprising contacting the cell with a provided agent or composition. In some embodiments, provided agents or compositions are utilized in amounts effective to provide desired effects. As described herein, in some embodiments, immune cells, such as various NK cells, may be utilized together with provided agents and/or compositions, and may be administered prior to, concurrently with, or subsequently to provided agents and/or compositions. In some embodiments, a provided method is performed/started during an early phase of an infection or an associated condition, disorder or disease (e.g., COVID-19). In some embodiments, a provided method is performed/started before a subject generates strong immune activities. In some embodiments, a provided method is performed/started before a subject has acute respiratory distress syndrome (ARDS). In some embodiments, an agent is administered during an early phase of an infection or an associated condition, disorder or disease (e.g., COVID-19). In some embodiments, an agent is administered before a subject generates strong immune activities. In some embodiments, an agent is administered before a subject has ARDS.

In some embodiments, the present disclosure provides prophylactic methods for disrupting, reducing or preventing infection. In some embodiments, the present disclosure provides a method for disrupting, reducing or preventing a viral infection, e.g., an SARS-CoV-2 infection, comprising contacting a virus, e.g., a SARS-CoV-2 virus, with an effective amount of an agent or a composition of the present disclosure. In some embodiments, the present disclosure provides prophylactic methods for disrupting, reducing or preventing infection in advance of exposure. In some embodiments, an agent or composition is administered to a subject before the subject is exposed to or contacts an infectious entity, e.g., before the subject is exposed to a virus like a SARS-CoV-2 virus. In some embodiments, an agent or composition is administered to a subject before the subject is infected. As appreciated by those skilled in the art, various technologies are available for assessing viral infection, e.g. SARS-CoV-2 infection, and/or conditions, disorders or diseases associated therewith (e.g., those based on nucleic acid and/or protein detection, imaging (e.g., X-ray, CT, etc.), those according to guidelines of various government and/or private organizations (e.g., US CDC, WHO, etc.), etc.). In some embodiments, the present disclosure provides a method for disrupting, reducing or preventing a viral infection, e.g., SARS-CoV-2 infection in a population, comprising administering to individual subjects in the population an effective amount of agent or a composition of the present disclosure. In some embodiments, the present disclosure provides a method for disrupting, reducing or preventing a viral infection, e.g., SARS-CoV-2 infection, comprising administering to a subject susceptible thereto an effective amount of an agent or a composition of the present disclosure. In some embodiments, the present disclosure provides a method for disrupting, reducing or preventing a viral infection, e.g., a SARS-CoV-2 infection, comprising administering to a subject susceptible thereto an effective amount of an agent or a composition of the present disclosure. In some embodiments, an infection is a re-infection. In some embodiments, a subject, e.g., a subject in a population, is more susceptible to infection, at higher risk of infection, or is more likely to develop serious illness when infected (e.g., senior people (e.g., with age of 60, 70, 80 or more), or those with underlying medical problems (e.g., high blood pressure, heart problems, diabetes, etc.)). In some embodiments, a subject is a healthcare provider. In some embodiments, a subject is a frontline healthcare worker. In some embodiments, a subject is in contact or is in close proximity to an infected subject. In some embodiments, a subject is a healthcare worker who treats an infected patient. In some embodiments, a subject is of age 50, 55, 60, 65, 70, 75, 80, 85, 90 or more. In some embodiments, a subject is a person who lives in a nursing home or long-term care facility. In some embodiments, a subject has one or more underlying medical conditions, e.g., asthma, diabetes, high blood pressure, heart disease, etc. In some embodiments, a condition is chronic lung disease. In some embodiments, a condition is moderate to severe asthma. In some embodiments, a condition is a heart condition. In some embodiments, a subject is immunocompromised (as appreciated by those skilled in the art, can be caused by many conditions/factors, e.g., a medical treatment (a cancer treatment), smoking, bone marrow or organ transplantation, immune deficiencies, HIV or AIDS (particularly if poorly controlled), prolonged use of certain medications (e.g., corticosteroids and other immune weakening medications), etc.). In some embodiments, a subject is a cancer patient (e.g., immunocompromised). In some embodiments, a condition is obesity. In some embodiments, a condition is severe obesity (BMI >=40). In some embodiments, a condition is renal failure. In some embodiments, a condition is a liver disease. In some embodiments, prophylactic uses may comprise one, two or more doses. In some embodiments, multiple doses are administered. In some embodiments, one or more dose intervals are not more than 1, 2, 3, 4, 5, 6, 7, 8, 9, or 10 times of the half-life of an administered agent (which, as appreciated by those skilled in the art, can be assessed using a number of technologies). In some embodiments, one or more dose intervals are about or not more than 1, 2, 3, 4, 5, 6, 7, 8, 9, 10, 11, 12, 13, 14, 15, 16, 17, 18, 19, 20, 21, 22, 23, or 24 hours, or about or no more than 1, 2, 3, 4, 5, 6, 7, 8, 9, or 10 days. In some embodiments, each dose interval is independently about or not more than 1, 2, 3, 4, 5, 6, 7, 8, 9, 10, 11, 12, 13, 14, 15, 16, 17, 18, 19, 20, 21, 22, 23, or 24 hours, or about or no more than 1, 2, 3, 4, 5, 6, 7, 8, 9, or 10 days. In some embodiments, an agent or composition is administered once, twice, or thrice per day, or once every 2, 3, 4, 5, 6, or 7 days.

In some embodiments, the present disclosure provide technologies are useful against various viruses, e.g., for providing immunity, inhibiting, killing or removing viruses and/or cells infected thereby, preventing and/or treating conditions, disorders or diseases associated with viral infections, disrupting, reducing or preventing infections, etc. as described herein. In some embodiments, provided technologies can target two or more viruses. In some embodiments, provided technologies can target two or more or all coronaviruses that infect humans as described herein, e.g., SARS-CoV, SARS-CoV-2 and/or MERS-CoV. In some embodiments, provided technologies are useful for against SARS-CoV. In some embodiments, provided technologies are useful for against SARS-CoV-2. In some embodiments, provided technologies are useful for against MERS-CoV. In some embodiments, provided technologies are useful for against SARS-CoV and SARS-CoV-2. In some embodiments, provided technologies are useful for against SARS-CoV, SARS-CoV-2 and MERS-CoV.

In some embodiments, cells are mammalian cells. In some embodiments, cells are human cells. In some embodiments, cells are of the respiratory system.

In some embodiments, the present disclosure provides pharmaceutical compositions comprising or delivering a provided agent or a pharmaceutically acceptable salt thereof and a pharmaceutically acceptable carrier. In some embodiments, provided technologies are administered to subjects in pharmaceutical compositions.

### Combination Therapies

In some embodiments, provided technologies are administered together with one or more additional therapeutic agents and/or technologies. In some embodiments, useful additional therapeutic agents and/or technologies for combination are those that have been utilized to treat a condition, disorder or disease associated with viral infection, particularly infection by SARS-CoV-2.

In some embodiments, an additional therapeutic agent is or comprises immune cells. In some embodiments, immune cells are or comprise macrophages. In some embodiments, immune cells are or comprise NK cells. In some embodiments, immune cells are engineered cells. In some embodiments, immune cells are prepared in vitro. For example, in some embodiments, NK cells are or comprise engineered cells. In some embodiments, NK cells are or comprise allogeneic NK cells. In some embodiments, NK cells are or comprises peripheral blood-derived NK cells. In some embodiments, NK cells are or comprises cord blood-derived NK cells. In some embodiments, immune cells are or comprise MG4101 cells. In some embodiments, immune cells are or comprises CB-NK cells.

In some embodiments, immune cells are administered concurrently with provided agents; in certain embodiments, in the same composition. In some embodiments, immune cells are administered prior to provided agents. In some embodiments, immune cells are administered subsequently to provided agents.

Various immune cells, particularly NK cells, may be utilized together with agents described herein to treat various conditions, disorders or diseases including cancer. Such cells may be administered prior to, concurrently with, and/or subsequent to agents described herein, e.g., ARMs. In some embodiments, such cells, e.g., NK cells, are administered concurrently with an agent, e.g., an ARM, in the same composition comprising both NK cells and an ARM. In some embodiments, such cells, e.g., NK cells, are administered concurrently with an agent, e.g., an ARM, in separate compositions, e.g., one composition comprising NK cells but no ARMs, and one composition comprising an ARM but no NK cells.

As appreciated by those skilled in the art, useful immune cells such as NK cells may be from various sources and/or be engineered in a number of ways. For example, in some embodiments, NK cells are derived from stem cells. In some embodiments, NK cells are derived from iPSC lines. In some embodiments, NK cells are derived from a clonal master iPSC line. In some embodiments, NK cells are engineered to express certain receptors, e.g., a high-affinity, optionally non-cleavable CD16 receptor. In some embodiments, NK cells are engineered to express chimeric antigen receptors (CARs). In some embodiments, NK cells are CAR-NK cells. In some embodiments, NK cells are engineered to express cytokine receptor. In some embodiments, NK cells comprise a IL-15 receptor fusion that enhances the persistence and expansion capabilities without requiring co-administration of cytokine support. In some embodiments, NK cells are engineered to prevent expression of certain cell proteins, e.g., certain cell surface proteins. In some embodiments, NK cells are or comprise memory-like NK cells. In some embodiments, NK cells are or comprise pre-activated, memory-like NK cells enriched for CD56 and depleted from CD3 expressing cells. In some embodiments, NK cells are derived from placenta. In some embodiments, NK cells are donor NK cells. In some embodiments, NK cells are haploidentical donor NK cells. In some embodiments, NK cells are mismatched donor NK cells. In some embodiments, NK cells are related donor NK cells, e.g., mismatched related donor NK cells. In some embodiments, NK cells are unrelated donor NK cells. In some embodiments, NK cells are derived from a subject, e.g., a patient. In some embodiments, provided technologies comprise an innate cell engager, e.g., an innate cell engager binding to innate cells (e.g., NK cells and macrophages) while binding simultaneously to specific virally infected cells. In some embodiments, NK cells are derived from cord blood stem and progenitor cells. In some embodiments, NK cells are derived with modulation of a signaling pathway, e.g., the Notch signaling pathway. In some embodiments, nanoparticles are utilized to improve and/or sustain growth of NK cells. In some embodiments, as described herein, NK cells are generated ex vivo. In some embodiments, NK cells may be cryopreserved and stored in multiple doses as off-the-shelf cell therapy. Examples of certain immune cell technologies (e.g., NK cell technologies) include those utilized by Fate Therapeutics, NantKwest Inc., Celularity, Inc., GC Pharma, Sorrento Therapeutics, Inc., Affimed GmbH / MD Anderson Cancer Center, Gamida Cell Ltd., Nohla Therapeutics, Kiadis Pharma N.V., NKMax, Glycostem Therapeutics BV, GC LabCell, etc. Those skilled in the art will appreciate that, which they can be optionally utilized, antibodies and/or CARs toward specific antigens utilized in certain such technologies may not be required in provided technologies comprising ARMs as described herein.

### EXEMPLIFICATION

Various agents are prepared utilizing available chemistry in accordance with the present disclosure. Agents are assessed using available assays, e.g., those described in Zhang et al., https://doi.org/10.1101/2020.03.19.999318; Xia, S., Zhu, Y., Liu, M. et al. Fusion mechanism of 2019-nCoV and fusion inhibitors targeting HR1 domain in spike protein. Cell Mol Immunol (2020). https://doi.org/10.1038/s41423-020-0374-2; etc. for their properties and activities, including binding to SARS-CoV-2 spike proteins, inhibition, reduction and prevention of binding and/or infection of cells, inhibition, killing, and removal of SARS-CoV-2 viruses and/or cells infected thereby, etc. Provided agents can provide various useful properties and/or activities.

For example, a number of agents and compositions thereof were prepared and they demonstrated useful binding in various assays. Among other things, various results confirm that provided technologies can bind to SARS-CoV-2 spike proteins. Certain data are presented below as examples. Applicant notes that B2 may exist as E or Z isomers, and produced two major peaks during HPLC purification; compositions corresponding to the two peaks provided comparable data in certain assays, e.g., those described below. Those skilled in the art appreciate that many other technologies are available or can be developed for assess properties and activities of provided technologies in accordance with the present disclosure.

### General Synthetic Methods

Unless otherwise specified all reagents are standard reagent grade materials used without additional purification. The following abbreviations are used in the synthetic procedures that follow.
AcOH Acetyl
Aib Aminoisobutyric acid
DCM Dichloromethane
DIC N,N′-Diisopropylcarbodiimide
DIEA N,N-Diisopropylethylamine
DMAP 4-Dimethylaminopyridine
DMF Dimethyl furan
DMSO Dimethyl sulfoxide
EDCI 1-Ethyl-3-(3-dimethylaminopropyl)carbodiimide
Fmoc Fluorenyl methyloxycarbonyl protecting group
HATU Hexafluorophosphate Azabenzotriazole Tetramethyl Uronium
HFIP Hexafluor-2-propanol
HOBt 1-Hydroxybenzotriazole
HBTU Hexafluorophosphate Benzotriazole Tetramethyl Uronium
HFIP Hexafluroisopropanol
HOAc Acetic Acid
MBHA 4-Methylbenzhydrylamine
NMM N-Methyl morpholine
SPPS Solid Phase Peptide Synthesis
TFA Trifluoroacetic Acid
HPLC General Method

The following conditions are for a general HPLC method used to purify at least Agents I-23 to 1-31. The method below is for TFA purification conditions. Changes to the method for particular salt purifications are noted below.

| | |
|---|---|
| Sample Preparation | Dissolve in MeCN/H₂O |
| Instrument | Gilson GX-281-A |
| Mobile Phase | A: H₂O (0.075% TFA in H₂O) |
| | B: MeCN |
| Gradient | 35-55%-60 min. |
| Column | Luna 25*200 mm, C 18 10 um,110 Å + Gemin 150*30 mm,C18 5 um,110 Å |
| Flow Rate | 20 mL/min |
| Wavelength | 220/254 nm |
| Oven Temp.. | Room temperature |

### Example 1. Synthesis of Agent 1-23

Synthesis of the agents of this disclosure is performed using solid phase peptide synthesis methods, which will be familiar to those of skill in the art. The following examples are illustrative. Those of skill will recognize the changes in starting materials and reaction conditions needed to produce the various agents of this disclosure. Peptides are synthesized using standard Fmoc chemistry. Resin preparation: Rink Amide MBHA (3.00 mmol, 1.56 g, 0.32 mmol/g) and DMF (50 mL) are combined in a vessel for 2 hrs with N₂ bubbling at 15° C. Then 20% piperidine in DMF (100 mL) is added and the mixture bubbled with N₂ for 30 mins. at 15° C. The mixture is filtered to obtain the resin. The resin is washed with DMF (100 mL) before proceeding to next step.

Coupling is effected with a solution of Fmoc-Aib-OH (3.00 eq), HBTU (2.85 eq) in DMF (40 mL) is added to the resin with N₂ bubbling. Then DIEA (6.00 eq) is added to the mixture dropwise and bubbled with N₂ for 30 mins at 15° C. The coupling reaction is monitored by ninhydrin test. When the ninhydrin test shows colorless, the coupling is complete. The resin is then washed with DMF (100 mL)

De-protection is performed using 20% piperidine in DMF (100 mL) which is added to the resin and the mixture bubbled with N₂ for 30 mins at 15° C. The resin is then washed with DMF (100 mL) . The De-protection reaction is monitored by ninhydrin test, if the test shows blue or brownish red, the reaction is complete.

The coupling and deprotection steps are repeated for the addition of each following amino acid. After the last amino acid is added, the resin is washed with DMF (100 mL) and MeOH (100 mL) then dried under a vacuum.

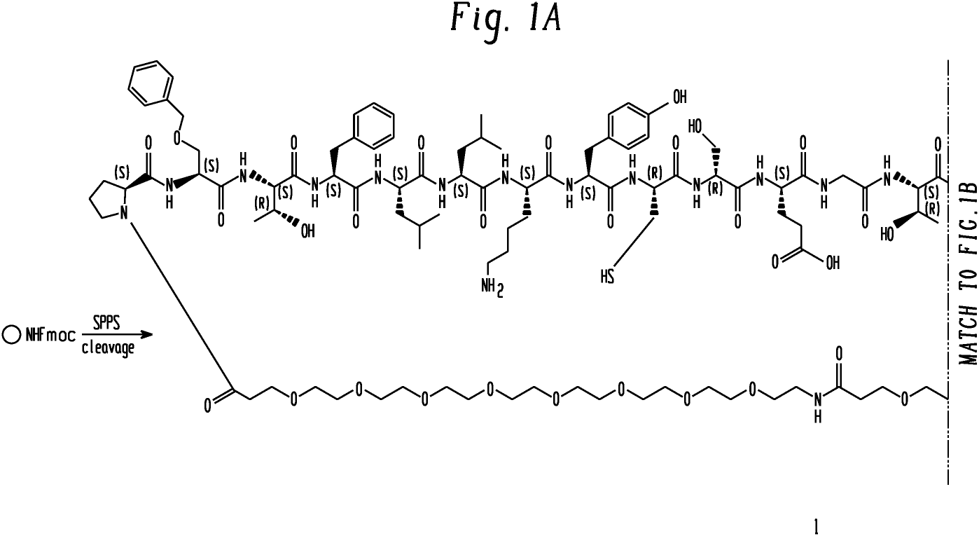

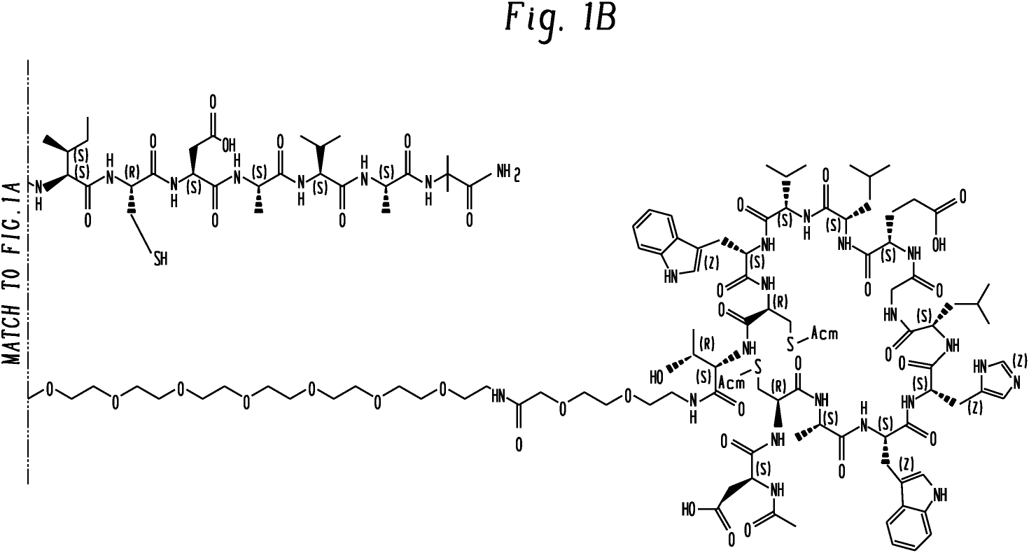

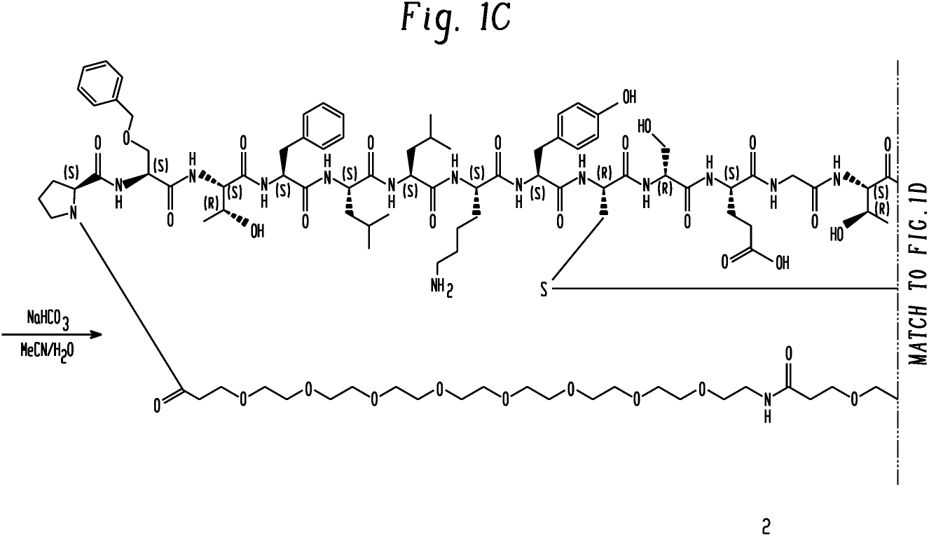

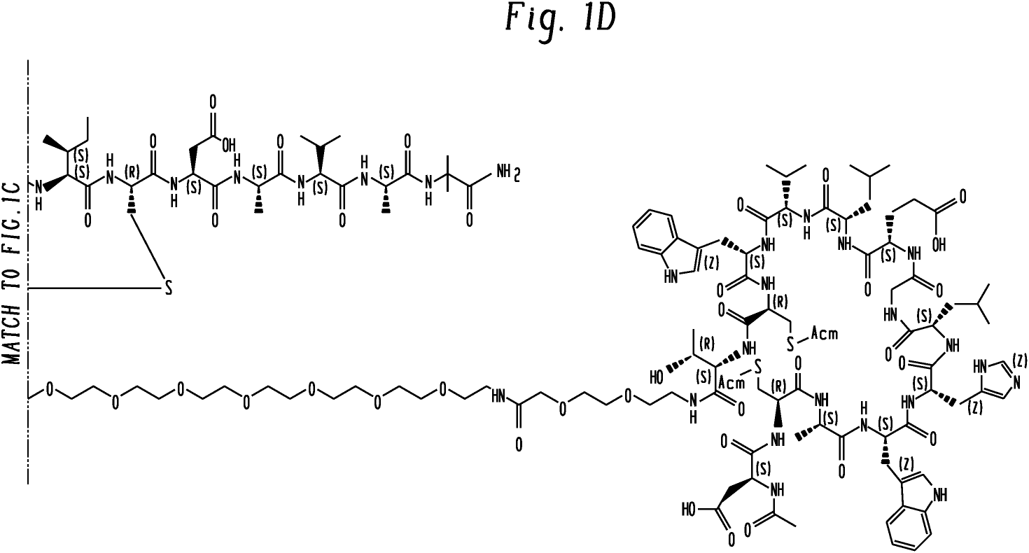

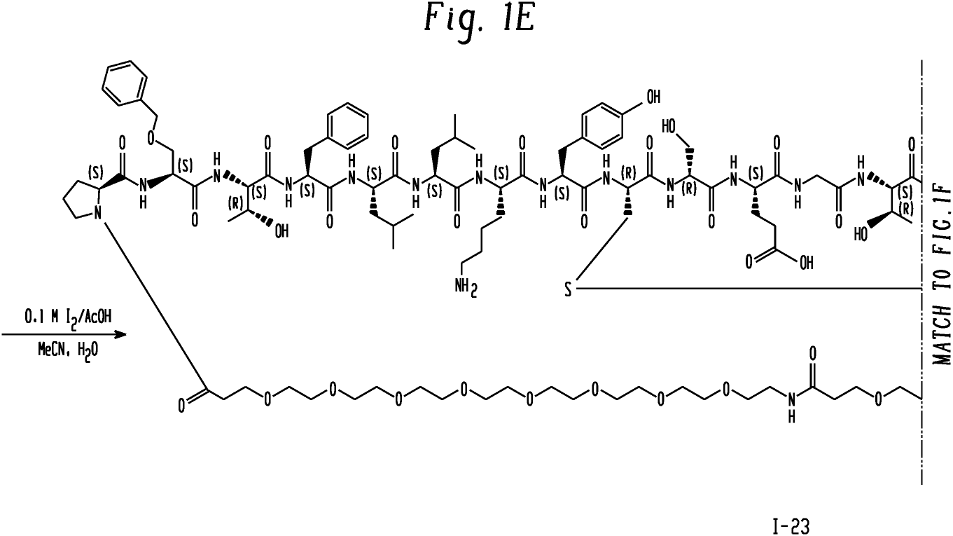

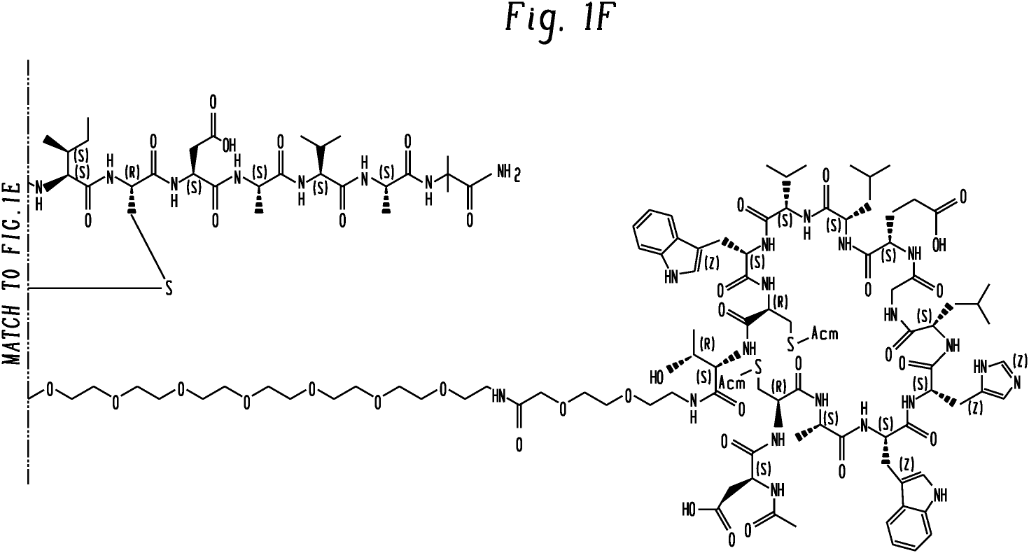

Following cleavage from the SPPS resin the peptide is cyclized in sodium carbonate buffered acetonitrile. The schematic for this synthesis of Agent I-23 is shown in FIG. 1.

Table 2 provides the sequence of solid phase coupling steps for the creation of agent I-23.

TABLE 2

Sequence of Solid Phase Coupling Reagents for 1-23 Synthesis

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Ala-OH (3.00 eq) | HBTU (2.85 eq) and DIEA (6.00 eq) |
| 3 | Fmoc-Val-OH (3.00 eq) | HBTU (2.85 eq) and DIEA (6.00 eq) |
| 4 | Fmoc-Ala-OH (3.00 eq) | HBTU (2.85 eq) and DIEA (6.00 eq) |
| 5 | Fmoc-Asp(OtBu)-OH (3.00 eq) | HBTU (2.85 eq) and DIEA (6.00 eq) |
| 6 | Fmoc-Cys(Trt)-OH (3.00 eq) | HBTU (2.85 eq) and DIEA (6.00 eq) |
| 7 | Fmoc-Ile-OH (3.00 eq) | HBTU (2.85 eq) and DIEA (6.00 eq) |
| 8 | Fmoc-Thr(tBu)-OH (3.00 eq) | HBTU (2.85 eq) and DIEA (6.00 eq) |
| 9 | Fmoc-Gly-OH (3.00 eq) | HBTU (2.85 eq) and DIEA (6.00 eq) |
| 10 | Fmoc-Glu(OtBu)-OH (3.00 eq) | HBTU (2.85 eq) and DIEA (6.00 eq) |
| 11 | Fmoc-D-Ser(tBu)-OH (3.00 eq) | HBTU (2.85 eq) and DIEA (6.00 eq) |
| 12 | Fmoc-Cys(Trt)-OH (3.00 eq) | HBTU (2.85 eq) and DIEA (6.00 eq) |
| 13 | Fmoc-Tyr(tBu)-OH (3.00 eq) | HBTU (2.85 eq) and DIEA (6.00 eq) |
| 14 | Fmoc-Lys(Boc)-OH (3.00 eq) | HBTU (2.85 eq) and DIEA (6.00 eq) |
| 15 | Fmoc-Leu-OH (3.00 eq) | HBTU (2.85 eq) and DIEA (6.00 eq) |
| 16 | Fmoc-Leu-OH (3.00 eq) | HBTU (2.85 eq) and DIEA (6.00 eq) |
| 17 | Fmoc-Phe-OH (3.00 eq) | HBTU (2.85 eq) and DIEA (6.00 eq) |
| 18 | Fmoc-Thr(tBu)-OH (3.00 eq) | HBTU (2.85 eq) and DIEA (6.00 eq) |
| 19 | Fmoc-Ser(Bzl)-OH (3.00 eq) | HBTU (2.85 eq) and DIEA (6.00 eq) |
| 20 | Fmoc-Pro-OH (3.00 eq) | HBTU (2.85 eq) and DIEA (6.00 eq) |
| 21 | Fmoc-PEG₈-CH₂CH₂COOH (1.50 eq) | HATU (1.42 eq) and DIEA (3.00 eq) |
| 22 | Fmoc-PEG₈-CH₂CH₂COOH (1.50 eq) | HATU (1.42 eq) and DIEA (3.00 eq) |
| 23 | Fmoc-AEEA-OH (3.00 eq) | HATU (2.85 eq) and DIEA (6.00 eq) |
| 24 | Fmoc-Thr(tBu)-OH (3.00 eq) | HATU (2.85 eq) and DIEA (6.00 eq) |
| 25 | Fmoc-Cys(Acm)-OH (2.00 eq) | HATU (1.90 eq) and DIEA (4.00 eq) |
| 26 | Fmoc-Trp-OH (3.00 eq) | HATU (2.85 eq) and DIEA (6.00 eq) |
| 27 | Fmoc-Val-OH (3.00 eq) | HATU (2.85 eq) and DIEA (6.00 eq) |
| 28 | Fmoc-Leu-OH (3.00 eq) | HATU (2.85 eq) and DIEA (6.00 eq) |
| 29 | Fmoc-Glu(OtBu)-OH (3.00 eq) | HATU (2.85 eq) and DIEA (6.00 eq) |
| 30 | Fmoc-Gly-OH (3.00 eq) | HATU (2.85 eq) and DIEA (6.00 eq) |
| 31 | Fmoc-Leu-OH (3.00 eq) | HATU (2.85 eq) and DIEA (6.00 eq) |
| 32 | Fmoc-His(Trt)-OH (3.00 eq) | HATU (2.85 eq) and DIEA (6.00 eq) |
| 33 | Fmoc-Trp-OH (3.00 eq) | HATU (2.85 eq) and DIEA (6.00 eq) |
| 34 | Fmoc-Ala-OH (3.00 eq) | HATU (2.85 eq) and DIEA (6.00 eq) |
| 35 | Fmoc-Cys(Acm)-OH (2.00 eq) | HATU (1.90 eq) and DIEA (4.00 eq) |
| 36 | Fmoc-Asp(OtBu)-OH (3.00 eq) | HATU (2.85 eq) and DIEA (6.00 eq) |
| 37 | Acetylation | 10% Ac₂O/ 5% NMM/ 85%DMF (100 mL) |

### Peptide Cleavage and Purification

Cleavage buffer (92.5%TFA/2.5%TIS/2.5%H₂O/2.5%3-mercaptopropanoic acid) is added to the flask containing the side chain protected peptide at room temperature and stirred for 2 hr. The peptide is filtered and collected the filtrate. The peptide is precipitated with cold isopropyl ether (1.5 L) and centrifuged (3 mins at 3000 rpm). The crude peptide is washed two additional times using isopropyl ether, and dried under vacuum for 2 hr to obtain crude peptide (compound 1 (FIG. 1)). A mixture of the crude peptide (compound 1) in MeCN/H₂O (1:1, 3.5 L) is adjusted to pH = 8 using 1 M NaHCO₃. Then the mixture is stirred at 15° C. for 48 hr for air oxidation. The reaction is quenched with 1 M HCl to adjust the pH = 6, followed by lyophilization to remove solvent. The residue was purified by prep-HPLC (acid condition, TFA). After lyophilization, compound 2 (FIG. 1) (3.27 g, 77.1% purity, 17.2% yield) was obtained as white solid.

### Formation of Agent 1-23

To the mixture of compound 2 (3.27 g, 670 umol) in H₂O (300 mL) and MeCN (300 mL) was added HOAc (30 mL), 1 M HCl (10 mL) to adjust pH = 1. Then 0.1 M I₂/AcOH (15 mL) was added for disulfide formation. The mixture was stirred at 15° C. for 3 hr. LCMS showed the reaction was complete. The solution was purified by prep-HPLC (acid condition, TFA) to afford Agent 1-23 (783 mg, 23.3% yield, 94.7% purity, TFA salt) as a white solid, retention time 40 minutes.

Agent I-23 was also prepared as an AcOH salt, (150 mg, 94.7% purity, TFA salt) was converted to (85.6 mg, 95.9% purity, AcOH salt), HPLC conditions: Phase A: H₂O (0.5% AcOH in H₂O), Phase B:MeCN. Retention time 21 min.

Agent I-23 was also prepared and an HCl salt. (150 mg, 94.7% purity, TFA salt) was converted to (113.7 mg, 97.1% purity, HCl salt) Phase A: H₂O (0.05% HCl in H₂O), Phase B: MeCN. Gradient 30-50%-60 min. Retention time 26 min.

### Example 2. Synthesis of Agent 1-24

Agent I-24 is prepared using the methods given above for the preparation of agent I-23, only the amount of starting material and sequence of solid state peptide coupling reagents for the solid state peptide synthesis are changed. Agent I-24 lacks the 2-position Alanine found in I-23 so the first two steps in the I-24 peptide synthesis are:

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Val-OH (3.00 eq) | HBTU (2.85 eq) and DIEA (6.00 eq) |

After the coupling to the 2-position Valine (which occurs at position 3 in I-23) the solid state peptide synthesis steps are the same. Following peptide synthesis and purification the resultant peptide is cyclized using the procedures given for I-23,to produce crude I-24. The solution was purified by prep-HPLC (acid condition, TFA) to get Agent I-24 (1.5 g, 14.6% yield, 97.5% purity, AcOH salt) as white solid, retention time 35 minutes. The AcOH salt is purified via HPLC, Phase A: H₂O (0.5% AcOH in H₂O), Phase B: MeCN, retention time 30 minutes.

### Example 3. Formation of Agent 1-25

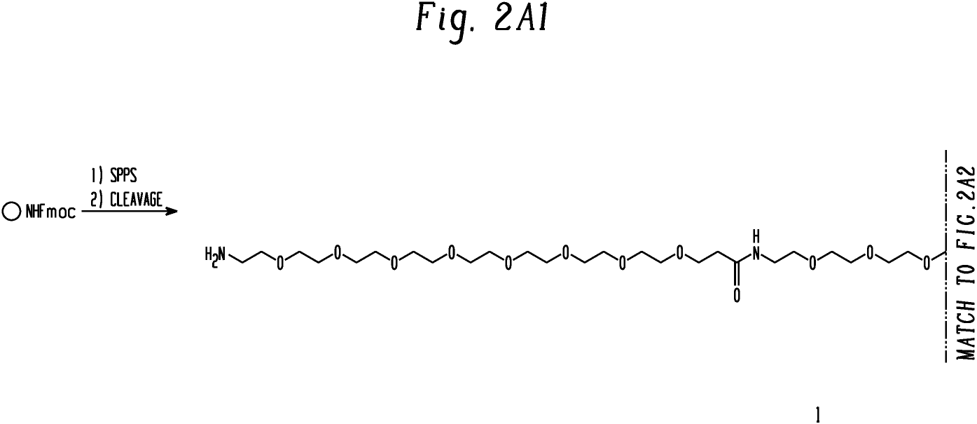

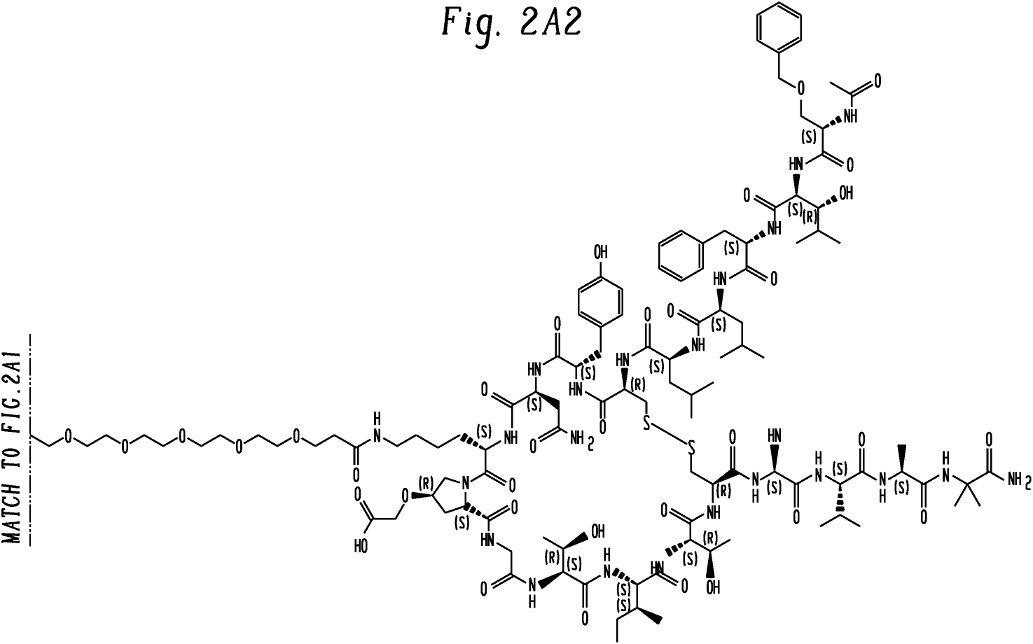

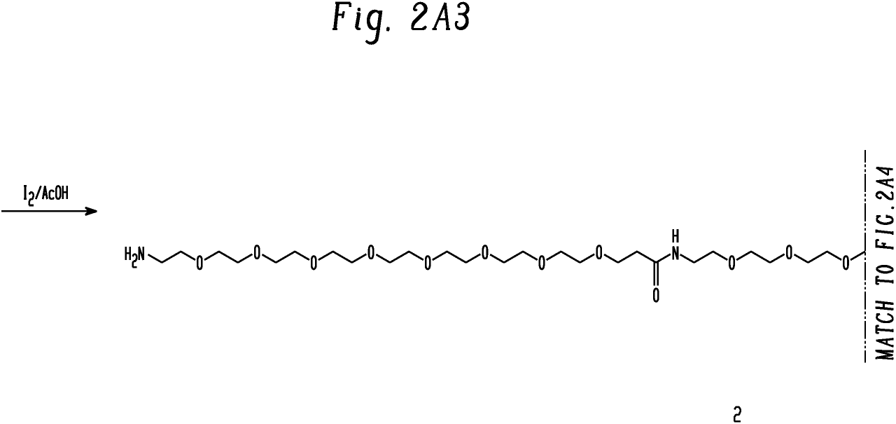

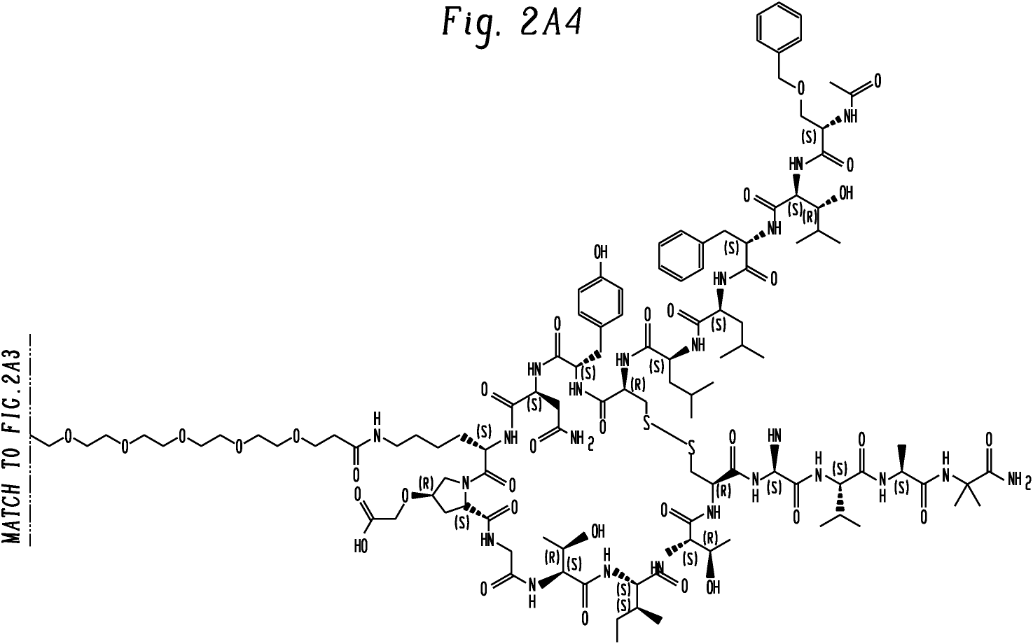

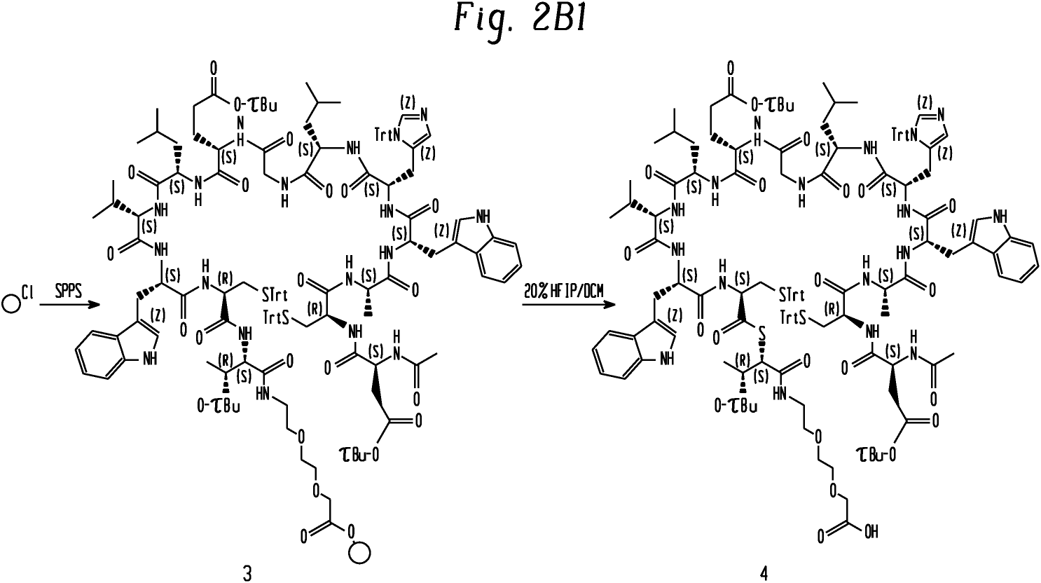

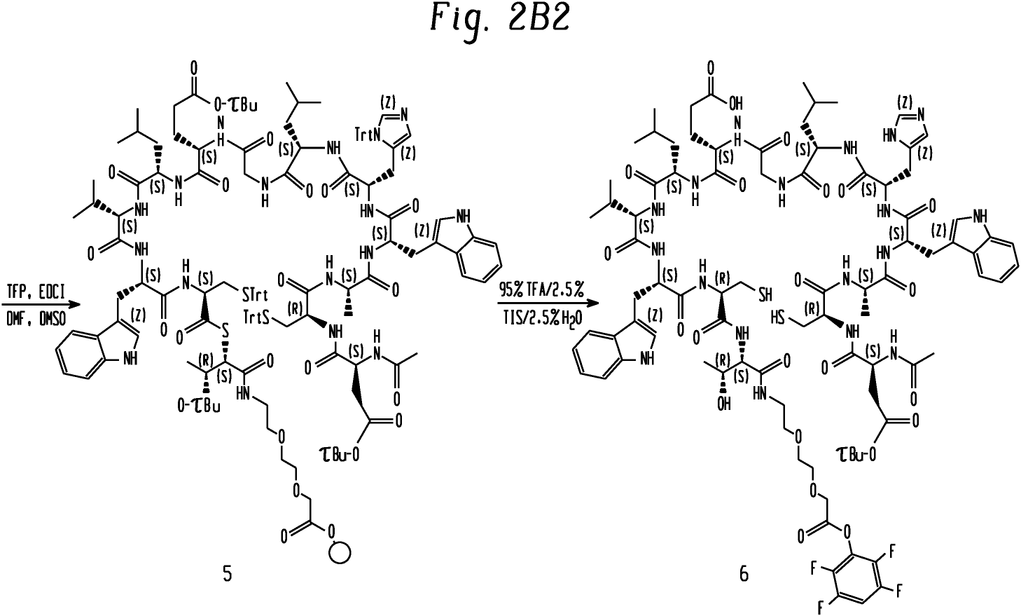

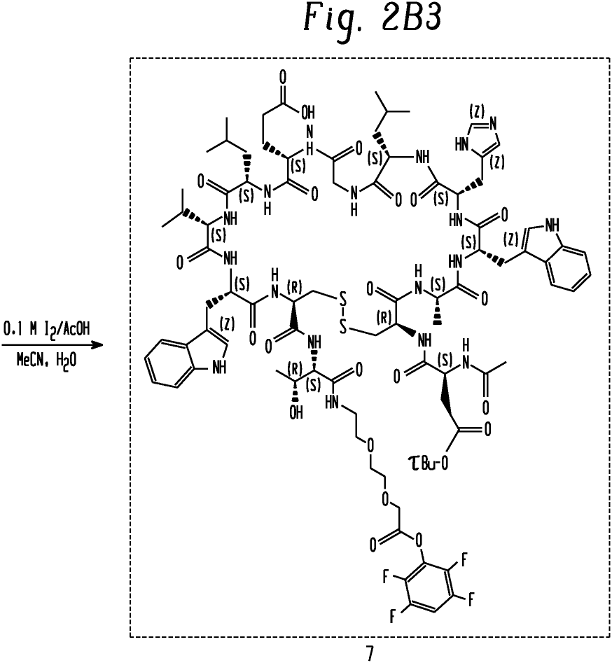

Agent I-25 is prepared by reacting two solid state peptide synthesis products (1) A COVID Spike protein binding moiety covalently bound to a linker and (2) and antibody binding moiety containing a tetrafluorophenyl group, to form the final Agent I-25 product. Preparation and treatment of the COVID Spike protein binding moiety/ linker and and antibody binding moiety that form Agent I-25 are shown in FIGS. 2A and 2B.

The solid phase peptide synthesis of the Spike protein binding moiety is performed according to the procedure given for I-23.There are a number of differences in the amino acid sequence between the Spike binding domain of Agent I-23 and Agent I-25 so the entire sequence of solid phase peptide coupling reactions is listed in Table 3.

TABLE 3

Sequence of Coupling Reagents for 1-25 Spike Protein Binding Moiety and Linker

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Ala-OH (3.00 eq) | HATU (2.85 eq) and DIEA (6.00 eq) |
| 3 | Fmoc-Val-OH (3.00 eq) | HBTU (2.85 eq) and DIEA (6.00 eq) |
| 4 | Fmoc-Ala-OH (3.00 eq) | HBTU (2.85 eq) and DIEA (6.00 eq) |
| 5 | Fmoc-Cys(Trt)-OH (3.00 eq) | HBTU (2.85 eq) and DIEA (6.00 eq) |
| 6 | Fmoc-Thr(tBu)-OH (3.00 eq) | HBTU (2.85 eq) and DIEA (6.00 eq) |
| 7 | Fmoc-Ile -OH (3.00 eq) | HBTU (2.85 eq) and DIEA (6.00 eq) |
| 8 | Fmoc-Thr(tBu)-OH (3.00 eq) | HBTU (2.85 eq) and DIEA (6.00 eq) |
| 9 | Fmoc-Gly-OH (3.00 eq) | HBTU (2.85 eq) and DIEA (6.00 eq) |
| 10 | (2S,4R)-1-(((9H-fluoren-9-yl)methoxy)carbonyl)-4-(2-(tert-butoxy)-2-oxoethoxy)pyrrolidine-2-carboxylic acid (2.00 eq) | HATU (1.9 eq) and DIEA (4.00 eq) |
| 11 | Fmoc-Lys(Dde)-OH (3.00 eq) | HATU (1.9 eq) and DIEA (4.00 eq) |
| 12 | Fmoc-Asn(Trt)-OH (3.00 eq) | HATU (2.85 eq) and DIEA (6.00 eq) |
| 13 | Fmoc-Tyr(tBu)-OH (3.00 eq) | HATU (2.85 eq) and DIEA (6.00 eq) |
| 14 | Fmoc-Cys(Trt)-OH (3.00 eq) | HATU (2.85 eq) and DIEA (6.00 eq) |
| 15 | Fmoc-Leu-OH (3.00 eq) | HATU (2.85 eq) and DIEA (6.00 eq) |
| 16 | Fmoc-Leu-OH (3.00 eq) | HATU (2.85 eq) and DIEA (6.00 eq) |
| 17 | Fmoc-Phe-OH (3.00 eq) | HATU (2.85 eq) and DIEA (6.00 eq) |
| 18 | Fmoc-Thr(tBu)-OH (3.00 eq) | HATU (2.85 eq) and DIEA (6.00 eq) |
| 19 | Fmoc-Ser(Bzl)-OH (3.00 eq) | HATU (2.85 eq) and DIEA (6.00 eq) |
| 20 | Acetylation | 10% Ac₂O/5% NMM/85% DMF (20.0 mL) |
| 21 | Fmoc-PEG₈-CH₂CH₂COOH (2.00 eq) | HATU (1.90 eq) and DIEA (4.00 eq) |
| 22 | Fmoc-PEG₈-CH₂CH₂COOH (2.00 eq) | HATU (1.90 eq) and DIEA (4.00 eq) |

After cycle 20, 3% N₂H₄·H₂O was used for Dde de-protection from Lys (Dde), and then the resin was washed with DMF, and continued with cycle #21 where Fmoc-PEG₈-CH₂CH₂COOH was coupled to the cleavLys sidechain. After cycle 22, the Fmoc was removed by 20% piperidine in DMF (20 mL).

### Peptide Cleavage and Purification

Cleavage buffer (92.5%TFA/2.5%TIS/2.5%H₂O/2.5%3-mercaptopropanoic acid) was added to the flask containing the side chain protected peptide at room temperature and stirred for 2 hr. The peptide was filtered and the filtrate collected. The peptide was precipitated with cold isopropyl ether (200 mL) and centrifuged (3 mins at 3000 rpm). The peptide is washed two additional times with isopropyl ether, and the crude peptide (compound 1, in FIG. 2A) was dried under vacuum for 2 hr. To a mixture of the crude peptide (compound 1) in MeCN/H₂O (500 mL) was added 0.1 M I₂/AcOH dropwise until the light yellow persisted, then the mixture was quenched with 0.1 M Na₂S₂O₃ dropwise until the light yellow disappeared. The mixture was dried via lyophilization. The reaction mixture was directly loaded to C18 column and purified by prep-HPLC (acid condition, TFA) to afford compound 2 (in FIG. 2A) (208 mg).

### Synthesis of 1-25 Antibody Binding Moiety

The antibody binding moiety for Agent I-25 is synthesized using the solid phase peptide synthesis procedure given for Agent I-25. The sequence of coupling reagents is provided in Table 4.

TABLE 4

Sequence of Coupling Reagents for Synthesis of 1-25 Antibody Binding Moiety

| # | Materials | Coupling reagents |
|---|---|---|
| 1 | Fmoc-AEEA-OH (3.00 eq) | DIEA (4.00 eq) |
| 2 | Fmoc-Thr(tBu)-OH (3.00 eq) | HBTU (2.85 eq) and DIEA (6.00 eq) |
| 3 | Fmoc-Cys(Trt)-OH (3.00 eq) | HBTU (2.85 eq) and DIEA (6.00 eq) |
| 4 | Fmoc-Trp-OH (3.00 eq) | HBTU (2.85 eq) and DIEA (6.00 eq) |
| 5 | Fmoc-Val-OH (3.00 eq) | HBTU (2.85 eq) and DIEA (6.00 eq) |
| 6 | Fmoc-Leu-OH (3.00 eq) | HBTU (2.85 eq) and DIEA (6.00 eq) |
| 7 | Fmoc-Glu(OtBu)-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | Fmoc-Leu-OH (3.00 eq) | HBTU (2.85 eq) and DIEA (6.00 eq) |
| 10 | Fmoc-His(Trt)-OH (3.00 eq) | HBTU (2.85 eq) and DIEA (6.00 eq) |
| 11 | Fmoc-Trp-OH (3.00 eq) | HBTU (2.85 eq) and DIEA (6.00 eq) |
| 12 | Fmoc-Ala-OH (3.00 eq) | HBTU (2.85 eq) and DIEA (6.00 eq) |
| 13 | Fmoc-Cys(Trt)-OH (3.00 eq) | HBTU (2.85 eq) and DIEA (6.00 eq) |
| 14 | Fmoc-Asp(OtBu)-OH (3.00 eq) | HBTU (2.85 eq) and DIEA (6.00 eq) |
| 15 | Ac₂O (4.00 eq) | NMM (8.00 eq) |

### Peptide Cleavage

Cleavage buffer (20%HFIP/DCM, 200 mL) was added to the flask containing the side chain protected peptide at room temperature and stirred for 1 hr twice. The peptide was filtered, and the filtrate collected. The Filtrate was concentrated and the residue was dried under lyophilization to five compound 4 FIG. 2B (10 g, crude) as a white solid.

### Synthesis of Agent 1-25 From Compounds 2 and 4

A mixture of compound 4 (FIG. 2B) (5.00 g, 1.91 mmol, 1.00 eq), 2,3,5,6-tetrafluorophenol (1.91 g, 11.47 mmol, 6.00 eq), EDCI (1.10 g, 5.74 mmol, 3.00 eq) in DMF (50 mL), DMSO (50 mL) was stirred at 15° C. for 16 hrs. DMF was removed under reduced pressure. The mixture was added to 0.1 M HCl (cold, 1 L) to precipitate white solid, after filtration, the crude compound 5 (5.3 g, crude) was obtained as a white solid.

A mixture of compound 5 (5.3 g, 361 umol) in 95%TFA/2.5%Tis/2.5%H₂O (100 mL) was stirred at 15° C. for 1 hr. The mixture was precipitated with cold isopropyl ether (1 L), after filtration, the solid was dried under reduced pressure to get compound 6 (3.5 g, crude) as a white solid.

A solution of compound 6 (3.5 g, crude) in TFA (20 mL) was added to MeCN (1.5 L) and H₂O (1.5 L). The mixture was added I₂/AcOH (0.1 M) dropwise at 15° C. until the color turned to light-yellow and stirred for 10 mins. The mixture was quenched with 0.1 M Na₂SO₃ dropwise until color turned to colorless, dried under lyophilization. The residue was purified by prep-HPLC (acid condition, TFA) to get compound 7 (250 mg, 134 umol, 7.1% yield, 90% purity) as a white solid.

To a mixture of compound 2 (50 mg, 16.46 umol, 1.00 eq) and compound 549 (36.86 mg, 19.75 umol, 1.20 eq) in DMF (0.5 mL) was added DIEA (8.51 mg, 65.84 umol, 11.47 uL, 4.00 eq) in one portion at 15° C. The mixture was stirred at15° C. for 1 hr. LCMS showed the reaction was completed. The solution was purified by prep-HPLC (TFA condition) directly to get Agent I-25-Lys (20.1 mg, 24.51% yield, 95.1% purity, TFA salt) as a white solid. Phase A: H₂O (0.075% TFA in H₂O), Phase B: MeCN. Gradient 25-55%-60 min. Retention time 48 min.

### Example 4. Synthesis of Agent 1-26

Agent I-26 is prepared using the methods given above for the preparation of agent I-23,only the amount of starting material and sequence of solid state peptide coupling reagents for the solid state peptide synthesis are changed. The sequence of solid state peptide coupling reagents is given in Table 5.

TABLE 5

Sequence of Peptide Coupling Reagents for I-26 Synthesis

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Val-OH (3.00 eq) | HBTU (2.85 eq) and DIEA (6.00 eq) |
| 3 | Fmoc-Ala-OH (3.00 eq) | HBTU (2.85 eq) and DIEA (6.00 eq) |
| 4 | Fmoc-Cys(Trt)-OH (3.00 eq) | HBTU (2.85 eq) and DIEA (6.00 eq) |
| 5 | Fmoc-Thr(tBu)-OH (3.00 eq) | HBTU (2.85 eq) and DIEA (6.00 eq) |
| 6 | Fmoc-Ile-OH (3.00 eq) | HBTU (2.85 eq) and DIEA (6.00 eq) |
| 7 | Fmoc-Thr(tBu)-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | (2S,4R)-1-(((9H-fluoren-9-yl)methoxy)carbonyl)-4-(2-(tert-butoxy)-2-oxoethoxy)pyrrolidine-2-carboxylic acid (1.50 eq) | HATU (1.42 eq) and DIEA (3.00 eq) |
| 10 | Fmoc-Lys(Dde)-OH (3.00 eq) | HATU (2.85 eq) and DIEA (6.00 eq) |
| 11 | Fmoc-Asn(Trt)-OH (3.00 eq) | HATU (2.85 eq) and DIEA (6.00 eq) |
| 12 | Fmoc-Tyr(tBu)-OH (3.00 eq) | HATU (2.85 eq) and DIEA (6.00 eq) |
| 13 | Fmoc-Cys(Trt)-OH (3.00 eq) | HATU (2.85 eq) and DIEA (6.00 eq) |
| 14 | Fmoc-Leu-OH (3.00 eq) | HATU (2.85 eq) and DIEA (6.00 eq) |
| 15 | Fmoc-Leu-OH (3.00 eq) | HATU (2.85 eq) and DIEA (6.00 eq) |
| 16 | Fmoc-Phe-OH (3.00 eq) | HATU (2.85 eq) and DIEA (6.00 eq) |
| 17 | Fmoc-Thr(tBu)-OH (3.00 eq) | HATU (2.85 eq) and DIEA (6.00 eq) |
| 18 | Fmoc-Ser(Bzl)-OH (2.00 eq) | HATU (1.90 eq) and DIEA (3.00 eq) |
| 19 | Acetylation | 10% Ac₂O/5% NMM/85% DMF (20 mL) |
| 20 | Fmoc-PEG₈-CH₂CH₂COOH (2.00 eq) | HATU (1.90 eq) and DIEA (4.00 eq) |
| 21 | Fmoc-PEG₈-CH₂CH₂COOH (2.00 eq) | HATU (1.90 eq) and DIEA (4.00 eq) |
| 22 | Fmoc-AEEA-OH (3.00 eq) | HATU (2.85 eq) and DIEA (6.00 eq) |
| 23 | Fmoc-Thr(tBu)-OH (3.00 eq) | HATU (2.85 eq) and DIEA (6.00 eq) |
| 24 | Fmoc-Cys(Acm)-OH (2.00 eq) | HATU (1.90 eq) and DIEA (4.00 eq) |
| 25 | Fmoc-Trp-OH (3.00 eq) | HATU (2.85 eq) and DIEA (6.00 eq) |
| 26 | Fmoc-Val-OH (3.00 eq) | HATU (2.85 eq) and DIEA (6.00 eq) |
| 27 | Fmoc-Leu-OH (3.00 eq) | HATU (2.85 eq) and DIEA (6.00 eq) |
| 28 | Fmoc-Glu(OtBu)-OH (3.00 eq) | HATU (2.85 eq) and DIEA (6.00 eq) |
| 29 | Fmoc-Gly-OH (3.00 eq) | HATU (2.85 eq) and DIEA (6.00 eq) |
| 30 | Fmoc-Leu-OH (3.00 eq) | HATU (2.85 eq) and DIEA (6.00 eq) |
| 31 | Fmoc-His(Trt)-OH (3.00 eq) | HATU (2.85 eq) and DIEA (6.00 eq) |
| 32 | Fmoc-Trp-OH (3.00 eq) | HATU (2.85 eq) and DIEA (6.00 eq) |
| 33 | Fmoc-Ala-OH (3.00 eq) | HATU (2.85 eq) and DIEA (6.00 eq) |
| 34 | Fmoc-Cys(Acm)-OH (2.00 eq) | HATU (1.90 eq) and DIEA (4.00 eq) |
| 35 | Fmoc-Asp(OtBu)-OH (3.00 eq) | HATU (2.85 eq) and DIEA (6.00 eq) |
| 36 | Acetylation | 10% Ac₂O/ 5% NMM/ 85%DMF (20 mL) |

After cycle 20, 3% N₂H₄·H₂O was used for Dde de-protection from Lys(Dde), and then the resin was washed with DMF*5, and continued with cycle #21 where Fmoc-PEG₈-CH₂CH₂COOH was coupled to Lys sidechain. Peptide Cleavage and cyclization follow the procedures set forth for Agent I-23. The crude peptide was purified by prep-HPLC (acid condition, TFA) directly to get Agent 1-26 (12.6 mg, 93.8% purity, 5.4% yield, TFA salt) as a white solid. Mobile phase A: H₂O (0.075% TFA in H₂O), Phase B: MeCN. 25-55% gradient, -60 min, retention time 52 minutes.

### Example 5. Synthesis of Agent 1-27

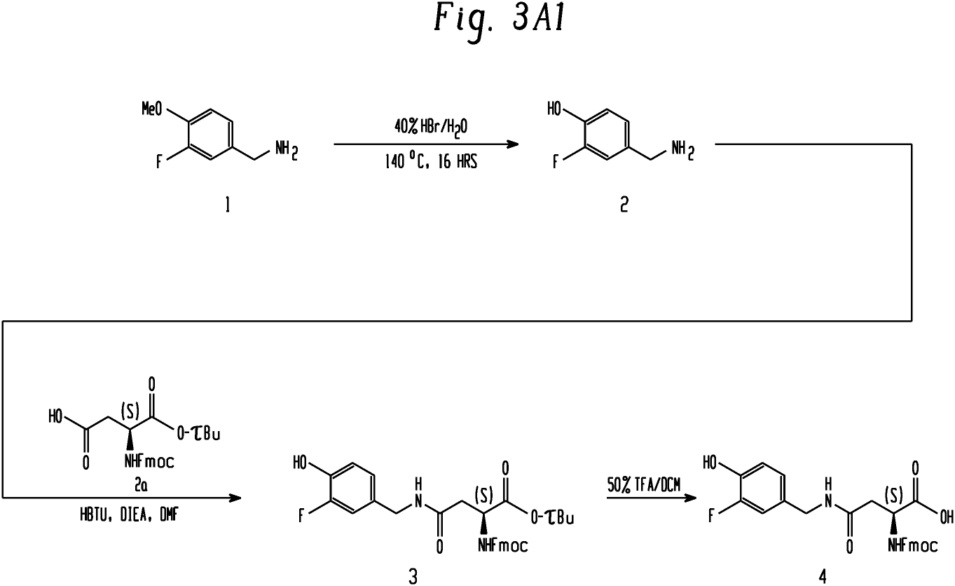

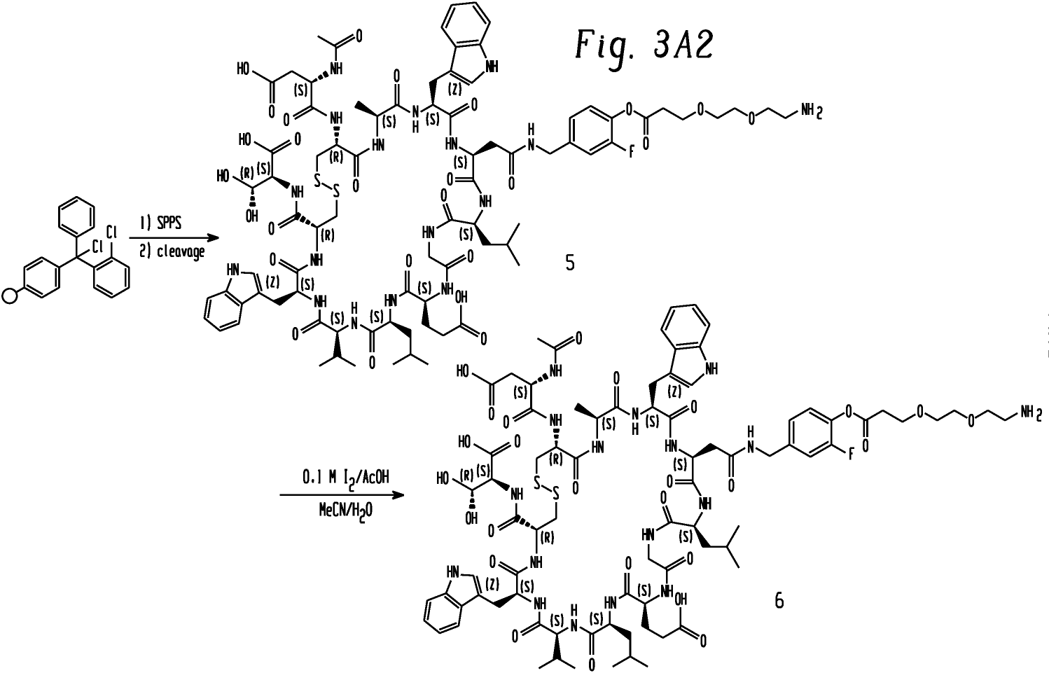

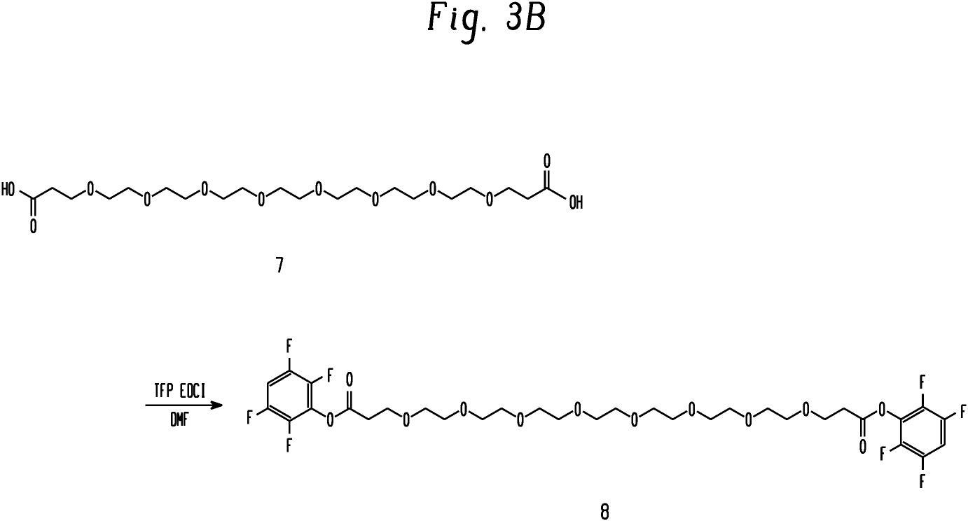

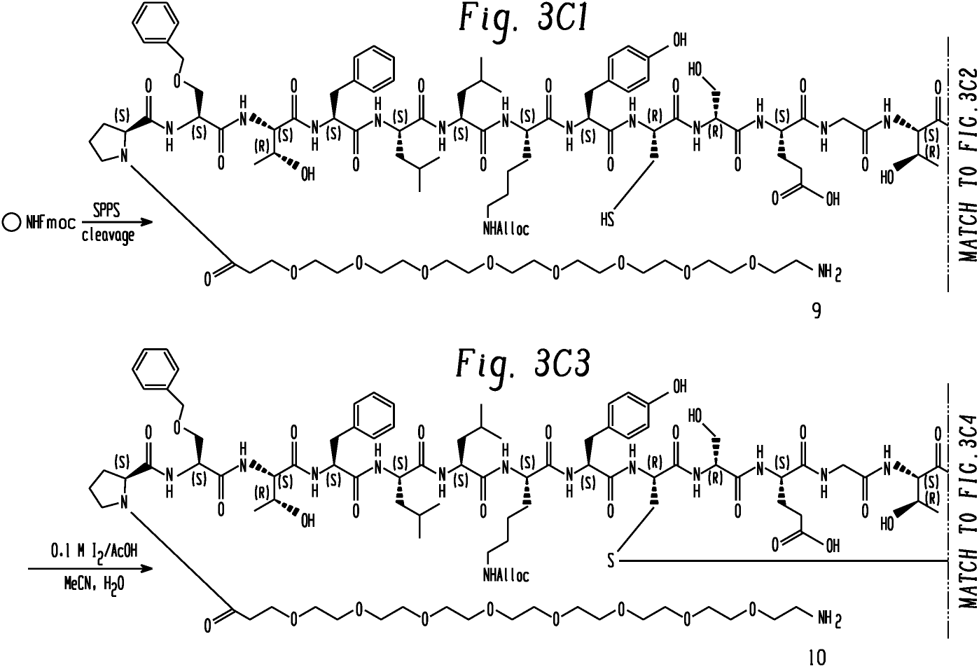

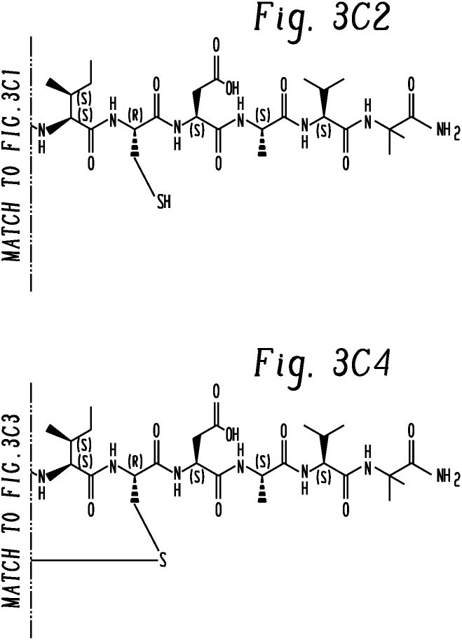

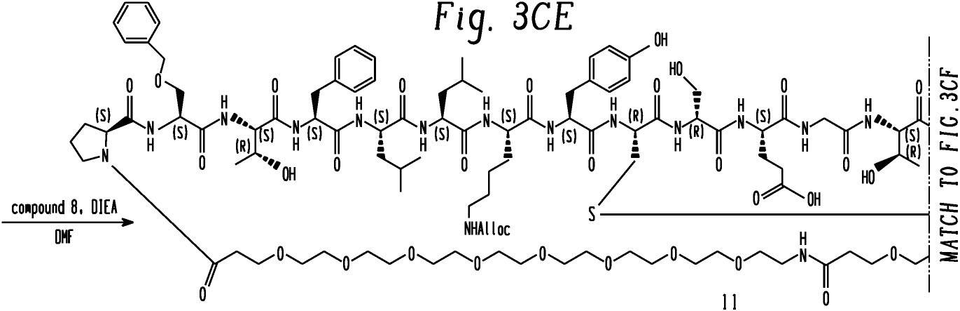

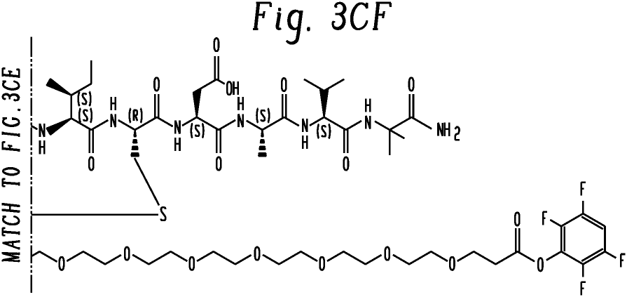

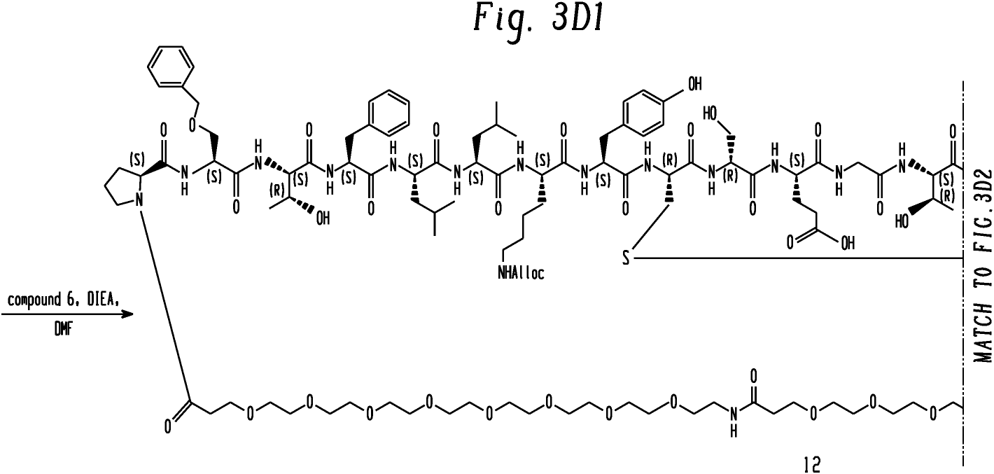

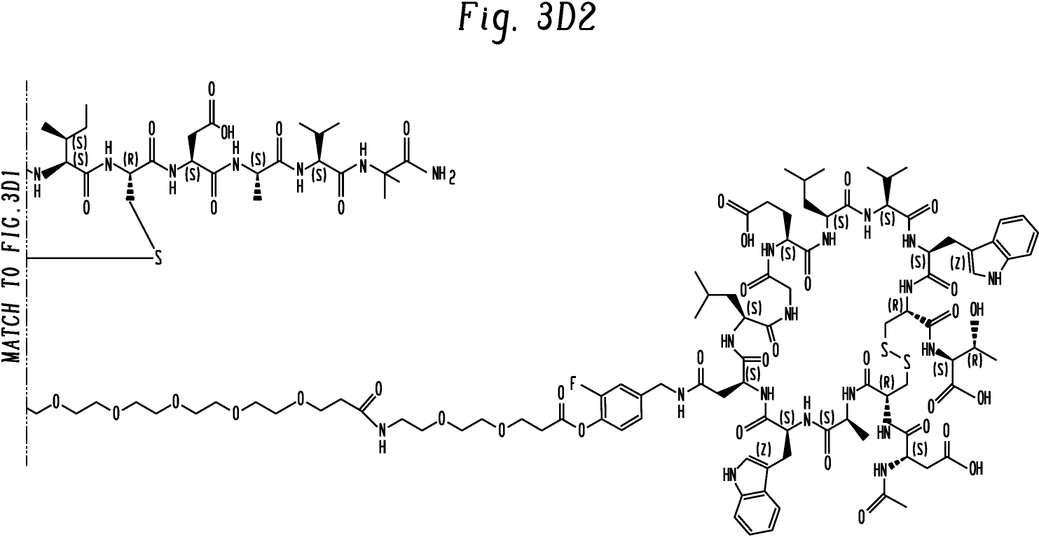

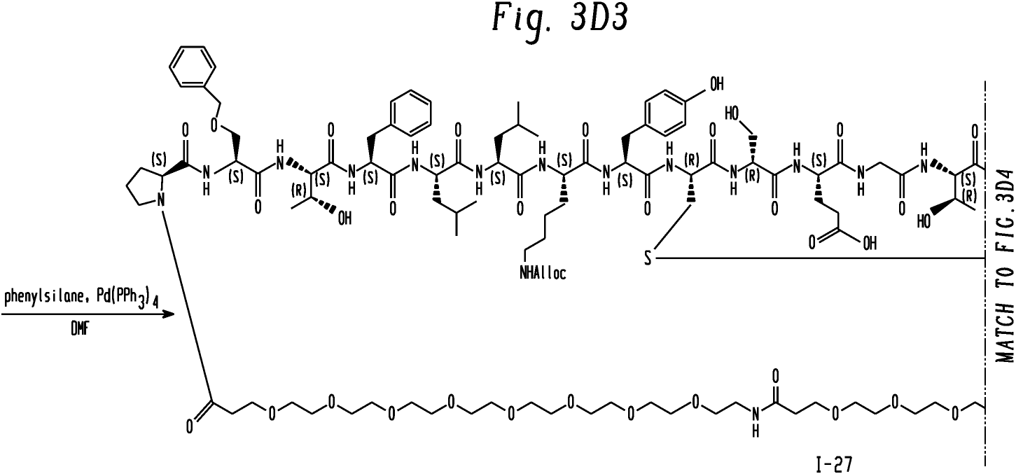

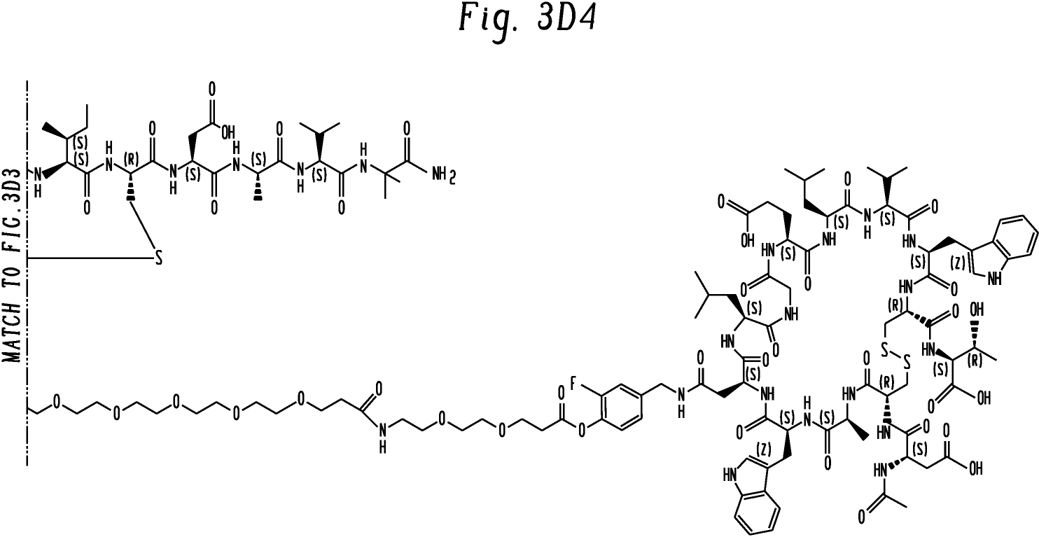

The synthesis of I-27 is depicted in FIGS. 3A, 3B, and 3C. A mixture of 4-(aminomethyl)- 2-flouro-3-methoxy (1, FIG. 3A) (50 g, 322.5 mmol) in HBr/H₂O (40% HBr, 500 mL in total) was stirred at 140° C. for 16 hrs. The solvent was removed at 70° C. under reduced pressure, the residue was triturated in MeCN (200 mL) for 10 mins. After filtering, the solid was dried under lyophilization to get compound 2 (65.0 g, 292.5 mmol, 90.8% yield, HBr) as a brown solid. ¹H NMR: (400 MHz DMSO-d₆) δ ppm 10.04 (s, 1 H) 8.18 (s, 3 H) 7.32 (dd, J= 12.17, 1.88 Hz, 1 H) 7.11 (dd, J= 8.28, 1.51 Hz, 1 H) 6.96 - 7.03 (m, 1 H) 3.93 (q, J = 5.52 Hz, 2 H).

To a mixture of compound 2 (65.0 g, 292.5 mmol, 1.00 eq, HBr), compound 2a (120.45 g, 292.5 mmol, 1.00 eq), DIEA (18.90 g, 146 mmol, 25.50 mL, 0.5 eq), HOBt (59.35 g, 439.1 mmol, 1.50 eq) in DMF (1000 mL) was added EDCI (61.75 g, 322.0 mmol, 1.10 eq) at 15° C., the mixture was stirred at 15° C. for 3 hr. The mixture was added to 0.5 M HCl (cold, 1 L) to precipitate crude product. After filtration, the solid was dissolved in DCM (3.00 L), washed with brine (1.00 L), dried over anhydrous Na₂SO₄, concentrated under reduced pressure. The residue was purified by silicagel column (DCM : MeOH = 10 : 1) to get compound 3 (120.0 g, 90% purity) as a white solid.

A mixture of compound 3 (120.0 g, 224.71 mmol) in TFA (800 mL) and DCM (800 mL) was stirred at 15° C. for 0.5 hr. The solvent was removed under reduced pressure. The residue was precipitated with cold isopropyl ether (1.50 L). After filtration, the solid was purified by silicagel column (DCM : MeOH = 20 : 1) to get compound 4 (90.0 g, 188.28 mmol, 83.7% yield) as a white solid.

The peptide was synthesized using standard Fmoc chemistry. The resin was prepared as follows. To the vessel containing CTC Resin (30.0 mmol, 30.0 g, 1.00 mmol/g) and Fmoc-Thr(tBu)-OH (11.91 g, 30.0 mmol, 1.00 eq) in DCM (150 mL) was added DIEA (4.00 eq) dropwise and mixed for 2 hrs with N₂ bubbling at 15° C. Then MeOH (30.0 mL)was added and bubbled with N₂ for another 30 mins. The resin was washed with DMF (600 mL). Then 20% piperidine in DMF (600 mL) was added and the mixture was bubbled with N₂ for 30 mins at 15° C. The mixture was filtered to obtain the resin. The resin was washed with DMF (600 mL) before proceeding to next step.

Coupling is effected by adding a solution of Fmoc-Cys(Trt)-OH (52.50 g, 3.00 eq), HBTU (30.24 g, 2.85 eq) in DMF (300 mL) to the resin with N₂ bubbling. Then DIEA (6.00 eq) was added to the mixture dropwise and bubbled with N₂ for 30 mins at 15° C. The coupling reaction was monitored by ninhydrin test, with a colorless test indicating the coupling is complete. The resin was then washed with DMF (600 mL).

The peptide was deprotected by adding 20% piperidine in DMF (600 mL) to the resin and the mixture was bubbled with N₂ for 30 mins at 15° C. The resin was then washed with DMF (600 mL). The De-protection reaction was monitored by ninhydrin test, if it showed blue or other brownish red, the reaction was completed. The coupling and deprotection step were repeated to add the remaining amino acids as listed in Table 6.

Following coupling and deprotections of amino acid 13 the peptide is acetylated. A solution of 10%Ac₂O/5%NMM/85%DMF (500 mL) was added to resin and the mixture was bubbled with N₂ for 20 mins. The coupling reaction was monitored by ninhydrin test, if it showed colorless, the coupling was completed. The resin was washed with DMF (600 mL) and dried under vacuum.

TABLE 6

Sequence of Peptide Coupling Reagents for 1-27 Synthesis

| # | Materials | Coupling reagents |
|---|---|---|
| 1 | Fmoc-Thr(tBu)-OH (1.00 eq) | DIEA (4.00 eq) |
| 2 | Fmoc-Cys(Trt)-OH (3.00 eq) | HBTU (2.85 eq) and DIEA (6.00 eq) |
| 3 | Fmoc-Trp-OH (3.00 eq) | HBTU (2.85 eq) and DIEA (6.00 eq) |
| 4 | Fmoc-Val-OH (3.00 eq) | HBTU (2.85 eq) and DIEA (6.00 eq) |
| 5 | Fmoc-Leu-OH (3.00 eq) | HBTU (2.85 eq) and DIEA (6.00 eq) |
| 6 | Fmoc-Glu(OtBu)-OH (3.00 eq) | HBTU (2.85 eq) and DIEA (6.00 eq) |
| 7 | Fmoc-Gly-OH (3.00 eq) | HBTU (2.85 eq) and DIEA (6.00 eq) |
| 8 | Fmoc-Leu-OH (3.00 eq) | HBTU (2.85 eq) and DIEA (6.00 eq) |
| 9 | compound 4 (2.00 eq) | DIC (2.00 eq) and HOBt (2.00 eq) |
| 10 | Fmoc-Trp-OH (3.00 eq) | DIC (3.00 eq) and HOBt (3.00 eq) |
| 11 | Fmoc-Ala-OH (3.00 eq) | DIC (3.00 eq) and HOBt (3.00 eq) |
| 12 | Fmoc-Cys(Trt)-OH (3.00 eq) | DIC (3.00 eq) and HOBt (3.00 eq) |
| 13 | Fmoc-Asp(OtBu)-OH (3.00 eq) | DIC (3.00 eq) and HOBt (3.00 eq) |
| 14 | Ac₂O | Ac₂O/NMM/DMF (10/5/85, 500 mL) |
| 15 | Boc-NH-PEG₂-CH₂CH₂COOH (3.00 eq) | DIC (3.00 eq), HOBt (3.00 eq) and DMAP (3.00 eq) |

The peptide is cleaved from the solid state resin with the addition of cleavage buffer (95%TFA/2.5%Tis/2.5%H₂O, 1.50 L) to the flask containing the side chain protected peptide at room temperature and stirred for 1 hr. The peptide is filtered and the filtrate collected. The peptide was precipitated with cold isopropyl ether (10.0 L). After filtration, the solid was washed with isopropyl ether (500 mL), and dried under vacuum for 2 hrs to give compound 5 (46.2 g, crude) as a white solid.

To a mixture of compound 5 (FIG. 3A) (9.0 g, crude) in MeCN/H₂O (5.0 L) was added 0.1 M I₂/HOAc dropwise until the yellow color persisted, then the mixture was stirred at 15° C. for 5 mins. The mixture was quenched with 0.1 M Na₂S₂O₃ dropwise until yellow color disappeared. The mixture was lyophilized to give the crude powder. The crude peptide was purified by prep-HPLC (acid condition, TFA) to get compound 6 (6.20 g, 99.0% purity, 11.2% yield, 5 batches) as a white solid.

To a mixture of compound 7 (FIG. 3B) (10.00 g, 21.30 mmol, 1.00 eq) and 2,3,5,6-tetrafluorophenol (21.20 g, 127.5 mmol, 6.00 eq), DMAP (519.3 mg, 4.25 mmol, 0.20 eq) in DMF (200 mL) was added EDCI (16.30 g, 85.0 mmol, 4.00 eq) at 15° C. The mixture was stirred at 15° C. for 3 hrs. The mixture was purified by C18 Flash (acid condition, TFA) to get compound 8 (12.0 g, 97.7% purity, 71.9% yield) as a colorless oil.

The synthesis of the Spike protein binding moiety is shown in FIGS. 3C and 3D. Peptide synthesis is carried out according to the above solid state peptide synthesis for compound 5, using the sequence of peptide coupling reagents listed in Table 7.

TABLE 7

Sequence of Peptide Coupling Reagents for Synthesis of Spike Binding Moiety of Agent 1-27

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Val-OH (3.00 eq) | HBTU (2.85 eq) and DIEA (6.00 eq) |
| 3 | Fmoc-Ala-OH (3.00 eq) | HBTU (2.85 eq) and DIEA (6.00 eq) |
| 4 | Fmoc-Asp(OtBu)-OH (3.00 eq) | HBTU (2.85 eq) and DIEA (6.00 eq) |
| 5 | Fmoc-Cys(Trt)-OH (3.00 eq) | HBTU (2.85 eq) and DIEA (6.00 eq) |
| 6 | Fmoc-Ile-OH (3.00 eq) | HBTU (2.85 eq) and DIEA (6.00 eq) |
| 7 | Fmoc-Thr(tBu)-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | Fmoc-Glu(OtBu)-OH (3.00 eq) | HBTU (2.85 eq) and DIEA (6.00 eq) |
| 10 | Fmoc-D-Ser(tBu)-OH (3.00 eq) | HBTU (2.85 eq) and DIEA (6.00 eq) |
| 11 | Fmoc-Cys(Trt)-OH (3.00 eq) | HBTU (2.85 eq) and DIEA (6.00 eq) |
| 12 | Fmoc-Tyr(tBu)-OH (3.00 eq) | HBTU (2.85 eq) and DIEA (6.00 eq) |
| 13 | Fmoc-Lys(Alloc)-OH (3.00 eq) | HBTU (2.85 eq) and DIEA (6.00 eq) |
| 14 | Fmoc-Leu-OH (3.00 eq) | HBTU (2.85 eq) and DIEA (6.00 eq) |
| 15 | Fmoc-Leu-OH (3.00 eq) | HBTU (2.85 eq) and DIEA (6.00 eq) |
| 16 | Fmoc-Phe-OH (3.00 eq) | HBTU (2.85 eq) and DIEA (6.00 eq) |
| 17 | Fmoc-Thr(tBu)-OH (3.00 eq) | HBTU (2.85 eq) and DIEA (6.00 eq) |
| 18 | Fmoc-Ser(Bzl)-OH (3.00 eq) | HBTU (2.85 eq) and DIEA (6.00 eq) |
| 19 | Fmoc-Pro-OH (3.00 eq) | HBTU (2.85 eq) and DIEA (6.00 eq) |
| 20 | Fmoc-PEG₈-CH₂CH₂COOH (2.00 eq) | HATU (1.90 eq) and DIEA (4.00 eq) |

Nascent peptide is cleaved from the solid state resin with the addition of cleavage buffer (92.5%TFA/2.5%TIS/2.5%H₂O/2.5%3-mercaptopropanoic acid) to the flask containing the side chain protected peptide at room temperature and stirred for 2 hrs. The peptide is filtered and the filtrate collected. The peptide was precipitated with cold isopropyl ether (5 L) and centrifuged (3 mins at 3000 rpm). Isopropyl ether is used to wash the peptide two additional times, and the crude peptide is dried under vacuum for 2 hrs to obtains compound 9 (FIG. 3C) (10 mmol, crude) as a white solid. To a mixture of the crude in MeCN/H₂O (1/1, 10 L) was added 0.1 M I₂/HOAc dropwise until the light yellow persisted, then The mixture was quenched with 0.1 M Na₂S₂O₃ dropwise until the light yellow disappeared. The mixture was dried under lyophilization. The residue was purified by Flash (acid condition, TFA) directly to get compound 10 (5.8 g, 95.9% purity, 21.0% yield) as a white solid.

The Spike binding moiety/ linker 10 is attached to the linker/ reactive group as follows. To a mixture of compound 8 (FIG. 3B) (5.28 g, 6.88 mmol, 5.00 eq) in DMF (50.0 mL) was added a mixture of compound 10 (3.62 g, 1.38 mmol, 1.00 eq). DIEA (712 mg, 5.51 mmol, 959.0 uL, 4.00 eq) in DMF (10.0 mL) is added dropwise at 0° C. over 1 min. Then the mixture was stirred at 0° C. for 5 mins. The mixture was purified by prep-HPLC (acid condition, TFA) directly to get compound 11 (FIG. 3C) (2.6 g, 90.4% purity, 58.7% yield) as a white solid.

A mixture of compound 11 (2.40 g, 718 umol, 1.00 eq), compound 6 (1.58 g, 811 umol, 1.13 eq), DIEA (186 mg, 1.44 mmol, 250 uL, 2.00 eq) in DMF (80.0 mL) was stirred at 15° C. for 30 mins. LCMS showed the reaction was complete. The solution was purified prep-HPLC (acid condition, TFA) to get compound 12 (2.40 g, 89.8% purity, 60.8% yield) as a white solid.

A mixture of compound 12 (2.40 g, 490.8 umol, 1.00 eq), Pd(PPh₃)₄ (113.3 mg, 98.1 umol, 0.20 eq), phenylsilane (529.5 mg, 4.90 mmol, 603 uL, 10.00 eq) in DMF (20.0 mL) was stirred under N₂ atmosphere at 15° C. for 1 hrs. The mixture was precipitated with isopropyl ether (cold, 200 mL) and centrifuged (3 mins at 3000 rpm), the solid was dried under reduced pressure and purified by prep-HPLC (acid condition, TFA) to provide Agent 1-27 (1.10 g, 213 umol, 48.6% yield, 93.6% purity, TFA salt) as a white solid. Mobile phase A: H₂O (0.075% TFA in H₂O), Phase B: MeCN, 25-55% gradient,-60 \. Min, retention time 50 min.

### Example 6. Synthesis of Agent 1-28

Agent I-28 is prepared by synthesis for I-27. The Spike binding moiety for I-28 includes an Alanine at the N-terminal that is not present in I-27. The sequence of peptide coupling reagents for the first two amino acids of Spike protein binding moiety is

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Ala-OH (3.00 eq) | HBTU (2.85 eq) and DIEA (6.00 eq) |

The remaining steps in the I-28 synthesis are performed by the procedure used to prepare I-27. The crude I-28 peptide mixture was precipitated with isopropyl ether (cold, 200 mL) and centrifuged (3 mins at 3000 rpm), the solid was dried under reduced pressure and purified by prep-HPLC (acid condition, TFA) to get agent I-28 (751.0 mg, 95.4% purity, 33.0% yield, TFA salt). Mobile Phase A: H₂O (0.075% TFA in H₂O), Phase B: MeCN, Gradient 25-55%-60 min. Retention time: 50 min.

### Example 7 Synthesis of Agent 1-29

Agent I-29 is prepared by methods set forth for the synthesis for I-27. The Spike binding moiety for I-29 differs. The sequence of peptide coupling reagents for the I-29 spike protein moieties is given in Table 8.

TABLE 8

Sequence of Peptide Coupling Reagents for Spike Protein Binding Moiety of I-29

| # | Materials | Coupling reagents |
|---|---|---|
| 1 | Fmoc-Aib-OH (3.00 eq) | HBTU (2.85 eq) and DIEA (6.00 eq) |
| 2 | Fmoc-Val-OH (3.00 eq) | HBTU (2.85 eq) and DIEA (6.00 eq) |
| 3 | Fmoc-Ala-OH (3.00 eq) | HBTU (2.85 eq) and DIEA (6.00 eq) |
| 4 | Fmoc-Cys(Trt)-OH (3.00 eq) | HBTU (2.85 eq) and DIEA (6.00 eq) |
| 5 | Fmoc-Thr(tBu)-OH (3.00 eq) | HBTU (2.85 eq) and DIEA (6.00 eq) |
| 6 | Fmoc-Ile-OH (3.00 eq) | HBTU (2.85 eq) and DIEA (6.00 eq) |
| 7 | Fmoc-Thr(tBu)-OH (3.00 eq) | HBTU (2.85 eq) and DIEA (6.00 eq) |
| 8 | Fmoc-Gly-OH (3.00 eq) | HBTU (2.85 eq) and DIEA (6.00 eq) |
| 9 | (2S,4R)-1-(((9H-fluoren-9-yl)methoxy)carbonyl)-4-(2-(tert-butoxy)-2-oxoethoxy)pyrrolidine-2-carboxylic acid (2.00 eq) | HATU (1.90 eq) and DIEA (4.00 eq) |
| 10 | Fmoc-Lys(Dde)-OH (2.00 eq) | HATU (1.90 eq) and DIEA (4.00 eq) |
| 11 | Fmoc-Asn(Trt)-OH (3.00 eq) | HBTU (2.85 eq) and DIEA (6.00 eq) |
| 12 | Fmoc-Tyr(tBu)-OH (3.00 eq) | HBTU (2.85 eq) and DIEA (6.00 eq) |
| 13 | Fmoc-Cys(Trt)-OH (3.00 eq) | HBTU (2.85 eq) and DIEA (6.00 eq) |
| 14 | Fmoc-Leu-OH (3.00 eq) | HBTU (2.85 eq) and DIEA (6.00 eq) |
| 15 | Fmoc-Leu-OH (3.00 eq) | HBTU (2.85 eq) and DIEA (6.00 eq) |
| 16 | Fmoc-Phe-OH (3.00 eq) | HBTU (2.85 eq) and DIEA (6.00 eq) |
| 17 | Fmoc-Thr(tBu)-OH (3.00 eq) | HBTU (2.85 eq) and DIEA (6.00 eq) |
| 18 | Fmoc-Ser(Bzl)-OH (3.00 eq) | HBTU (2.85 eq) and DIEA (6.00 eq) |
| 19 | Acetylation | 10%Ac₂0/5%NMM/85%DMF (20 mL) |
| 20 | Fmoc-PEG₈-CH₂CH₂COOH (2.00 eq) | HATU (1.90 eq) and DIEA (4.00 eq) |

After cycle #19, 3% N₂H₄·H₂O/DMF was used for Dde de-protection on Lys(Dde), and then the resin was washed with DMF, and continued for cycle #20 to couple on Lys sidechain. The Fmoc was removed by 20% piperidine in DMF. The peptide is cleaved from the solid state resin by a procedure similar to that for the Spike protein binding moiety for I-27 and I-28. The remaining steps for I-29 synthesis are given in the synthesis for I-27. Crude I-29 was purified prep_HPLC (acid condition, TFA) to get (27.8 mg, 96.9% purity, 33.0% yield) as a white solid. Mobile Phase A: H₂O (0.075% TFA in H₂O), Phase B:MeCN, gradient 30-60%-60 min. Retention time: 50 min.

### Example 8. Synthesis of Agent 1-30

Agent I-30 is prepared by methods set forth for the synthesis for I-27. The Spike binding protein moiety contains differences. The sequence of peptide coupling reagent for the I-30 Spike has an alanine at the second position, but is otherwise the same at the spike protein binding moiety for I-27. I-30 is purified from crude peptide by prep-HPLC (acid condition, TFA) to get 1-30 (6.7 mg, 1.20 umol, 86.3% purity, 17.5% yield) as a white solid. Mobile Phase A: H₂O (0.075% TFA in H₂O), Phase B: MeCN, Gradient 25-55%-60 min. Retention time: 48 min.

### Example 9. Synthesis of 1-31

Agent I-31 is prepared by methods set forth for the synthesis for I-25. I-31 (Orn) is obtained from crude peptide by prep-HPLC (1^{st} TFA, 2^{nd} CH₃COONH₄) directly to the desired compound (15.2 mg, 17.81% yield, 91.5% purity, CH₃COONH₄ salt) as a white solid.Mobile Phase A: H₂O (0.075% TFA in H₂O, Phase B: MeCN, Gradient 25-55%, 60 minutes, retention time 48 min.

### Example 10. Biological Data

Certain data from BLI binding assay: and ELISA binding assay

| | | | | | |
|---|---|---|---|---|---|
| Cpd. # | Octet Spike Trimer K_{D} | Octet Spike RBD K_{D} | Elisa Spike Trimer IC₅₀ | Elisa Spike RBD IC₅₀ | SARS-CoV-2 (live) neutralization (plaque) assay IC50 |
| B-1 | 109 nM | | | | 2.42 µM |
| B-2, B2′, B-2″ | | 349 nM | | | 10 µM |
| B-2, B2′, B-2″ | | 457 nM | | | |
| B-3 | 42 nM (n=2) | 156 nM (n=2) | | | 2.79 µM |
| B4 | 208 nM | | | | |
| I-15 | | | 147 nM, 152 nM | | |
| I-17 | 3635 nM | | 118 nM (n=2) | | 1.25 µM |
| I-18 | | | 6.9, 10.6, 499 | 743 nM, 1265 nM | 1.63 µM |
| I-24 | specific binding detected at high conc. | | | | |
| I-27 | n.a. | | n.a. | | n.a. |
| I-28 | n.a. | | n.a. | | n.a. |
| I-29 | n.a. | | n.a. | | n.a. |
| 1-30 | n.a. | | n.a. | | n.a. |
| I-31 | n.a. | | n.a. | | n.a. |
| 1-33 | 38 nM | | | | |
| I-34 | 373 nM | 278 nM | | | 1.1 µM |

Certain assay conditions useful for accessing provided technologies are described below. Those skilled in the art appreciate that various steps and/or conditions can be appropriately adjusted in accordance with the present disclosure.

BLI Binding assay. A ForteBio Octet® RED96e Bio-Layer Interferometry (BLI) system (Octet RED96e, ForteBio, CA) was used to determine affinity of peptide binders to target spike protein. Streptavidin tips were dipped into 50 nM of an agent (e.g., B-1, B-3, etc.) (PBS pH 7.4, 0.05% Tween 20, 0.1% BSA). After loading step is completed, agent loaded tips were treated with the range of concentrations of proteins, e.g., spike protein trimer (Acro Biosystems SPN-C52H). After association, the tips were dipped into buffer (PBS pH 7.4, 0.05% Tween 20, 0.1% BSA) for dissociation. Obtained assay curves were fitted with ForteBio Biosystems (1:1 binding model) to derive K_{D} (e.g., based on kₒₙ and k_{off}).

ELISA binding assays. Spike protein binding analysis to various agents was measured by ELISA. Briefly, high binding 96-well plate (Perkin Elmer 6005580) was coated with agents (e.g., I-17, I-18, etc.), washed with PBS buffer containing 0.05% Tween 20, and blocked with BSA. Immobilized compounds were treated with different concentrations of proteins, e.g., spike protein (trimer Acro Biosystems SPN-C52H or RBD Sino Biologicals 40592-V08B) at various concentrations. Bound spike proteins were detected in PBS (pH 7.4, 0.05% Tween 20) using anti-6xHis antibody conjugated with HRP (Abcam ab178563). Detection reagent was SuperSignal ELISA Pico Chemiluminescent Substrate (Thermo fisher, 37069) followed by luminescence read on Biotek Synergy H1 microplate reader.

While we have described a number of embodiments, it is apparent that our basic examples may be altered to provide other embodiments that technologies (e.g., compounds, agents, compositions, methods, etc.) of the present disclosure. Therefore, it will be appreciated that the scope of an invention is to be defined by claims rather than by the specific embodiments that have been represented by way of example.

1. An agent having the structure of: 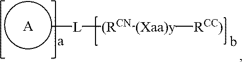
or a pharmaceutically acceptable salt thereof, wherein:
the agent inhibits, kills, or removes a cell infected by SARS-CoV-2
each of a and b is 1;
A is chosen from ABT, AT, and PT;
ABT is an antibody binding moiety that has the structure: R^{c}-(Xaa)z- or 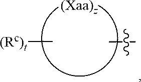
wherein t is an integer from 1 to 20, and z is an integer from 1 to 50;
AT is an antibody moiety that is immunoglobulin G (IgG), a derivative of IgG, pooled IgG, or intravenous immune globulin (IVIG);
PT is independently a partner moiety selected from a diagnostic agent or detectablemoiety;
L is a linker moiety;
each Xaa is independently an amino acid or an amino acid analog;
y is 5-50;
R^{CN} and R^{CC} are independently R^{C};
each R^{C} is independently -L^{a}-R′;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the aliphatic or heteroaliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent monocyclic, bicyclic-, or polycyclic group, wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently -R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or
two or more R groups on the same atom are optionally and independently taken together with the atom they are attached to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic, or polycyclic ring having, in addition to the intervening atoms, 0-10 heteroatoms;
wherein -(Xaa)y- is or comprises:
-(Xaa^{T0})y0-(Xaa^{T1})y1-Xaa^{T2}-(Xaa^{T3})y3-Xaa^{T4}-(Xaa^{T5})y5-(Xaa^{T6})y6-(Xaa^{T7})y7-(Xaa^{T8})y8-Xaa^{T9}-(Xaa^{T10})y10-(Xaa^{T11})y11-(Xaa^{T12})y12-, wherein:
y0 is an integer from 0 to 20;
each Xaa^{T0} is independently an amino acid or an amino acid analog;
y1 is 0, 1, or 2;
each Xaa^{T1} is independently an amino acid or an amino acid analog;
Xaa^{T2} is an amino acid or an amino acid analog whose side chain comprises 3 or more non-hydrogen atoms;
y3 is an integer from 0 to 10;
each of Xaa^{T3} is independently an amino acid or an amino acid analog;
each of Xaa^{T4} and Xaa^{T9} is independently an amino acid or an amino acid analog, wherein Xaa^{T4} is optionally connected to Xaa^{T9} through a linker;
y5 is an integer from 0 to 10;
each Xaa^{T5} is independently an amino acid or an amino acid analog;
y6 is 0, 1, or 2;
each Xaa^{T6} is independently an amino acid or an amino acid analog;
y7 is 0 or 144;
Xaa^{T7} is a negatively-charged amino acid or a negatively charged amino acid analog;
y8 is an integer from 0 to 10;
each Xaa^{T8} is independently an amino acid or an amino acid analog;
y10 is an integer from 0 to 10;
each of Xaa^{T10} is independently an amino acid or an amino acid analog;
y11 is 1, 2, 3, 4, or 5;
each Xaa^{T11} is independently an amino acid or an amino acid analog;
y12 is an integer from 0 to 20;
each Xaa^{T12} is independently an amino acid or an amino acid analog.

2. The agent of claim 1, wherein each of Xaa^{T0}, Xaa^{T1}, Xaa^{T2}, Xaa^{T3}, Xaa^{T4}, Xaa^{T5}, Xaa^{T6}, Xaa^{T7}, Xaa^{T8}, Xaa^{T9}, Xaa^{T10}, Xaa^{T11}, and Xaa^{T12} is independently an amino acid having the structure of formula A-I: 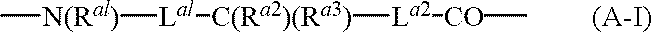
or a salt thereof, wherein:
\each of R^{a1}, R^{a2}, and R^{a3} is independently —L^{a}—R′;
each of L^{a1} and L^{a2} is independently L^{a};
\\each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the aliphatic or heteroaliphatic group are optionally and independently replaced with —C(R′)₂—, —Cy—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each —Cy— is independently an optionally substituted bivalent monocyclic, bicyclic, or polycyclic group, wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently -R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or-=
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

3. The agent of claim 2, wherein
y0 is 0, y1 is 1 or 2; and -(Xaa^{T1})y1- is or comprises a dipeptide or an amino acid that is suitable for forming a turn;
-Xaa^{T2}- is or comprises an amino acid having the structure of formula A-I, wherein R^{a2} is hydrophobic, neutral or negatively charged and is a group —L^{a}—R′, wherein L^{a} is an optionally substituted bivalent group selected from C₃-C₁₀ aliphatic or C₃-C₁₀ heteroaliphatic having 1-5 heteroatoms, wherein one or more methylene units of the group are optionally and independently replaced with —C(R′)₂—, —O—, —S—, —N(R′)—, —C(O)—, —C(S)—, —C(NR′)—, —S(O)—, or S(O)₂—;
y3 is 1-10;
Xaa^{T4} is connected to Xaa^{T9} through a linker, wherein the linker is L^{a} and is bonded to a backbone carbon atom of Xaa^{T4} and a backbone carbon atom of Xaa^{T9;} wherein L^{a} is or comprises —CH₂—CH₂—, —O—, —S— or —S—S—;
-(Xaa^{T5})y5- is or comprises YNK and -(Xaa^{T5})y5- is connected to an antibody moiety or an antibody binding moiety optionally through a linker bound to the lysine (K) amino acid in -(Xaa^{T5})y5;
y5 is 0 and-(Xaa^{T6})y6- is or comprises a D-Ser;
y6 is 1or 2;
y7 is 1 and comprises —COOH or y7 is 0;
-(Xaa^{T8})y8- is or comprises GTI or -G-Xaa^{T8}-IT-Xaa^{T8}-, wherein each Xaa^{T8} is independently an alpha amino acid;
y10 is 3 or 4;
-(Xaa^{T11})y11- is or comprises L-Ala, D-Ala, Aib, Gly, or negatively charged; or
a combination thereof.

4. The agent of claim 1, wherein
y1 is 0;
-(Xaa^{T1})y1- is or comprises L-proline, D-proline, a proline derivative, L-serine, D-serine, glycine, L-pseudoproline, or D-psuedoproline; -(Xaa^{T1})y1- is or comprises an amino acid having the structure of formula A-I wherein R^{a1} and R^{a2} are taken together with their intervening atoms to form an optionally substituted, saturated or partially unsaturated, 3-6 membered, monocyclic or bicyclic ring having, in addition to the intervening atoms, 0-1 heteroatoms selected from oxygen, nitrogen, and sulfur: or
-(Xaa^{T1})y1- is or comprises a L-proline.

5–9. (canceled)

10. The agent of claim 3 wherein L^{a} is —CH₂—CH₂—CH₂—, or —CH₂—O—CH₂—, or —CH₂—S—CH₂—, R′ is optionally substituted phenyl, or a combination thereof.

11–17. (canceled)

18. The agent of claim 1, wherein
-(Xaa^{T6})y6- is or comprises a dipeptide or an amino acid that is suitable for forming a turn;
-(Xaa^{T6})y6- is or comprises L-proline, D-proline, a proline derivative, L-serine, D-serine, glycine, L-pseudoproline, or D-psuedoproline:
-(Xaa^{T6})y6- is or comprises an amino acid that comprises or is further substituted with —L^{a}—COOH; or
a combination thereof.

19–30. (canceled)

31. The agent of claim 1, wherein
-(Xaa^{T1})y1- is or comprises 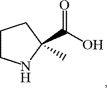 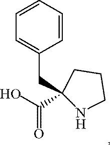 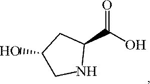 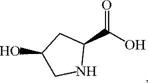 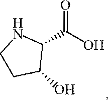 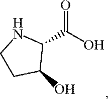 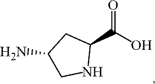 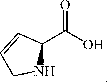 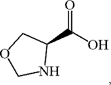 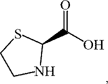 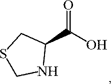 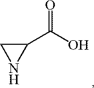 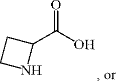 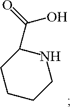
-Xaa^{T2}- is 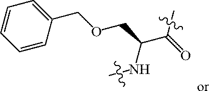 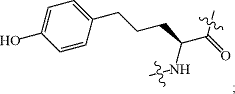
-(Xaa^{T2})y2- is or comprises AVAD; -(Xaa^{T3})y3- is or comprises TF, TFLL, or TFLLKY; XAA^{T4} is Cys;
-(Xaa^{T6})y6- is or comprises a D-Ser,
-(Xaa^{T6})y6- is or comprises has the structure 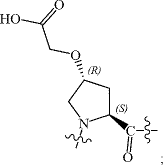
Xaa^{T7} is D or E:
-(Xaa^{T8})y8- is or comprises GTI-Xaa^{T8}-;
Xaa^{T4} and Xaa^{T9} are independently Cys and form a disulfide bond —S—S—;
-(Xaa^{T10})y10- is or comprises AV; or
a combination thereof.

32–33. (canceled)

34. The agent of claim 1, wherein.
-(Xaa)y- is or comprises IEEQAKTFLDKFNHEAEDLFYQS;
-(Xaa)y- is or comprises DISGINASVVNIQKEIDRLNEVAKNLNESLIDLQEL, or a fragment thereof;
-(Xaa)y- is or comprises a sequence selected from, or a sequence designed based on a sequence selected from: Number Sequence

| | |
|---|---|
| P1 | FKLPLGIN(K)ITNFRAILTAFS(L)\| |
| P2 | PTT(K)FMLKYDENGTITDAVDC |
| P3 | VLYNSTP(S)FSTFKCYGVSATK |
| P4 | PALNCYWPLN(K)DYGFYTTSGI |
| P5 | RDVSDP(I)TDSVRDPKTSEILD |
| P6 | YQDVNCTDVS(P)TAIHADQLTP |
| P7 | SNNTIAIPTNFS(L)ISITTEVM |
| P8 | OYGSFCT(A)QLNRALSGIAA(V)EQ |
| P9 | GIGVT(A)QNVLYENQKQIANQF |
| P10 | IQK(E)EIDRLNEVAKNLNESLI |

-(Xaa)y- is or comprises 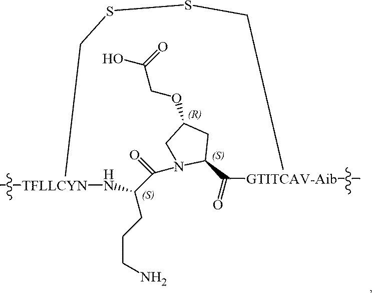 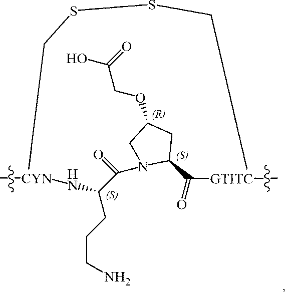 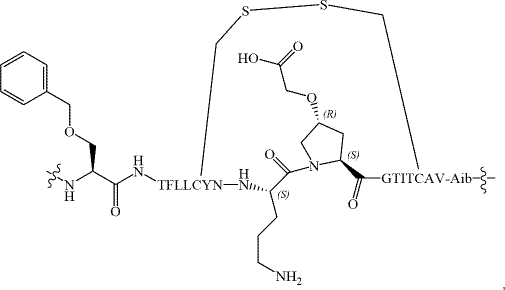 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCNC(=O)CCOCCOCCOCCOCCNC(=O)CCCC[C@H]2SC[C@H]3NC(=O)N[C@H]32)NC(=O)[C@H](CC(N)=O)NC(=O)[C@H](Cc2ccc(O)cc2)NC(=O)[C@@H](NC(=O)[C@H](CC(C)C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O] 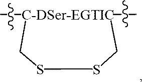 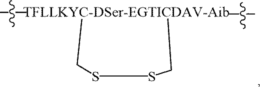 [SMILES: CC[C@H](C)[C@@H]1NC(=O)[C@H]([C@@H](C)O)NC(=O)CNC(=O)[C@@H]2C[C@@H](OCC(=O)O)CN2C(=O)[C@H](CCCCN)NC(=O)[C@H](CC(N)=O)NC(=O)[C@H](Cc2ccc(O)cc2)NC(=O)[C@@H](NC(=O)[C@H](CC(C)C)NC(=O)[C@H](CC(C)C)NC(=O)[C@H](Cc2ccccc2)NC(=O)[C@@H](NC(=O)[C@H](COCc2ccccc2)NC(C)=O)[C@@H](C)O)CSSC[C@@H](C(=O)N[C@@H](C)C(=O)N[C@H](C(=O)N[C@@H](C)C(=O)NC(C)(C)C(N)=O)C(C)C)NC(=O)[C@H]([C@@H](C)O)NC1=O] 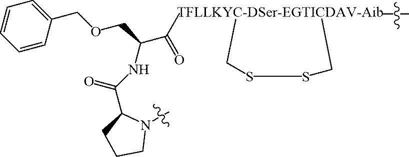 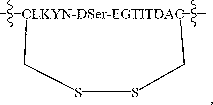 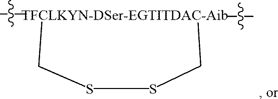 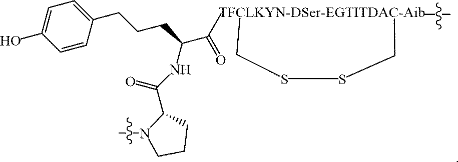
.

35–36. (canceled)

37. The agent of claim 1, wherein -(Xaa)y- is or comprises.

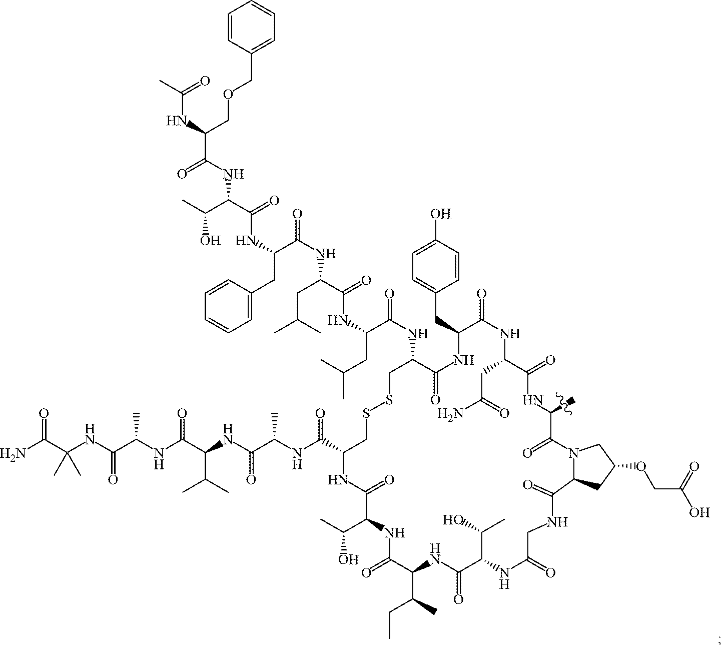

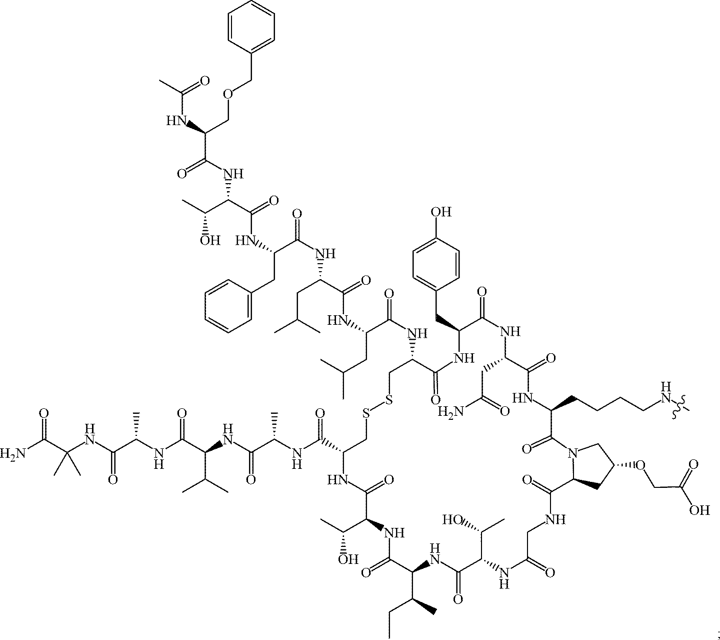

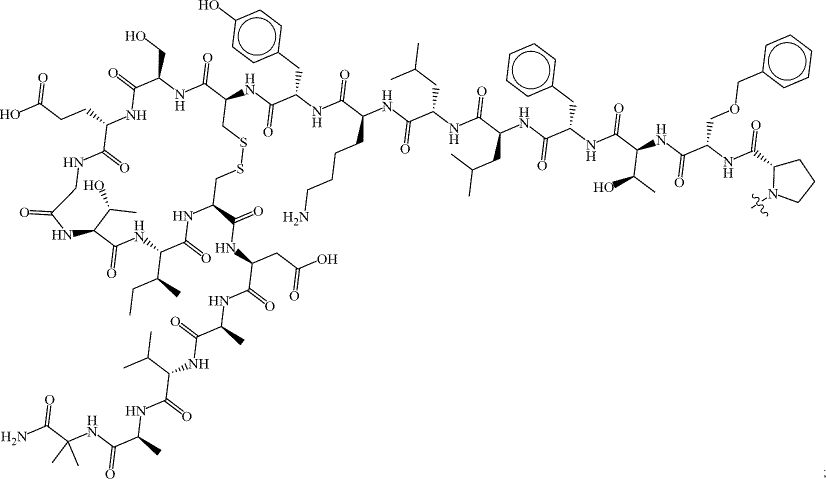

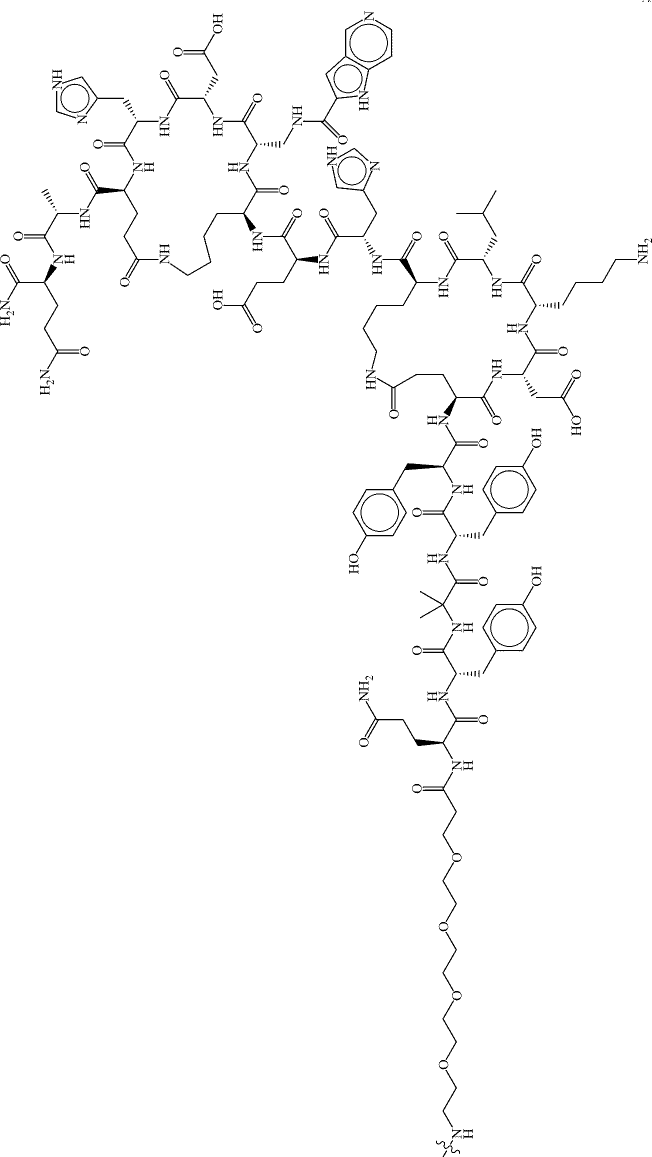

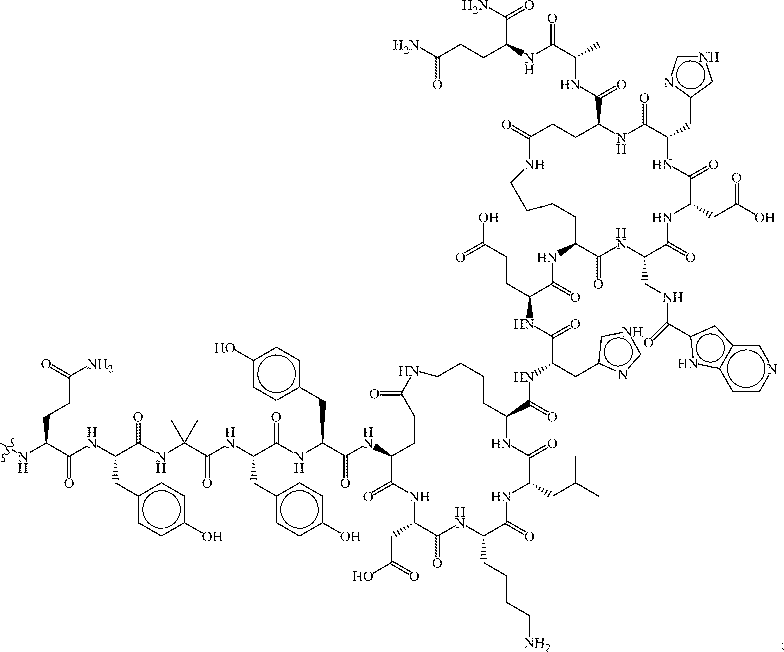

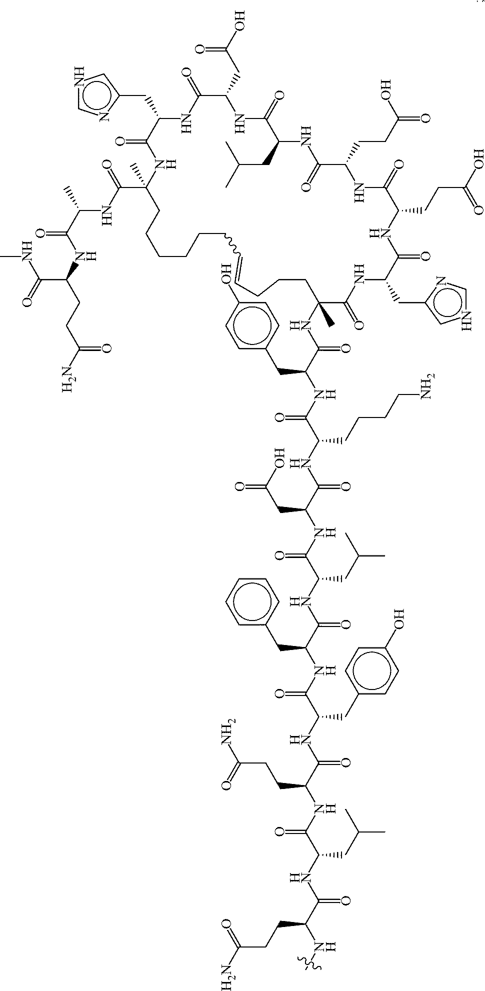

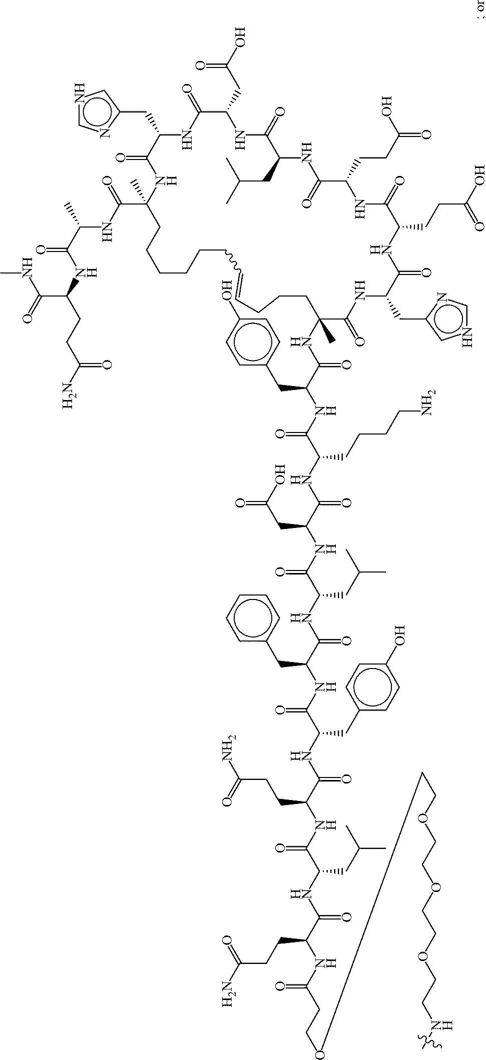

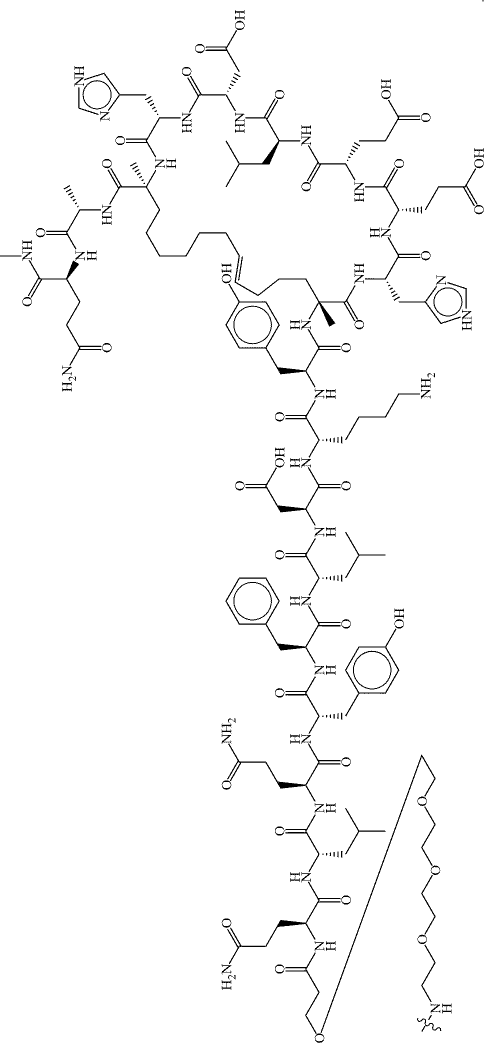

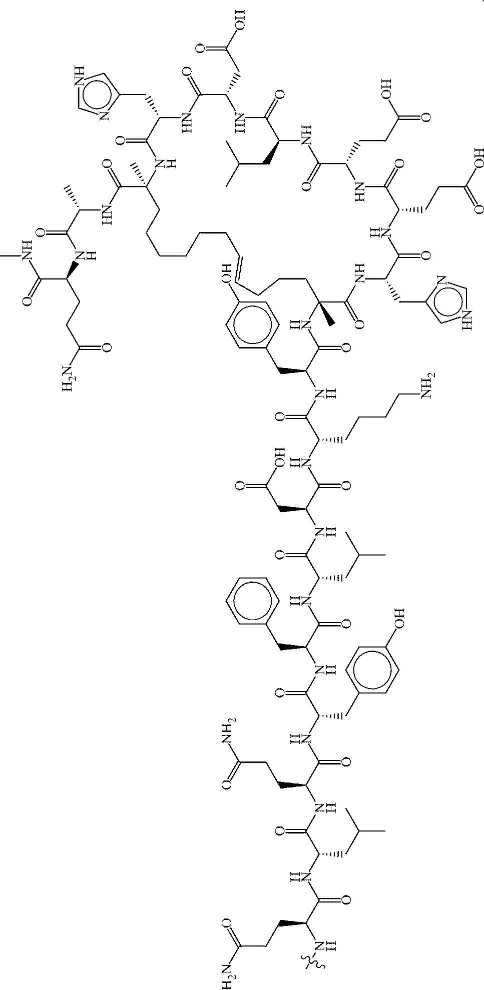

38–39. (canceled)

40. The agent of claim 1, wherein the linker moiety comprises one or more —[(CH₂)₂—O]m— groups, wherein m is an integer from 7 to 12.

41–42. (canceled)

43. The agent of claim 1, wherein the agent comprises a single A that is an antibody binding moiety.

44. (canceled)

45. The agent of claim 1, wherein the antibody binding moiety comprises or has the structure selected from A-1 to A-50 or
the antibody binding moiety comprises or has the amino acid sequence DCAWHLGELVWCT, wherein the two cystein amino acids are linked by a —S—S—.

46. The agent of claim 1, wherein the agent has the structure 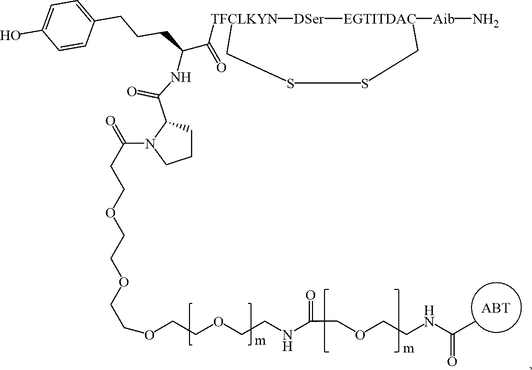
or a salt thereof; 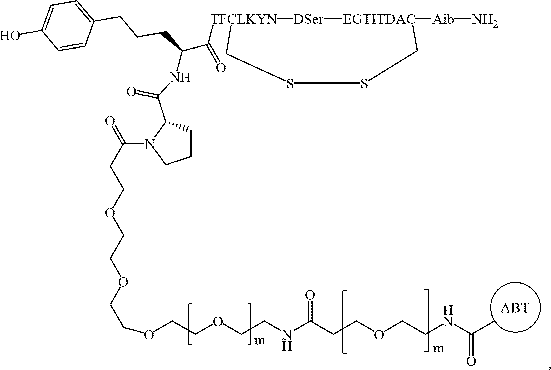
or a salt thereof; 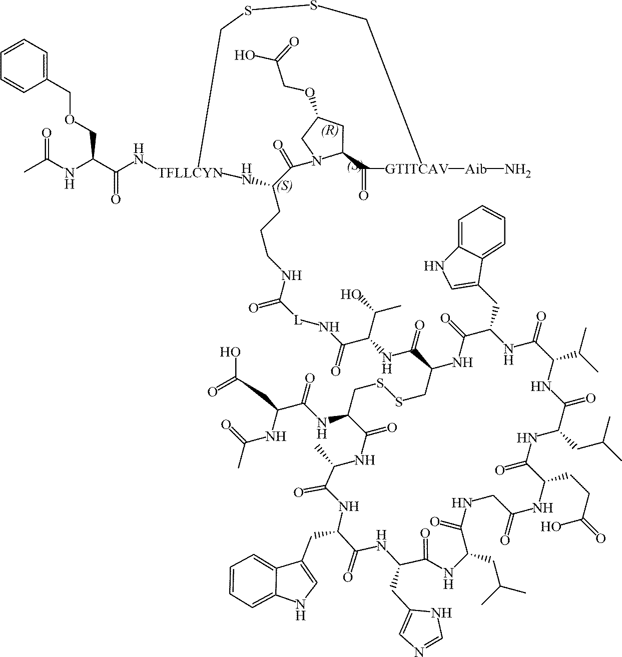
or a salt thereof; ;or 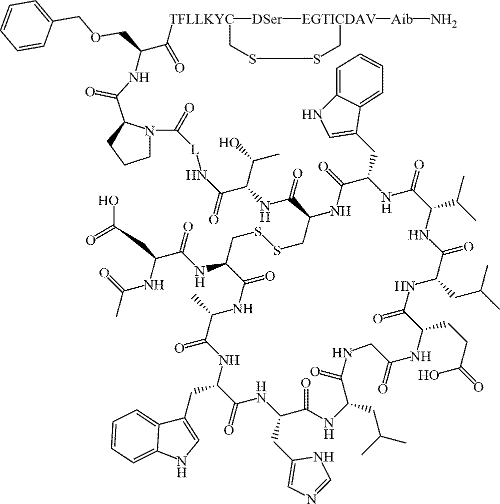
, or a salt thereof.

47. The agent of claim 1, wherein the agent has the structure of 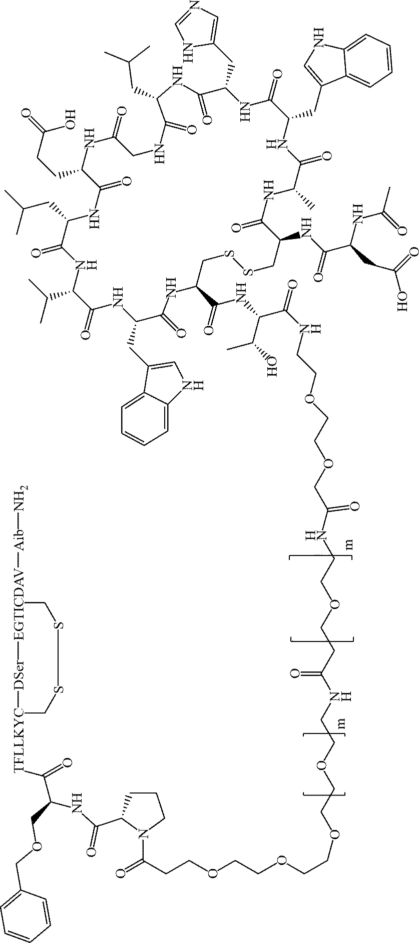 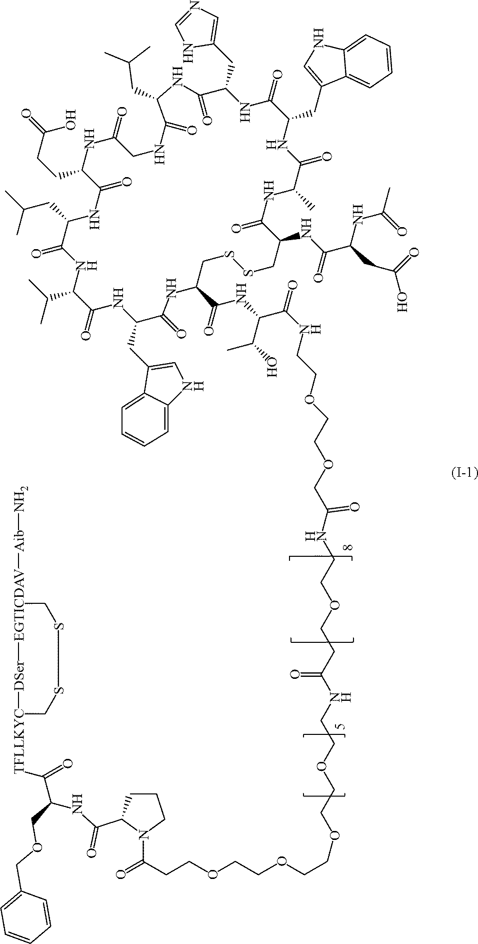
or a salt thereof; 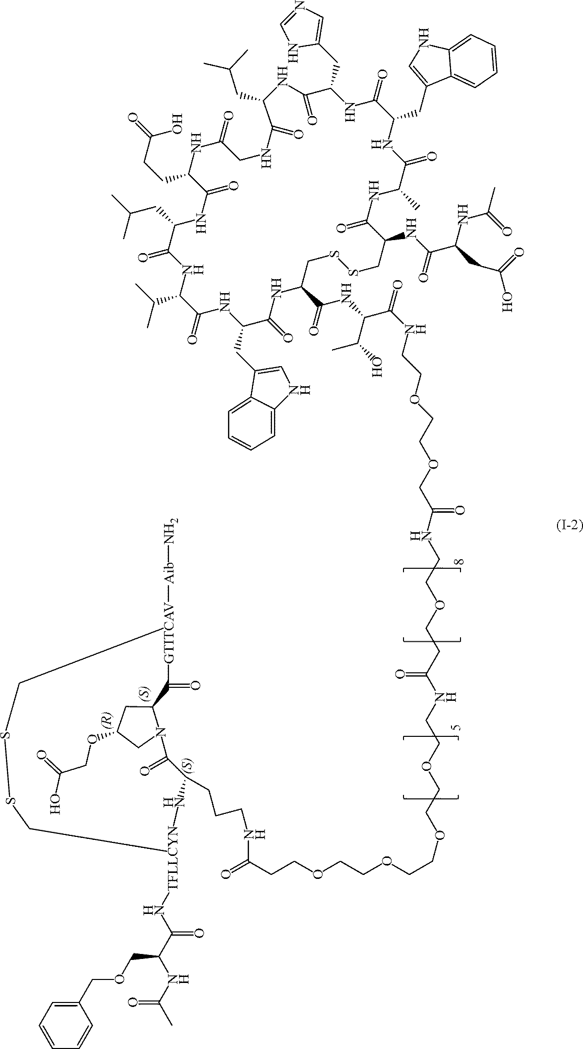 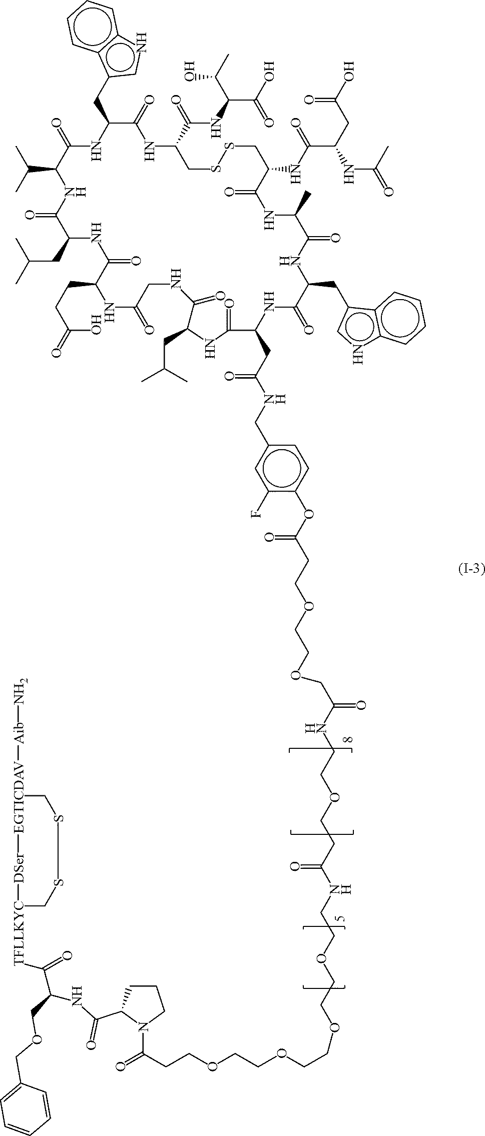 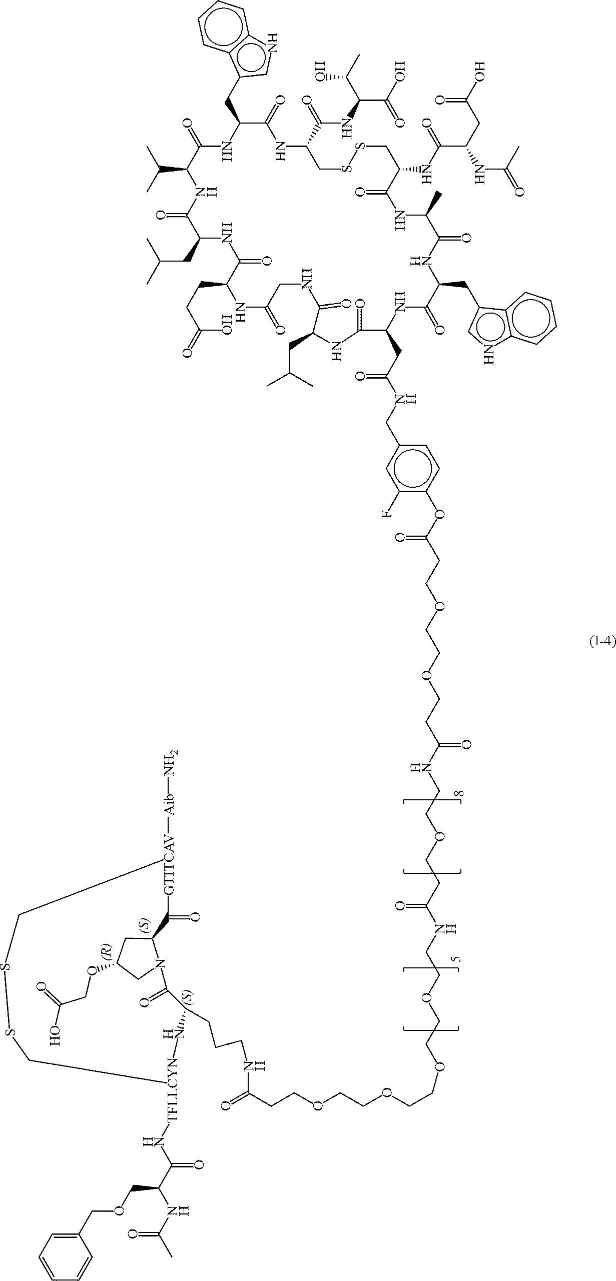 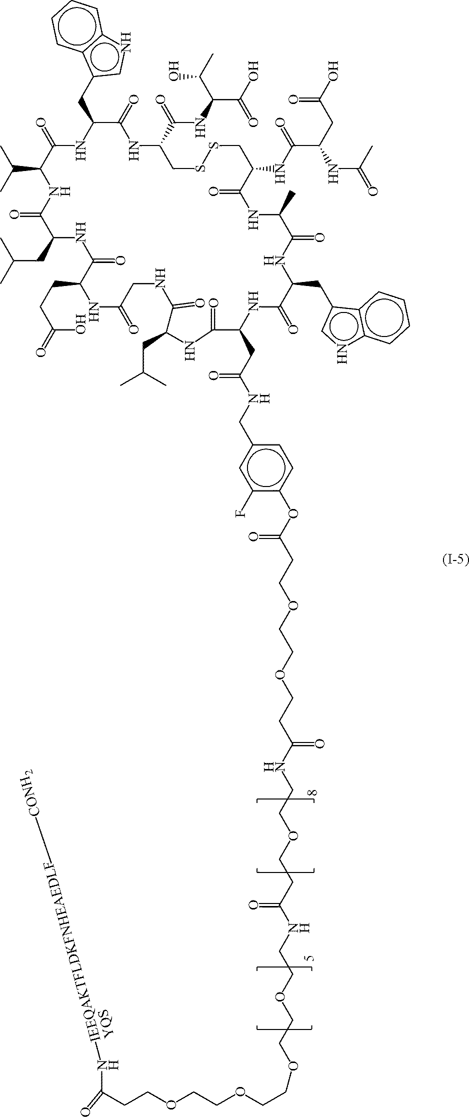
or a salt thereof; 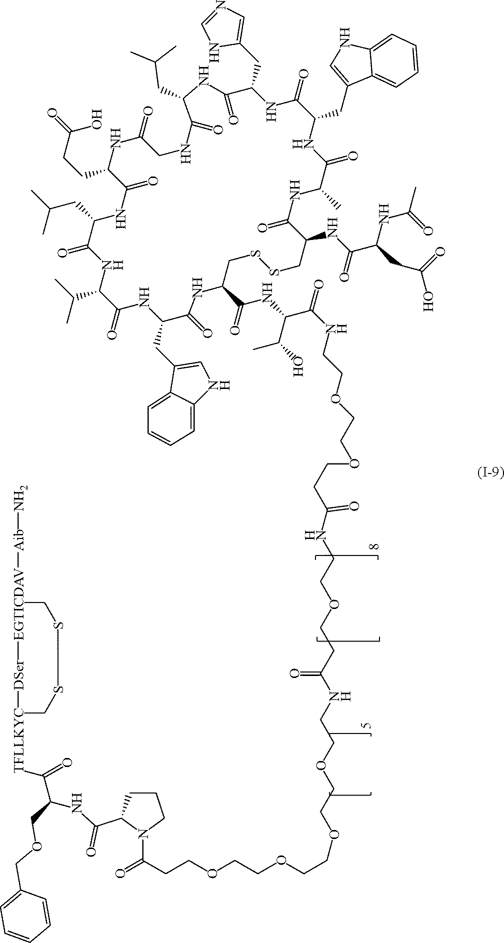 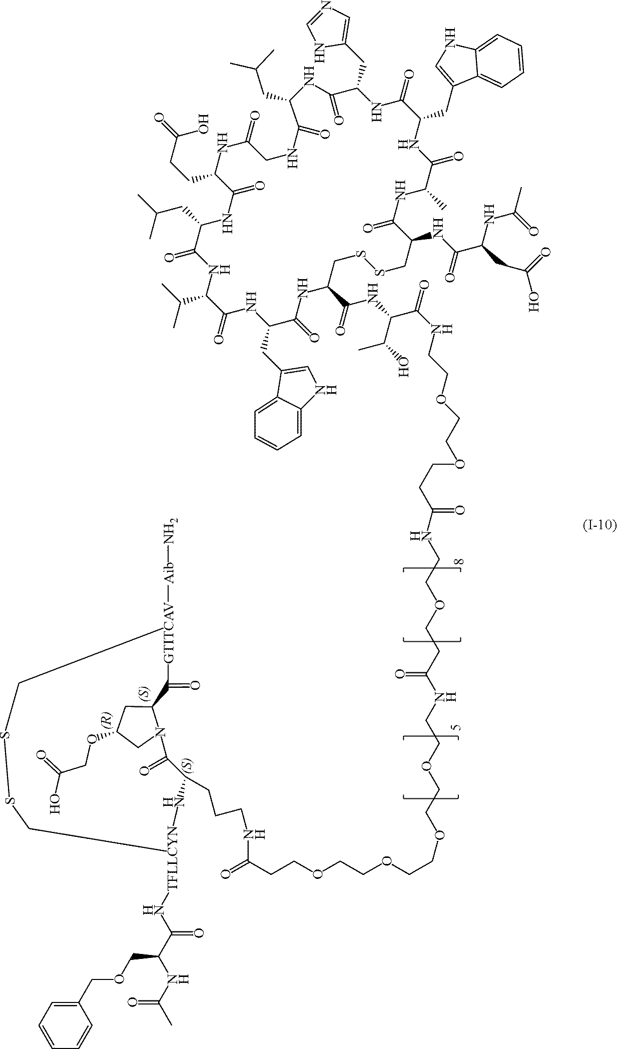 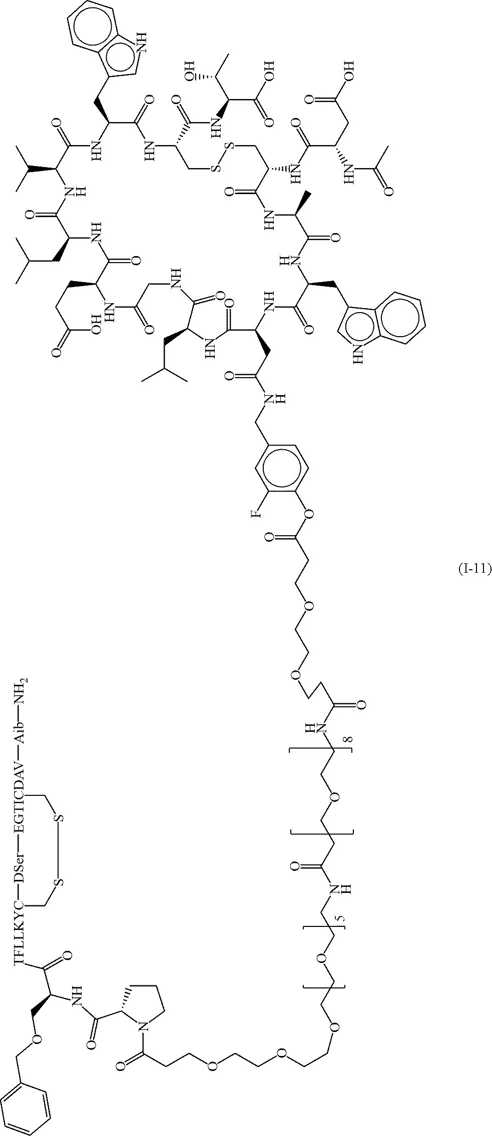
(I-11) or a salt thereof; 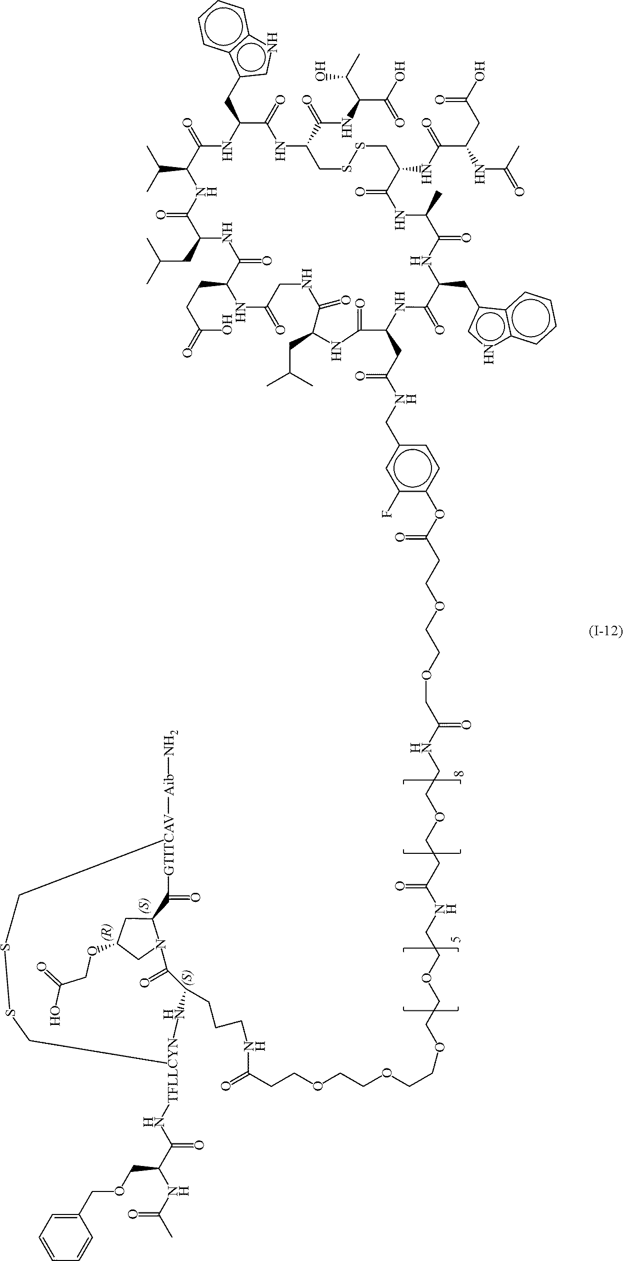 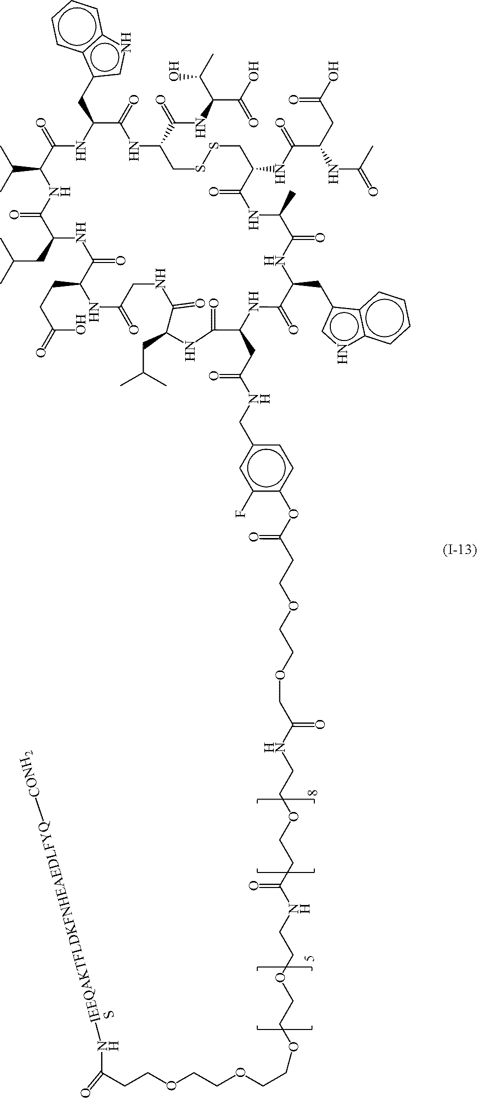
or a salt thereof; 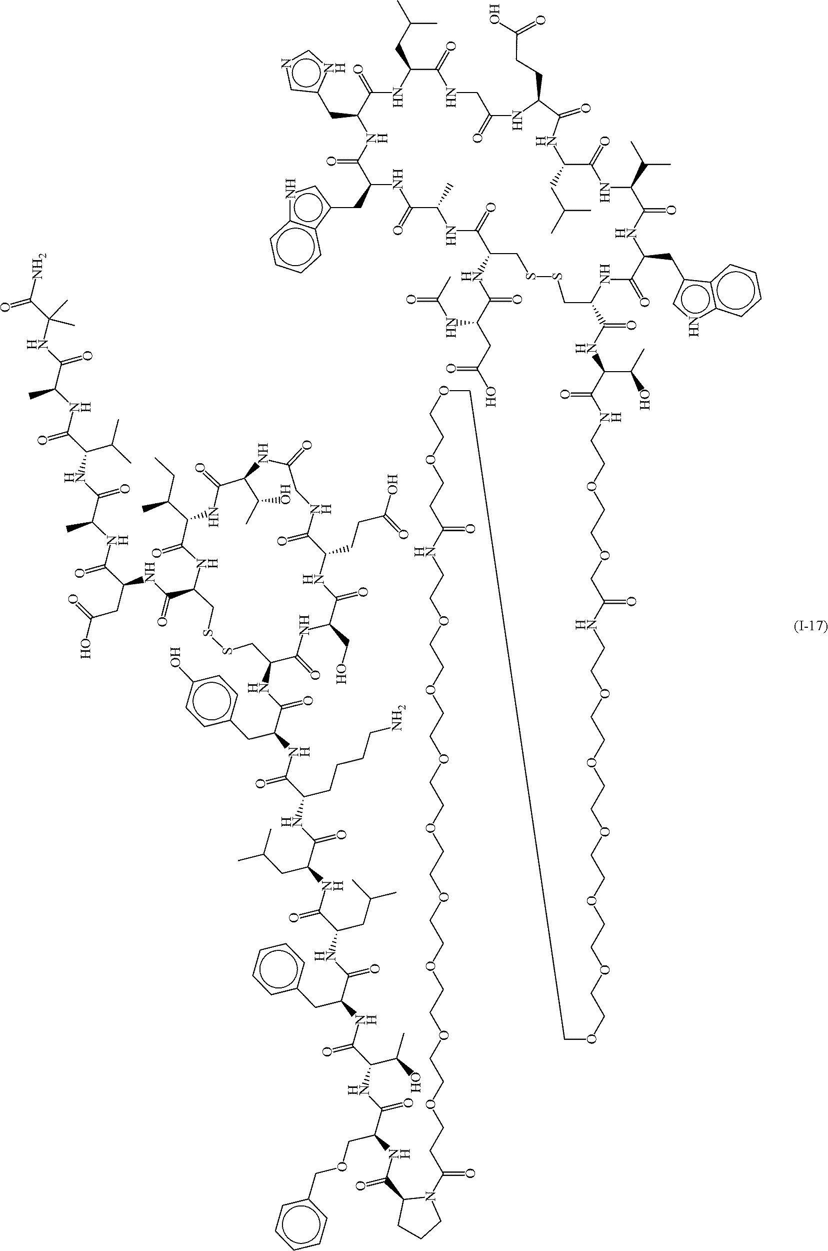 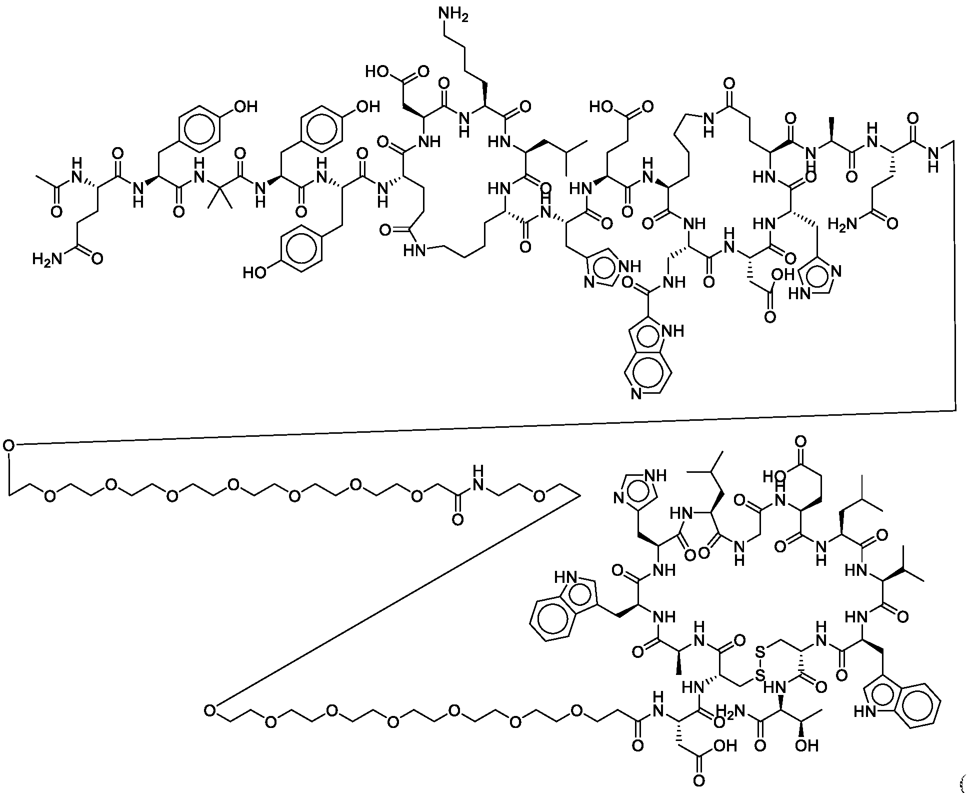 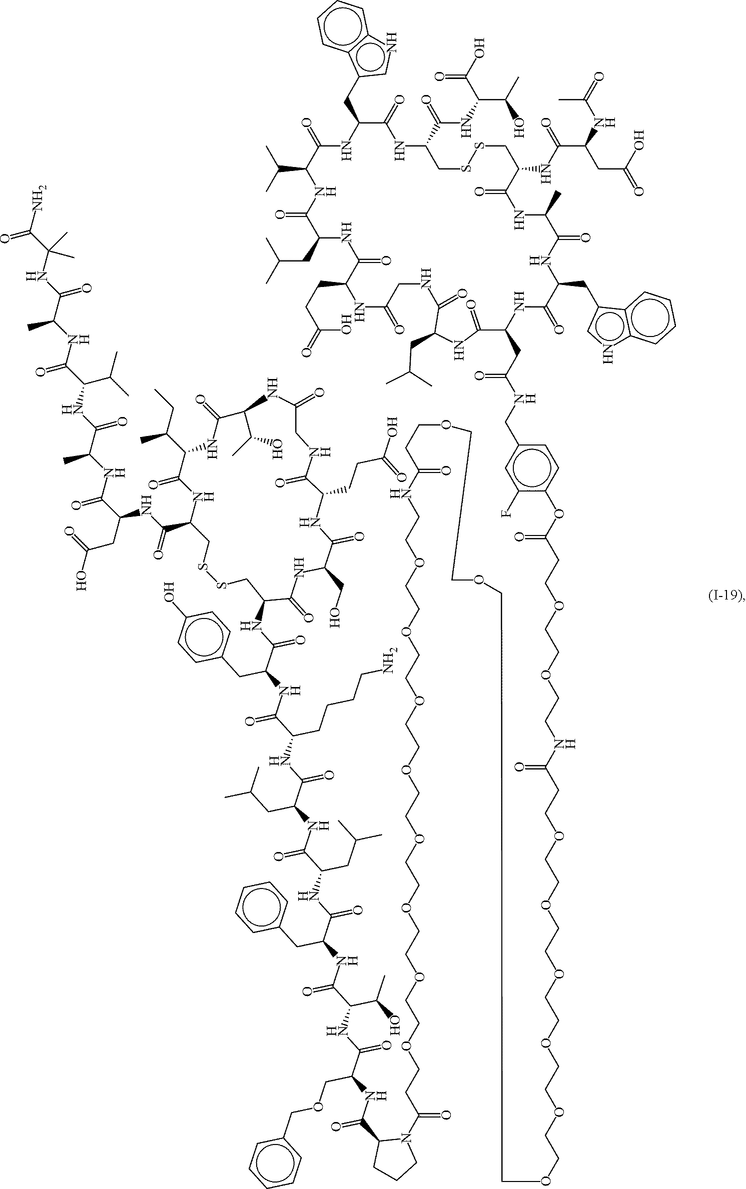
or a salt thereof; 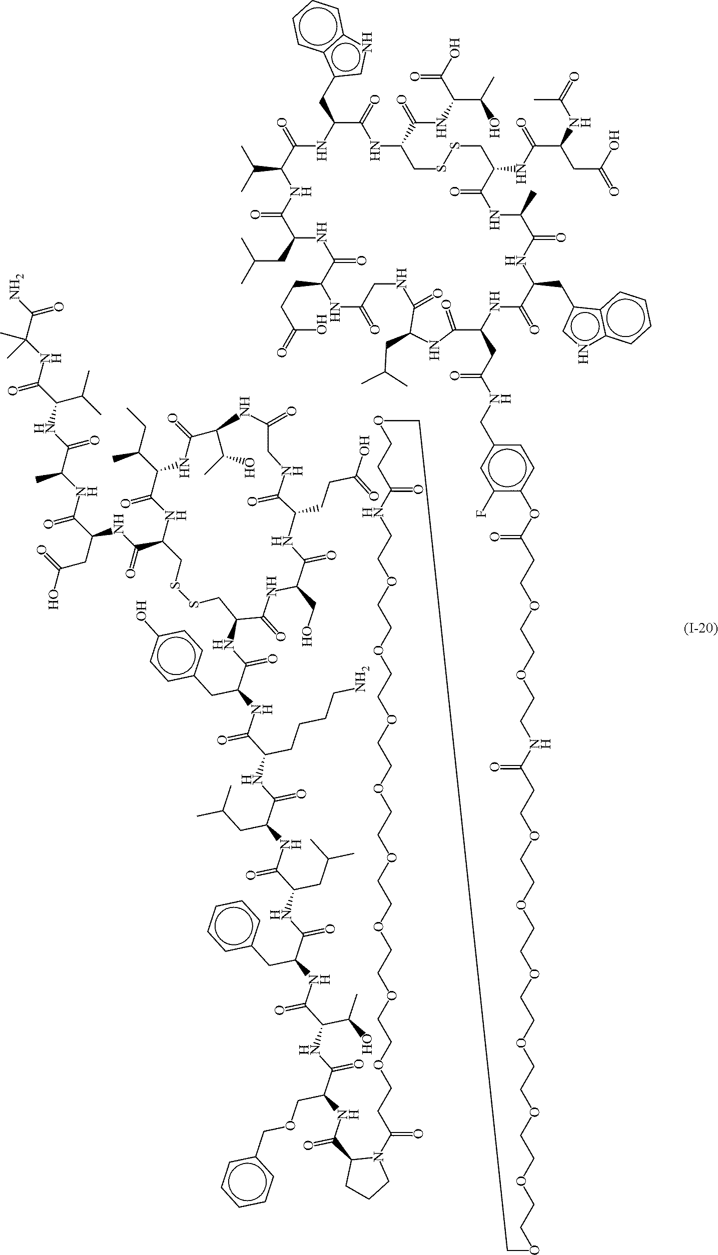
or a salt thereof; 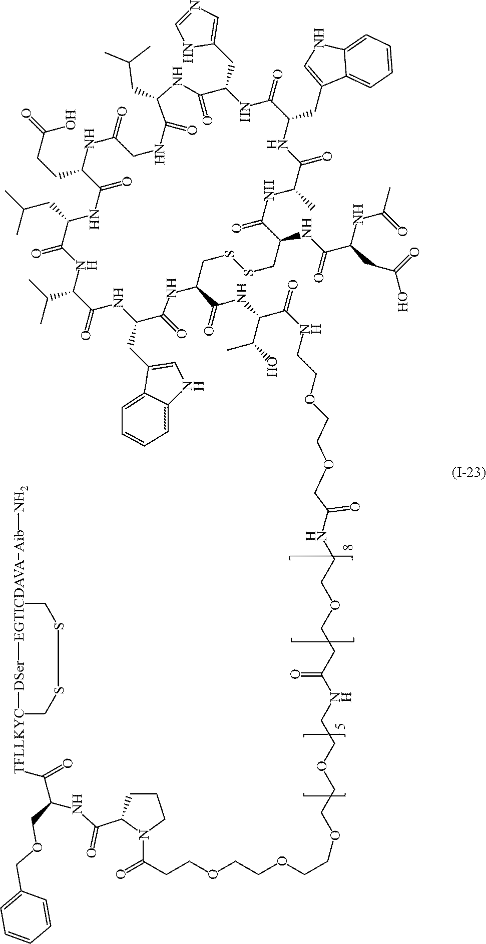 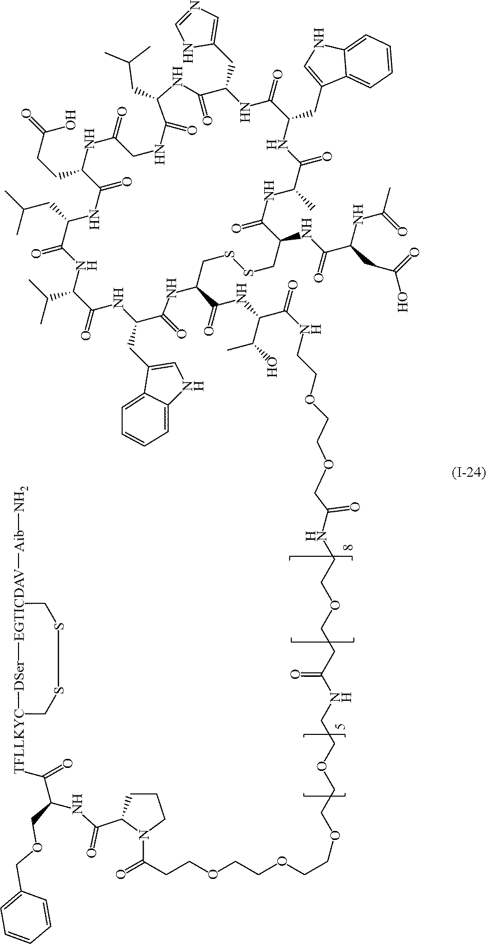 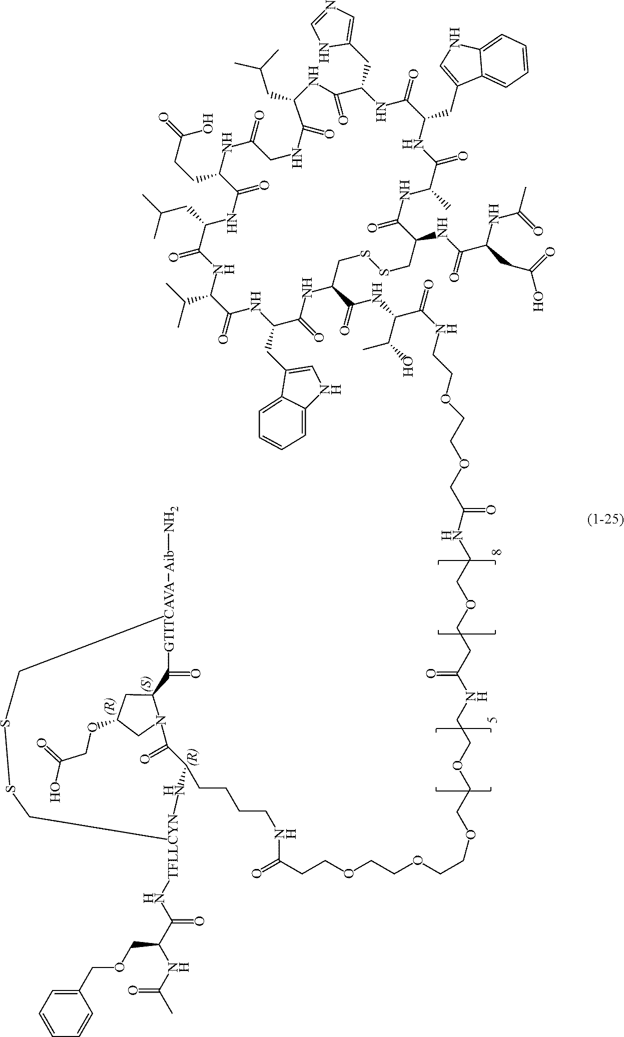 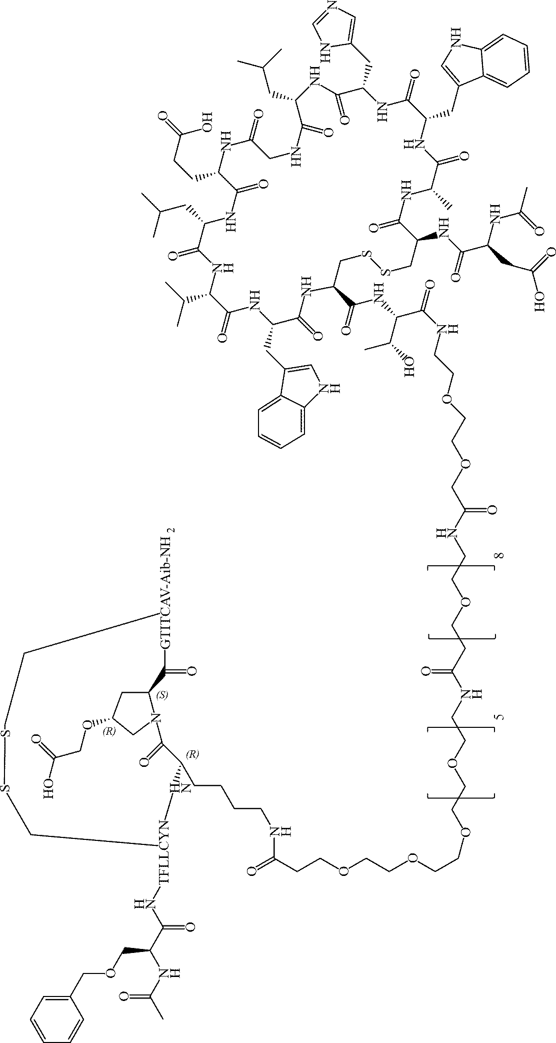
or a salt thereof; 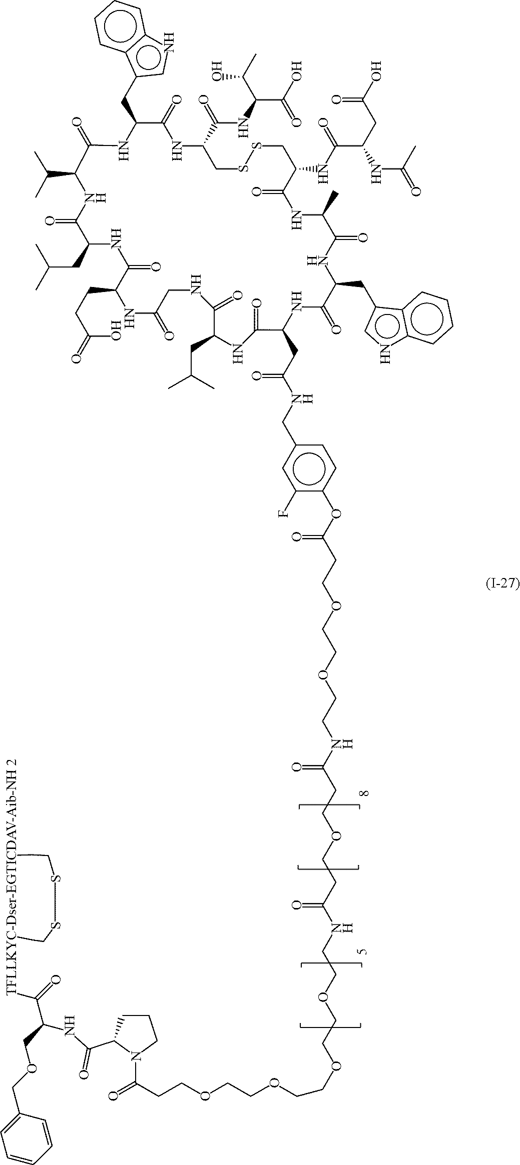 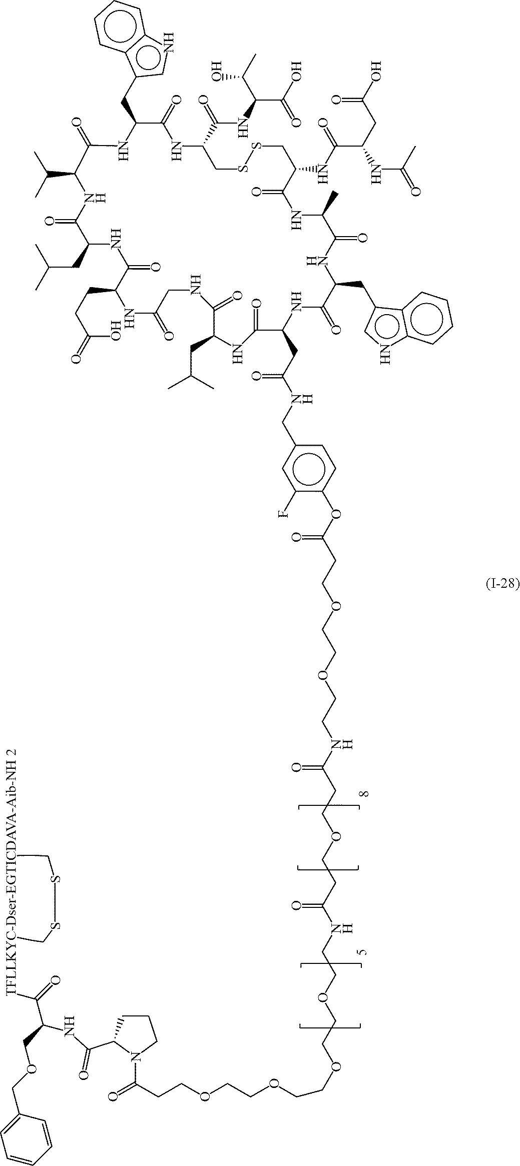
(I-11) or a salt thereof; 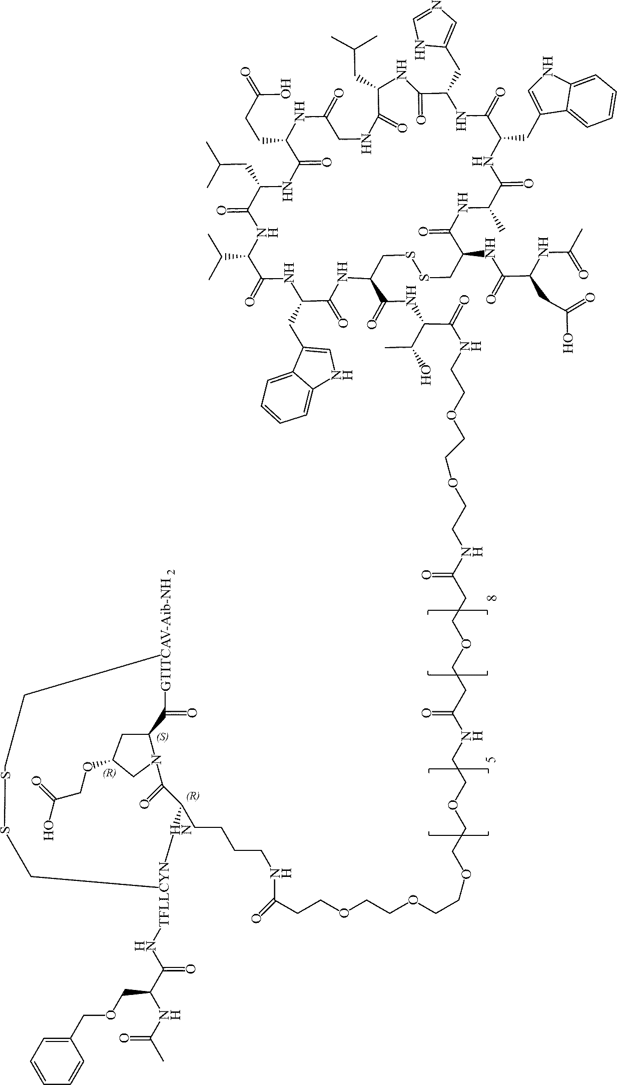
(I-29) or a salt thereof; 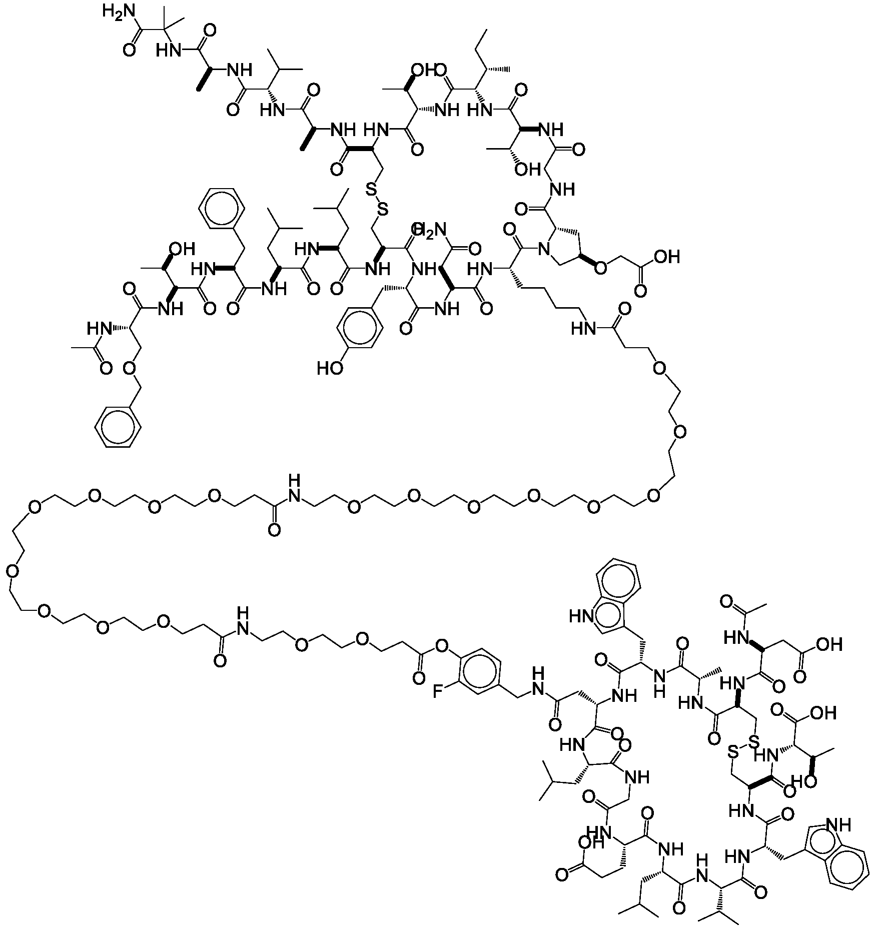 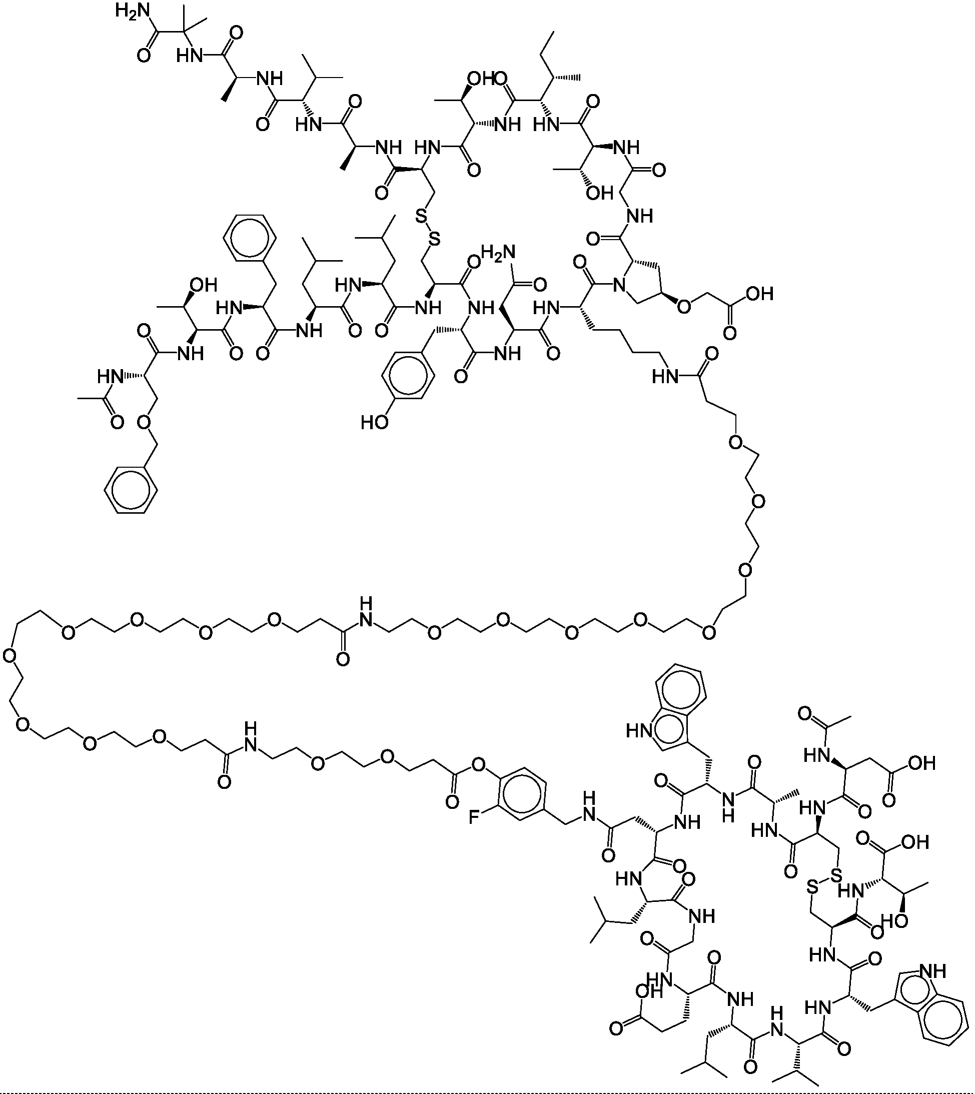
(I-31), or a salt thereof; 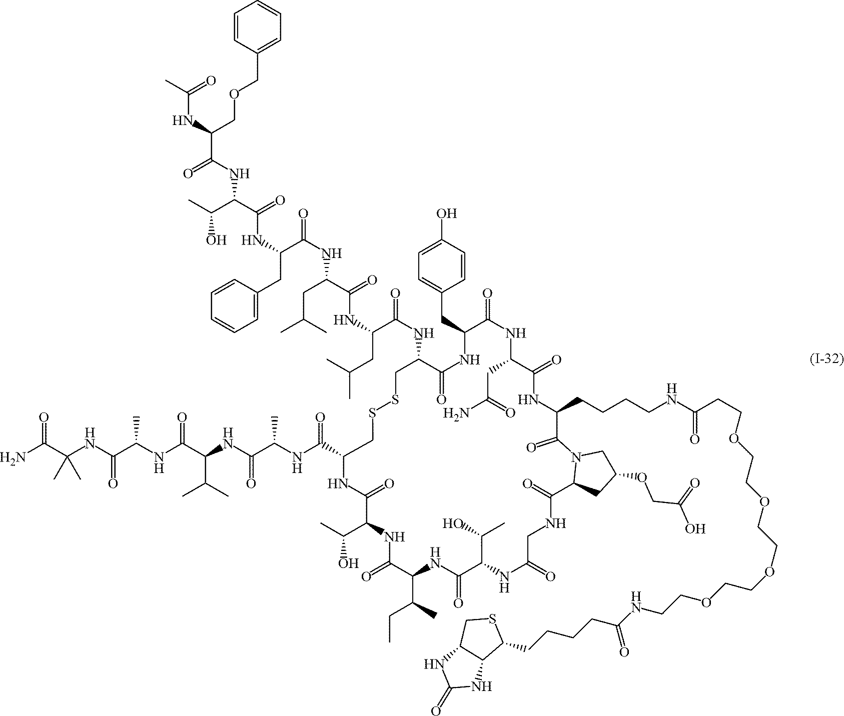
(I-32), or a salt thereof; 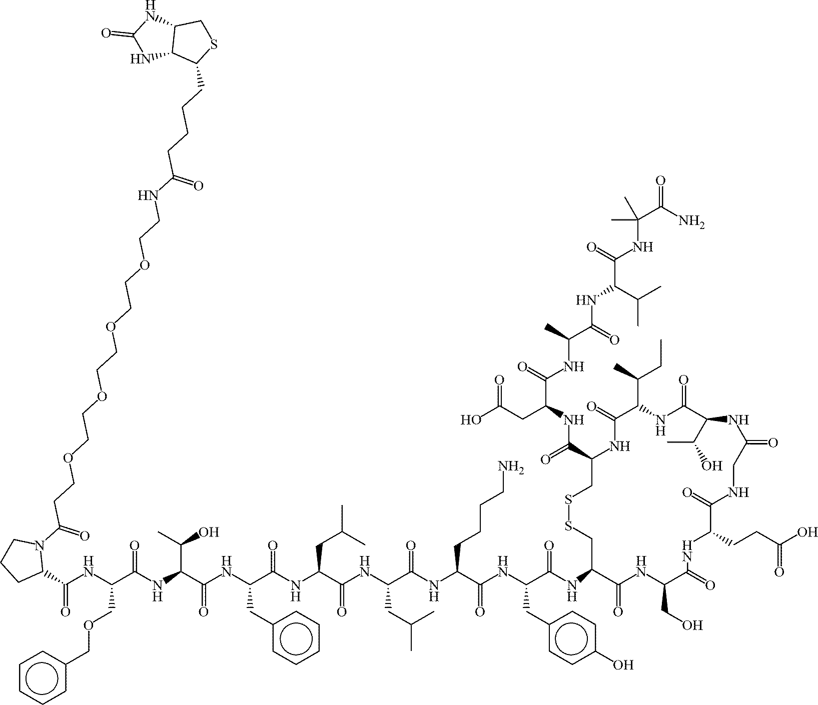
(I-33), or a salt thereof; or 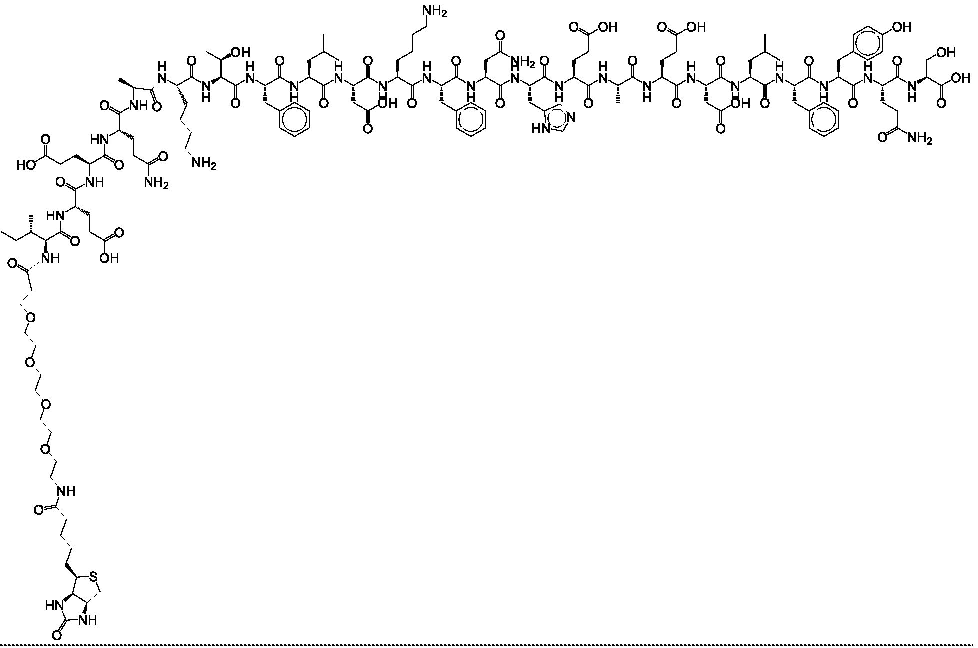
(I-34), or a salt thereof.

48-72. (canceled)

73. An agent having any of the following structures or a salt thereof.

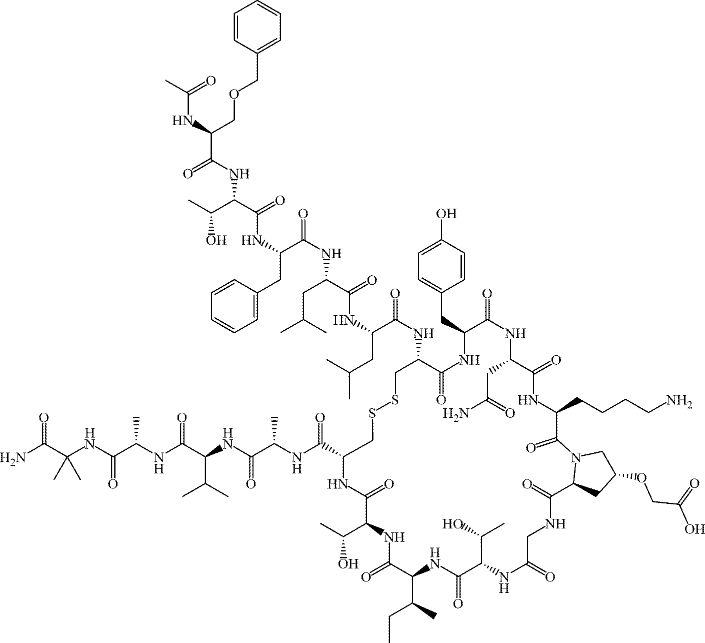

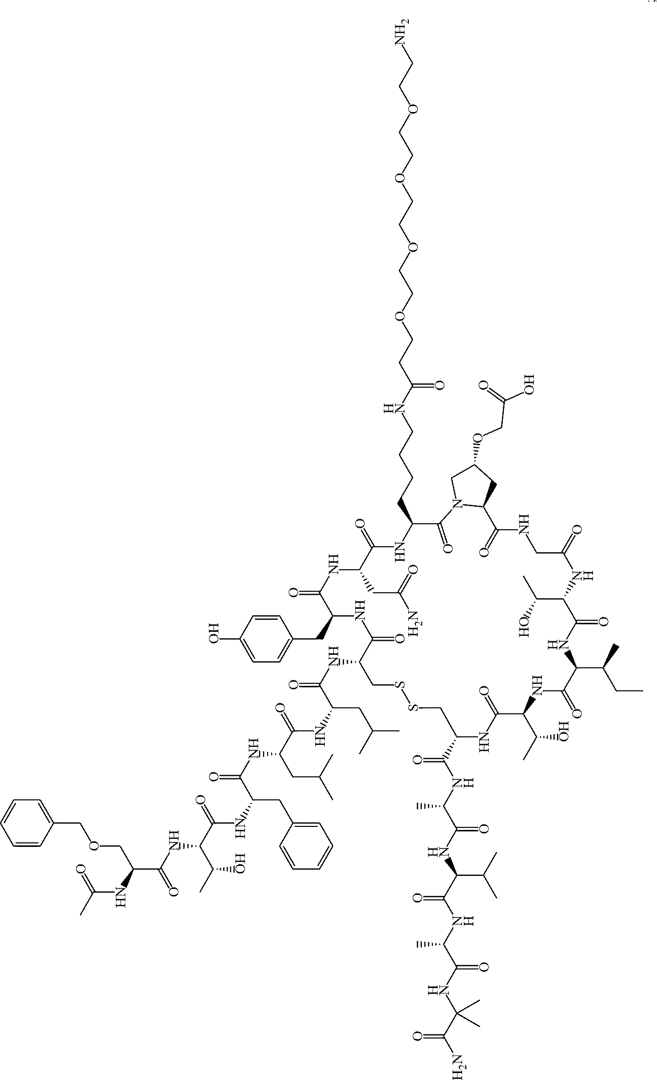

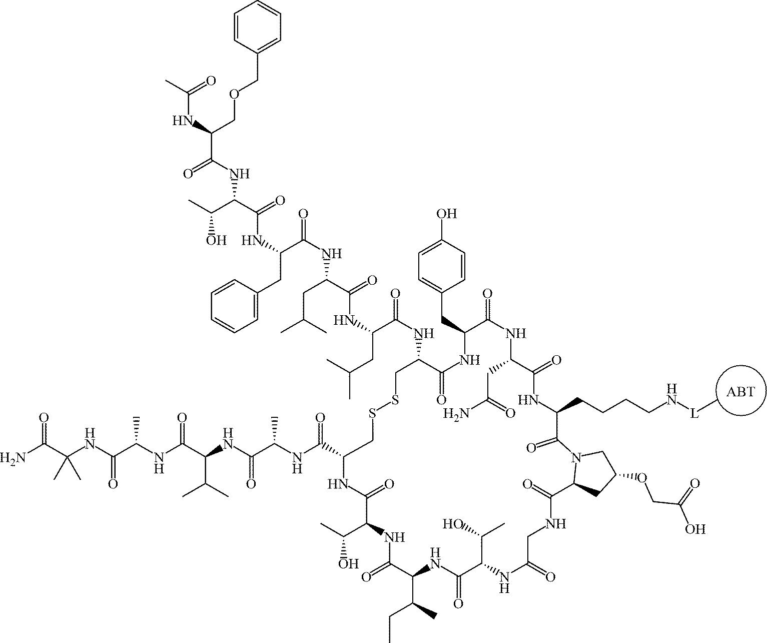

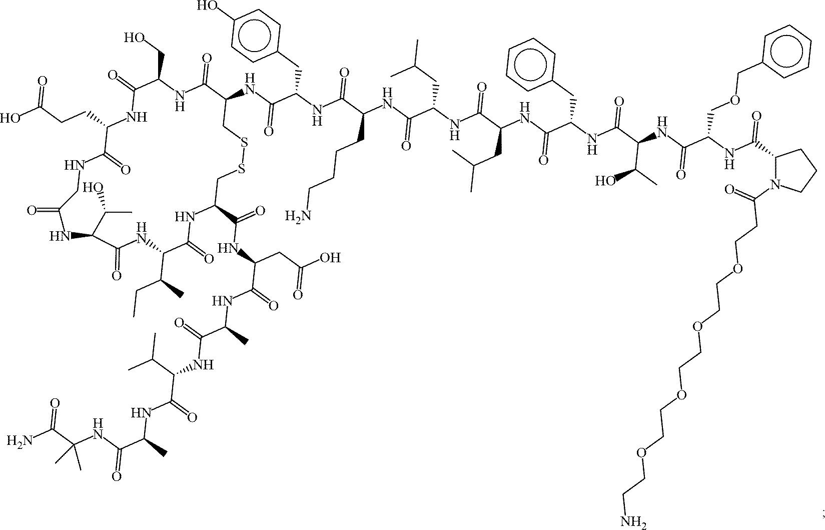

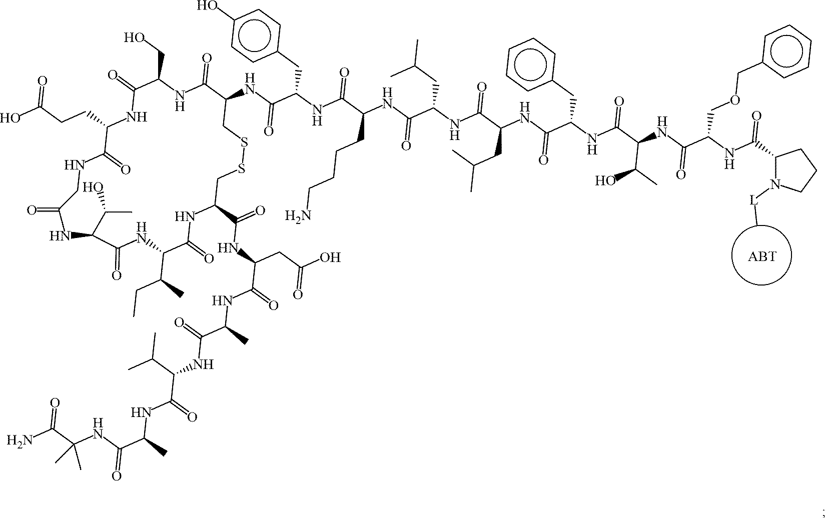

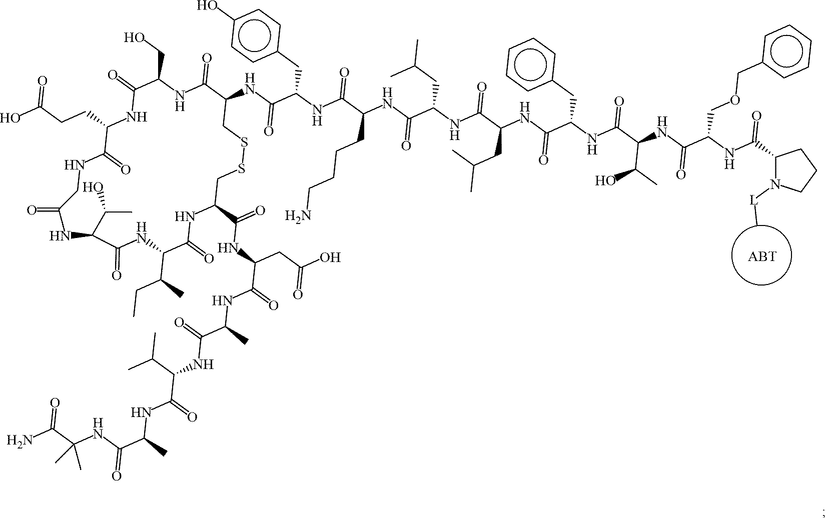

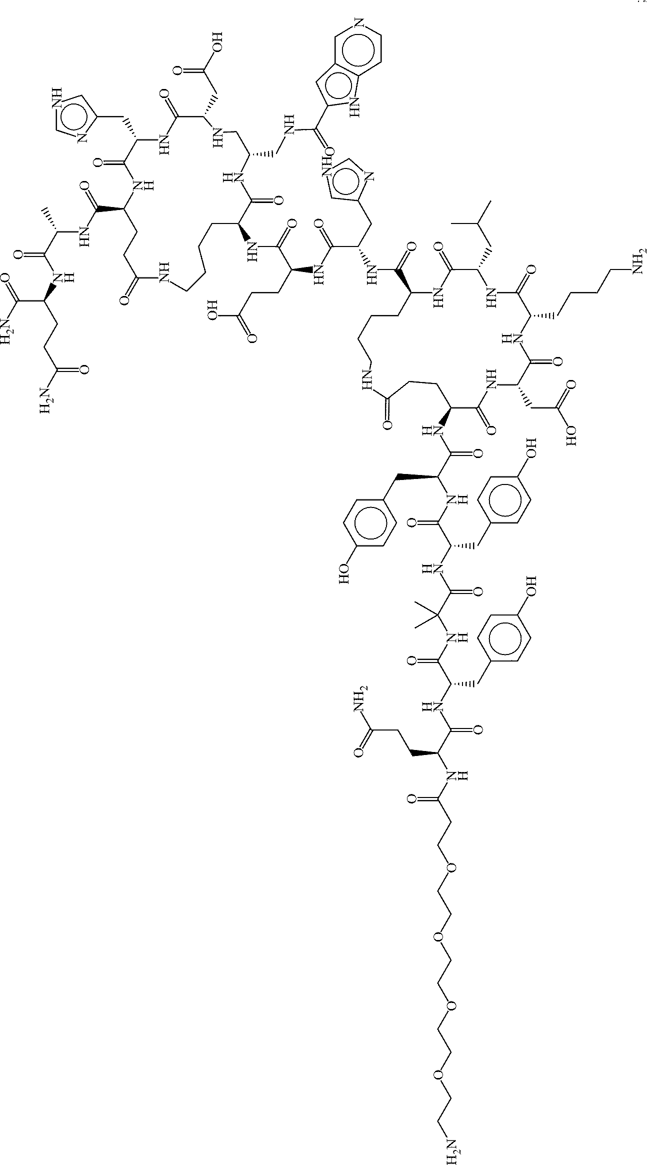

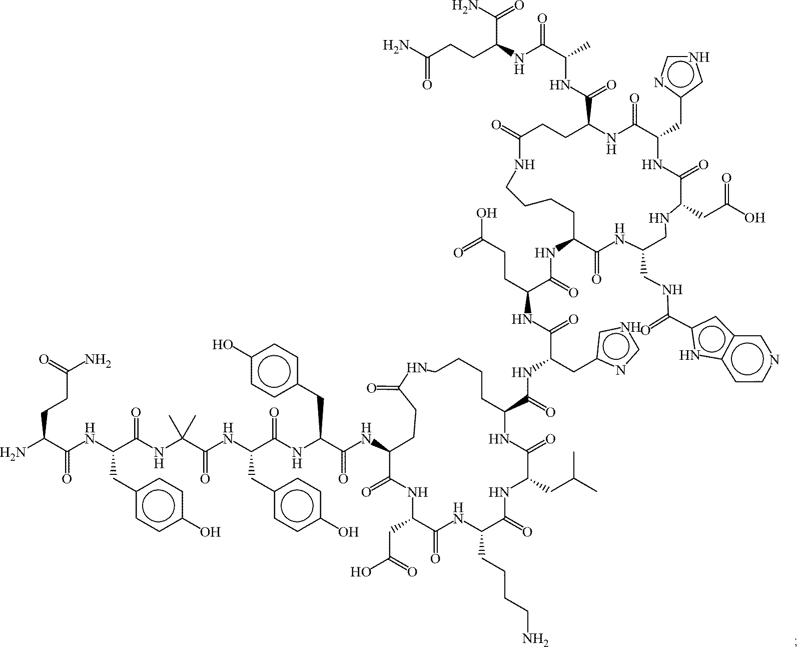

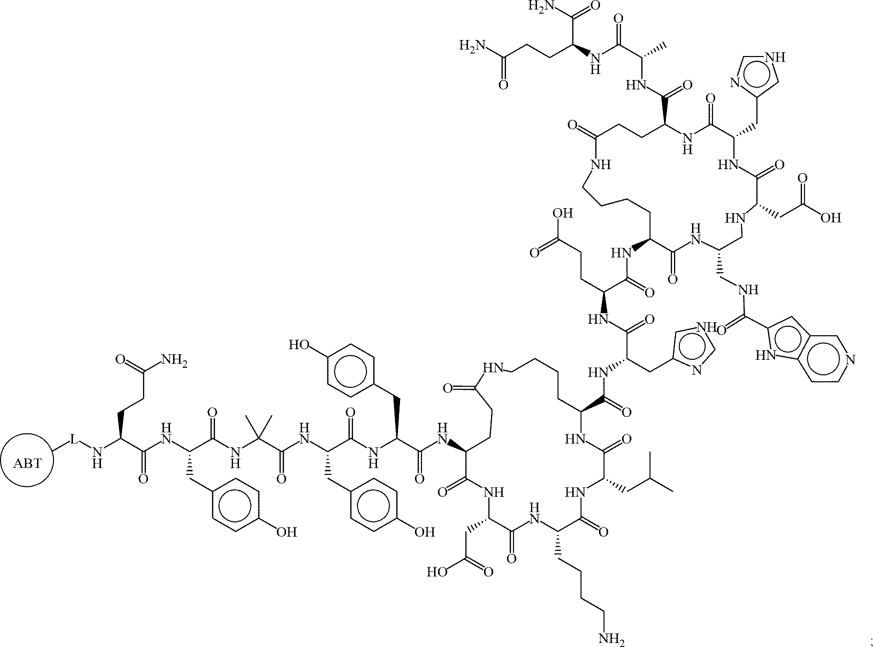

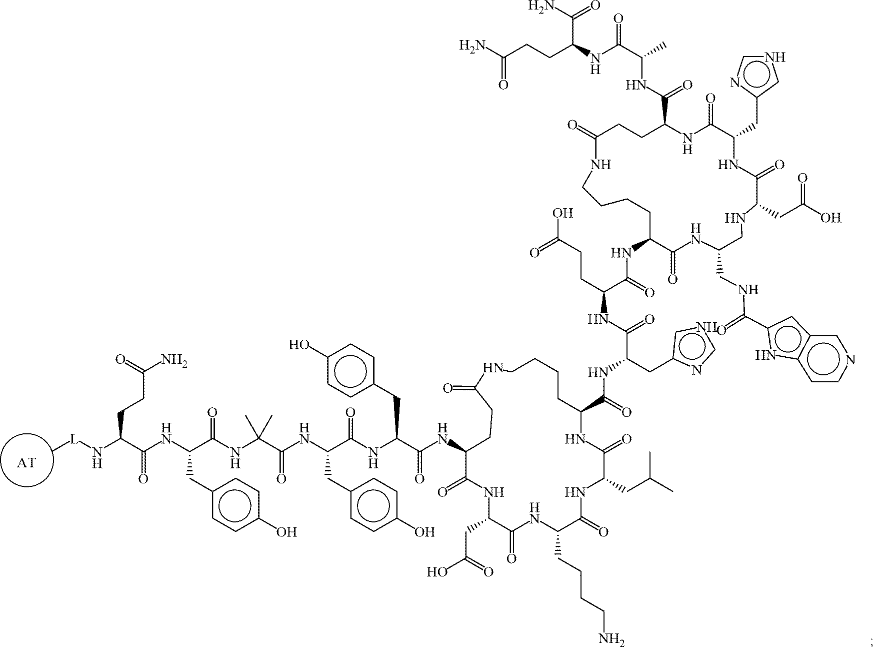

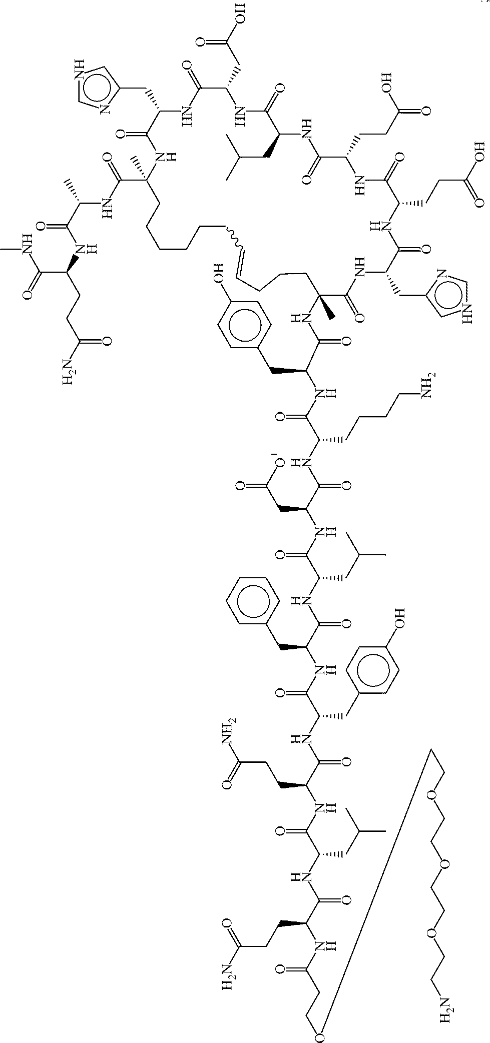

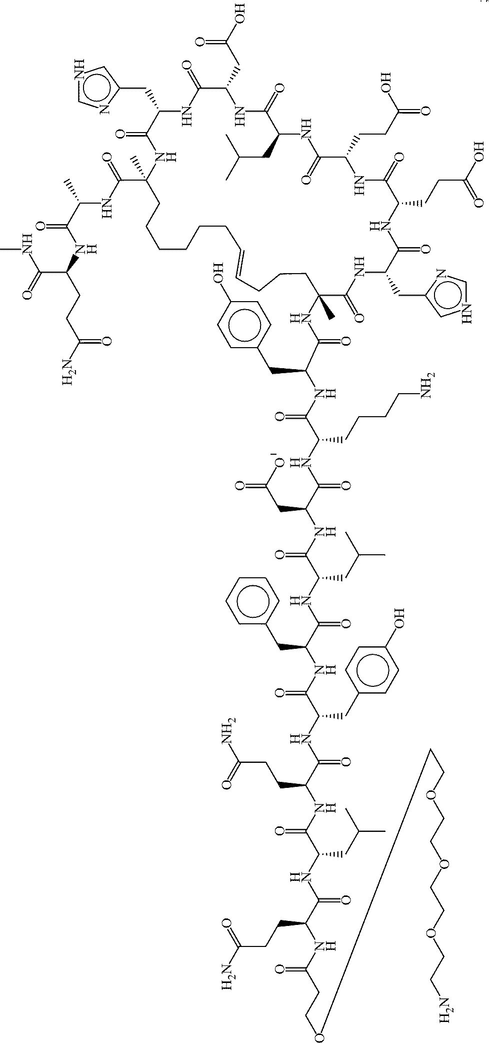

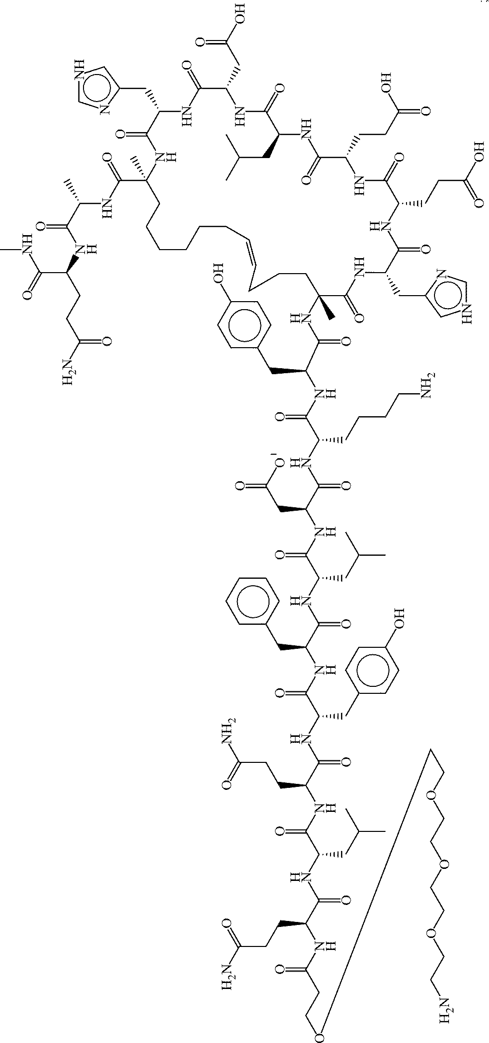

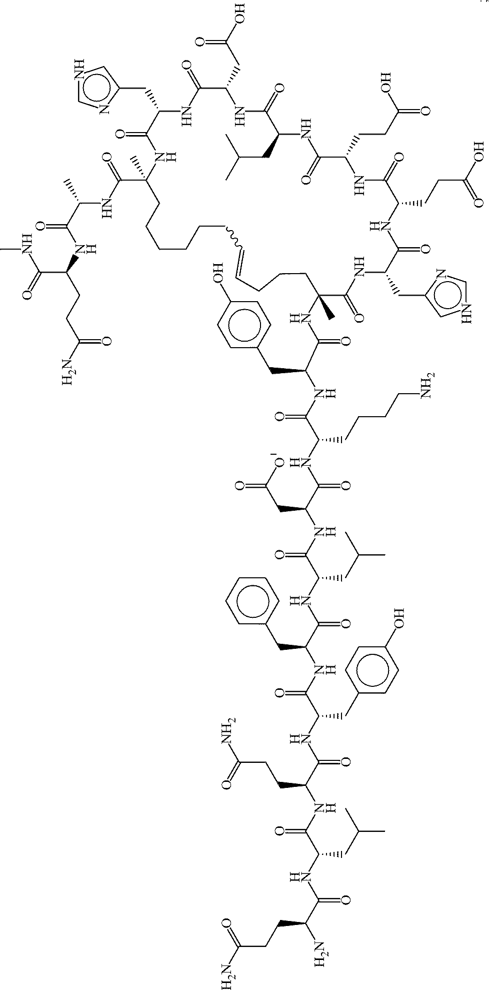

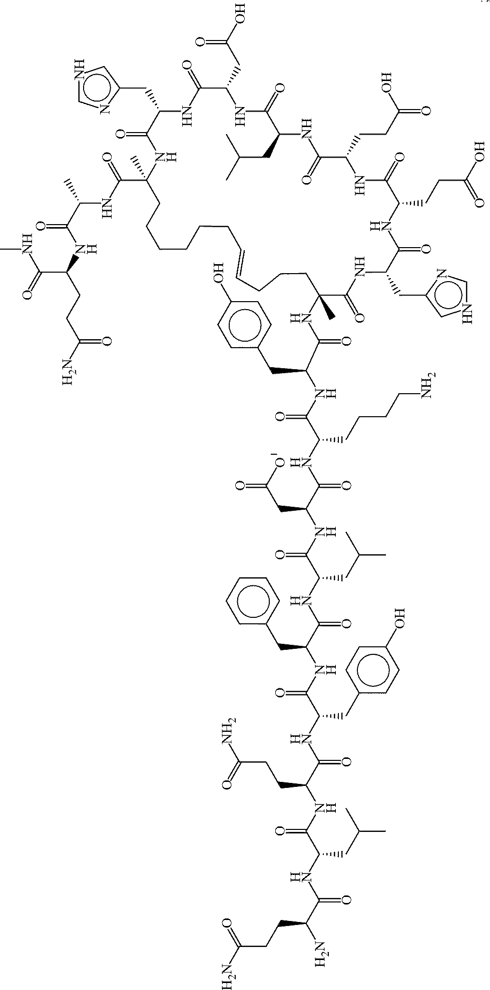

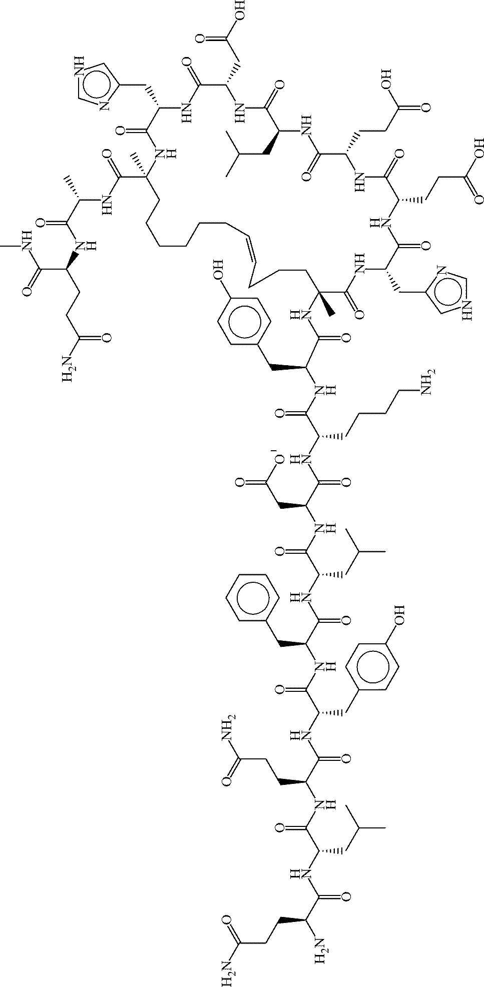

74–75. (canceled)

76. A pharmaceutical composition, comprising an agent of claim 1, or a pharmaceutically acceptable salt thereof, and a pharmaceutically acceptable carrier.

77. (canceled)

78. A method for treating a condition, disorder or disease associated with SARS-CoV-2 infection, comprising administering to a subject suffering therefrom an effective amount of an agent or a composition of claim 1.

79. A method for inducing, promoting, encouraging, enhancing, triggering, or generating an immune response toward SARS-CoV-2, comprising administering to a subject infected thereby an effective amount of agent or a composition of claim 1.

80. The method of claim 78, wherein the effective amount is an amount sufficient to
(i) induce or generate ADCC or ADCP toward SARS-CoV-2;
(ii) induce or generate long-term immunity toward SARS-CoV-2; or
(iii) provide memory T and/or B cells against SARS-CoV-2 in the subject.

81. The method of claim 78, further comprising administering a population of immune cells, wherein the population of immune cells comprises NK cells, selected from allogeneic NK cells, peripheral blood-derived NK cells, MG4101 NK cells, CB-NK NK cells, and cord blood-derived NK cells).

82. (canceled)

83. The agent of claim 1, wherein the agent has the structure:.

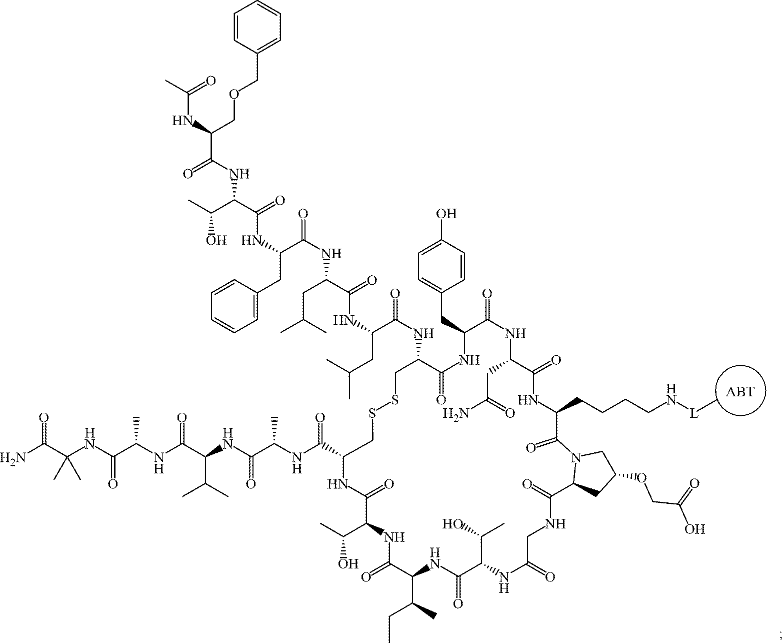

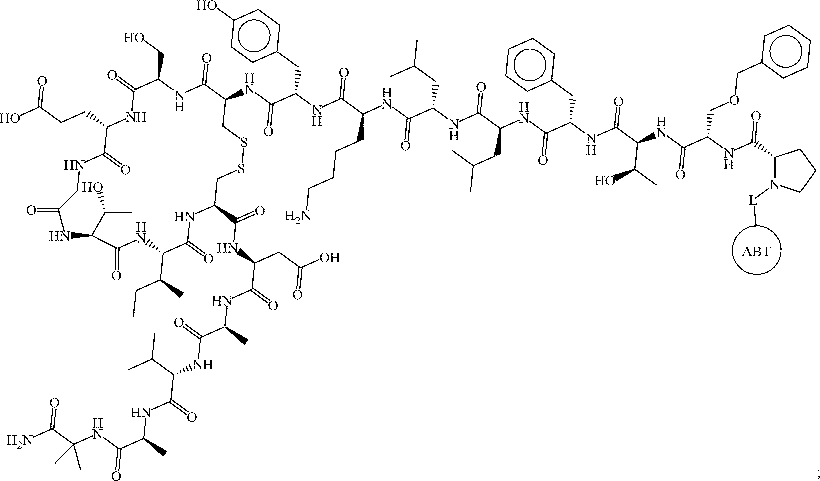

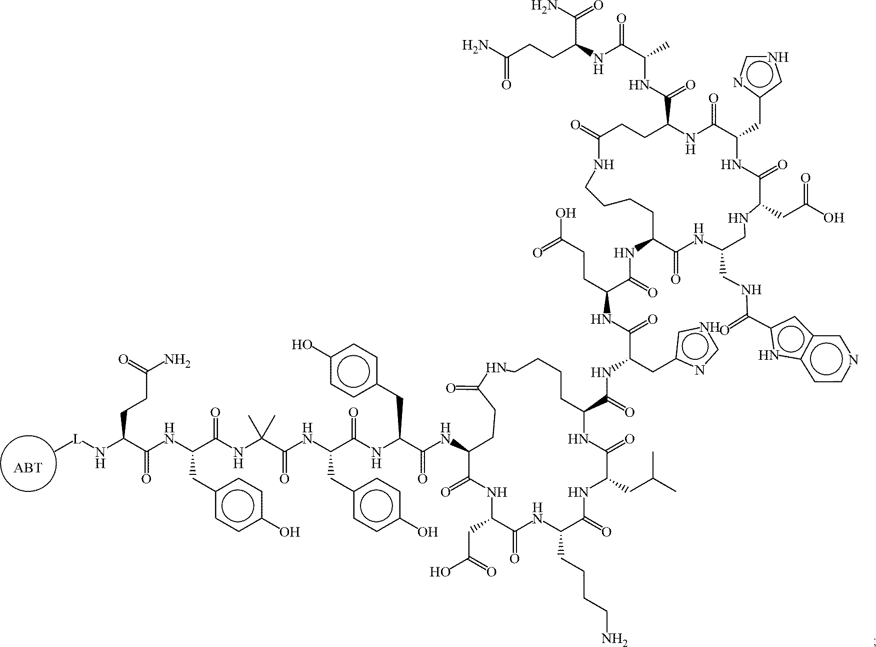

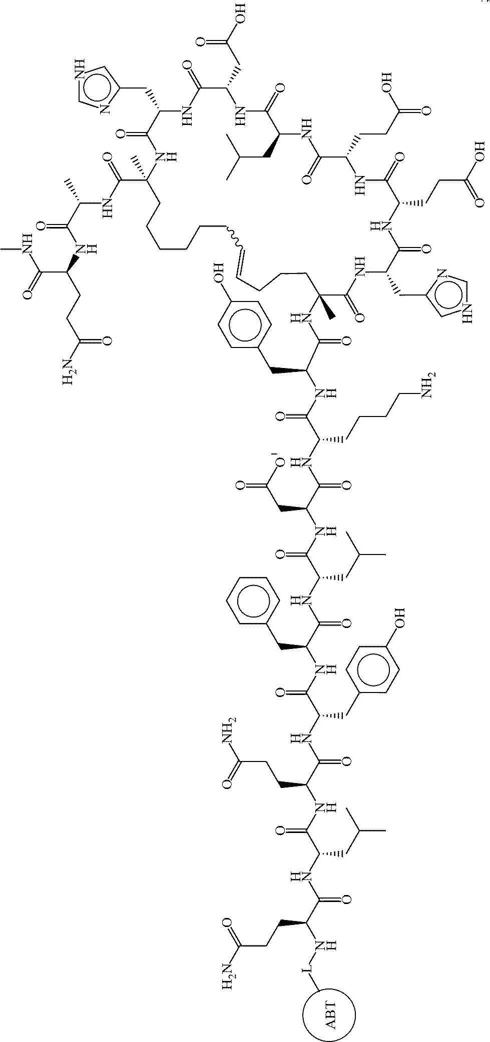

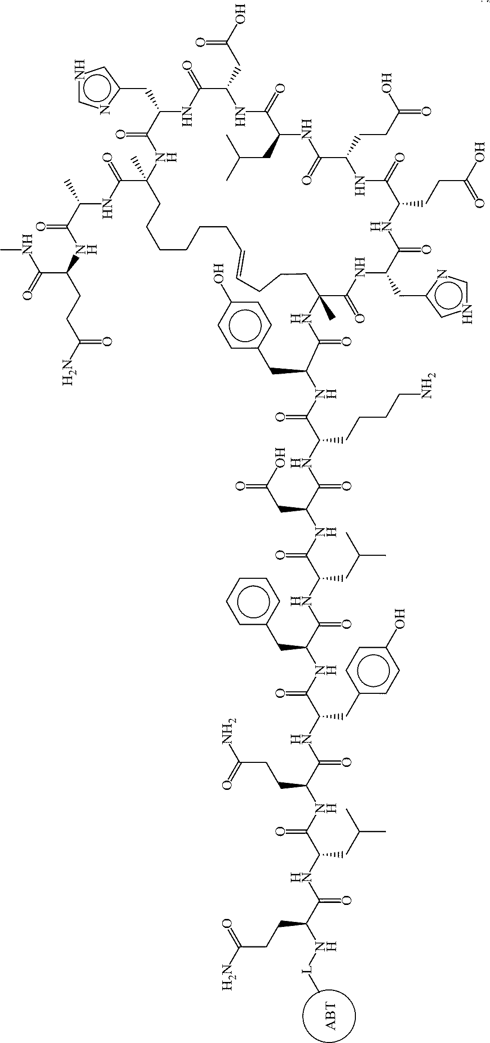

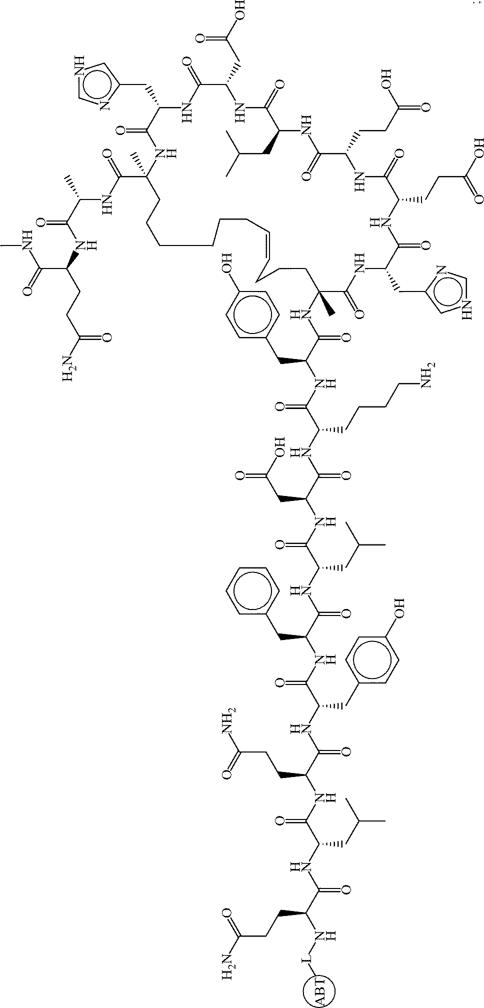

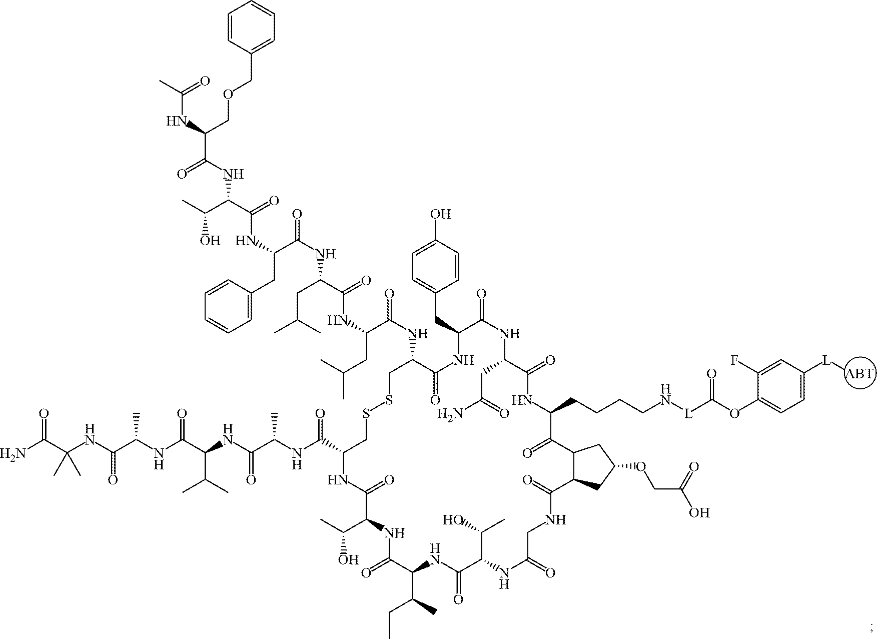

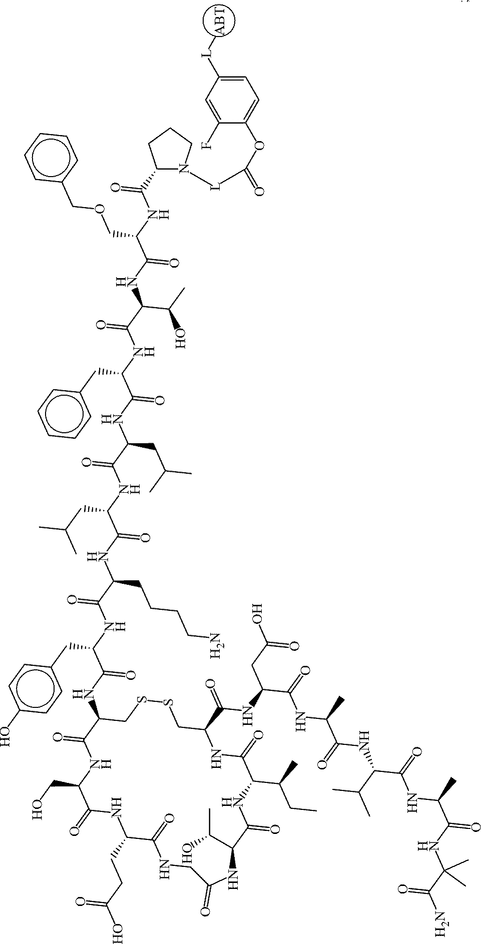

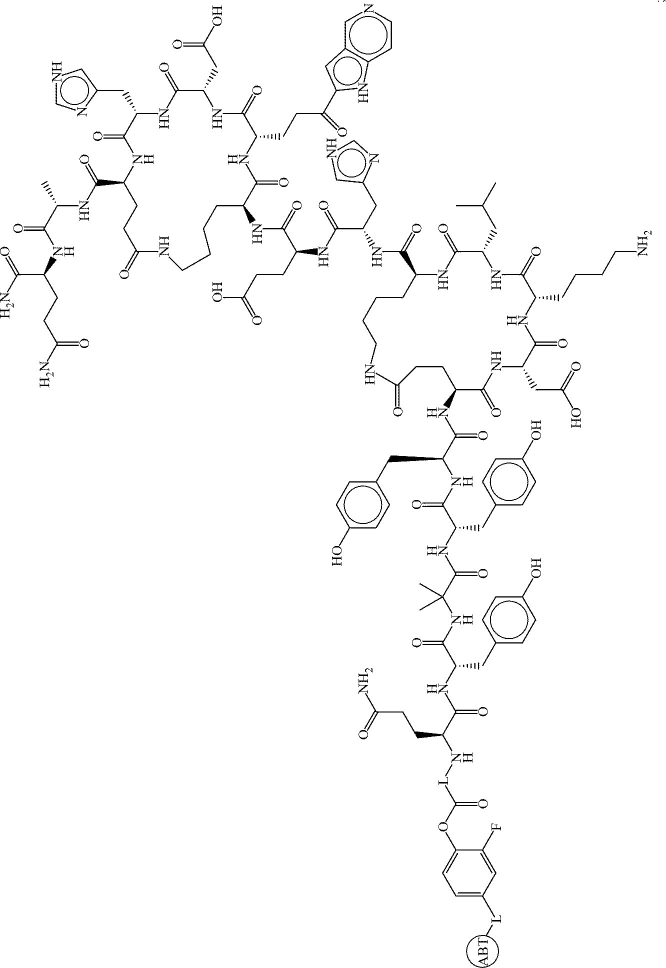

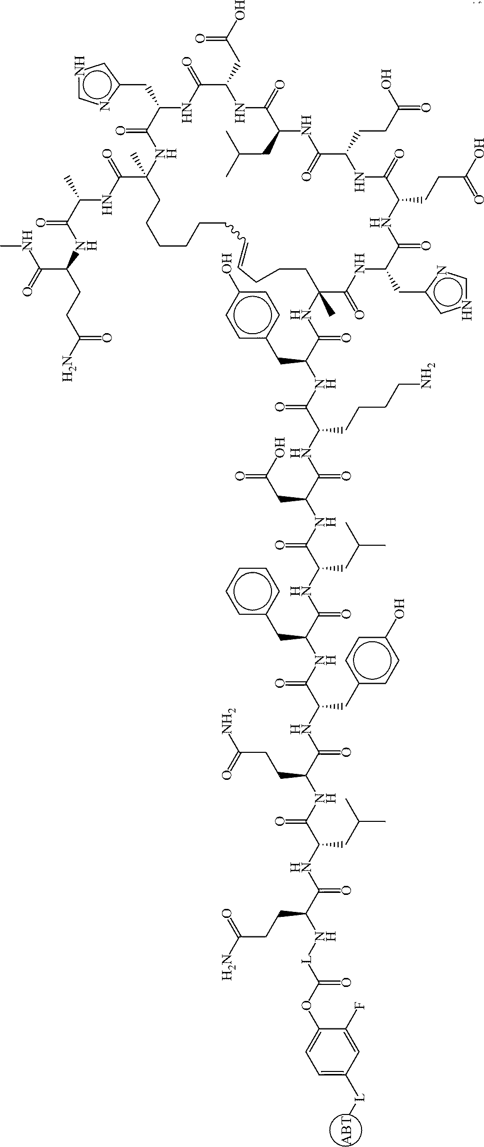

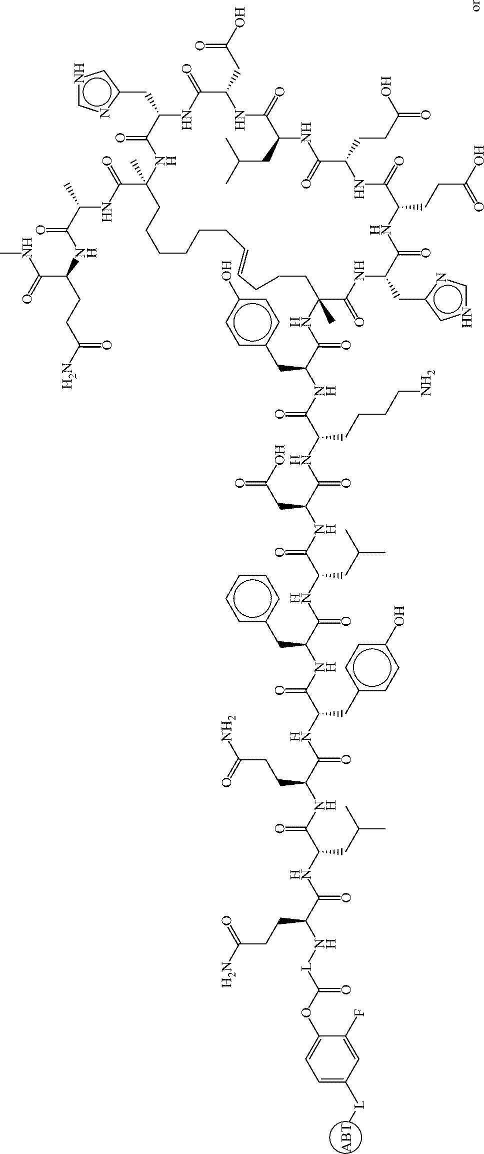

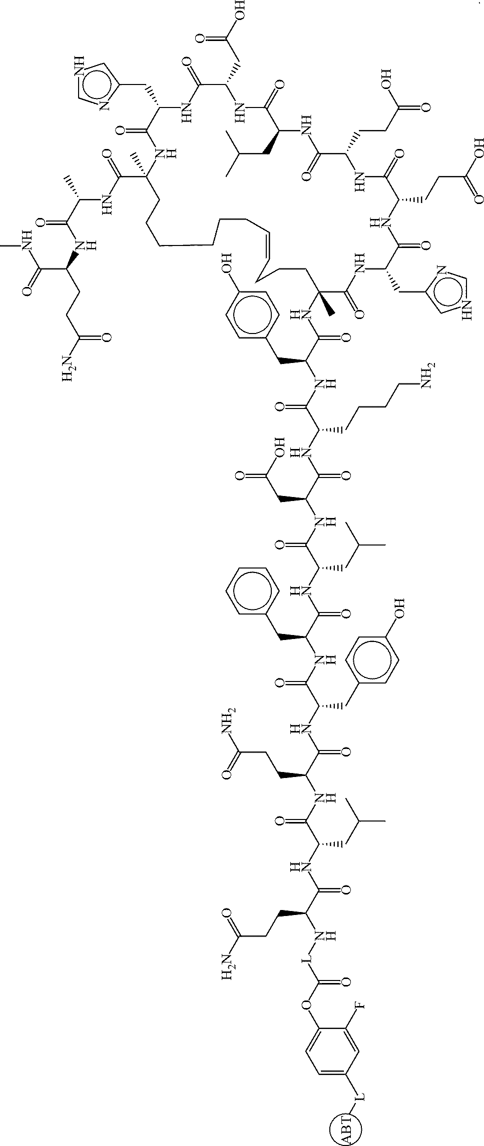

84. The agent of claim 1, wherein the agent has the structure:. 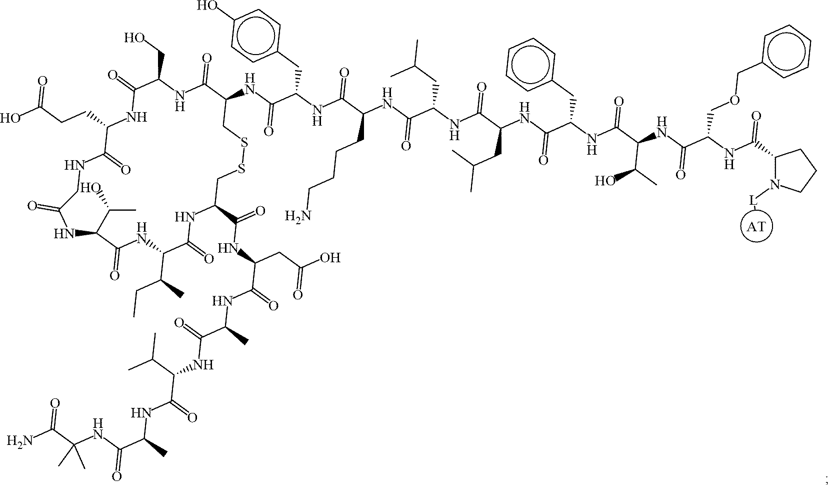 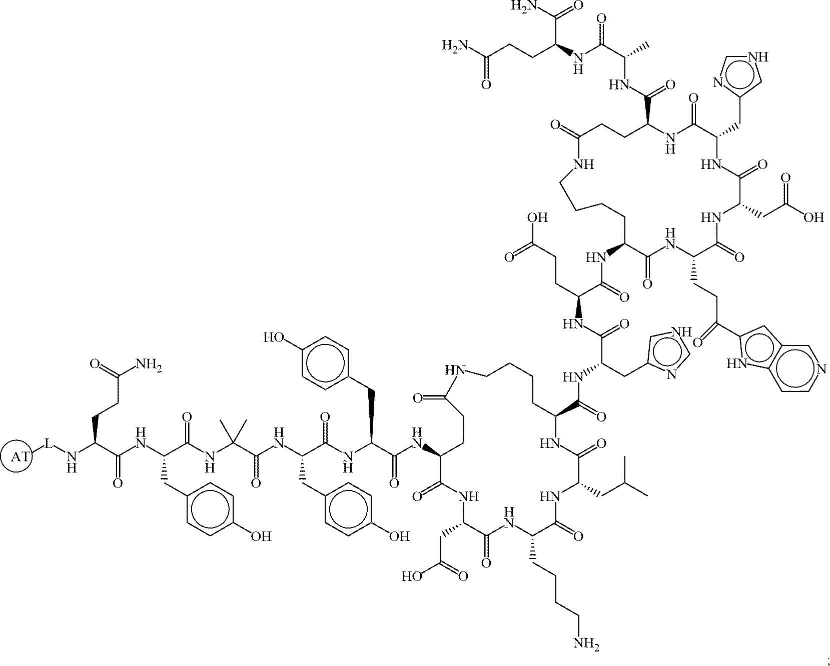 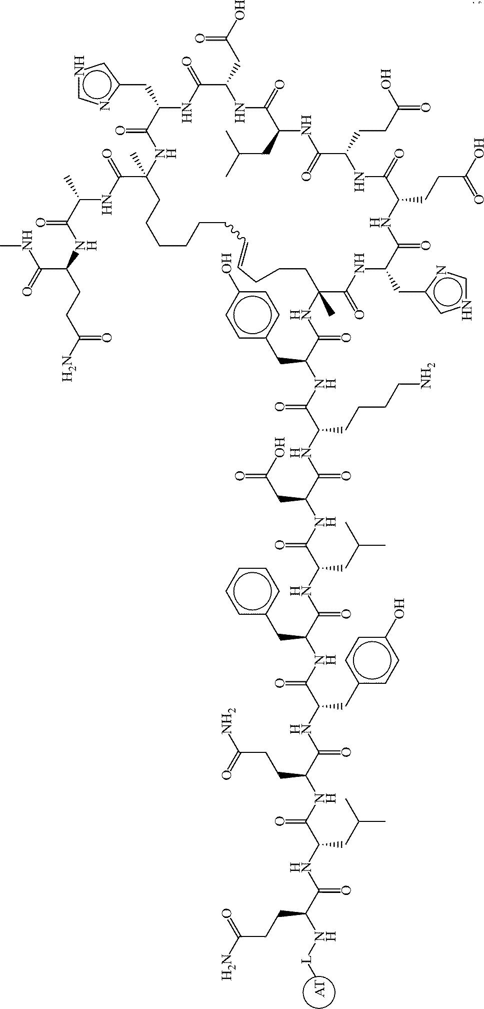 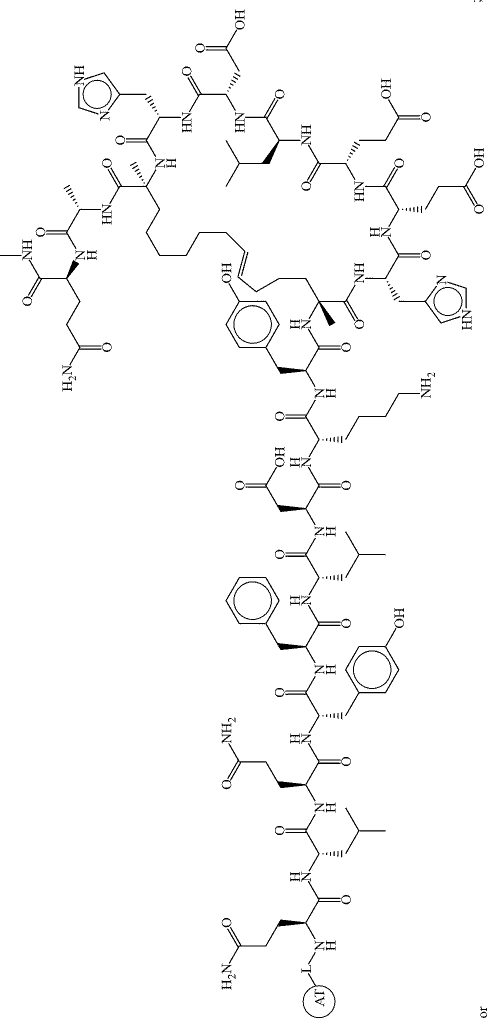 [chemical structure]
